SEQUENCE LISTING (129 sequences)

SEQ ID NO: 1 - protein, 13 aa, synthetic construct
  REGION 1..13: note = Spacer (IgG4 hinge) (aa)
ESKYGPPCPPCPM

SEQ ID NO: 2 - protein, 39 aa, synthetic construct
  REGION 1..39: note = Spacer (IgG4 hinge)(nt)
GAGTCTAAATACGGACCGCCTTGTCCTCCTTGTCCCATG

SEQ ID NO: 3 - protein, 12 aa, synthetic construct
  REGION 1..12: note = Spacer (IgG4 hinge) (aa)
ESKYGPPCPPCP

SEQ ID NO: 4 - protein, 36 aa, synthetic construct
  REGION 1..36: note = Spacer (IgG4 hinge)(nt)
GAGTCTAAATACGGACCGCCTTGTCCTCCTTGTCCC

SEQ ID NO: 5 - protein, 119 aa, synthetic construct
  REGION 1..119: note = Hinge-CH3 spacer (aa)
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - DNA, 357 bp, synthetic construct
  misc_feature 1..357: note = Hinge-CH3 spacer (nt)
  source 1..357: mol_type = other DNA
gaatctaagtacggaccgccttgtcctccatgtcctggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaag

SEQ ID NO: 7 - protein, 282 aa, synthetic construct
  REGION 1..282: note = IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 8 - protein, 24 aa, synthetic construct
  REGION 1..24: note = T2A
LEGGGEGRGSLLTCGDVEENPGPR

SEQ ID NO: 9 - protein, 357 aa, synthetic construct
  REGION 1..357: note = tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 10 - protein, 27 aa, Homo sapiens
  REGION 1..27: note = Human CD28 transmembrane (aa)
FWVLVVVGGVLACYSLLVTVAFIIFWV

SEQ ID NO: 11 - DNA, 81 bp, Homo sapiens
  misc_feature 1..81: note = Human CD28 transmembrane (nt)
  source 1..81: mol_type = unassigned DNA
ttctgggtgctcgtggtcgttggcggagtgctggcctgttacagcctgctggttaccgtggccttcatcatcttttgggtc

SEQ ID NO: 12 - protein, 28 aa, Homo sapiens
  REGION 1..28: note = Human CD28 transmembrane (aa)
MFWVLVVVGGVLACYSLLVTVAFIIFWV

SEQ ID NO: 13 - DNA, 84 bp, Homo sapiens
  misc_feature 1..84: note = Human CD28 transmembrane (nt)
  source 1..84: mol_type = unassigned DNA
atgttctgggtgctcgtggtcgttggcggagtgctggcctgttacagcctgctggttaccgtggccttcatcatcttttgggtc

SEQ ID NO: 14 - protein, 66 aa, synthetic construct
  REGION 1..66: note = CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 15 - protein, 41 aa, synthetic construct
  REGION 1..41: note = CD28 (amino acids 180-220 of P10747)
RSKRSRLLHSDYMNMTPRRPGPTRKHYQPYAPPRDFAAYRS

SEQ ID NO: 16 - protein, 41 aa, synthetic construct
  REGION 1..41: note = CD28 (LL to GG)
RSKRSRGGHSDYMNMTPRRPGPTRKHYQPYAPPRDFAAYRS

SEQ ID NO: 17 - protein, 42 aa, Homo sapiens
  REGION 1..42: note = Human 4-1BB endo domain (aa)
KRGRKKLLYIFKQPFMRPVQTTQEEDGCSCRFPEEEEGGCEL

SEQ ID NO: 18 - DNA, 126 bp, Homo sapiens
  misc_feature 1..126: note = Human 4-1BB endo domain (nt)
  source 1..126: mol_type = unassigned DNA
aagcggggcagaaagaagctgctctacatcttcaagcagcccttcatgcggcccgtgcagaccacacaagaggaagatggctgctcctgcagattccccgaggaagaagaaggcggctgcgagctg

SEQ ID NO: 19 - protein, 112 aa, Homo sapiens
  REGION 1..112: note = Human CD3-zeta endodomain (aa)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - DNA, 336 bp, Homo sapiens
  misc_feature 1..336: note = Human CD3-zeta endodomain (nt)
  source 1..336: mol_type = unassigned DNA
agagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 21 - protein, 112 aa, synthetic construct
  REGION 1..112: note = CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 112 aa, synthetic construct
  REGION 1..112: note = CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 335 aa, synthetic construct
  REGION 1..335: note = tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 24 - protein, 18 aa, synthetic construct
  REGION 1..18: note = T2A
EGRGSLLTCGDVEENPGP

SEQ ID NO: 25 - protein, 22 aa, synthetic construct
  REGION 1..22: note = P2A
GSGATNFSLLKQAGDVEENPGP

SEQ ID NO: 26 - protein, 19 aa, synthetic construct
  REGION 1..19: note = P2A
ATNFSLLKQAGDVEENPGP

SEQ ID NO: 27 - protein, 20 aa, synthetic construct
  REGION 1..20: note = E2A
QCTNYALLKLAGDVESNPGP

SEQ ID NO: 28 - protein, 22 aa, synthetic construct
  REGION 1..22: note = F2A
VKQTLNFDLLKLAGDVESNPGP

SEQ ID NO: 29 - protein, 29 aa, synthetic construct
  REGION 1..29: note = linker
PGGGSGGGGSGGGGSGGGGSGGGGSGGGG

SEQ ID NO: 30 - protein, 17 aa, synthetic construct
  REGION 1..17: note = linker
GSADDAKKDAAKKDGKS

SEQ ID NO: 31 - protein, 18 aa, synthetic construct
  REGION 1..18: note = linker
GSTSGSGKPGSGEGSTKG

SEQ ID NO: 32 - skipped

SEQ ID NO: 33 - protein, 15 aa, synthetic construct
  REGION 1..15: note = Hinge
EPKSCDKTHTCPPCP

SEQ ID NO: 34 - protein, 12 aa, synthetic construct
  REGION 1..12: note = Hinge
ERKCCVECPPCP

SEQ ID NO: 35 - protein, 61 aa, synthetic construct
  REGION 1..61: note = Hinge
ELKTPLGDTHTCPRCPEPKSCDTPPPCPRCPEPKSCDTPPPCPRCPEPKSCDTPPPCPRCP

SEQ ID NO: 36 - protein, 12 aa, synthetic construct
  REGION 1..12: note = Hinge
ESKYGPPCPSCP

SEQ ID NO: 37 - protein, 9 aa, synthetic construct
  REGION 1..9: note = Hinge
YGPPCPPCP

SEQ ID NO: 38 - protein, 10 aa, synthetic construct
  REGION 1..10: note = Hinge
KYGPPCPPCP

SEQ ID NO: 39 - protein, 14 aa, synthetic construct
  REGION 1..14: note = Hinge
EVVVKYGPPCPPCP

SEQ ID NO: 40 - DNA, 684 bp, synthetic construct
  misc_feature 1..684: note = Hinge-CH2-CH3 spacer (nt)
  source 1..684: mol_type = other DNA
gaatctaagtacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaag

SEQ ID NO: 41 - protein, 229 aa, synthetic construct
  REGION 1..229: note = Hinge-CH2-CH3 spacer (aa)
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 228 aa, synthetic construct
  REGION 1..228: note = IgG4/IgG2 hinge- IgG2/IgG4 CH2- IgG4 CH3 spacer (aa)
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - DNA, 684 bp, synthetic construct
  misc_feature 1..684: note = IgG4/IgG2 hinge- IgG2/IgG4 CH2- IgG4 CH3 spacer (nt)
  source 1..684: mol_type = other DNA
gaatctaagtacggaccgccctgccctccctgccctgctcctcctgtggctggaccaagcgtgttcctgtttccacctaagcctaaagataccctgatgatttcccgcacacctgaagtgacttgcgtggtcgtggacgtgagccaggaggatccagaagtgcagttcaactggtacgtggacggcgtggaagtccacaatgctaagactaaaccccgagaggaacagtttcagtcaacttaccgggtcgtgagcgtgctgaccgtcctgcatcaggattggctgaacgggaaggagtataagtgcaaagtgtctaataagggactgcctagctccatcgagaaaacaattagtaaggcaaaagggcagcctcgagaaccacaggtgtataccctgccccctagccaggaggaaatgaccaagaaccaggtgtccctgacatgtctggtcaaaggcttctatccaagtgacatcgccgtggagtgggaatcaaatgggcagcccgagaacaattacaagaccacaccacccgtgctggactctgatggaagtttctttctgtattccaggctgaccgtggataaatctcgctggcaggagggcaacgtgttctcttgcagtgtcatgcacgaagccctgcacaatcattatacacagaagtcactgagcctgtccctgggcaaa

SEQ ID NO: 44 - protein, 5 aa, synthetic construct
  REGION 1..5: note = 4GS linker (aa)
GGGGS

SEQ ID NO: 45 - protein, 4 aa, synthetic construct
  REGION 1..4: note = 3GS linker (aa)
GGGS

SEQ ID NO: 46 - protein, 15 aa, synthetic construct
  REGION 1..15: note = (4GS)3 linker (aa)
GGGGSGGGGSGGGGS

SEQ ID NO: 47 - DNA, 45 bp, synthetic construct
  misc_feature 1..45: note = (4GS)3 linker (nt)
  source 1..45: mol_type = other DNA
ggaggcggaggatctggtggcggaggaagtggcggaggcggttct

SEQ ID NO: 48 - protein, 16 aa, Homo sapiens
  REGION 1..16: note = Human CD33 Signal Peptide (aa)
MPLLLLLPLLWAGALA

SEQ ID NO: 49 - DNA, 48 bp, Homo sapiens
  misc_feature 1..48: note = Human CD33 Signal Peptide (nt)
  source 1..48: mol_type = unassigned DNA
atgccgctgctgctactgctgcccctgctgtgggcaggggctctagct

SEQ ID NO: 50 - protein, 20 aa, Homo sapiens
  REGION 1..20: note = human IgG-kappa signal peptide (aa)
MVLQTQVFISLLLWISGAYG

SEQ ID NO: 51 - DNA, 60 bp, Homo sapiens
  misc_feature 1..60: note = human IgG-kappa signal sequence (nt)
  source 1..60: mol_type = unassigned DNA
atggtgctgcagacccaggtgttcatcagcctgctgctgtggatctccggagcatacgga

SEQ ID NO: 52 - protein, 22 aa, synthetic construct
  REGION 1..22: note = GMCSFR alpha chain signal peptide (aa)
MLLLVTSLLLCELPHPAFLLIP

SEQ ID NO: 53 - DNA, 66 bp, synthetic construct
  misc_feature 1..66: note = GMCSFR alpha chain signal sequence (nt)
  source 1..66: mol_type = other DNA
atgcttctcctggtgacaagccttctgctctgtgagttaccacacccagcattcctcctgatccca

SEQ ID NO: 54 - protein, 18 aa, synthetic construct
  REGION 1..18: note = CD8 alpha signal peptide
MALPVTALLLPLALLLHA

SEQ ID NO: 55 - protein, 326 aa, Homo sapiens
  REGION 1..326: note = Human IgG2 Fc (Uniprot P01859)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 56 - protein, 327 aa, Homo sapiens
  REGION 1..327: note = Human IgG4 Fc (Uniprot P01861)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - DNA, 357 bp, synthetic construct
  misc_feature 1..357: note = Hinge-CH3 spacer (nt)
  source 1..357: mol_type = other DNA
gagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaag

SEQ ID NO: 58 - protein, 120 aa, synthetic construct
  REGION 1..120: note = Hinge-CH3 spacer (aa)
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 59 - DNA, 360 bp, synthetic construct
  misc_feature 1..360: note = Hinge-CH3 spacer (nt)
  source 1..360: mol_type = other DNA
gagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatg

SEQ ID NO: 60 - DNA, 684 bp, synthetic construct
  misc_feature 1..684: note = IgG4/IgG2 hinge- IgG2/IgG4 CH2- IgG4 CH3 spacer (nt)
  source 1..684: mol_type = other DNA
gagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaag

SEQ ID NO: 61 - DNA, 72 bp, synthetic construct
  misc_feature 1..72: note = T2A (nt)
  source 1..72: mol_type = other DNA
cttgaaggtggtggcgaaggcagaggcagcctgcttacatgcggagatgtggaagagaaccccggacctaga

SEQ ID NO: 62 - DNA, 84 bp, synthetic construct
  misc_feature 1..84: note = CD28 transmembrane domain (nt)
  source 1..84: mol_type = other DNA
atgttttgggtgctggtcgtggtcggaggggtgctggcctgttacagcctgctggtgacagtcgctttcatcatcttctgggtg

SEQ ID NO: 63 - DNA, 360 bp, synthetic construct
  misc_feature 1..360: note = Hinge-CH3 spacer (nt)
  source 1..360: mol_type = other DNA
gaatctaagtacggaccgccttgtcctccatgtcctggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatg

SEQ ID NO: 64 - DNA, 1074 bp, synthetic construct
  misc_feature 1..1074: note = truncated EGFR (tEGFR) sequence (nt)
  source 1..1074: mol_type = other DNA
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 65 - DNA, 1074 bp, synthetic construct
  misc_feature 1..1074: note = truncated EGFR (tEGFR) sequence (nt)
  source 1..1074: mol_type = other DNA
atgctgctcctcgtgacaagcctgctcctgtgtgaactccctcatccagcttttctgctcattcctcggaaagtgtgcaacggcatcggcatcggagagttcaaggacagcctgagcatcaatgccaccaacatcaagcacttcaagaattgcaccagcatcagcggcgacctgcacattctgcctgtggcctttagaggcgacagcttcacccacacacctccactggatccccaagagctggatatcctgaaaaccgtgaaagagattaccggattcctcctgatccaagcctggccagagaacagaaccgatctgcacgccttcgagaacctcgagatcatcagaggccggaccaaacagcacggccagtttagcctggctgtggtgtctctgaacatcaccagtctgggcctgagaagcctgaaagaaatctccgacggcgacgtgatcatctccggaaacaagaacctgtgctacgccaacaccatcaactggaagaagctgttcggcacctccggccagaaaacaaagatcatctctaaccggggcgagaacagctgcaaggccaccggacaagtttgtcacgccctgtgtagccctgaaggctgttggggacccgaacctagagactgtgtgtcctgccggaatgtgtcccggggcagagaatgtgtggataagtgcaacctgctggaaggcgagccccgcgagtttgtggaaaacagcgagtgcatccagtgtcaccccgagtgtctgccccaggccatgaacattacatgcaccggcagaggccccgacaactgtattcagtgcgcccactacatcgacggccctcactgcgtgaaaacatgtccagctggcgtgatgggagagaacaacaccctcgtgtggaagtatgccgacgccggacatgtgtgccacctgtgtcaccctaattgcacctacggctgtaccggacctggcctggaaggatgccctacaaacggccctaagatccccagcattgccaccggaatggttggagccctgctgcttctgttggtggtggccctcggaatcggcctgttcatgtga

SEQ ID NO: 66 - protein, 357 aa, synthetic construct; identical to SEQ ID NO: 9
  REGION 1..357: note = truncated EGFR (tEGFR) sequence (aa)

SEQ ID NO: 67 - DNA, 126 bp, synthetic construct
  misc_feature 1..126: note = 4-1BB intracellular co-signaling sequence (nt)
  source 1..126: mol_type = other DNA
aagcgggggagaaagaaactgctgtatattttcaaacagccctttatgagacctgtgcagactacccaggaggaagacggatgcagctgtaggtttcccgaggaagaggaaggaggctgtgagctg

SEQ ID NO: 68 - protein, 21 aa, synthetic construct
  REGION 1..21: note = linker
SRGGGGSGGGGSGGGGSLEMA

SEQ ID NO: 69 - protein, 22 aa, synthetic construct
  REGION 1..22: note = linker
GSRGGGGSGGGGSGGGGSLEMA

SEQ ID NO: 70 - DNA, 544 bp, synthetic construct
  misc_feature 1..544: note = EF1alpha promoter with HTLV1 ehancer
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 71 - DNA, 589 bp, synthetic construct
  misc_feature 1..589: note = Woodchuck Hepatitis Virus (WHP) Posttranscriptional Regulatory Element (WPRE)
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 72 - protein, 21 aa, synthetic construct
  REGION 1..21: note = T2A peptide (aa)
GSGEGRGSLLTCGDVEENPGP

SEQ ID NO: 73 - protein, 23 aa, synthetic construct
  REGION 1..23: note = E2A peptide (aa)
GSGQCTNYALLKLAGDVESNPGP

SEQ ID NO: 74 - protein, 25 aa, synthetic construct
  REGION 1..25: note = F2A peptide (aa)
GSGVKQTLNFDLLKLAGDVESNPGP

SEQ ID NO: 75 - DNA, 72 bp, synthetic construct
  misc_feature 1..72: note = T2A peptide (nt)
  source 1..72: mol_type = other DNA
ctcgagggcggcggagagggcagaggaagtcttctaacatgcggtgacgtggaggagaatcccggccctagg

SEQ ID NO: 76 - DNA, 336 bp, synthetic construct
  misc_feature 1..336: note = CD3-zeta derived intracellular signaling domain (nt)
  source 1..336: mol_type = other DNA
agagtcaagttttccaggtccgccgacgctccagcctaccagcaggggcagaaccagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 77 - DNA, 123 bp, synthetic construct
  misc_feature 1..123: note = CD28 endodomain (nt)
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 78 - DNA, 48 bp, synthetic construct
  misc_feature 1..48: note = CD33 signal sequence (nt)
  source 1..48: mol_type = other DNA
atgcctctgctgctgcttctgcctcttctttgggctggtgctctggct

SEQ ID NO: 79 - DNA, 48 bp, synthetic construct
  misc_feature 1..48: note = CD33 signal sequence (nt) GenBank: M23197.1
  source 1..48: mol_type = other DNA
atgccgctgctgctactgctgcccctgctgtgggcaggggccctggct

SEQ ID NO: 80 - DNA, 39 bp, synthetic construct
  misc_feature 1..39: note = Spacer (IgG4 hinge)(nt)
  source 1..39: mol_type = other DNA
gaatctaagtacggaccgccctgccccccttgccctatg

SEQ ID NO: 81 - DNA, 357 bp, synthetic construct; identical to SEQ ID NO: 6
  misc_feature 1..357: note = Hinge-CH3 spacer (nt)
  source 1..357: mol_type = other DNA

SEQ ID NO: 82 - protein, 229 aa, synthetic construct
  REGION 1..229: note = IgG4/IgG2 hinge- IgG2/IgG4 CH2- IgG4 CH3 spacer (aa)
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 83 - DNA, 687 bp, synthetic construct
  misc_feature 1..687: note = IgG4/IgG2 hinge- IgG2/IgG4 CH2- IgG4 CH3 spacer (nt)
  source 1..687: mol_type = other DNA
gaatctaagtacggaccgccctgccctccctgccctgctcctcctgtggctggaccaagcgtgttcctgtttccacctaagcctaaagataccctgatgatttcccgcacacctgaagtgacttgcgtggtcgtggacgtgagccaggaggatccagaagtgcagttcaactggtacgtggacggcgtggaagtccacaatgctaagactaaaccccgagaggaacagtttcagtcaacttaccgggtcgtgagcgtgctgaccgtcctgcatcaggattggctgaacgggaaggagtataagtgcaaagtgtctaataagggactgcctagctccatcgagaaaacaattagtaaggcaaaagggcagcctcgagaaccacaggtgtataccctgccccctagccaggaggaaatgaccaagaaccaggtgtccctgacatgtctggtcaaaggcttctatccaagtgacatcgccgtggagtgggaatcaaatgggcagcccgagaacaattacaagaccacaccacccgtgctggactctgatggaagtttctttctgtattccaggctgaccgtggataaatctcgctggcaggagggcaacgtgttctcttgcagtgtcatgcacgaagccctgcacaatcattatacacagaagtcactgagcctgtccctgggcaaaatg

SEQ ID NO: 84 - DNA, 687 bp, synthetic construct
  misc_feature 1..687: note = IgG4/IgG2 hinge- IgG2/IgG4 CH2- IgG4 CH3 spacer (nt)
  source 1..687: mol_type = other DNA
gagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatg

SEQ ID NO: 85 - DNA, 81 bp, synthetic construct
  misc_feature 1..81: note = CD28 transmembrane domain (nt)
  source 1..81: mol_type = other DNA
ttttgggtgctggtcgtggtcggaggggtgctggcctgttacagcctgctggtgacagtcgctttcatcatcttctgggtg

SEQ ID NO: 86 - DNA, 723 bp, synthetic construct
  misc_feature 1..723: note = DLL3-mAb-1 scFv (VH-VL) (nt)
  source 1..723: mol_type = other DNA
caggttcagctgcaagagtctggccctggcctggtcaagcctagccaaacactgagcctgacctgtaccgtgtccggcgatagcatcagcagcgacggctactactggtcctggatcagacagcaccctggcaaaggcctggaatggatcggctacatctactacagcggcaacacctactacaaccccagcctgaagtccagagtgaccatcagcgtggacaccagcaagaaccagttctccctgaagctgaccagcgtgacagccgccgataccgccatctactattgcgccatcgataccgctagcgccctggcttattggggccagggaacactggtcacagtgtctagcggaggcggaggatctggtggcggaggaagtggcggaggcggttctgagattgtgatgacacagagccccgccacactgtccgtttctcccggcgaaagagccacactgagctgcagagccagccagagcgtgtcctctaacctggcttggtatcagcagaagcccggacaggctcccagactgctgatctatggcgcctctacaagagccaccggcattcccgcccggttttctggctctggaagcggcaccgagttcaccctgacaatcagcagcctgcagagcgaggacttcgccgtgtactactgccagcagtacaacaactggccttggacctttggccagggcaccaaagtggaaatcaagcgg

SEQ ID NO: 87 - DNA, 354 bp, synthetic construct
  misc_feature 1..354: note = DLL3-mAb-1 VH (nt)
  source 1..354: mol_type = other DNA
caggttcagctgcaagagtctggccctggcctggtcaagcctagccaaacactgagcctgacctgtaccgtgtccggcgatagcatcagcagcgacggctactactggtcctggatcagacagcaccctggcaaaggcctggaatggatcggctacatctactacagcggcaacacctactacaaccccagcctgaagtccagagtgaccatcagcgtggacaccagcaagaaccagttctccctgaagctgaccagcgtgacagccgccgataccgccatctactattgcgccatcgataccgctagcgccctggcttattggggccagggaacactggtcacagtgtctagc

SEQ ID NO: 88 - DNA, 324 bp, synthetic construct
  misc_feature 1..324: note = DLL3-mAb-1 VL (nt)
  source 1..324: mol_type = other DNA
gagattgtgatgacacagagccccgccacactgtccgtttctcccggcgaaagagccacactgagctgcagagccagccagagcgtgtcctctaacctggcttggtatcagcagaagcccggacaggctcccagactgctgatctatggcgcctctacaagagccaccggcattcccgcccggttttctggctctggaagcggcaccgagttcaccctgacaatcagcagcctgcagagcgaggacttcgccgtgtactactgccagcagtacaacaactggccttggacctttggccagggcaccaaagtggaaatcaagcgg

SEQ ID NO: 89 - protein, 241 aa, synthetic construct
  REGION 1..241: note = DLL3-mAb-1 scFv (VH-VL) (aa)
QVQLQESGPGLVKPSQTLSLTCTVSGDSISSDGYYWSWIRQHPGKGLEWIGYIYYSGNTYYNPSLKSRVTISVDTSKNQFSLKLTSVTAADTAIYYCAIDTASALAYWGQGTLVTVSSGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKR

SEQ ID NO: 90 - protein, 118 aa, synthetic construct
  REGION 1..118: note = DLL3-mAb-1 VH (aa)
QVQLQESGPGLVKPSQTLSLTCTVSGDSISSDGYYWSWIRQHPGKGLEWIGYIYYSGNTYYNPSLKSRVTISVDTSKNQFSLKLTSVTAADTAIYYCAIDTASALAYWGQGTLVTVSS

SEQ ID NO: 91 - protein, 108 aa, synthetic construct
  REGION 1..108: note = DLL3-mAb-1 VL (aa)
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKR

SEQ ID NO: 92 - protein, 7 aa, synthetic construct
  REGION 1..7: note = DLL3-mAb-1 CDR-H1 (aa) (Kabat)
SDGYYWS

SEQ ID NO: 93 - protein, 16 aa, synthetic construct
  REGION 1..16: note = DLL3-mAb-1 CDR-H2 (aa) (Kabat)
YIYYSGNTYYNPSLKS

SEQ ID NO: 94 - protein, 8 aa, synthetic construct
  REGION 1..8: note = DLL3-mAb-1 CDR-H3 (aa) (Kabat)
DTASALAY

SEQ ID NO: 95 - protein, 11 aa, synthetic construct
  REGION 1..11: note = DLL3-mAb-1 CDR-L1 (aa) (Kabat)
RASQSVSSNLA

SEQ ID NO: 96 - protein, 7 aa, synthetic construct
  REGION 1..7: note = DLL3-mAb-1 CDR-L2 (aa) (Kabat)
GASTRAT

SEQ ID NO: 97 - protein, 9 aa, synthetic construct
  REGION 1..9: note = DLL3-mAb-1 CDR-L3 (aa) (Kabat)
QQYNNWPWT

SEQ ID NO: 98 - protein, 9 aa, synthetic construct
  REGION 1..9: note = DLL3-mAb-1 CDR-H1 (aa) (Chothia)
GDSISSDGY

SEQ ID NO: 99 - protein, 5 aa, synthetic construct
  REGION 1..5: note = DLL3-mAb-1 CDR-H2 (aa) (Chothia)
YYSGN

SEQ ID NO: 100 - protein, 8 aa, synthetic construct
  REGION 1..8: note = DLL3-mAb-1 CDR-H3 (aa) (Chothia)
DTASALAY

SEQ ID NO: 101 - protein, 11 aa, synthetic construct
  REGION 1..11: note = DLL3-mAb-1 CDR-L1 (aa) (Chothia)
RASQSVSSNLA

SEQ ID NO: 102 - protein, 7 aa, synthetic construct
  REGION 1..7: note = DLL3-mAb-1 CDR-L2 (aa) (Chothia)
GASTRAT

SEQ ID NO: 103 - protein, 9 aa, synthetic construct
  REGION 1..9: note = DLL3-mAb-1 CDR-L3 (aa) (Chothia)
QQYNNWPWT

SEQ ID NO: 104 - protein, 9 aa, synthetic construct
  REGION 1..9: note = DLL3-mAb-1 CDR-H1 (aa) (IMGT)
GDSISSDGY

SEQ ID NO: 105 - protein, 7 aa, synthetic construct
  REGION 1..7: note = DLL3-mAb-1 CDR-H2 (aa) (IMGT)
IYYSGNT

SEQ ID NO: 106 - protein, 10 aa, synthetic construct
  REGION 1..10: note = DLL3-mAb-1 CDR-H3 (aa) (IMGT)
AIDTASALAY

SEQ ID NO: 107 - protein, 6 aa, synthetic construct
  REGION 1..6: note = DLL3-mAb-1 CDR-L1 (aa) (IMGT)
QSVSSN

SEQ ID NO: 108 - skipped

SEQ ID NO: 109 - protein, 9 aa, synthetic construct
  REGION 1..9: note = DLL3-mAb-1 CDR-L3 (aa) (IMGT)
QQYNNWPWT

SEQ ID NO: 110 - protein, 12 aa, synthetic construct
  REGION 1..12: note = DLL3-mAb-1 CDR-H1 (aa) (AbM)
GDSISSDGYYWS

SEQ ID NO: 111 - protein, 9 aa, synthetic construct
  REGION 1..9: note = DLL3-mAb-1 CDR-H2 (aa) (AbM)
YIYYSGNTY

SEQ ID NO: 112 - protein, 8 aa, synthetic construct
  REGION 1..8: note = DLL3-mAb-1 CDR-H3 (aa) (AbM)
DTASALAY

SEQ ID NO: 113 - protein, 11 aa, synthetic construct
  REGION 1..11: note = DLL3-mAb-1 CDR-L1 (aa) (AbM)
RASQSVSSNLA

SEQ ID NO: 114 - protein, 7 aa, synthetic construct
  REGION 1..7: note = DLL3-mAb-1 CDR-L2 (aa) (AbM)
GASTRAT

SEQ ID NO: 115 - protein, 9 aa, synthetic construct
  REGION 1..9: note = DLL3-mAb-1 CDR-L3 (aa) (AbM)
QQYNNWPWT

SEQ ID NO: 116 - DNA, 1300 bp, synthetic construct
  misc_feature 1..1300: note = CAR A (nt)
  source 1..1300: mol_type = other DNA
caggttcagctgcaagagtctggccctggcctggtcaagcctagccaaacactgagcctgacctgtaccgtgtccggcgatagcatcagcagcgacggctactactggtcctggatcagacagcactggcaaaggcctggaatggatcggctacatctactacagcggcaacacctactacaaccccagcctgaagtccagagtgaccatcagcgtggacaccagcaagaaccagttctccctgaagctgaccgcgtgacagccgccgataccgccatctactattgcgccatcgataccgctagcgccctggcttattggggccagggaacactggtcacagtgtctagcggaggcggaggatctggtggcggaggaagtggcggaggcggttctgagattgtgatgacacagagccccgccacactgtccgtttctcccggcgaaagagccacactgagctgcagagccagccagagcgtgtcctctaacctggcttggtatcagcagaagcccggacaggctcccagactgctgatctatggcgcctctacaagagccaccggcattcccgcccggttttctggctctggaagcggcaccgagttcaccctgacaatcagcagcctgcagagcgaggacttcgccgtgtactactgccagcagtacaacaactggccttggacctttggccagggcaccaaagtggaaatcaagcgggagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaagcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 117 - protein, 435 aa, synthetic construct
  REGION 1..435: note = CAR A (aa)
QVQLQESGPGLVKPSQTLSLTCTVSGDSISSDGYYWSWIRQHPGKGLEWIGYIYYSGNTYYNPSLKSRVTISVDTSKNQFSLKLTSVTAADTAIYYCAIDTASALAYWGQGTLVTVSSGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - DNA, 1353 bp, synthetic construct
  misc_feature 1..1353: note = CAR A including signal sequence (nt)
  source 1..1353: mol_type = other DNA
atgccgctgctgctactgctgcccctgctgtgggcaggggctctagctcaggttcagctgcaagagtctggccctggcctggtcaagcctagccaaacactgagcctgacctgtaccgtgtccggcgatagcatcagcagcgacggctactactggtcctggatcagacagcaccctggcaaaggcctggaatggatcggctacatctactacagcggcaacacctactacaaccccagcctgaagtccagagtgaccatcagcgtggacaccagcaagaaccagttctccctgaagctgaccagcgtgacagccgccgataccgccatctactattgcgccatcgataccgctagcgccctggcttattggggccagggaacactggtcacagtgtctagcggaggcggaggatctggtggcggaggaagtggcggaggcggttctgagattgtgatgacacagagccccgccacactgtccgtttctcccggcgaaagagccacactgagctgcagagccagccagagcgtgtcctctaacctggcttggtatcagcagaagcccggacaggctcccagactgctgatctatggcgcctctacaagagccaccggcattcccgcccggttttctggctctggaagcggcaccgagttcaccctgacaatcagcagcctgcagagcgaggacttcgccgtgtactactgccagcagtacaacaactggccttggacctttggccagggcaccaaagtggaaatcaagcgggagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 119 - protein, 451 aa, synthetic construct
  REGION 1..451: note = CAR A including signal sequence (aa)
MPLLLLLPLLWAGALAQVQLQESGPGLVKPSQTLSLTCTVSGDSISSDGYYWSWIRQHPGKGLEWIGYIYYSGNTYYNPSLKSRVTISVDTSKNQFSLKLTSVTAADTAIYYCAIDTASALAYWGQGTLVTVSSGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 618 aa, Homo sapiens
  REGION 1..618: note = Human DLL3 isoform 1 precursor (aa)
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 121 - protein, 587 aa, Homo sapiens
  REGION 1..587: note = Human DLL3 isoform 2 precursor (aa)
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREA

SEQ ID NO: 122 - protein, 592 aa, Homo sapiens
  REGION 1..592: note = Human DLL3 isoform 1 mature (aa)
AGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 123 - protein, 561 aa, Homo sapiens
  REGION 1..561: note = Human DLL3 isoform 2 mature (aa)
AGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREA

SEQ ID NO: 124 - protein, 585 aa, Mus musculus
  REGION 1..585: note = Mus musculus DLL3 (aa)
MVSLQVSPLSQTLILAFLLPQALPAGVFELQIHSFGPGPGLGTPRSPCNARGPCRLFFRVCLKPGVSQEATESLCALGAALSTSVPVYTEHPGESAAALPLPDGLVRVPFRDAWPGTFSLVIETWREQLGEHAGGPAWNLLARVVGRRRLAAGGPWARDVQRTGTWELHFSYRARCEPPAVGAACARLCRSRSAPSRCGPGLRPCTPFPDECEAPSVCRPGCSPEHGYCEEPDECRCLEGWTGPLCTVPVSTSSCLNSRVPGPASTGCLLPGPGPCDGNPCANGGSCSETSGSFECACPRGFYGLRCEVSGVTCADGPCFNGGLCVGGEDPDSAYVCHCPPGFQGSNCEKRVDRCSLQPCQNGGLCLDLGHALRCRCRAGFAGPRCEHDLDDCAGRACANGGTCVEGGGSRRCSCALGFGGRDCRERADPCASRPCAHGGRCYAHFSGLVCACAPGYMGVRCEFAVRPDGADAVPAAPRGLRQADPQRFLLPPALGLLVAAGLAGAALLVIHVRRRGPGQDTGTRLLSGTREPSVHTLPDALNNLRLQDGAGDGPSSSADWNHPEDGDSRSIYVIPAPSIYAREA

SEQ ID NO: 125 - protein, 618 aa, Macaca fascicularis
  REGION 1..618: note = Macaca fascicularis DLL3 (aa)
MVSPRMSRLLSQTVILALIFIPQARPAGVFELQIHSFGPGPGPGAPRSPCSARGPCRLFFRVCLKPGLSEEAAESPCALGAALSARGPVYTEQPEAPAPDLPLPNGLLQVPFRDAWPGTFSLIIETWREELGDQIGGPAWSLLARVTRRRRLAAGGPWARDIQRAGAWELRFSYRARCELPAVGTACTRLCRPRSAPSRCGPGLRPCAPLEDECEAPPVCRAGCSLEHGFCEQPGECRCLEGWTGPLCMVPVSTSSCLGLRGPSSTTTGCLVPGPGPCDGNPCANGGSCSETPGSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRNCRERADPCAARPCAHGGRCYAHFSGLVCACAPGYMGARCEFPVHPDGVSALPAAPPGLRPGDPQRYLLPPALGLLVAAGVAGAALLLVHVRRRGHAQDAGSRLLAGTPEPSVHALPDALNNLRTQEGPGDVPSSSVDWNRPEDVDSRGIYVISAPSIYAREVAMPLFPPLHTGRAGQRQNLLFPFPSSILSVK

SEQ ID NO: 126 - protein, 330 aa, synthetic construct
  REGION 1..330: note = IgG1 heavy chain constant
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 107 aa, synthetic construct [sequence omitted]

SEQ ID NO: 128 - protein, 448 aa, synthetic construct
  REGION 1..448: note = DLL3-mAb-1 IgG1 full length heavy chain
QVQLQESGPGLVKPSQTLSLTCTVSGDSISSDGYYWSWIRQHPGKGLEWIGYIYYSGNTYYNPSLKSRVTISVDTSKNQFSLKLTSVTAADTAIYYCAIDTASALAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 214 aa, synthetic construct
  REGION 1..214: note = DLL3-mAb-1 IgG1 full length light chain
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0061051 A1
(43) Pub. Date: Mar. 5, 2026

(54) ANTIBODIES AND CHIMERIC ANTIGEN RECEPTORS SPECIFIC FOR DELTA-LIKE LIGAND 3 (DLL3)

(71) Applicant: Juno Therapeutics, Inc., Seattle, WA (US)

(72) Inventors: Brian Joshua BELMONT, Seattle, WA (US); Madeline WILLIAMS, Seattle, WA (US); Ashwini BALAKRISHNAN, Seattle, WA (US); Samriti BEDI, Newcastle, WA (US); Dean Y. TOY, Newcastle, WA (US); Stephanie BUSCH, Seattle, WA (US); Tiffany HUELAR, Seattle, WA (US); Dan ZHU, San Diego, CA (US)

(73) Assignee: Juno Therapeutics, Inc., Seattle, WA (US)

(21) Appl. No.: 19/106,172

(22) PCT Filed: Aug. 25, 2023
(86) PCT No.: PCT/US2023/072961

§ 371 Date: Feb. 24, 2025

Related U.S. Application Data
(60) Provisional application No. 63/401,559, filed on Aug. 26, 2022.

(51) Int. Cl. A61K 40/11; A61K 31/7088; A61K 35/17; A61K 38/00; A61K 40/15; A61K 40/31; A61P 35/00; C07K 14/705; C07K 14/725; C07K 16/28; C12N 15/62; C12N 15/867

(52) U.S. Cl. CPC A61K 40/11; A61K 31/7088; A61K 35/17; A61K 38/00; A61K 40/15; A61K 40/31; A61P 35/00; C07K 14/7051; C07K 14/70521; C07K 16/28; C12N 15/625; C12N 15/867; C07K 2317/51; C07K 2317/515; C07K 2317/53; C07K 2317/565; C07K 2317/622; C07K 2319/02; C07K 2319/03; C12N 2740/10043; C12N 2740/15043; C12N 2750/14143

(57) ABSTRACT

Provided are Delta-like ligand 3 (DLL3)-binding molecules, in particular, to human antibodies specific for DLL3, including antibody fragments. Also provided are recombinant receptors, including chimeric antigen receptors (CARs) that contain such antibodies or fragments, and polynucleotides that encode the antibodies, antigen-binding fragments or receptors specific for DLL3. Also provided are genetically engineered cells, containing such DLL3-binding proteins and receptors, and related methods and uses thereof in adoptive cell therapy.

### CROSS-REFERENCE TO RELATED APPLICATIONS

This application claims priority from U.S. provisional application No. 63/401,559, filed Aug. 26, 2022, entitled “ANTIBODIES AND CHIMERIC ANTIGEN RECEPTORS SPECIFIC FOR DELTA-LIKE LIGAND 3 (DLL3),” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042022240SeqList, created Aug. 25, 2023, which is 184,247 bytes in size. The information in the electronic format of the Sequence Listing is incorporated by reference in its entirety.

### FIELD

The present disclosure relates in some aspects to Delta-like ligand 3 (DLL3)-binding molecules, in particular, to human antibodies specific for DLL3, including antibody fragments. The present disclosure also relates to recombinant receptors, including chimeric antigen receptors (CARs) that contain such antibodies or fragments, and polynucleotides that encode the antibodies, antigen-binding fragments or receptors specific for DLL3. The disclosure also relates to genetically engineered cells, containing such DLL3-binding proteins and receptors, and related methods and uses thereof in adoptive cell therapy.

### BACKGROUND

Delta-like ligand 3 (DLL3) is a type 1 transmembrane Delta-like protein and an inhibitory Notch ligand normally expressed on intracellular membranes but is highly expressed on the surface of small cell lung cancer (SCLC) cells and other neuroendocrine cancer cells. DLL3 is aberrantly expressed on the cell surface in high-grade pulmonary neuroendocrine tumors, melanoma, and glioma. In normal adult tissues, DLL3 is generally expressed at low levels. SCLC often occurs in the central lung airways, grows aggressively, and is often metastatic. SCLC has a poor prognosis and few treatment options. Embodiments described herein provide DLL3 binding molecules and targeting therapies useful for treating cancers associated with DLL3 expression.

### SUMMARY

Provided herein are binding molecules, such as antibodies or antigen-binding fragments thereof, that bind to Delta-like ligand 3 (DLL3). Also among the binding molecules are polypeptides containing such antibodies, including single chain cell surface proteins, e.g., recombinant receptors such as chimeric antigen receptors (CARs), fusion proteins or conjugates, containing such antibodies. Also provided are recombinant receptors, such as chimeric antigen receptors (CARs) that bind to DLL3. Also provided are conjugates, such as immunoconjugates, or fusion proteins, such as a T cell engager, comprising any of the antibodies or antigen-binding fragment thereof described herein. Also provided are polynucleotides containing nucleic acids sequences encoding all or a portion of such antibodies, antigen-binding fragments, recombinant receptors, such as CARs, fusion proteins or conjugates described herein. Also provided are cells that comprise any of the provided binding molecules, such as antibodies or antigen-binding fragments thereof, CARs, fusion proteins or conjugates. Also provided are compositions comprising any of the binding molecules, such as antibodies or antigen-binding fragments thereof, CARs, fusion proteins, conjugates, polynucleotides or cells, and related methods and uses, such as in therapeutic applications, of any of the described binding molecules, such as antibodies or antigen-binding fragments thereof, CARs, fusion proteins, conjugates, polynucleotides, cells or compositions.

Provided herein is an anti-Delta-like ligand 3 (DLL3) antibody or antigen-binding fragment thereof comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:90, and the V_{L} region comprises a light chain complementarity determining region 1 (CDR-L1), a light chain complementarity determining region 2 (CDR-L2) and a light chain complementarity determining region 3 (CDR-L3) contained within SEQ ID NO:91.

Provided herein is an anti-DLL3 antibody or antigen-binding fragment thereof, comprising a heavy chain variable (V_{H}) region, and a light chain variable (V_{L}) region, wherein: the V_{H} region comprises a heavy chain complementarity determining region 1 (CDR-H1) comprising the sequence set forth in SEQ ID NO:92, a heavy chain complementarity determining region 2 (CDR-H2) comprising the sequence set forth in SEQ ID NO:93, and a heavy chain complementarity determining region 3 (CDR-H3) comprising the sequence set forth in SEQ ID NO:94, and the V_{L} region comprises a light chain complementarity determining region 1 (CDR-L1) comprising the sequence set forth in SEQ ID NO:95, a light chain complementarity determining region 2 (CDR-L2) comprising the sequence set forth in SEQ ID NO:96, and a light chain complementarity determining region 3 (CDR-L3) comprising the sequence set forth in SEQ ID NO:97.

In some of any embodiments, the V_{H} region comprises an amino acid sequence having at least at or about 95%, 96%, 97%, 98% or 99% sequence identity to SEQ ID NO:90, and the V_{L} region comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:91.

Provided herein is an anti-DLL3 antibody or antigen-binding fragment thereof comprising a heavy chain variable (V_{H}) region and a light chain variable (V_{L}) region, wherein: the V_{H} region comprises an amino acid sequence having at least at or about 95%, 96%, 97%, 98% or 99% sequence identity to SEQ ID NO:90, and the V_{L} region comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:91.

In some of any embodiments, the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:90, and the V_{L} region comprises a light chain complementarity determining region 1 (CDR-L1), a light chain complementarity determining region 2 (CDR-L2) and a light chain complementarity determining region 3 (CDR-L3) contained within SEQ ID NO:91.

In some of any embodiments, the V_{H} region comprises a heavy chain complementarity determining region 1 (CDR-H1) comprising the sequence set forth in SEQ ID NO:92, a heavy chain complementarity determining region 2 (CDR-H2) comprising the sequence set forth in SEQ ID NO:93, and a heavy chain complementarity determining region 3 (CDR-H3) comprising the sequence set forth in SEQ ID NO:94, and the V_{L} region comprises a light chain complementarity determining region 1 (CDR-L1) comprising the sequence set forth in SEQ ID NO:95, a light chain complementarity determining region 2 (CDR-L2) comprising the sequence set forth in SEQ ID NO:96, and a light chain complementarity determining region 3 (CDR-L3) comprising the sequence set forth in SEQ ID NO:97.

In some of any embodiments, the V_{H} region comprises the sequence set forth in SEQ ID NO:90, and the V_{L} region comprises the sequence set forth in SEQ ID NO:91.

Provided herein is an anti-DLL3 antibody or antigen-binding fragment thereof, comprising a heavy chain variable (V_{H}) region and a light chain variable (V_{L}) region, wherein: the V_{H} region comprises the sequence set forth in SEQ ID NO:90, and the V_{L} region comprises the sequence set forth in SEQ ID NO:91.

In some of any embodiments, the anti-DLL3 antibody or antigen-binding fragment thereof is recombinant. In some of any embodiments, the V_{H} region and the V_{L} region is human or is from a human protein.

In some of any embodiments, the antibody further comprises a heavy chain constant region (C_{H}) and/or a light chain constant region (C_{L}). In some of any embodiments, the C_{H} comprises the sequence set forth in SEQ ID NO:126, or an amino acid sequence having at least at or about 95%, 96%, 97%, 98% or 99% sequence identity to SEQ ID NO:126; and/or the C_{L} comprises the sequence set forth in SEQ ID NO:127, or an amino acid sequence having at least at or about 95%, 96%, 97%, 98% or 99% sequence identity to SEQ ID NO:127. In some of any embodiments, the antibody or antigen-binding fragment thereof comprises: a heavy chain comprising the sequence set forth in SEQ ID NO:128; and/or a light chain comprising the sequence set forth in SEQ ID NO:129. In some of any embodiments, the antibody or antigen-binding fragment thereof comprises: a heavy chain comprising the sequence set forth in SEQ ID NO:128; and a light chain comprising the sequence set forth in SEQ ID NO:129.

In some of any embodiments, the antibody is a full-length antibody.

In some of any embodiments, the antibody is an antigen-binding fragment.

In some of any embodiments, the antigen-binding fragment thereof comprises a single chain Fv (scFv).

In some of any embodiments, the V_{H} region is amino-terminal to the V_{L} region.

In some of any embodiments, the V_{H} region is carboxy-terminal to the V_{L} region.

In some of any embodiments, the V_{H} region and the V_{L} region are joined by a flexible linker. In some of any embodiments, the flexible linker comprises the sequence set forth in SEQ ID NO:46.

In some of any embodiments, the scFv comprises the sequence set forth in SEQ ID NO:89.

In some of any embodiments, the anti-DLL3 antibody or antigen-binding fragment thereof specifically binds to a human DLL3. In some of any embodiments, the human DLL3 comprises an amino acid sequence set forth in SEQ ID NO:120 or 121.

In some of any embodiments, the anti-DLL3 antibody or antigen-binding fragment thereof does not bind to, is not cross-reactive to, or binds at a lower level or degree or affinity to a DLL1 or a DLL4 protein, such as a human DLL1 or a human DLL4. In some of any embodiments, the extent, level or degree or affinity of binding of the anti-DLL3 antibody or antigen-binding fragment thereof to a human DLL1 or a human DLL4 is at least at or about 75%, 80%, 90%, 95% or 99% less than the extent, level or degree or affinity of binding to a human DLL3.

In some of any embodiments, the antibody or antigen-binding fragment thereof binds to human DLL3 with an equilibrium dissociation constant (K_{D}) of from about 1×10⁻⁷ M to about 1×10⁻⁸ M.

Also provided is a single chain cell surface protein, comprising any of the anti-DLL3 antibodies or antigen-binding fragment thereof provided herein.

Also provided herein is a conjugate comprising any of the anti-DLL3 antibodies or antigen-binding fragment thereof provided herein and a heterologous molecule or moiety. In some of any embodiments, the heterologous molecule or moiety is a therapeutic moiety. In some of any embodiments, the heterologous molecule or moiety is a small molecule chemical compound, protein, peptide, or toxin.

Provided herein is an anti-DLL3 chimeric antigen receptor (CAR) comprising an extracellular antigen-binding domain comprising any of the anti-DLL3 antibodies or antigen-binding fragment thereof provided herein, a transmembrane domain and an intracellular signaling region. In some of any embodiments, the anti-DLL3 CAR also includes a spacer between the extracellular antigen-binding domain and the transmembrane domain.

In some of any embodiments, the spacer comprises at least a portion of an immunoglobulin or a variant thereof.

In some of any embodiments, the spacer comprises at least a portion of a hinge region of an immunoglobulin or a variant thereof. In some of any embodiments, the at least a portion of a hinge region comprises all or a portion of an IgG4 hinge region, such as a human IgG4 hinge region, or a variant thereof. In some of any embodiments, the variant IgG4 hinge region comprises a substitution of amino acids CPSC to CPPC compared to the wild-type IgG4 hinge region.

In some of any embodiments, the spacer is less than at or about 15 amino acids in length. In some of any embodiments, the spacer is between 12 and 15 amino acids in length.

In some of any embodiments, the spacer comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:1. In some of any embodiments, the spacer comprises the sequence set forth in SEQ ID NO:1.

In some of any embodiments, the spacer comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:3. In some of any embodiments, the spacer comprises the sequence set forth in SEQ ID NO:3.

In some of any embodiments, the spacer comprises at least a portion of a hinge region and at least a portion of a CH3 region of an immunoglobulin or a variant thereof. In some of any embodiments, the spacer is at or about 111, 112, 113, 114, 115, 116, 117, 118, 119, 120, 121, 122, 123, 124 or 125 amino acids in length, or has a length between any of the foregoing. In some of any embodiments, the spacer comprises the sequence set forth in SEQ ID NO:5 or 58 or an amino acid sequence having at least at or about 85%, 86%, 87%, 88%, 89%, 90%, 91%, 92%, 93%, 94%, 95%, 96%, 97%, 98% or 99% sequence identity to SEQ ID NO:5 or 58.

In some of any embodiments, the spacer comprises at least a portion of a hinge region, at least a portion of a CH2 and at least a portion of a CH3 region of an immunoglobulin or a variant thereof. In some of any embodiments, the spacer is at or about 130, 140, 150, 160, 170, 180, 190, 200, 210, 220, 221, 222, 223, 224, 225, 226, 227, 228, 229 or 230 amino acids in length, or has a length between any of the foregoing. In some of any embodiments, the spacer comprises the sequence set forth in SEQ ID NO:42 or 82 or an amino acid sequence having at least at or about 85%, 86%, 87%, 88%, 89%, 90%, 91%, 92%, 93%, 94%, 95%, 96%, 97%, 98% or 99% sequence identity to SEQ ID NO:42 or 82.

In some of any embodiments, the transmembrane domain comprises a transmembrane domain from CD4, CD28, or CD8.

In some of any embodiments, the transmembrane domain comprises a transmembrane domain from CD28, such as a human CD28. In some of any embodiments, the transmembrane domain comprises SEQ ID NO:10 or 12 or an amino acid sequence having at least at or about 85%, 86%, 87%, 88%, 89%, 90%, 91%, 92%, 93%, 94%, 95%, 96%, 97%, 98% or 99% sequence identity to SEQ ID NO:10 or 12.

In some of any embodiments, the transmembrane domain comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:10. In some of any embodiments, the transmembrane domain comprises the sequence set forth in SEQ ID NO:10.

In some of any embodiments, the transmembrane domain comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:12. In some of any embodiments, the transmembrane domain comprises the sequence set forth in SEQ ID NO:12.

In some of any embodiments, the intracellular signaling region comprises an intracellular signaling domain capable of inducing a primary activation signal in a T cell, is a T cell receptor (TCR) component and/or comprises an immunoreceptor tyrosine-based activation motif (ITAM). In some of any embodiments, the intracellular signaling domain comprises a cytoplasmic signaling domain of a CD3-zeta (CD3ζ) chain, such as a human CD3ζ chain. In some of any embodiments, the intracellular signaling domain comprises the sequence set forth in SEQ ID NO:19, or an amino acid sequence having at least at or about 90%, 91%, 92%, 93%, 94%, 95%, 96%, 97%, 98% or 99% sequence identity to SEQ ID NO:19. In some of any embodiments, the intracellular signaling domain comprises the sequence set forth in SEQ ID NO:19.

In some of any embodiments, the intracellular signaling region further comprises a costimulatory domain. In some of any embodiments, the costimulatory domain is between the transmembrane domain and the intracellular signaling domain. In some of any embodiments, the costimulatory domain comprises an intracellular signaling domain of a T cell costimulatory molecule or a signaling portion thereof. In some of any embodiments, the costimulatory domain comprises an intracellular signaling domain of CD28, 4-1BB, or ICOS.

In some of any embodiments, the costimulatory domain comprises an intracellular signaling domain of 4-1BB, such as a human 4-1BB. In some of any embodiments, the costimulatory domain comprises the sequence set forth in SEQ ID NO:17 or an amino acid sequence having at least at or about 90%, 91%, 92%, 93%, 94%, 95%, 96%, 97%, 98% or 99% sequence identity to SEQ ID NO:17.

In some of any embodiments, the costimulatory domain comprises an intracellular signaling domain of CD28, such as a human CD28. In some of any embodiments, the costimulatory domain comprises the sequence set forth in SEQ ID NO:15 or 16 or an amino acid sequence having at least at or about 90%, 91%, 92%, 93%, 94%, 95%, 96%, 97%, 98% or 99% sequence identity to SEQ ID NO:15 or 16.

In some of any embodiments, the CAR comprises from its N to C terminus in order: the extracellular antigen-binding domain, the spacer, the transmembrane domain and the intracellular signaling region.

In some of any embodiments, the CAR comprises, from its N to C terminus in order: an extracellular antigen-binding domain comprising the scFv; a spacer comprising a modified IgG4 hinge, for example comprising the sequence set forth in SEQ ID NO:1; a transmembrane domain, such as a transmembrane domain from a human CD28, for example comprising the sequence set forth in SEQ ID NO:10; and an intracellular signaling region comprising a cytoplasmic signaling domain of a CD3-zeta (CD3ζ) chain and an intracellular signaling domain of a costimulatory molecule, for example comprising an intracellular signaling domain of 4-1BB.

In some of any embodiments, the CAR comprises, from its N to C terminus in order: an extracellular antigen-binding domain comprising the scFv; a spacer comprising a modified IgG4 hinge, for example comprising the sequence set forth in SEQ ID NO:3; a transmembrane domain, such as a transmembrane domain from a human CD28, for example comprising the sequence set forth in SEQ ID NO:12; and an intracellular signaling region comprising a cytoplasmic signaling domain of a CD3-zeta (CD3ζ) chain and an intracellular signaling domain of a costimulatory molecule, for example comprising an intracellular signaling domain of 4-1BB.

Provided herein is an anti-DLL3 chimeric antigen receptor (CAR) comprising: an extracellular antigen-binding domain comprising an scFv comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:90, and the V_{L} region comprises a light chain complementarity determining region 1 (CDR-L1), a light chain complementarity determining region 2 (CDR-L2) and a light chain complementarity determining region 3 (CDR-L3) contained within SEQ ID NO:91, a spacer comprising the sequence set forth in SEQ ID NO:1; a transmembrane domain comprising the sequence set forth in SEQ ID NO:10; and an intracellular signaling region comprising a cytoplasmic signaling domain of a CD3-zeta (CD3ζ) chain comprising the sequence set forth in SEQ ID NO:19, and an intracellular signaling domain of a costimulatory molecule comprising the sequence set forth in SEQ ID NO:17.

Provided herein is an anti-DLL3 chimeric antigen receptor (CAR) comprising: an extracellular antigen-binding domain comprising an scFv comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:90, and the V_{L} region comprises a light chain complementarity determining region 1 (CDR-L1), a light chain complementarity determining region 2 (CDR-L2) and a light chain complementarity determining region 3 (CDR-L3) contained within SEQ ID NO:91, a spacer comprising the sequence set forth in SEQ ID NO:3; a transmembrane domain comprising the sequence set forth in SEQ ID NO:12; and an intracellular signaling region comprising a cytoplasmic signaling domain of a CD3-zeta (CD3ζ) chain comprising the sequence set forth in SEQ ID NO:19, and an intracellular signaling domain of a costimulatory molecule comprising the sequence set forth in SEQ ID NO:17.

In some of any embodiments, the CAR comprises, from its N to C terminus in order: an extracellular antigen-binding domain comprising the scFv comprising the sequence set forth in SEQ ID NO:89; a spacer comprising the sequence set forth in SEQ ID NO:1; a transmembrane domain comprising the sequence set forth in SEQ ID NO:10; and an intracellular signaling region comprising a cytoplasmic signaling domain of a CD3-zeta (CD3ζ) chain comprising the sequence set forth in SEQ ID NO:19, and an intracellular signaling domain of a costimulatory molecule comprising the sequence set forth in SEQ ID NO:17.

In some of any embodiments, the CAR comprises, from its N to C terminus in order: an extracellular antigen-binding domain comprising the scFv comprising the sequence set forth in SEQ ID NO:89; a spacer comprising the sequence set forth in SEQ ID NO:3; a transmembrane domain comprising the sequence set forth in SEQ ID NO:12; and an intracellular signaling region comprising a cytoplasmic signaling domain of a CD3-zeta (CD3ζ) chain comprising the sequence set forth in SEQ ID NO:19, and an intracellular signaling domain of a costimulatory molecule comprising the sequence set forth in SEQ ID NO:17.

In some of any embodiments, the anti-DLL3 CAR comprises the sequence set forth in SEQ ID NO:117 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO:117. In some of any embodiments, the anti-DLL3 CAR comprises the sequence set forth in SEQ ID NO:117.

In some of any embodiments, the anti-DLL3 CAR comprises or consists of the sequence set forth in SEQ ID NO:119 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO:119. In some of any embodiments, the anti-DLL3 CAR comprises or consists of the sequence set forth in SEQ ID NO:119.

Also provided is a polynucleotide comprising a nucleic acid encoding any of the anti-DLL3 antibodies or antigen-binding domain thereof provided herein.

Also provided is a polynucleotide comprising a nucleic acid encoding any of the single chain cell surface proteins provided herein.

Also provided is a polynucleotide comprising a nucleic acid encoding any of the conjugates provided herein.

Also provided is a polynucleotide comprising a nucleic acid encoding any of the anti-DLL3 CARs provided herein.

In some of any embodiments, the polynucleotide comprises: a nucleic acid encoding the V_{H}, comprising the sequence set forth in SEQ ID NO:87, or a nucleic acid sequence having at least at or about 90%, 91%, 92%, 93%, 94%, 95%, 96%, 97%, 98% or 99% sequence identity to SEQ ID NO:87, and a nucleic acid encoding the V_{L}, comprising the sequence set forth in SEQ ID NO:88, or a nucleic acid sequence having at least at or about 90%, 91%, 92%, 93%, 94%, 95%, 96%, 97%, 98% or 99% sequence identity to SEQ ID NO:88. In some of any embodiments, the polynucleotide comprises: a nucleic acid encoding the V_{H}, comprising the sequence set forth in SEQ ID NO:87, and a nucleic acid encoding the V_{L}, comprising the sequence set forth in SEQ ID NO:88. In some of any embodiments, the antigen-binding fragment is a single chain Fv (scFv), and the polynucleotide comprises a nucleic acid encoding the scFv, comprising the sequence set forth in SEQ ID NO:86 or a nucleic acid sequence having at least at or about 90%, 91%, 92%, 93%, 94%, 95%, 96%, 97%, 98% or 99% sequence identity to SEQ ID NO:86. In some of any embodiments, the antigen-binding fragment is a single chain Fv (scFv), and the polynucleotide comprises a nucleic acid encoding the scFv, comprising the sequence set forth in SEQ ID NO:86.

In some of any embodiments, the polynucleotide comprises a nucleic acid encoding the spacer comprising the sequence set forth in SEQ ID NO:2 or a nucleic acid sequence having at least at or about 90%, 91%, 92%, 93%, 94%, 95%, 96%, 97%, 98% or 99% sequence identity to SEQ ID NO:2. In some of any embodiments, the polynucleotide comprises a nucleic acid encoding the spacer comprising the sequence set forth in SEQ ID NO:2.

In some of any embodiments, the polynucleotide comprises a nucleic acid encoding the spacer comprising the sequence set forth in SEQ ID NO:4 or a nucleic acid sequence having at least at or about 90%, 91%, 92%, 93%, 94%, 95%, 96%, 97%, 98% or 99% sequence identity to SEQ ID NO:4. In some of any embodiments, the polynucleotide comprises a nucleic acid encoding the spacer comprising the sequence set forth in SEQ ID NO:4.

In some of any embodiments, the polynucleotide comprises a nucleic acid encoding the transmembrane domain comprising the sequence set forth in SEQ ID NO:11 or a nucleic acid sequence having at least at or about 90%, 91%, 92%, 93%, 94%, 95%, 96%, 97%, 98% or 99% sequence identity to SEQ ID NO:11. In some of any embodiments, the polynucleotide comprises a nucleic acid encoding the transmembrane domain, comprising the sequence set forth in SEQ ID NO:11.

In some of any embodiments, the polynucleotide comprises a nucleic acid encoding the transmembrane domain comprising the sequence set forth in SEQ ID NO:13 or a nucleic acid sequence having at least at or about 90%, 91%, 92%, 93%, 94%, 95%, 96%, 97%, 98% or 99% sequence identity to SEQ ID NO:13. In some of any embodiments, the polynucleotide comprises a nucleic acid encoding the transmembrane domain comprising the sequence set forth in SEQ ID NO:13.

In some of any embodiments, the nucleic acid encoding the anti-DLL3 chimeric antigen receptor comprises the sequence set forth in SEQ ID NO:116 or a sequence that exhibits at least 90%, 91%, 92%, 93%, 94%, 95%, 96%, 97%, 98%, or 99% sequence identity to the sequence set forth in SEQ ID NO:116. In some of any embodiments, the nucleic acid encoding the anti-DLL3 chimeric antigen receptor comprises the sequence set forth in SEQ ID NO:116.

In some of any embodiments, the polynucleotide is optimized by splice site elimination.

In some of any embodiments, the polynucleotide is codon-optimized for expression in a human cell.

Also provided herein is a vector, comprising any of the polynucleotides provided herein. In some of any embodiments, the vector is a viral vector. In some of any embodiments, the viral vector is a retroviral vector or a lentiviral vector. In some of any embodiments, the viral vector is an adeno-associated virus (AAV) vector.

Also provided herein is a cell comprising any of the anti-DLL3 antibodies or antigen-binding fragment thereof provided herein, any of the single chain cell surface proteins provided herein or any of the conjugates provided herein.

Also provided herein is a cell comprising any of the anti-DLL3 CARs provided herein.

Also provided herein is a cell comprising any of the polynucleotides provided herein, or any of the vectors provided herein.

In some of any embodiments, the cell is a lymphocyte. In some of any embodiments, the cell is an NK cell or a T cell. In some of any embodiments, the cell is a T cell and the T cell is a CD4+ T cell or a CD8+ T cell.

In some of any embodiments, the cell is a primary cell obtained from a subject.

In some of any embodiments, among a plurality of the cells, less than at or about 10%, at or about 9%, at or about 8%, at or about 7%, at or about 5%, at or about 4%, at or about 3%, at or about 2% or at or about 1% of the cells in the plurality comprise an anti-DLL3 chimeric antigen receptor that exhibits tonic signaling and/or antigen independent activity or signaling.

Also provided herein is a composition comprising any of the cells provided herein.

Also provided herein is a composition comprising any of the anti-DLL3 antibodies or antigen-binding fragment thereof provided herein, any of the single chain cell surface proteins provided herein, any of the conjugates provided herein or any of the anti-DLL3 CARs provided herein.

In some of any embodiments, the composition also includes a pharmaceutically acceptable excipient.

In some of any embodiments, the composition comprises CD4+ and CD8+ T cells and the ratio of CD4+ to CD8+ T cells is from at or about 1:3 to 3:1, such as at or about 1:2 to 2:1, such as at or about 1:1.

In some of any embodiments, among a plurality of the cells in the composition, less than at or about 10%, at or about 9%, at or about 8%, at or about 7%, at or about 5%, at or about 4%, at or about 3%, at or about 2% or at or about 1% of the cells in the plurality comprise an anti-DLL3 chimeric antigen receptor that exhibits tonic signaling and/or antigen independent activity or signaling.

Also provided herein is a method of treatment, that involves administering any of the anti-DLL3 antibodies or antigen-binding fragment thereof provided herein, any of the single chain cell surface proteins provided herein, any of the conjugates provided herein, any of the anti-DLL3 CARs provided herein, any of the polynucleotides provided herein, any of the vectors provided herein, any of the cells provided herein, or any of the compositions provided herein to a subject having a disease or disorder associated with DLL3.

In some of any embodiments, the disease or disorder associated with DLL3 is a cancer. In some of any embodiments, the cancer is a DLL3-expressing cancer. In some of any embodiments, the disease or disorder associated with DLL3 is selected from among a neuroendocrine tumor, a small cell lung cancer (SCLC), a large cell neuroendocrine carcinoma (LCNEC), a melanoma, a glioma, or a glioblastoma. In some of any embodiments, the disease or disorder associated with DLL3 is a neuroendocrine tumor. In some of any embodiments, the disease or disorder associated with DLL3 is a small cell lung cancer (SCLC). In some of any embodiments, the disease or disorder associated with DLL3 is a large cell neuroendocrine carcinoma (LCNEC).

Also provided herein are any of the anti-DLL3 antibodies or antigen-binding fragment thereof provided herein, any of the single chain cell surface proteins provided herein, any of the conjugates provided herein, any of the anti-DLL3 CARs provided herein, any of the polynucleotides provided herein, any of the vectors provided herein, any of the cells provided herein, or any of the compositions provided herein for use in treating a disease or disorder associated with DLL3.

Also provided herein are uses of any of the anti-DLL3 antibodies or antigen-binding fragment thereof provided herein, any of the single chain cell surface proteins provided herein, any of the conjugates provided herein, any of the anti-DLL3 CARs provided herein, any of the polynucleotides provided herein, any of the vectors provided herein, any of the cells provided herein, or any of the compositions provided herein for the manufacture of a medicament for treating a disease or disorder associated with DLL3.

Also provided herein are uses of any of the anti-DLL3 antibodies or antigen-binding fragment thereof provided herein, any of the single chain cell surface proteins provided herein, any of the conjugates provided herein, any of the anti-DLL3 CARs provided herein, any of the polynucleotides provided herein, any of the vectors provided herein, any of the cells provided herein, or any of the compositions provided herein for the treatment of a disease or disorder associated with DLL3.

In some of any embodiments, the disease or disorder associated with DLL3 is a cancer. In some of any embodiments, the cancer is a DLL3-expressing cancer. In some of any embodiments, the disease or disorder associated with DLL3 is selected from among a neuroendocrine tumor, a small cell lung cancer (SCLC), a large cell neuroendocrine carcinoma (LCNEC), a melanoma, a glioma, or a glioblastoma. In some of any embodiments, the disease or disorder associated with DLL3 is a neuroendocrine tumor. In some of any embodiments, the disease or disorder associated with DLL3 is a small cell lung cancer (SCLC). In some of any embodiments, the disease or disorder associated with DLL3 is a large cell neuroendocrine carcinoma (LCNEC).

Also provided herein is a kit comprising any of the anti-DLL3 antibodies or antigen-binding fragment thereof provided herein, any of the single chain cell surface proteins provided herein, any of the conjugates provided herein, any of the anti-DLL3 CARs provided herein, any of the polynucleotides provided herein, any of the vectors provided herein, any of the cells provided herein or any of the compositions provided herein, and instructions for use.

In some of any embodiments, the instructions are for administering the anti-DLL3 antibody or antigen-binding fragment thereof, the single chain cell surface protein, the conjugate, the anti-DLL3 chimeric antigen receptor, the cell or the composition to a subject having a disease or disorder associated with DLL3.

Provided herein is an article of manufacture comprising any of the anti-DLL3 antibodies or antigen-binding fragment thereof provided herein, any of the single chain cell surface proteins provided herein, any of the conjugates provided herein, any of the anti-DLL3 CARs provided herein, any of the polynucleotides provided herein, any of the vectors provided herein, any of the cells provided herein, any of the compositions provided herein, or any of the kits provided herein.

### BRIEF DESCRIPTION OF THE DRAWINGS

FIG. 1 shows exemplary results for antigen-dependent and antigen-independent signaling, based on a Jurkat T cell line containing a Nur77 tdTomato knock-in reporter and expressing one of several anti-DLL3 chimeric antigen receptors (CARs) comprising DLL3-binding single chain Fv (scFv) obtained from immunized rats, and in the presence of human DLL1-, DLL3-, DLL4-, or cynomolgus DLL3-expressing K562 cells, or DLL3-expressing target cells NCI-H69.

FIG. 2 shows the DLL3 expression levels in DLL3-expressing small cell lung carcinoma target cell lines NCI-H69 (low DLL3; also referred to as “H69”) or NCI-H₈₂ (medium DLL3; also referred to as “H82”).

FIG. 3 shows levels of IL-2 production after a co-culture of primary T cells from 3 different donors engineered to express one of 18 candidate anti-DLL3 CARs with DLL3-expressing H69 (low DLL3) target cells at an effector:target (E:T) ratio of 1:4 for 24 hours.

FIG. 4 shows levels of IL-2 production after a co-culture of primary T cells from 3 different donors engineered to express one of 18 candidate anti-DLL3 CARs with DLL3-expressing H82 (medium DLL3) target cells at an effector:target (E:T) ratio of 1:4 for 24 hours.

FIG. 5 shows levels of IL-2 production after a co-culture of primary T cells from 2 different donors engineered to express one of 5 anti-DLL3 CARs with H69 (low DLL3) or H82 (medium DLL3) target cells.

FIG. 6 shows target cell death in a spheroid killing assay, after a co-culture of primary T cells from 2 different donors engineered to express one of 5 anti-DLL3 CARs with tumor spheroids of H69 (low DLL3) or H82 (medium DLL3) target cells.

FIG. 7 shows the mean tumor volume over time in a tumor xenograft mouse model subcutaneously injected with human H82 cells (medium DLL3), and administered primary T cells engineered to express one of 5 anti-DLL3 CARs at two dose levels: 1×10⁶ cells (low dose) or 4×10⁶ cells (high dose).

FIG. 8 shows expansion of administered T cells engineered to express CAR, as assessed by CD3+ cells count per 1 μL blood, in H82 tumor xenograft mice administered T cells expressing one of 5 anti-DLL3 CARs at two dose levels, at days 7 and 21 after administration.

FIG. 9 shows the mean tumor volume over time in a tumor xenograft mouse model subcutaneously injected with human H69 cells (low DLL3), and administered primary T cells engineered to express CAR-A or CAR-B at three dose levels: 1×10⁶ cells (low), 2.5×10⁶ cells (mid), or 5×10⁶ cells (high).

FIG. 10 shows the Kaplan-Meier survival curves for H69 tumor xenograft mice administered T cells expressing CAR-A or CAR-B at three dose levels.

FIG. 11 shows expansion of administered T cells engineered to express CAR, as assessed by CD3+ cells count per 1 μL blood, in H69 tumor xenograft mice administered T cells expressing CAR-A or CAR-B at three dose levels, at days 7, 14, 21, and 28 after administration.

### DETAILED DESCRIPTION

Provided are Delta-like ligand 3 (DLL3)-binding molecules. In some aspects, the provided DLL3-binding molecules include antibodies (including antigen-binding antibody fragments, such as single chain fragments, including single chain Fv fragments (scFvs)), recombinant receptors, including chimeric receptors containing such antibodies or fragments, and fusion proteins and conjugates, such as immunoconjugates. Also provided are polynucleotides encoding such antibodies, fragments or recombinant receptors. In some aspects, provided embodiments relate to antibodies (including antigen-binding fragments) that bind to (or target) DLL3, and other binding molecules incorporating the same, such as fusion proteins, conjugates, T cell engagers (TCEs), and chimeric antigen receptors (CARs). In particular, among provided embodiments are CARs that bind to or target DLL3 and engineered T cells expressing the same. In some embodiments, the DLL3 that is targeted is expressed a DLL3-expressing cells, such as associated with a disease or condition.

In some aspects, DLL3 is expressed in cells or tissues associated with certain diseases and conditions, such as malignancies, e.g., on small cell lung cancer (SCLC) cells and high-grade neuroendocrine tumors. In some aspects, DLL3 exhibits minimal or no expression in normal adult tissues.

Among the provided embodiments are approaches useful in the treatment of diseases and conditions and/or for targeting such cell types, including nucleic acid molecules that encode DLL3-binding antibodies, fragments, fusion proteins or receptors and the encoded antibodies or antigen-binding fragments, fusion proteins and receptors.

Among the provided embodiments are approaches useful in the treatment of diseases and conditions associated with DLL3 expression, including adoptive cell therapies involving cells expressing a chimeric antigen receptor (CAR) targeting DLL3, such as a CAR that comprises a binding molecule as an extracellular antigen-binding domain.

Also provided are compositions and articles of manufacture comprising any one or more of the polypeptides and nucleic acids provided herein.

In some aspects, the provided chimeric antigen receptors (CARs) generally can contain antibodies (including antigen-binding antibody fragments, such as heavy chain variable (V_{H}) regions, single domain antibody fragments and single chain fragments, including scFvs) specific for DLL3, for example as the antigen-binding domain such as the extracellular antigen-binding domain. Also provided are cells, such as engineered or recombinant cells, expressing such DLL3-binding receptors, e.g., anti-DLL3 chimeric antigen receptors (CARs) and/or containing nucleic acids encoding such receptors, and compositions and articles of manufacture and therapeutic doses containing such cells. Also provided are methods of making and using the antibodies and fragments as well as cells expressing or containing the antibodies and fragments, such as for production of the antibodies or fragments thereof. Also provided are compositions, including pharmaceutical compositions, containing such antibodies, antigen-binding fragments, receptors or cells, and conjugates comprising such antibodies or fragments. In some aspects, the provided compositions, antibodies, antigen-binding fragments, receptors or cells can be used in connection with a therapy or a method of treatment.

In some contexts, properties of particular target antigens that the antibodies or recombinant receptors containing antigen-binding domains specifically bind, recognize or target, can affect the activity of the receptor. In some contexts, DLL3 is expressed by certain cancers and is an attractive therapeutic target for cell therapy. Improved strategies are needed for optimal responses DLL3-targeting therapies.

In some contexts, optimal response to therapy such as cell therapy can also depend on the ability of the engineered recombinant receptors such as CARs, to be consistently and reliably expressed on the surface of the cells and/or bind the target antigen. For example, in some cases, heterogeneity of the transcribed RNA from an introduced transgene (e.g., encoding the recombinant receptor) can affect the expression and/or activity of the recombinant receptor, in some cases when expressed in a cell, such as a human T cell, used in cell therapy. In some contexts, the length and type of spacer in the recombinant receptor, such as a CAR, can affect the expression, activity and/or function of the receptor.

Provided are embodiments that meet such needs. The provided embodiments, such as those incorporating provided anti-DLL3 antibodies, can be used as an effective therapy for targeting DLL3, such as in the treatment of cancer and other diseases and disorders, such as those associated with the expression or overexpression of DLL3. In some embodiments, provided embodiments include cells (e.g. T cells) engineered with a CAR incorporating an anti-DLL3 antibody or antigen-binding fragment in the extracellular antigen-binding domain, that can be used to target and kill cells associated with a disease or disorder, such as malignancies, tumors or cancers, that express DLL3, for the treatment of the disease or disorder. In some aspects, results herein show that exemplary CAR-engineered T cells exhibit effective antigen-dependent killing of tumor cells expressing DLL3. In some aspects, the provided embodiments are based on observations that administration of engineered cells expressing the provided DLL3 binding molecules, such as chimeric antigen receptors (CARs), exhibit antigen-specific activity, signaling and function, high anti-tumor activity, antigen-dependent activity or signaling, and/or prolonged in vivo expansion when administered. Such antibodies, and recombinant receptors such as CARs incorporating same, can be used to facilitate safe and effective treatment of particular diseases and disorders, such as those associated with expression of DLL3, for example, small cell lung cancer (SCLC).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. DLL3-BINDING MOLECULES

Provided in some aspects are DLL3-binding molecules, such as DLL3-binding polypeptides. Such binding molecules include antibodies (including antigen-binding fragments) that specifically bind to DLL3 proteins, such as a human DLL3. Also among the binding molecules are polypeptides containing such antibodies, including single chain cell surface proteins, e.g., recombinant receptors such as chimeric antigen receptors (CARs), fusion proteins or conjugates, containing such antibodies. Provided in some aspects are DLL3-binding recombinant receptors, such as chimeric antigen receptors (CARs) that bind DLL3 molecules and polynucleotides encoding DLL3 binding recombinant receptors (e.g., CARs), and cells expressing such receptors. Also provided are polynucleotides containing nucleic acids sequences encoding all or a portion of such antibodies, antigen-binding fragments, recombinant receptors, such as CARs, fusion proteins or conjugates, such as those described in Section I.A-I.E. Exemplary of such polynucleotides include those described in Section I.F. In some aspects, the polynucleotides can be introduced into a cell to generate an engineered cell that comprises or expresses the provided binding molecules, e.g., DLL3-binding antibodies, fragments, fusion proteins, conjugates, T cell engagers (TCEs), and recombinant receptors, such as CARs.

In some embodiments, the provided antibodies or antigen-binding fragment thereof specifically bind to a Delta-like ligand 3 (DLL3) protein. DLL3 is a type I transmembrane Delta-like protein typically expressed exclusively on intracellular membranes, such as the Golgi apparatus. DLL3 is a member of the delta protein ligand family, which functions as Notch ligands in the Notch signaling pathways. The Notch pathway is a highly conserved cellular signaling pathway involved in a variety of aspects of developmental biology, including somite segmentation and neurogenesis during early embryonic development, and the development of pulmonary neuroendocrine cells. Typically, extracellular expression of DLL3 is highest in fetal brain and minimal in normal adult human tissues, except in the brain.

DLL3 is an inhibitory Notch pathway ligand, which typically inhibits Notch pathway activation by binding and retaining Notch and DLL1 in the cytoplasm. Notch signaling is downregulated during neuroendocrine tumor growth and is inhibited by DLL3 expression. DLL3 is one of the direct downstream targets of Achaete-Scute Family BHLH Transcription Factor 1 (ASCL1), a key transcriptional factor associated with small cell lung cancer (SCLC) oncogenesis. DLL3 is highly upregulated and aberrantly expressed on the cell surface in SCLC and other high-grade neuroendocrine tumors, including large cell neuroendocrine carcinoma (LCNEC), melanomas, gliomas, and glioblastomas. In some aspects, DLL3 was observed to be expressed on the surface of SCLC tumor cells, and is associated with tumors and oncogenesis, for example, for SCLC or LCNEC. In some embodiments, the disease or disorder associated with DLL3 is a cancer. In some embodiments, the cancer is a DLL3-expressing cancer. In some embodiments, the cancer is associated with a DLL3-expressing solid tumor. In some embodiments, the DLL3-expressing solid tumor is a neuroendocrine tumor.

Due to the role of DLL3 in various diseases and conditions, including various types of cancers, DLL3 is a therapeutic target. The provided embodiments include binding molecules that bind to or target DLL3. In some embodiments, the provided embodiments, such as DLL3-binding molecules, such as antibodies or receptors, cells expressing the DLL3-binding molecules cells, or compositions thereof, are administered to the subject, such as a subject having or at risk for the disease or condition, such as a cancer. In some aspects, the administration thereby treat, e.g., ameliorate one or more symptom of the disease or condition, such as by lessening tumor burden in a DLL3-expressing cancer.

In some cases, DLL3 expression was observed to be associated with SCLC migration and invasion through a mechanism that involves control of the epithelial-mesenchymal transition protein Snail. In some aspects, DLL3 is also observed to be expressed on other tumor types of neuroendocrine origin, including melanoma, glioblastoma multiforme, small cell bladder cancer, metastatic castration-resistant prostate cancer, castration-resistant neuroendocrine prostate cancer and neuroendocrine lung tumors. In some embodiments, other tumors or cancers that can be treated with the provided embodiments, such as provided DLL3-binding molecules, include neuroendocrine tumors, such as neuroendocrine tumor of the anterior pituitary, neuroendocrine thyroid tumors, parathyroid tumors, pulmonary neuroendocrine tumors (e.g., bronchus tumors, pulmonary carcinoid tumors), gastroenteropancreatic neuroendocrine tumors (e.g., foregut gastroenteropancreatic neuroendocrine tumors including pancreatic endocrine tumors, midgut gastroenteropancreatic neuroendocrine tumors, hindgut gastroenteropancreatic neuroendocrine tumors), liver and gallbladder tumors, adrenal tumors (e.g., adrenomedullary tumors), peripheral nervous system tumors (e.g., Schwannoma, paraganglioma, neuroblastoma), neuroendocrine tumor of the cervix, prostate tumor, gliomas (e.g. astrocytoma, central neurocytoma, ependymoma, mixed glioma, oligondendroglioma, optic nerve glioma, subependymal giant cell astrocytoma, glioblastoma).

In some embodiments, diseases or disorders associated with the expression of DLL3 that can be treated with embodiments described herein include medullary carcinoma, lung cancer, extrapulmonary small cell carcinomas, goblet cell carcinoma, breast cancer, stomach cancer, glioma, glioblastoma, thyroid cancer, carcinoids, pancreatic cancer, bladder cancer, prostate cancer, urinary tract carcinoid tumor and neuroendocrine carcinoma, ovarian cancer, testicular cancer, and Merkel cell carcinoma. In some embodiments, the disease or disorder associated with the expression of DLL3 that can be treated with any of the provided embodiments is a large cell neuroendocrine carcinoma (LCNEC). In some embodiments, the disease or disorder associated with the expression of DLL3 that can be treated with any of the provided embodiments is a small cell lung cancer (SCLC).

### A. DLL3-Targeting Antibodies and Antigen-Binding Fragment Thereof

Provided are anti-DLL3 polypeptides, including antibodies and functional antigen-binding fragments. Among the DLL3-binding polypeptides are antibodies, such as single-chain antibodies (e.g., antigen-binding antibody fragments), such as those containing a heavy chain variable (V_{H}) region and/or a light chain variable (V_{L}) region, or a portion thereof. In some embodiments, the antibodies or antigen-binding fragments include a V_{H} and a V_{L}, such as single chain Fv fragments (scFvs). The antibodies include antibodies that specifically bind to DLL3, e.g., human DLL3. Among the provided anti-DLL3 antibodies are human antibodies, or antibodies that are modified from or variant of human antibodies. The antibodies include isolated antibodies. Also provided are DLL3-binding molecules containing such antibodies, such as single-chain proteins, fusion proteins, conjugates, T cell engagers (TCEs), and/or recombinant receptors such as chimeric receptors, including antigen receptors. In some aspects, the DLL3-binding molecules include isolated molecules.

Also provided are DLL3-binding cell surface proteins, such as DLL3-binding recombinant receptors, such as chimeric antigen receptors (CARs). The DLL3-binding recombinant receptors can contain the provided antibodies (e.g., antigen-binding antibody fragments) that specifically bind to DLL3, such as to DLL3 proteins, such as human DLL3. In some aspects, the provided binding molecules bind to an extracellular portion of DLL3. In some examples, the recombinant receptors are CARs, such as those containing anti-DLL3 antibodies or antigen-binding fragments thereof.

Also provided are polynucleotides containing nucleic acids sequences encoding all or a portion of such antibodies, antigen-binding fragments and binding molecules. The provided polynucleotides can be incorporated into constructs, such as deoxyribonucleic acid (DNA) or ribonucleic acid (RNA) constructs, such as those that can be introduced into cells for expression of the encoded DLL3-binding antibodies, antigen-binding fragments, fusion proteins, conjugates, T cell engagers (TCEs), or receptors, e.g., anti-DLL3 CARs. In some aspects, the encoded antibodies, antigen-binding fragments, fusion proteins, conjugates and receptors, such as those containing DLL3-binding polypeptides, and compositions and articles of manufacture and uses of the same, also are provided. The term “antibody” herein is used in the broadest sense and includes polyclonal and monoclonal antibodies, including intact antibodies and functional (antigen-binding) antibody fragments, including fragment antigen-binding (Fab) fragments, F(ab′)₂ fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD. Typically, a naturally occurring immunoglobulin has heavy (H) chains and light (L) chains interconnected by disulfide bonds. There are two types of light chain, lambda (λ) and kappa (k). There are five main heavy chain classes, or isotypes, which determine the functional activity of an antibody molecule: IgM, IgD, IgG, IgA and IgE.

The terms “complementarity determining region,” and “CDR,” synonymous with “hypervariable region” or “HVR,” are known to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., J Mol Biol, 1997; 273(4):927-48 (“Chothia” numbering scheme); MacCallum et al., J. Mol. Biol, 1996; 262:732-745.” (“Contact” numbering scheme); Lefranc M P et al., Dev Comp Immunol, 2003; 27(1):55-77 (“IMGT” numbering scheme); Honegger A and Pluckthun A, J Mol Biol, 2001; 309(3):657-70, (“Aho” numbering scheme); Martin et al., PNAS, 1989; 86(23):9268-9272, (“AbM” numbering scheme); and Ye et al., Nucleic Acids Res. 2013; 41(Web Server issue):W34-40, (“IgBLAST numbering scheme). Details regarding various numbering schemes are also described in, for example, Jarasch et al., Proteins, 2017; 85(1):65-71; Martin et al., Bioinformatics tools for antibody engineering. In: Dubel, S. (editor) Handbook of Therapeutic Antibodies, Vol. 1. Wiley-VCH, Weinheim, Germany; Martin, A. C. R. (2010). Protein Sequence and Structure Analysis of Antibody Variable Domains. In: Kontermann, R., Dubel, S. (eds) Antibody Engineering. Springer Protocols Handbooks. Springer, Berlin, Heidelberg, and Martin, ACR, Antibody Information: How to identify the CDRs by looking at a sequence [online]<http://www.bioinf.org.uk/abs/info.html>, all of which are incorporated by reference in their entireties. Various prediction algorithm tools are available and known for numbering antibody residues and CDRs (e.g., AbYsis, Abnum, AbYmod, AbRSA, IgBLAST, IMGT, or ANARCI).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, in some cases with insertions. Insertions in the sequence relative to the standard numbering scheme are indicated using insertion letter codes. For example, residues that are inserted between residues L30 and L31 are indicated as L31A, L31B, etc. Deletions in the sequence relative to the standard scheme are accommodated by skipping numbers. The two schemes place certain insertions and deletions (“indels”) at different positions, resulting in differential numbering. For instance, the Chothia numbering scheme is nearly identical to the Kabat numbering scheme, except that insertions are placed at structural positions and topologically equivalents residues do get assigned the same numbers. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software. The IgBLAST scheme is based on matching to germline V, D and J genes, and can be determined using National Center for Biotechnology Information (NCBI)'s IgBLAST tool.

In some embodiments, Kabat numbering can be determined by known sequence rules as described in, for example, Kabat et al. (1991), “Sequences of Proteins of Immunological Interest,” 5th Ed. Public Health Svice, National Institutes of Health, Bethesda, MD. In some embodiments, the Kabat numbering scheme in some aspects can include any of the following rules to designate CDRs: CDR-L1 starts at approximately residue 24 of the light chain, always has a preceding C residue, and always has a following W residue; the end of CDR-L1 is defined by a stretch of 3 residues, where the W residue can be followed by Y, L, or F, followed by Q or L; CDR-1 has a length of 10 to 17 residues; CDR-L2 always starts 16 residues after the end of CDR-L1; the two residues before CDR-L2 are I and Y but can also be V and Y, I and K, or I and F; CDR-L2 is always 7 residues long; CDR-L3 always starts 33 residues after the end of CDR-L2, always has a preceding C residue, and is strictly followed by a F-G-X-G sequence motif, where X is any amino acid; CDR-L3 has a length of 7 to 11 residues; CDR-H1 starts at approximately position 26 of the heavy chain; the first amino acid in CDR-H1 is always 9 residues after a conserved C residue; CDR-H1 is followed by an invariant W residue followed by V, I, or A; CDR-H1 has a length of 5 to 7 residues; CDR-H2 always starts at 15 residues after the end of CDR-H1; the first residue in CDR-H2 is usually preceded by the sequence motif L-E-W-I-G but a number of variations exist; the end of CDR-H2 is defined by a motif of 3 residues—the first residue of the motif of 3 residues can be either K or R, the second residue of the motif of 3 residues can be L, I, V, F, T, or A, the third residue of the motif of 3 residues can be T, S, I, or A; CDR-H2 has a length of 16 to 19 residues; CDR-H3 always starts 33 residues after the end of CDR-H2 and is always 3 residues after a C residue—the first residue of CDR-H3 is preceded by the conserved C residue followed by two residues, which are usually A-R; the residues following CDR-H3 is strictly followed by a W-G-X-G sequence motif, where the X is any amino acid; CDR-H3 typically has a length of 3 to 25 residues; CDR-H3 can be much longer than 25 residues.

In some cases, according to the Chothia numbering scheme, exact boundary positions of certain CDRs can differ based on different definitions for the CDRs (See e.g., Martin, ACR, Antibody Information: How to identify the CDRs by looking at a sequence [online]<http://www.bioinf.org.uk/abs/info.html>). For example, in some instances, the boundary positions for CDR-L1 according to Chothia numbering can be L26-L32 (Chothia et al., Science, 1986; 233(4765):755-8 and Chothia C. and Lesk A. M. J Mol Biol, 1987; 196(4):901-17). In some instances, the boundary positions for CDR-L1 can be L25-L32 (Al-Lazikani et al., J Mol Biol, 1997; 273(4):927-48). In some instances, the boundary positions for CDR-L2 can be L50-L52 and for CDR-L3 can be L91-L96 (Chothia et al., Science, 1986; 233(4765):755-8; Chothia C. and Lesk A. M. J Mol Biol, 1987; 196(4):901-17; Al-Lazikani et al., J Mol Biol, 1997; 273(4):927-48). In some instances, the boundary positions for CDR-H1 according to Chothia numbering can be H26-H32 (Chothia et al., Science, 1986; 233(4765):755-8; Chothia C. and Lesk A. M. J Mol Biol, 1987; 196(4):901-17; Al-Lazikani et al., J Mol Biol, 1997; 273(4):927-48). In some instances, the boundary positions for CDR-H2 can be H53-H55 (Chothia et al., Science, 1986; 233(4765):755-8 and Chothia C. and Lesk A. M. J Mol Biol, 1987, 196(4):901-17); H52a-H55 (Tramontano et al., J Mol Biol, 1990, 215(1): 175-82). In some instances, the boundary positions for CDR-H2 can be H52-H56 (Al-Lazikani et al., J Mol Biol., 1997; 273(4):927-48). In some instances, the boundary positions for CDR-H3 can be H96-H101 (Chothia et al., Science, 1986; 233(4765):755-8 and Chothia C. and Lesk A. M. J Mol Biol., 1987; 196(4):901-17). In some instances, the boundary positions for CDR-H3 can be H92-H104 (Morea et al., Biophys Chem, 1997; 68(1-3): 9-16 and Morea et al., J Mol Biol., 1998; 275(2): 269-94).

Table 1, below, exemplifies exemplary numbering and lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 . . . 34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) comprises the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, the provided binding molecule, e.g., an anti-DLL3 antibody or antigen-binding fragment thereof or a recombinant receptor (e.g., CAR) comprising an antibody or an antigen-binding fragment thereof, comprises a CDR-H1, a CDR-H2, and a CDR-H3 as contained within a given V_{H} region amino acid sequence and defined by any of the aforementioned schemes, such as Kabat, Chothia, AbM, IgBLAST, IMGT, or Contact method, or other known schemes, and a CDR-L1, a CDR-L2, and a CDR-L3 as contained within a given V_{L} region amino acid sequence and defined by any of the aforementioned schemes, such as Kabat, Chothia, AbM, IgBLAST, IMGT, or Contact method, or other known schemes. In some embodiments, specific CDR sequences are specified. Exemplary CDR sequences of provided antibodies are described using various numbering schemes, such as Kabat, although it is understood that a provided antibody can include CDRs as described according to any of the other aforementioned numbering schemes or other known numbering schemes.

Likewise, unless otherwise specified, a FR or individual specified FR(s) (e.g., FR-H1, FR-H2, FR-H3, FR-H4, FR-L1, FR-L2, FR-L3, and/or FR-L4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IgBLAST, IMGT, or Contact method, or other known schemes. In some embodiments, the provided binding molecule, e.g., an anti-DLL3 antibody or antigen-binding fragment thereof or a recombinant receptor (e.g., CAR) comprising an antibody or an antigen-binding fragment thereof, comprises a FR-H1, a FR-H2, a FR-H3, and a FR-H4 as contained within a given V_{H} region amino acid sequence and defined by any of the aforementioned schemes, such as Kabat, Chothia, AbM, IgBLAST, IMGT, or Contact method, or other known schemes, and a FR-L1, a FR-L2, a FR-L3, and a FR-L4 as contained within a given V_{L} region amino acid sequence and defined by any of the aforementioned schemes, such as Kabat, Chothia, AbM, IgBLAST, IMGT, or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the provided antibodies are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multispecific antibodies formed from antibody fragments. In some embodiments, the antibody is or comprises an antibody fragment comprising a variable heavy chain (V_{H}) and a variable light chain (V_{L}) region. In particular embodiments, the antibodies are single-chain antibody fragments comprising a heavy chain variable (V_{H}) region and/or a light chain variable (V_{L}) region, such as scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided anti-DLL3 antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

The terms “polypeptide” and “protein” are used interchangeably to refer to a polymer of amino acid residues, and are not limited to a minimum length. Polypeptides, including the provided antibodies and antibody chains and other polypeptides, e.g., linkers and DLL3-binding polypeptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

In some of any of the provided anti-DLL3 antibodies or antigen-binding fragments thereof, contain a heavy and/or light chain variable (V_{H} or V_{L}) region sequence as described, or a sufficient antigen-binding portion thereof. In some embodiments, the antibody, e.g., the anti-DLL3 antibody, e.g., antigen-binding antibody fragment, comprises a heavy chain variable region (V_{H}) sequence and/or a light chain variable region (V_{L}) sequence as described, or a sufficient antigen-binding portion thereof. In some embodiments, the antibody, e.g., the anti-DLL3 antibody, e.g., antigen-binding antibody fragment, is a single chain fragment, such as a single chain Fv (scFv) fragment. In some aspects, the scFv comprises a V_{H} region and a V_{L} region. In some embodiments, the antibody, e.g., the anti-DLL3 antibody, e.g., antigen-binding antibody fragment, is a single domain antibody (sdAb), such as an antibody that comprises a V_{H} region only.

In some embodiments, the antibody or antigen-binding fragment, such as an extracellular antigen-binding domain, comprised in any of the provided recombinant receptors, such as a chimeric antigen receptor (CAR; e.g., anti-DLL3 CAR), comprises a heavy chain or a sufficient antigen-binding portion thereof. For example, in some embodiments, the antibody or antigen-binding fragment thereof comprises a variable heavy chain (V_{H}) region, or a sufficient antigen-binding portion of a V_{H} region. In some embodiments, the antibody or antigen-binding fragment, such as an extracellular antigen-binding domain, comprised in any of the provided recombinant receptors, such as a chimeric antigen receptor (CAR; e.g., anti-DLL3 CAR), comprises a light chain or a sufficient antigen-binding portion thereof. For example, in some embodiments, the antibody or antigen-binding fragment thereof comprises a variable light chain (V_{L}) region, or a sufficient antigen-binding portion of a V_{L} region. In some embodiments, the antibody or antigen-binding fragment, such as an extracellular antigen-binding domain, comprised in any of the provided recombinant receptors, such as a chimeric antigen receptor (CAR; e.g., anti-DLL3 CAR), comprises a V_{H} region or a sufficient antigen-binding portion of a V_{H} region, and a V_{L} region or a sufficient antigen-binding portion of a V_{L} region. In some embodiments, the antibody or antigen-binding fragment thereof, such as the antigen-binding fragment of a recombinant receptor, e.g., CAR, comprises a V_{H} region and a V_{L} region, or a sufficient antigen-binding portion of a V_{H} and V_{L} region. In any such embodiments, a V_{H} region sequence can be any of the V_{H} region sequence described herein. In any such embodiments, a V_{L} region sequence can be any of the V_{L} region sequence described herein. In any such embodiments, any of the V_{H} region sequence and any of the V_{L} region sequence described herein can be used in combination. In some of any such embodiments, any one or more of the CDR-H1, the CDR-H2 and/or the CDR-H3 sequences described herein, and any one or more of the CDR-L1, the CDR-L2 and/or the CDR-L3 sequences described herein can be used in combination. In some such embodiments, the antibody is an antigen-binding fragment, such as a Fab or an scFv. In some embodiments, the antibody or antigen-binding fragment further comprises at least a portion of an immunoglobulin constant region or a variant thereof, such as the antigen-binding fragment of a recombinant receptor, e.g., CAR,. In some such embodiments, the antibody is a full-length antibody that also comprises a constant region.

In some embodiments, a recombinant receptor, e.g., a CAR provided herein, comprises an antibody such as an anti-DLL3 antibody, or antigen-binding fragment thereof that comprises any of comprises a V_{H} region or a sufficient antigen-binding portion thereof. In some embodiments, a recombinant receptor, e.g., a CAR provided herein, comprises an antibody such as an anti-DLL3 antibody, or antigen-binding fragment thereof that comprises any of comprises a V_{L} region or a sufficient antigen-binding portion thereof. For example, in some embodiments, the CAR comprises an antibody or antigen-binding fragment thereof that comprises a V_{H} region and a V_{L} region, or a sufficient antigen-binding portion of a V_{H} and V_{L} region. In any such embodiments, a V_{L} region sequence can be any of the V_{L} region sequence described herein. In any such embodiments, a V_{H} region sequence can be any of the V_{H} region sequence described herein. In any such embodiments, any of the V_{H} region sequence and any of the V_{L} region sequence described herein can be used in combination. In some of any such embodiments, any one or more of the CDR-H1, the CDR-H2 and/or the CDR-H3 sequences described herein, and any one or more of the CDR-L1, the CDR-L2 and/or the CDR-L3 sequences described herein can be used in combination. In some such embodiments, the antibody contained in the provided recombinant receptor is an antigen-binding fragment, such as a Fab or an scFv. In some such embodiments, the receptor, e.g., CAR, further comprises a spacer, such as a portion of an immunoglobulin constant region or a variant thereof, for example, as described below in Section I.B.

In some embodiments, the anti-DLL3 antibody, e.g., antigen-binding antibody fragment, comprises a V_{H} region sequence or sufficient antigen-binding portion thereof that comprises a heavy chain complementarity determining region 1 (CDR-H1), a heavy chain complementarity determining region 2 (CDR-H2) and/or a heavy chain complementarity determining region 3 (CDR-H3) contained therein and/or as described. In some embodiments, the anti-DLL3 antibody, e.g., antigen-binding antibody fragment, comprises a V_{H} region sequence or sufficient antigen-binding portion thereof that comprises a CDR-H1, a CDR-H2 and a CDR-H3 contained therein and/or as described. In some embodiments, the anti-DLL3 antibody, e.g., antigen-binding antibody fragment, comprises a V_{L} region sequence or sufficient antigen-binding portion that comprises a light chain complementarity determining region 1 (CDR-L1), a light chain complementarity determining region 2 (CDR-L2), and/or a light chain complementarity determining region 3 (CDR-L3) contained therein and/or as described. In some embodiments, the anti-DLL3 antibody, e.g., antigen-binding antibody fragment, comprises a V_{L} region sequence or sufficient antigen-binding portion that comprises a CDR-L1, a CDR-L2 and a CDR-L3 contained therein and/or as described.

In some embodiments, the anti-DLL3 antibody, e.g., antigen-binding antibody fragment, comprises a V_{H} region sequence that comprises a CDR-H1, a CDR-H2 and/or a CDR-H3 as described and comprises a V_{L} region sequence that comprises a CDR-L1, a CDR-L2 and/or a CDR-L3 as described. In some embodiments, the anti-DLL3 antibody, e.g., antigen-binding antibody fragment, comprises a V_{H} region sequence that comprises a CDR-H1, a CDR-H2 and a CDR-H3 as described and comprises a V_{L} region sequence that comprises a CDR-L1, a CDR-L2 and a CDR-L3 as described. Also among the provided antibodies and fragment thereof are those having sequences at least at or about 95%, at or about 96%, at or about 97%, at or about 98%, or at or about 99% identical to such a sequence, e.g., any of the CDR-H1, CDR-H2, CDR-H3, CDR-L1, CDR-L2, CDR-L3, V_{H}, V_{L}, scFv sequences or other sequences of the antibodies of fragment thereof described herein. In some aspects, the provided antibodies or fragment thereof have at least at or about 95% sequence identity to any such sequences. In some aspects, the provided antibodies or fragment thereof have at least at or about 96% sequence identity to any such sequences. In some aspects, the provided antibodies or fragment thereof have at least at or about 97% sequence identity to any such sequences. In some aspects, the provided antibodies or fragment thereof have at least at or about 98% sequence identity to any such sequences. In some aspects, the provided antibodies or fragment thereof have at least at or about 99% sequence identity to any such sequences.

In some embodiments, the antibody is an sdAb comprising only a V_{H} region sequence or a sufficient antigen-binding portion thereof, such as any of the V_{H} sequences described herein (e.g., a CDR-H1, a CDR-H2, a CDR-H3 and/or a CDR-H4). In some embodiments, the antibodies or antigen-binding fragments include those that are single domain antibodies, containing a V_{H} region that, without pairing with a V_{L} region) and/or without any additional antibody domain or binding site, are capable of specifically binding to DLL3.

In some embodiments, the V_{H} region of an antibody or antigen-binding fragment thereof comprises a CDR-H1, a CDR-H2 and/or a CDR-H3 according to Kabat numbering. In some embodiments, the V_{H} region of an antibody or antigen-binding fragment thereof comprises a CDR-H1, a CDR-H2 and/or a CDR-H3 according to Chothia numbering. In some embodiments, the V_{H} region of an antibody or antigen-binding fragment thereof comprises a CDR-H1, a CDR-H2 and/or a CDR-H3 according to AbM numbering. In some embodiments, the V_{H} region of an antibody or antigen-binding fragment thereof comprises a CDR-H1, a CDR-H2 and/or a CDR-H3 according to Kabat, Chothia, AbM, or IMGT numbering, or other numbering schemes.

In some of any of the provided embodiments, the binding molecule, such as an antibody or an antigen-binding fragment thereof, has a heavy chain variable (V_{H}) region that comprises a heavy chain complementarity determining region 1 (CDR-H1), a heavy chain complementarity determining region 2 (CDR-H2) and/or a heavy chain complementarity determining region 3 (CDR-H3) present in SEQ ID NO:90, such as a CDR-H1, a CDR-H2 and/or a CDR-H3 according to Kabat, Chothia, AbM, or IMGT numbering, or other numbering schemes. In some of any of the provided embodiments, the binding molecule, such as an antibody or an antigen-binding fragment thereof, has a V_{H} that comprises a CDR-H1, a CDR-H2, and a CDR-H3 present in SEQ ID NO:90, such as a CDR-H1, a CDR-H2 and a CDR-H3 according to Kabat, Chothia, AbM, or IMGT numbering, or other numbering schemes.

In some of any of the provided embodiments, the binding molecule, such as an antibody or an antigen-binding fragment thereof, comprises a V_{H} region having the amino acid sequence set forth in SEQ ID NO:90, or an amino acid sequence that has at least at or about 95%, 96%, 97%, 98%, or 99% sequence identity to the V_{H} region amino acid sequence set forth in SEQ ID NO:90, and/or comprises a CDR-H1, a CDR-H2, and a CDR-H3 present in any of the foregoing V_{H} sequences, such as one that comprises a CDR-H1, a CDR-H2, and a CDR-H3 present in any of the foregoing V_{H} sequences. In some of any of the provided embodiments, the V_{H} region comprises an amino acid sequence that has at least at or about 95%, 96%, 97%, 98%, or 99% sequence identity to the V_{H} region amino acid sequence set forth in SEQ ID NO:90. In some of any of the provided embodiments, the binding molecule, such as an antibody or an antigen-binding fragment thereof, comprises a V_{H} region having the amino acid sequence set forth in SEQ ID NO:90, or an amino acid sequence that has at least at or about 95%, 96%, 97%, 98%, or 99% sequence identity to the V_{H} region amino acid sequence set forth in SEQ ID NO:90, or comprises a CDR-H1, a CDR-H2, and a CDR-H3 present in SEQ ID NO:90, such as one that comprises a CDR-H1, a CDR-H2, and a CDR-H3 present in SEQ ID NO:90. In some of any of the provided embodiments, the V_{H} region comprises a CDR-H1, a CDR-H2 and a CDR-H3, respectively, comprising the amino acid sequence of a CDR-H1, a CDR-H2 and a CDR-H3 contained within the V_{H} region amino acid sequence set forth in SEQ ID NO:90. In some of any of the provided embodiments, the V_{H} region comprises a CDR-H1, a CDR-H2 and a CDR-H3 contained within SEQ ID NO:90.

In some of any of the provided embodiments, the V_{H} region comprises a CDR-H1 comprising the sequence set forth in SEQ ID NO:92. In some of any of the provided embodiments, the V_{H} region comprises a CDR-H2 comprising the sequence set forth in SEQ ID NO:93. In some of any of the provided embodiments, the V_{H} region comprises a CDR-H3 comprising the sequence set forth in SEQ ID NO:94. In some of any of the provided embodiments, the V_{H} region comprises a CDR-H1 comprising the sequence set forth in SEQ ID NO:92, a CDR-H2 comprising the sequence set forth in SEQ ID NO:93, and a CDR-H3 comprising the sequence set forth in SEQ ID NO:94. In some of any of the provided embodiments, the V_{H} region comprises a CDR-H1, a CDR-H2 and a CDR-H3 comprising the sequence set forth in SEQ ID NOS:92, 93, and 94, respectively.

In some of any of the provided embodiments, the V_{H} region comprises a CDR-H1 comprising the sequence set forth in SEQ ID NO:98. In some of any of the provided embodiments, the V_{H} region comprises a CDR-H2 comprising the sequence set forth in SEQ ID NO:99. In some of any of the provided embodiments, the V_{H} region comprises a CDR-H3 comprising the sequence set forth in SEQ ID NO:100. In some of any of the provided embodiments, the V_{H} region comprises a CDR-H1 comprising the sequence set forth in SEQ ID NO:98, a CDR-H2 comprising the sequence set forth in SEQ ID NO:99, and a CDR-H3 comprising the sequence set forth in SEQ ID NO:100. In some of any of the provided embodiments, the V_{H} region comprises a CDR-H1, a CDR-H2 and a CDR-H3 comprising the sequence set forth in SEQ ID NOS:98, 99, and 100, respectively.

In some of any of the provided embodiments, the V_{H} region comprises a CDR-H1 comprising the sequence set forth in SEQ ID NO:104. In some of any of the provided embodiments, the V_{H} region comprises a CDR-H2 comprising the sequence set forth in SEQ ID NO:105. In some of any of the provided embodiments, the V_{H} region comprises a CDR-H3 comprising the sequence set forth in SEQ ID NO:106. In some of any of the provided embodiments, the V_{H} region comprises a CDR-H1 comprising the sequence set forth in SEQ ID NO:104, a CDR-H2 comprising the sequence set forth in SEQ ID NO:105, and a CDR-H3 comprising the sequence set forth in SEQ ID NO:106. In some of any of the provided embodiments, the V_{H} region comprises a CDR-H1, a CDR-H2 and a CDR-H3 comprising the sequence set forth in SEQ ID NOS:104, 105, and 106, respectively.

In some of any of the provided embodiments, the V_{H} region comprises a CDR-H1 comprising the sequence set forth in SEQ ID NO:110. In some of any of the provided embodiments, the V_{H} region comprises a CDR-H2 comprising the sequence set forth in SEQ ID NO:111. In some of any of the provided embodiments, the V_{H} region comprises a CDR-H3 comprising the sequence set forth in SEQ ID NO:112. In some of any of the provided embodiments, the V_{H} region comprises a CDR-H1 comprising the sequence set forth in SEQ ID NO:110, a CDR-H2 comprising the sequence set forth in SEQ ID NO:111, and a CDR-H3 comprising the sequence set forth in SEQ ID NO:112. In some of any of the provided embodiments, the V_{H} region comprises a CDR-H1, a CDR-H2 and a CDR-H3 comprising the sequence set forth in SEQ ID NOS:110, 111, and 112, respectively.

In some of any of the provided embodiments, the V_{H} region comprises any of the CDR-H1, the CDR-H2 and the CDR-H3 as described and comprises a framework region 1 (FR1), a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{H} region amino acid sequence set forth in SEQ ID NO:90.

In some of any embodiments, the V_{H} region comprises a FR1, a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{H} region amino acid sequence set forth in SEQ ID NO:90.

In some of any of the provided embodiments, the V_{H} region comprises the amino acid sequence set forth in SEQ ID NO:90. In some of any of the provided embodiments, the V_{H} region has the amino acid sequence set forth in SEQ ID NO:90.

In some of any of the provided embodiments, the V_{H} region comprises the amino acid sequence encoded by SEQ ID NO:87 or a nucleic acid sequence having at least at or about 90%, 91%, 92%, 93%, 94%, 95%, 96%, 97%, 98% or 99% sequence identity to SEQ ID NO:87.

In some of any of the provided embodiments, the V_{H} region comprises the amino acid sequence encoded by SEQ ID NO:87.

In some embodiments, the V_{L} region of an antibody or antigen-binding fragment thereof comprises a CDR-L1, a CDR-L2 and/or a CDR-L3 according to Kabat numbering. In some embodiments, the V_{L} region of an antibody or antigen-binding fragment thereof comprises a CDR-L1, a CDR-L2 and/or a CDR-L3 according to Chothia numbering. In some embodiments, the V_{L} region of an antibody or antigen-binding fragment thereof comprises a CDR-L1, a CDR-L2 and/or a CDR-L3 according to AbM numbering. In some embodiments, the V_{L} region of an antibody or antigen-binding fragment thereof comprises a CDR-L1, a CDR-L2 and/or a CDR-L3 according to Kabat, Chothia, AbM, or IMGT numbering, or other numbering schemes.

In some of any of the provided embodiments, the binding molecule, such as an antibody or an antigen-binding fragment thereof, has a light chain variable (V_{L}) region that comprises a light chain complementarity determining region 1 (CDR-L1), a light chain complementarity determining region 2 (CDR-L2) and/or a light chain complementarity determining region 3 (CDR-L3) present in SEQ ID NO:91, such as a CDR-L1, a CDR-L2 and/or a CDR-L3 according to Kabat, Chothia, AbM, or IMGT numbering, or other numbering schemes. In some of any of the provided embodiments, the binding molecule, such as an antibody or an antigen-binding fragment thereof, has a V_{L} that comprises a CDR-L1, a CDR-L2, and a CDR-L3 present in SEQ ID NO:91, such as a CDR-L1, a CDR-L2 and a CDR-L3 according to Kabat, Chothia, AbM, or IMGT numbering, or other numbering schemes.

In some of any of the provided embodiments, the binding molecule, such as an antibody or an antigen-binding fragment thereof, comprises a V_{L} region having the amino acid sequence set forth in SEQ ID NO:91, or an amino acid sequence that has at least at or about 85%, 86%, 87%, 88%, 89%, 90%, 91%, 92%, 93%, 94%, 95%, 96%, 97%, 98% or 99% sequence identity to the V_{L} region amino acid sequence set forth in SEQ ID NO:91, and/or comprises a CDR-L1, a CDR-L2, and a CDR-L3 present in any of the foregoing V_{L} sequences, such as one that comprises a CDR-L1, a CDR-L2, and a CDR-L3 present in any of the foregoing V_{L} sequences. In some of any of the provided embodiments, the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to the V_{L} region amino acid sequence set forth in SEQ ID NO:91. In some of any of the provided embodiments, the binding molecule, such as an antibody or an antigen-binding fragment thereof, comprises a V_{L} region having the amino acid sequence set forth in SEQ ID NO:91, or an amino acid sequence that has at least at or about 85%, 86%, 87%, 88%, 89%, 90%, 91%, 92%, 93%, 94%, 95%, 96%, 97%, 98% or 99% sequence identity to the V_{L} region amino acid sequence set forth in SEQ ID NO:91, or comprises a CDR-L1, a CDR-L2, and a CDR-L3 present in SEQ ID NO:91, such as one that comprises a CDR-L1, a CDR-L2, and a CDR-L3 present in SEQ ID NO:91. In some of any of the provided embodiments, the V_{L} region comprises a CDR-L1, a CDR-L2 and a CDR-L3, respectively, comprising the amino acid sequence of a CDR-L1, a CDR-L2 and a CDR-L3 contained within the V_{L} region amino acid sequence set forth in SEQ ID NO:91. In some of any of the provided embodiments, the V_{L} region comprises a CDR-L1, a CDR-L2 and a CDR-L3 contained within SEQ ID NO:91.

In some of any of the provided embodiments, the V_{L} region comprises a CDR-L1 comprising the sequence set forth in SEQ ID NO:95. In some of any of the provided embodiments, the V_{L} region comprises a CDR-L2 comprising the sequence set forth in SEQ ID NO:96. In some of any of the provided embodiments, the V_{L} region comprises a CDR-L3 comprising the sequence set forth in SEQ ID NO:97. In some of any of the provided embodiments, the V_{L} region comprises a CDR-L1 comprising the sequence set forth in SEQ ID NO:95, a CDR-L2 comprising the sequence set forth in SEQ ID NO:96, and a CDR-L3 comprising the sequence set forth in SEQ ID NO:97. In some of any of the provided embodiments, the V_{L} region comprises a CDR-L1, a CDR-L2 and a CDR-L3 comprising the sequence set forth in SEQ ID NOS:95, 96, and 97, respectively.

In some of any of the provided embodiments, the V_{L} region comprises a CDR-L1 comprising the sequence set forth in SEQ ID NO:101. In some of any of the provided embodiments, the V_{L} region comprises a CDR-L2 comprising the sequence set forth in SEQ ID NO:102. In some of any of the provided embodiments, the V_{L} region comprises a CDR-L3 comprising the sequence set forth in SEQ ID NO:103. In some of any of the provided embodiments, the V_{L} region comprises a CDR-L1 comprising the sequence set forth in SEQ ID NO:101, a CDR-L2 comprising the sequence set forth in SEQ ID NO:102, and a CDR-L3 comprising the sequence set forth in SEQ ID NO:103. In some of any of the provided embodiments, the V_{L} region comprises a CDR-L1, a CDR-L2 and a CDR-L3 comprising the sequence set forth in SEQ ID NOS:101, 102, and 103, respectively.

In some of any of the provided embodiments, the V_{L} region comprises a CDR-L1 comprising the sequence set forth in SEQ ID NO:107. In some of any of the provided embodiments, the V_{L} region comprises a CDR-L2 comprising the amino acid sequence GA as set forth in SEQ ID NO:108. In some of any of the provided embodiments, the V_{L} region comprises a CDR-L3 comprising the sequence set forth in SEQ ID NO:109. In some of any of the provided embodiments, the V_{L} region comprises a CDR-L1 comprising the sequence set forth in SEQ ID NO:107, a CDR-L2 comprising the amino acid sequence GA as set forth in SEQ ID NO:108, and a CDR-L3 comprising the sequence set forth in SEQ ID NO:109. In some of any of the provided embodiments, the V_{L} region comprises a CDR-L1, a CDR-L2 and a CDR-L3 comprising the sequence set forth in SEQ ID NO:107, the sequence GA set forth in SEQ ID NO: 108, and the sequence set forth in SEQ ID NO: 109, respectively.

In some of any of the provided embodiments, the V_{L} region comprises a CDR-L1 comprising the sequence set forth in SEQ ID NO:113. In some of any of the provided embodiments, the V_{L} region comprises a CDR-L2 comprising the sequence set forth in SEQ ID NO:114. In some of any of the provided embodiments, the V_{L} region comprises a CDR-L3 comprising the sequence set forth in SEQ ID NO:115. In some of any of the provided embodiments, the V_{L} region comprises a CDR-L1 comprising the sequence set forth in SEQ ID NO:113, a CDR-L2 comprising the sequence set forth in SEQ ID NO:114, and a CDR-L3 comprising the sequence set forth in SEQ ID NO:115. In some of any of the provided embodiments, the V_{L} region comprises a CDR-L1, a CDR-L2 and a CDR-L3 comprising the sequence set forth in SEQ ID NOS:113, 114, and 115, respectively.

In some of any of the provided embodiments, the V_{L} region comprises any of the CDR-L1, the CDR-L2 and the CDR-L3 as described and comprises a framework region 1 (FR1), a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{L} region amino acid sequence set forth in SEQ ID NO:91.

In some of any embodiments, the V_{L} region comprises a FR1, a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{L} region amino acid sequence set forth in SEQ ID NO:91.

In some of any of the provided embodiments, the V_{L} region comprises the amino acid sequence set forth in SEQ ID NO:91. In some of any of the provided embodiments, the V_{L} region has the amino acid sequence set forth in SEQ ID NO:91.

In some of any of the provided embodiments, the V_{L} region comprises the amino acid sequence encoded by SEQ ID NO:88 or a nucleic acid sequence having at least at or about 80%, 85%, 86%, 87%, 88%, 89%, 90%, 91%, 92%, 93%, 94%, 95%, 96%, 97%, 98% or 99% sequence identity to SEQ ID NO:88. In some of any of the provided embodiments, the V_{L} region comprises the amino acid sequence encoded by SEQ ID NO:88.

In some embodiments, the V_{H} region of the antibody or antigen-binding fragment thereof, of any of the provided recombinant receptors, e.g., CAR, conjugates or binding molecules comprise the amino acid sequence of SEQ ID NO:90 and the V_{L} regions of the antibody or antigen-binding fragment thereof, of any of the provided recombinant receptors, e.g., CAR, conjugates or binding molecules comprise the amino acid sequence of SEQ ID NO:91.

In some embodiments, the V_{H} and V_{L} regions of the antibody or antigen-binding fragment thereof, of any of the provided recombinant receptors, e.g., CAR, conjugates or binding molecules comprise the amino acid sequences of SEQ ID NOS:90 and 91, respectively, respectively, or a sequence that has at least at or about 90% sequence identity to any of the above V_{H} and V_{L}, such as at least at or about 90%, 91%, 92%, 93%, 94%, 95%, 96%, 97%, 98%, or 99% sequence identity thereto. For example, the V_{H} and V_{L} regions of the antibody or antigen-binding fragment thereof provided therein comprise the amino acid sequences set forth in SEQ ID NOS:90 and 91, respectively.

In some of any of the provided embodiments, the V_{H} region and the V_{L} region are or comprise the sequence set forth in SEQ ID NOS:90 and 91, respectively.

In some of any of the provided embodiments, the binding molecule, such as an antibody or an antigen-binding fragment thereof, has a heavy chain variable (V_{H}) region that comprises a heavy chain complementarity determining region 1 (CDR-H1), a heavy chain complementarity determining region 2 (CDR-H2) and/or a heavy chain complementarity determining region 3 (CDR-H3) present in SEQ ID NO:90, such as a CDR-H1, a CDR-H2 and/or a CDR-H3 according to Kabat, Chothia, AbM, or IMGT numbering, or other numbering schemes, and a light chain variable (V_{L}) region that comprises a light chain complementarity determining region 1 (CDR-L1), a light chain complementarity determining region 2 (CDR-L2) and/or a light chain complementarity determining region 3 (CDR-L3) present in SEQ ID NO:91, such as a CDR-L1, a CDR-L2 and/or a CDR-L3 according to Kabat, Chothia, AbM, or IMGT numbering, or other numbering schemes. In some of any of the provided embodiments, the binding molecule, such as an antibody or an antigen-binding fragment thereof, has a V_{H} that comprises a CDR-H1, a CDR-H2, and a CDR-H3 present in SEQ ID NO:90, such as a CDR-H1, a CDR-H2 and a CDR-H3 according to Kabat, Chothia, AbM, or IMGT numbering, or other numbering schemes, and a V_{L} that comprises a CDR-L1, a CDR-L2, and a CDR-L3 present in SEQ ID NO:91, such as a CDR-L1, a CDR-L2 and a CDR-L3 according to Kabat, Chothia, AbM, or IMGT numbering, or other numbering schemes.

In some of any of the provided embodiments, the binding molecule, such as an antibody or an antigen-binding fragment thereof, comprises a V_{H} region having the amino acid sequence set forth in SEQ ID NO:90, or an amino acid sequence that has at least at or about 95%, 96%, 97%, 98%, or 99% sequence identity to the V_{H} region amino acid sequence set forth in SEQ ID NO:90, and/or comprises a CDR-H1, a CDR-H2, and a CDR-H3 present in any of the foregoing V_{H} sequences, such as one that comprises a CDR-H1, a CDR-H2, and a CDR-H3 present in any of the foregoing V_{H} sequences, and a V_{L} region having the amino acid sequence set forth in SEQ ID NO:91, or an amino acid sequence that has at least at or about 85%, 86%, 87%, 88%, 89%, 90%, 91%, 92%, 93%, 94%, 95%, 96%, 97%, 98% or 99% sequence identity to the V_{L} region amino acid sequence set forth in SEQ ID NO:91, and/or comprises a CDR-L1, a CDR-L2, and a CDR-L3 present in any of the foregoing V_{L} sequences, such as one that comprises a CDR-L1, a CDR-L2, and a CDR-L3 present in any of the foregoing V_{L} sequences. In some of any of the provided embodiments, the V_{H} region comprises an amino acid sequence that has at least at or about 95%, 96%, 97%, 98%, or 99% sequence identity to the V_{H} region amino acid sequence set forth in SEQ ID NO:90, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to the V_{L} region amino acid sequence set forth in SEQ ID NO:91. In some of any of the provided embodiments, the binding molecule, such as an antibody or an antigen-binding fragment thereof, comprises a V_{H} region having the amino acid sequence set forth in SEQ ID NO:90, or an amino acid sequence that has at least at or about 95%, 96%, 97%, 98%, or 99% sequence identity to the V_{H} region amino acid sequence set forth in SEQ ID NO:90, or comprises a CDR-H1, a CDR-H2, and a CDR-H3 present in SEQ ID NO:90, such as one that comprises a CDR-H1, a CDR-H2, and a CDR-H3 present in SEQ ID NO:90, and a V_{L} region having the amino acid sequence set forth in SEQ ID NO:91, or an amino acid sequence that has at least at or about 85%, 86%, 87%, 88%, 89%, 90%, 91%, 92%, 93%, 94%, 95%, 96%, 97%, 98% or 99% sequence identity to the V_{L} region amino acid sequence set forth in SEQ ID NO:91, or comprises a CDR-L1, a CDR-L2, and a CDR-L3 present in SEQ ID NO:91, such as one that comprises a CDR-L1, a CDR-L2, and a CDR-L3 present in SEQ ID NO:91. In some of any of the provided embodiments, the V_{H} region comprises a CDR-H1, a CDR-H2 and a CDR-H3, respectively, comprising the amino acid sequence of a CDR-H1, a CDR-H2 and a CDR-H3 contained within the V_{H} region amino acid sequence set forth in SEQ ID NO:90. In some of any of the provided embodiments, the V_{H} region comprises a CDR-H1, a CDR-H2 and a CDR-H3 contained within SEQ ID NO:90, and the V_{L} region comprises a CDR-L1, a CDR-L2 and a CDR-L3, respectively, comprising the amino acid sequence of a CDR-L1, a CDR-L2 and a CDR-L3 contained within the V_{L} region amino acid sequence set forth in SEQ ID NO:91. In some of any of the provided embodiments, the V_{L} region comprises a CDR-L1, a CDR-L2 and a CDR-L3 contained within SEQ ID NO:91.

In some of any of the provided embodiments, the V_{H} region comprises a CDR-H1 comprising the sequence set forth in SEQ ID NO:92, and the V_{L} region comprises a CDR-L1 comprising the sequence set forth in SEQ ID NO:95. In some of any of the provided embodiments, the V_{H} region comprises a CDR-H2 comprising the sequence set forth in SEQ ID NO:93, and the V_{L} region comprises a CDR-L2 comprising the sequence set forth in SEQ ID NO:96. In some of any of the provided embodiments, the V_{H} region comprises a CDR-H3 comprising the sequence set forth in SEQ ID NO:94, and the V_{L} region comprises a CDR-L3 comprising the sequence set forth in SEQ ID NO:97. In some of any of the provided embodiments, the V_{H} region comprises a CDR-H1 comprising the sequence set forth in SEQ ID NO:92, a CDR-H2 comprising the sequence set forth in SEQ ID NO:93, and a CDR-H3 comprising the sequence set forth in SEQ ID NO:94, and the V_{L} region comprises a CDR-L1 comprising the sequence set forth in SEQ ID NO:95, a CDR-L2 comprising the sequence set forth in SEQ ID NO:96, and a CDR-L3 comprising the sequence set forth in SEQ ID NO:97. In some of any of the provided embodiments, the V_{H} region comprises a CDR-H1, a CDR-H2 and a CDR-H3 comprising the sequence set forth in SEQ ID NOS:92, 93, and 94, respectively, and the V_{L} region comprises a CDR-L1, a CDR-L2 and a CDR-L3 comprising the sequence set forth in SEQ ID NOS:95, 96, and 97, respectively.

In some of any of the provided embodiments, the V_{H} region comprises a CDR-H1 comprising the sequence set forth in SEQ ID NO:98, and the V_{L} region comprises a CDR-L1 comprising the sequence set forth in SEQ ID NO:101. In some of any of the provided embodiments, the V_{H} region comprises a CDR-H2 comprising the sequence set forth in SEQ ID NO:99, and the V_{L} region comprises a CDR-L2 comprising the sequence set forth in SEQ ID NO:102. In some of any of the provided embodiments, the V_{H} region comprises a CDR-H3 comprising the sequence set forth in SEQ ID NO:100, and the V_{L} region comprises a CDR-L3 comprising the sequence set forth in SEQ ID NO:103. In some of any of the provided embodiments, the V_{H} region comprises a CDR-H1 comprising the sequence set forth in SEQ ID NO:98, a CDR-H2 comprising the sequence set forth in SEQ ID NO:99, and a CDR-H3 comprising the sequence set forth in SEQ ID NO:100, and the V_{L} region comprises a CDR-L1 comprising the sequence set forth in SEQ ID NO:101, a CDR-L2 comprising the sequence set forth in SEQ ID NO:102, and a CDR-L3 comprising the sequence set forth in SEQ ID NO:103. In some of any of the provided embodiments, the V_{H} region comprises a CDR-H1, a CDR-H2 and a CDR-H3 comprising the sequence set forth in SEQ ID NOS:98, 99, and 100, respectively, and the V_{L} region comprises a CDR-L1, a CDR-L2 and a CDR-L3 comprising the sequence set forth in SEQ ID NOS:101, 102, and 103, respectively.

In some of any of the provided embodiments, the V_{H} region comprises a CDR-H1 comprising the sequence set forth in SEQ ID NO:104, and the V_{L} region comprises a CDR-L1 comprising the sequence set forth in SEQ ID NO:107. In some of any of the provided embodiments, the V_{H} region comprises a CDR-H2 comprising the sequence set forth in SEQ ID NO:105, and the V_{L} region comprises a CDR-L2 comprising the amino acid sequence GA as set forth in SEQ ID NO:108. In some of any of the provided embodiments, the V_{H} region comprises a CDR-H3 comprising the sequence set forth in SEQ ID NO:106, and the V_{L} region comprises a CDR-L3 comprising the sequence set forth in SEQ ID NO:109. In some of any of the provided embodiments, the V_{H} region comprises a CDR-H1 comprising the sequence set forth in SEQ ID NO:104, a CDR-H2 comprising the sequence set forth in SEQ ID NO:105, and a CDR-H3 comprising the sequence set forth in SEQ ID NO:106, and the V_{L} region comprises a CDR-L1 comprising the sequence set forth in SEQ ID NO:107, a CDR-L2 comprising the amino acid sequence GA as set forth in SEQ ID NO:108, and a CDR-L3 comprising the sequence set forth in SEQ ID NO:109. In some of any of the provided embodiments, the V_{H} region comprises a CDR-H1, a CDR-H2 and a CDR-H3 comprising the sequence set forth in SEQ ID NOS:104, 105, and 106, respectively, and the V_{L} region comprises a CDR-L1, a CDR-L2 and a CDR-L3 comprising the sequence set forth in SEQ ID NO:107, the sequence GA set forth in SEQ ID NO:108, and the sequence set forth in SEQ ID NO: 109, respectively.

In some of any of the provided embodiments, the V_{H} region comprises a CDR-H1 comprising the sequence set forth in SEQ ID NO:110, and the V_{L} region comprises a CDR-L1 comprising the sequence set forth in SEQ ID NO:113. In some of any of the provided embodiments, the V_{H} region comprises a CDR-H2 comprising the sequence set forth in SEQ ID NO:111, and the V_{L} region comprises a CDR-L2 comprising the sequence set forth in SEQ ID NO:114. In some of any of the provided embodiments, the V_{H} region comprises a CDR-H3 comprising the sequence set forth in SEQ ID NO:112, and the V_{L} region comprises a CDR-L3 comprising the sequence set forth in SEQ ID NO:115. In some of any of the provided embodiments, the V_{H} region comprises a CDR-H1 comprising the sequence set forth in SEQ ID NO:110, a CDR-H2 comprising the sequence set forth in SEQ ID NO:111, and a CDR-H3 comprising the sequence set forth in SEQ ID NO:112, and the V_{L} region comprises a CDR-L1 comprising the sequence set forth in SEQ ID NO:113, a CDR-L2 comprising the sequence set forth in SEQ ID NO:114, and a CDR-L3 comprising the sequence set forth in SEQ ID NO:115. In some of any of the provided embodiments, the V_{H} region comprises a CDR-H1, a CDR-H2 and a CDR-H3 comprising the sequence set forth in SEQ ID NOS:110, 111, and 112, respectively, and the V_{L} region comprises a CDR-L1, a CDR-L2 and a CDR-L3 comprising the sequence set forth in SEQ ID NOS:113, 114, and 115, respectively.

In some of any of the provided embodiments, the V_{H} region comprises any of the CDR-H1, the CDR-H2 and the CDR-H3 as described and comprises a framework region 1 (FR1), a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{H} region amino acid sequence set forth in SEQ ID NO:90. In some of any embodiments, the V_{H} region comprises a FR1, a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{H} region amino acid sequence set forth in SEQ ID NO:90, and the V_{L} region comprises any of the CDR-L1, the CDR-L2 and the CDR-L3 as described and comprises a framework region 1 (FR1), a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{L} region amino acid sequence set forth in SEQ ID NO:91. In some of any embodiments, the V_{L} region comprises a FR1, a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{L} region amino acid sequence set forth in SEQ ID NO:91.

In some of any of the provided embodiments, the V_{H} region comprises the amino acid sequence set forth in SEQ ID NO:90, and the V_{L} region comprises the amino acid sequence set forth in SEQ ID NO:91. In some of any of the provided embodiments, the V_{H} region has the amino acid sequence set forth in SEQ ID NO:90, and the V_{L} region has the amino acid sequence set forth in SEQ ID NO:91.

In some of any of the provided embodiments, the V_{H} region comprises the amino acid sequence encoded by SEQ ID NO:87 or a nucleic acid sequence having at least at or about 90%, 91%, 92%, 93%, 94%, 95%, 96%, 97%, 98% or 99% sequence identity to SEQ ID NO:87, and the V_{L} region comprises the amino acid sequence encoded by SEQ ID NO:88 or a nucleic acid sequence having at least at or about 90%, 91%, 92%, 93%, 94%, 95%, 96%, 97%, 98% or 99% sequence identity to SEQ ID NO:88. In some of any of the provided embodiments, the V_{H} region comprises the amino acid sequence encoded by SEQ ID NO:87, and the V_{L} region comprises the amino acid sequence encoded by SEQ ID NO:88.

Also provided are polynucleotides that contain any of the nucleotide sequences described herein, e.g., encoding all of a portion of the provided binding molecules.

In some embodiments, the antibody or antigen-binding fragment thereof, for example, in any of the recombinant receptors provided herein, such as a CAR, is a single-chain antibody fragment, such as a single chain variable fragment (scFv) or a diabody or a single domain antibody (sdAb). In some embodiments, the antibody or antigen-binding fragment is a multi-domain antibody, such as an scFv comprising a heavy chain variable (V_{H}) region and a light chain variable (V_{L}) region. In some embodiments, the single-chain antibody fragment (e.g., scFv) includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline.

Accordingly, the provided CARs contain anti-DLL3 antibodies that include single-chain antibody fragments, such as scFvs and diabodies, particularly human single-chain antibody fragments, typically comprising linker(s) joining two antibody domains or regions, such V_{H} and V_{L} regions. The linker typically is a peptide linker, e.g., a flexible and/or soluble peptide linker, such as one rich in glycine and serine.

In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between 5 and 50 amino acids in length, typically between 10 and 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO:44) or GGGS (3GS; SEQ ID NO:45), such as between 2, 3, 4 and 5 repeats of such a sequence. Exemplary linkers include those having or consisting of the sequence set forth in SEQ ID NO:46 (GGGGSGGGGSGGGGS). Exemplary linkers further include those having or consisting of the sequence set forth in SEQ ID NO:31 (GSTSGSGKPGSGEGSTKG). Exemplary linkers further include those having or consisting of the sequence set forth in SEQ ID NO:68 (SRGGGGSGGGGSGGGGSLEMA). An exemplary linker includes those having or consisting of the sequence set forth in SEQ ID NO:69 (GSRGGGGSGGGGSGGGGSLEMA). Exemplary linkers further include those having or consisting of the sequence set forth in SEQ ID NOs: 29 and 30.

Accordingly, in some embodiments, the provided embodiments include single-chain antibody fragments, e.g., scFvs, comprising one or more of the aforementioned linkers, such as glycine/serine rich linkers, including linkers having repeats of GGGS (SEQ ID NO:45) or GGGGS (SEQ ID NO:44), such as a linker set forth in any one of SEQ ID NO:46, 68 or 69. In some embodiments, the linker may be encoded in DNA and comprise the sequence set forth in SEQ ID NO: 47

In some embodiments, the V_{H} region may be amino terminal to the V_{L} region. In some embodiments, the V_{H} region may be carboxy terminal to the V_{L} region. In particular embodiments, the fragment, e.g., scFv, may include a V_{H} region or portion thereof, followed by the linker, followed by a V_{L} region or portion thereof. In other embodiments, the fragment, e.g., the scFv, may include the V_{L} region or portion thereof, followed by the linker, followed by the V_{H} region or portion thereof.

In some of any of the provided embodiments, the scFv comprises the sequence set forth in SEQ ID NO:89, or an amino acid sequence having at least at or about 95%, 96%, 97%, 98% or 99% sequence identity to SEQ ID NO:89. In some of any of the provided embodiments, the scFv comprises the sequence set forth in SEQ ID NO:89. In some of any of the provided embodiments, the scFv has the sequence set forth in SEQ ID NO:89. In some embodiments, the provided recombinant receptor, such as a CAR, comprises an extracellular antigen-binding domain comprising an scFv, and the scFv comprises the sequence set forth in SEQ ID NO:89, or an amino acid sequence having at least at or about 95%, 96%, 97%, 98% or 99% sequence identity to SEQ ID NO:89. In some embodiments, the provided recombinant receptor, such as a CAR, comprises an extracellular antigen-binding domain comprising an scFv, and the scFv comprises the sequence set forth in SEQ ID NO:89. In some embodiments, the provided recombinant receptor, such as a CAR, comprises an extracellular antigen-binding domain comprising an scFv, and the scFv has the sequence set forth in SEQ ID NO:89.

In some of any of the provided embodiments, the scFv comprises the amino acid sequence encoded by SEQ ID NO:86, or a nucleic acid sequence having at least at or about 90%, 91%, 92%, 93%, 94%, 95%, 96%, 97%, 98% or 99% sequence identity to SEQ ID NO:86. In some of any of the provided embodiments, the scFv comprises the amino acid sequence encoded by SEQ ID NO:86. In some of any of the provided embodiments, the scFv has the amino acid sequence encoded by SEQ ID NO:86. In some embodiments, the provided recombinant receptor, such as a CAR, comprises an extracellular antigen-binding domain comprising an scFv, and the scFv comprises the amino acid sequence encoded by SEQ ID NO:86, or a nucleic acid sequence having at least at or about 90%, 91%, 92%, 93%, 94%, 95%, 96%, 97%, 98% or 99% sequence identity to SEQ ID NO:86. In some embodiments, the provided recombinant receptor, such as a CAR, comprises an extracellular antigen-binding domain comprising an scFv, and the scFv comprises the amino acid sequence encoded by SEQ ID NO:86. In some embodiments, the provided recombinant receptor, such as a CAR, comprises an extracellular antigen-binding domain comprising an scFv, and the scFv has the amino acid sequence encoded by SEQ ID NO:86.

Table 2 provides the SEQ ID NOS: of exemplary provided antibodies or antigen-binding fragments, such as scFvs. In some aspects, the exemplary provided antibody fragments can be comprised in the provided DLL3-binding receptors, such as anti-DLL3 chimeric antigen receptors (CARs). In some embodiments, the DLL3-binding antibody or fragment thereof, such as an scFv, comprises a V_{H} region that comprises the CDR-H1, the CDR-H2 and the CDR-H3 sequence and a V_{L} region that comprises the CDR-L1, the CDR-L2 and the CDR-L3 sequence set forth in the SEQ ID NOS: listed in each row of Table 2 below (by Kabat, Chothia, AbM and IMGT numbering schemes). In some embodiments, the DLL3-binding antibody or fragment thereof, such as an scFv, comprises a V_{H} region sequence and a V_{L} region sequence set forth in the SEQ ID NOS: listed in each row of Table 2 below, or an antibody comprising a V_{H} region and a V_{L} region amino acid sequence that has at least at or about 95%, 96%, 97%, 98%, or 99% sequence identity to the V_{H} region sequence and the V_{L} region sequence set forth in the SEQ ID NOS: listed in each row of Table 2 below. In some embodiments, the DLL3-binding antibody or fragment thereof, such as an scFv, comprises a V_{H} region sequence and a V_{L} region sequence set forth in the SEQ ID NOS: listed in each row of Table 2 below. In some embodiments, the DLL3-binding antibody or fragment thereof comprises an scFv sequence set forth in the SEQ ID NOS: listed in each row of Table 2 below, or an antibody comprising an scFv amino acid sequence that has at least at or about 95%, 96%, 97%, 98%, or 99% sequence identity to the scFv sequence set forth in the SEQ ID NOS: listed in each row of Table 2 below. In some embodiments, the DLL3-binding antibody or fragment thereof comprises an scFv sequence set forth in the SEQ ID NOS: listed in each row of Table 2 below. In some embodiments, any of the antibody or antigen-binding fragment thereof, such as scFv, listed in each row of Table 2 can be comprised in a receptor, such as a chimeric antigen receptor (CAR), for example, as the extracellular antigen-binding domain.

In some embodiments, the provided antibody or antigen-binding fragment thereof comprises a V_{H} region and a V_{L} region, wherein the V_{H} region of the antibody or antigen-binding fragment thereof can contain a combination of any of the CDR-H1, the CDR-H2 and the CDR-H3 amino acid sequences set forth in Table 2, and the V_{L} region of the antibody or antigen-binding fragment thereof can contain a combination of any of the CDR-L1, the CDR-L2 and the CDR-L3 amino acid sequences set forth in Table 2. In some embodiments, the provided antibody or antigen-binding fragment thereof comprises a V_{H} region and/or a V_{L} region set forth in Table 2, in any combination, orientation or containing a different linker. In some aspects, the antibody or antigen-binding fragment thereof comprises a V_{H} region described in Table 2. In some aspects, the antibody or antigen-binding fragment thereof comprises a V_{L} region described in Table 2. In some embodiments, the provided antibody or antigen-binding fragment thereof is a single-domain antibody (sdAb), comprising a V_{H} region set forth in Table 2, or a V_{L} region set forth in Table 2. In some embodiments, the provided antibody or antigen-binding fragment thereof is a single-domain antibody (sdAb), comprising a V_{H} region containing a CDR-H1, a CDR-H2 and/or a CDR-H3 set forth in Table 2, or a V_{L} region containing a CDR-L1, a CDR-L2 and/or a CDR-L3 set forth in Table 2.

TABLE 2
Sequence identifier (SEQ ID NO:) for antibody fragments

| | CDR- | CDR- | CDR- | CDR- | CDR- | CDR- | | | | | |
|---|---|---|---|---|---|---|---|---|---|---|---|
| scFv | H1 | H2 | H3 | L1 | L2 | L3 | V_{H} | V_{L} | Linker | Order | scFv |
| | Kabat | | | Kabat | | | | | | | |
| DLL3- | 92 | 93 | 94 | 95 | 96 | 97 | 90 | 91 | 46 | V_{H}-V_{L} | 89 |
| mAb-1 | | | | | | | | | | | |
| | Chothia | | | Chothia | | | | | | | |
| DLL3- | 98 | 99 | 100 | 101 | 102 | 103 | 90 | 91 | 46 | V_{H}-V_{L} | 89 |
| mAb-1 | | | | | | | | | | | |
| | AbM | | | AbM | | | | | | | |
| DLL3- | 110 | 111 | 112 | 113 | 114 | 115 | 90 | 91 | 46 | V_{H}-V_{L} | 89 |
| mAb-1 | | | | | | | | | | | |
| | IGMT | | | IGMT | | | | | | | |
| DLL3- | 104 | 105 | 106 | 107 | 108 | 109 | 90 | 91 | 46 | V_{H}-V_{L} | 89 |
| mAb-1 | | | | | | | | | | | |

Among the provided antibodies, e.g., antigen-binding fragments, are human antibodies. In some embodiments of a provided human anti-DLL3 antibody, e.g., antigen-binding fragments, the human antibody comprises a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and/or comprises a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment.

Among the provided antibodies, e.g., antigen-binding fragments, are human antibodies. In some embodiments of a provided human anti-DLL3 antibody, e.g., antigen-binding fragments, the human antibody comprises a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and comprises a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment. In some embodiments, the portion of the V_{H} region corresponds to the CDR-H1, the CDR-H2 and/or the CDR-H3. In some embodiments, the portion of the V_{H} region corresponds to the CDR-H1, the CDR-H2 and the CDR-H3. In some embodiments, the portion of the V_{H} region corresponds to the framework region 1 (FR1), FR2, FR2 and/or FR4. In some embodiments, the portion of the V_{L} region corresponds to the CDR-L1, the CDR-L2 and/or the CDR-L3. In some embodiments, the portion of the V_{L} region corresponds to the CDR-L1, the CDR-L2 and the CDR-L3. In some embodiments, the portion of the V_{L} region corresponds to the FR1, FR2, FR2 and/or FR4.

In some embodiments, the human antibody or antigen-binding fragment thereof, comprises a CDR-H1 having at least 95%, 96%, 97%, 98%, 99%, or 100% sequence identity to an amino acid sequence of the corresponding CDR-H1 region within a sequence encoded by a germline nucleotide human heavy chain V segment. For example, the human antibody in some embodiments comprises a CDR-H1 having a sequence that is 100% identical or with no more than one, two or three amino acid differences as compared to the corresponding CDR-H1 region within a sequence encoded by a germline nucleotide human heavy chain V segment.

In some embodiments, the human antibody or antigen-binding fragment thereof, comprises a CDR-H2 having at least 95%, 96%, 97%, 98%, 99%, or 100% sequence identity to an amino acid sequence of the corresponding CDR-H2 region within a sequence encoded by a germline nucleotide human heavy chain V segment. For example, the human antibody in some embodiments comprises a CDR-H2 having a sequence that is 100% identical or with no more than one, two or three amino acid difference as compared to the corresponding CDR-H2 region within a sequence encoded by a germline nucleotide human heavy chain V segment.

In some embodiments, the human antibody or antigen-binding fragment thereof, comprises a CDR-H3 having at least 95%, 96%, 97%, 98%, 99%, or 100% sequence identity to an amino acid sequence of the corresponding CDR-H3 region within a sequence encoded by a germline nucleotide human heavy chain V segment, D segment and J segment. For example, the human antibody in some embodiments comprises a CDR-H3 having a sequence that is 100% identical or with no more than one, two or three amino acid differences as compared to the corresponding CDR-H3 region within a sequence encoded by a germline nucleotide human heavy chain V segment, D segment and J segment.

In some embodiments, the human antibody or antigen-binding fragment thereof, comprises a CDR-L1 having at least 95%, 96%, 97%, 98%, 99%, or 100% sequence identity to an amino acid sequence of the corresponding CDR-L1 region within a sequence encoded by a germline nucleotide human light chain V segment. For example, the human antibody in some embodiments comprises a CDR-L1 having a sequence that is 100% identical or with no more than one, two or three amino acid differences as compared to the corresponding CDR-L1 region within a sequence encoded by a germline nucleotide human light chain V segment.

In some embodiments, the human antibody or antigen-binding fragment thereof, comprises a CDR-L2 having at least 95%, 96%, 97%, 98%, 99%, or 100% sequence identity to an amino acid sequence of the corresponding CDR-L2 region within a sequence encoded by a germline nucleotide human light chain V segment. For example, the human antibody in some embodiments comprises a CDR-L2 having a sequence that is 100% identical or with no more than one, two or three amino acid difference as compared to the corresponding CDR-L2 region within a sequence encoded by a germline nucleotide human light chain V segment.

In some embodiments, the human antibody or antigen-binding fragment thereof, comprises a CDR-L3 having at least 95%, 96%, 97%, 98%, 99%, or 100% sequence identity to an amino acid sequence of the corresponding CDR-L3 region within a sequence encoded by a germline nucleotide human light chain V segment and J segment. For example, the human antibody in some embodiments comprises a CDR-L3 having a sequence that is 100% identical or with no more than one, two or three amino acid differences as compared to the corresponding CDR-L3 region within a sequence encoded by a germline nucleotide human light chain V segment and J segment.

In some embodiments, the human antibody or antigen-binding fragment thereof, comprises a framework region that comprises human germline gene segment sequences. For example, in some embodiments, the human antibody comprises a V_{H} region in which the framework region, e.g. FR1, FR2, FR3 and FR4, has at least 95%, 96%, 97%, 98%, 99%, or 100% sequence identity to a framework region encoded by a human germline antibody segment, such as a V segment and/or J segment. In some embodiments, the human antibody comprises a V_{L} region in which the framework region e.g. FR1, FR2, FR3 and FR4, has at least 95%, 96%, 97%, 98%, 99%, or 100% sequence identity to a framework region encoded by a human germline antibody segment, such as a V segment and/or J segment. For example, in some such embodiments, the framework region sequence contained within the V_{H} region and/or V_{L} region differs by no more than 10 amino acids, such as no more than 9, 8, 7, 6, 5, 4, 3, 2 or 1 amino acid, compared to the framework region sequence encoded by a human germline antibody segment.

The antibody or antigen-binding fragment thereof, may contain at least a portion of an immunoglobulin constant region, such as one or more constant region domain. In some embodiments, the antibody or antigen-binding fragment thereof comprises a light chain constant region. In some embodiments, the antibody or antigen-binding fragment thereof comprises a lambda (λ) light chain constant region. In some embodiments, the antibody or antigen-binding fragment thereof comprises a kappa (k) light chain constant region. In some embodiments, the antibody or antigen-binding fragment thereof comprises a heavy chain constant region. In some embodiments, the antibody or antigen-binding fragment thereof comprises a heavy chain constant region for an isotype class selected from among IgG, IgM, IgD, IgA and IgE. In some embodiments, the antibody or antigen-binding fragment thereof comprises an IgG heavy chain constant region. In some embodiments, the antibody or antigen-binding fragment thereof comprises an IgG1, IgG2, IgG3, or IgG4 heavy chain constant region. In some embodiments, the antibody or antigen-binding fragment thereof comprises an IgG1 heavy chain constant region.

In some embodiments, the constant regions include a light chain constant region and/or a heavy chain constant region 1 (C_{H}1). In some embodiments, the antibody includes at least a portion of a hinge region or a variant thereof. In some embodiments, the antibody includes a C_{H}2 and/or C_{H}3 domain, such as an Fc region. In some embodiments, the Fc region is an Fc region of a human IgG, such as an IgG1, IgG2, IgG3, or IgG4. In some embodiments, the Fc region is an Fc region of a human IgG1. In some embodiments, the Fc region is an Fc region of a human IgG2. In some embodiments, the Fc region is an Fc region of a human IgG3. In some embodiments, the Fc region is an Fc region of a human IgG4.

In some embodiments, the antibody or antigen-binding fragment thereof comprises a heavy chain constant region. In some embodiments, the antibody or antigen-binding fragment thereof comprises an IgG1 heavy chain constant region. In some embodiments, an exemplary human IgG1 heavy chain constant region comprises the sequence set forth in SEQ ID NO:126. In some embodiments, the antibody or antigen-binding fragment thereof comprises a light constant region. In some embodiments, the antibody or antigen-binding fragment thereof comprises a kappa light constant region. In some embodiments, an exemplary human kappa light chain constant region comprises the sequence set forth in SEQ ID NO:127.

In some of any of the provided embodiments, the antibody or antigen-binding fragment thereof comprises a heavy chain variable region (V_{H}) and a heavy chain constant region (C_{H}). In some aspects, the antibody or antigen-binding fragment thereof comprises a V_{H} comprising the sequence set forth in SEQ ID NO:90, or an amino acid sequence having at least at or about 95%, 96%, 97%, 98% or 99% sequence identity to SEQ ID NO:90, and a C_{H} comprising the sequence set forth in SEQ ID NO:126, or an amino acid sequence having at least at or about 95%, 96%, 97%, 98% or 99% sequence identity to SEQ ID NO:126. In some aspects, the antibody or antigen-binding fragment thereof comprises a heavy chain comprising the sequence set forth in SEQ ID NO:128, or an amino acid sequence having at least at or about 95%, 96%, 97%, 98% or 99% sequence identity to SEQ ID NO:128.

In some of any of the provided embodiments, the antibody or antigen-binding fragment thereof comprises a light chain variable region (V_{L}) and a light chain constant region (C_{L}). In some aspects, the antibody or antigen-binding fragment thereof comprises a V_{L} comprising the sequence set forth in SEQ ID NO:91, or an amino acid sequence having at least at or about 95%, 96%, 97%, 98% or 99% sequence identity to SEQ ID NO:91, and a C_{L} comprising the sequence set forth in SEQ ID NO:127, or an amino acid sequence having at least at or about 95%, 96%, 97%, 98% or 99% sequence identity to SEQ ID NO:127. In some aspects, the antibody or antigen-binding fragment thereof comprises a light chain comprising the sequence set forth in SEQ ID NO:129, or an amino acid sequence having at least at or about 95%, 96%, 97%, 98% or 99% sequence identity to SEQ ID NO:129.

In some of any of the provided embodiments, the antibody or antigen-binding fragment thereof comprises a full-length heavy chain comprising a heavy chain variable region (V_{H}) and a heavy chain constant region (C_{H}), and a full-length light chain comprising a light chain variable region (V_{L}) and a light chain constant region (C_{L}). In some embodiments, the antibody or antigen-binding fragment thereof comprises a V_{H} comprising the sequence set forth in SEQ ID NO:90, or an amino acid sequence having at least at or about 95%, 96%, 97%, 98% or 99% sequence identity to SEQ ID NO:90, and a C_{H} comprising the sequence set forth in SEQ ID NO:126, or an amino acid sequence having at least at or about 95%, 96%, 97%, 98% or 99% sequence identity to SEQ ID NO:126; and a V_{L} comprising the sequence set forth in SEQ ID NO:91, or an amino acid sequence having at least at or about 95%, 96%, 97%, 98% or 99% sequence identity to SEQ ID NO:91, and a C_{L} comprising the sequence set forth in SEQ ID NO:127, or an amino acid sequence having at least at or about 95%, 96%, 97%, 98% or 99% sequence identity to SEQ ID NO:127. In some embodiments, the antibody or antigen-binding fragment thereof comprises a heavy chain comprising the sequence set forth in SEQ ID NO:128, or an amino acid sequence having at least at or about 95%, 96%, 97%, 98% or 99% sequence identity to SEQ ID NO:128, and a light chain comprising the sequence set forth in SEQ ID NO:129, or an amino acid sequence having at least at or about 95%, 96%, 97%, 98% or 99% sequence identity to SEQ ID NO:129. In some embodiments, the antibody or antigen-binding fragment thereof comprises a heavy chain comprising the sequence set forth in SEQ ID NO:128, and a light chain comprising the sequence set forth in SEQ ID NO:129.

### 1. Variants

In certain embodiments, the antibodies include one or more amino acid variations, e.g., substitutions, deletions, insertions, and/or mutations, compared to the sequence of an antibody described herein. Exemplary variants include those designed to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, the antibodies include one or more amino acid substitutions, e.g., as compared to an antibody sequence described herein and/or compared to a sequence of a natural repertoire, e.g., human repertoire. Sites of interest for substitutional mutagenesis include the CDRs and FRs. Amino acid substitutions may be introduced into an antibody of interest and the products screened for a desired activity, e.g., retained/improved antigen-binding, decreased immunogenicity, improved half-life, and/or improved effector function, such as the ability to promote antibody-dependent cellular cytotoxicity (ADCC) or complement-dependent cytotoxicity (CDC).

In some embodiments, one or more residues within a CDR of a parent antibody (e.g. a humanized or human antibody) is/are substituted. In some embodiments, the substitution is made to revert a sequence or position in the sequence to a germline sequence, such as an antibody sequence found in the germline (e.g., human germline), for example, to reduce the likelihood of immunogenicity, e.g., upon administration to a human subject.

In some embodiments, alterations are made in CDR “hotspots,” residues encoded by codons that undergo mutation at high frequency during the somatic maturation process (see, e.g., Chowdhury, Methods Mol. Biol. 207:179-196 (2008)), and/or residues that contact antigen, with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen-binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant V_{H} and V_{L} sequences provided above, each CDR either is unaltered, or comprises no more than one, two or three amino acid substitutions.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

### 2. Modifications

In certain embodiments, the antibody or antigen-binding fragment thereof is altered to increase or decrease the extent to which the antibody or antigen-binding fragment thereof is glycosylated, for example, by removing or inserting one or more glycosylation sites by altering the amino acid sequence and/or by modifying the oligosaccharide(s) attached to the glycosylation sites, e.g., using certain cell lines.

In some embodiments, an N-linked glycosylation, which is a glycosylation site that occurs at asparagines in the consensus sequence -Asn-Xaa-Ser/Thr is removed or inserted. In some embodiments, one or more re replaced with another amino acid to remove the glycosylation site.

Exemplary modifications, variants, and cell lines are described, e.g., in Patent Publication Nos. US 2003/0157108, US 2004/0093621, US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Ripka et al. Arch. Biochem. Biophys. 249:533-545 (1986); US Pat Appl No US 2003/0157108 A1, Presta, L; and WO 2004/056312 A1, Yamane-Ohnuki et al. Biotech. Bioeng. 87: 614 (2004); Kanda, Y. et al., Biotechnol. Bioeng., 94(4):680-688 (2006); and WO2003/085107); WO 2003/011878; U.S. Pat. No. 6,602,684; and US 2005/0123546; WO 1997/30087; WO 1998/58964; and WO 1999/22764.

Among the modified antibodies are those having one or more amino acid modifications in the Fc region, such as those having a human Fc region sequence or other portion of a constant region (e.g., a human IgG1, IgG2, IgG3 or IgG4 Fc region) comprising an amino acid modification (e.g. a substitution) at one or more amino acid positions. Such modifications can be made, e.g., to improve half-life, alter binding to one or more types of Fc receptors, and/or alter effector functions.

Also among the variants are cysteine engineered antibodies such as “thioMAbs” and other cysteine engineered variants, in which one or more residues of an antibody are substituted with cysteine residues, in order to generate reactive thiol groups at accessible sites, e.g., for use in conjugation of agents and linker-agents, to produce immunoconjugates. Cysteine engineered antibodies are described, e.g., in U.S. Pat. Nos. 7,855,275 and 7,521,541.

In some embodiments, the antibodies are modified to contain additional nonproteinaceous moieties, including water soluble polymers. Exemplary polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### 3. Exemplary Features

In some aspects, the provided antibodies have one or more specified functional features, such as binding properties, including binding to particular epitopes or exhibiting lower or reduced binding to a related but non-specific antigen. In some aspects, the provided antibodies can bind to an epitope that is similar to or overlaps with epitopes of other antibodies, such as reference antibodies, and/or exhibit particular binding affinities. In some aspects, the provided antibodies can bind to an epitope that is different from epitopes of other antibodies, e.g., binding a conformational epitope.

In some embodiments, the provided antibodies or antigen-binding fragment thereof specifically bind to a Delta-like ligand 3 (DLL3) protein. In some of any of the embodiments provided herein, DLL3 refers to human DLL3. The observation that an antibody or other binding molecule binds to DLL3 or specifically binds to DLL3 does not necessarily mean that it binds to DLL3 from every species. For example, in some embodiments, features of binding to DLL3, such as the ability to specifically bind thereto and/or to compete for binding thereto with a reference antibody, and/or to bind with a particular affinity or compete to a particular degree, in some embodiments, refers to the ability with respect to a human DLL3 and the antibody may not have this feature with respect to a DLL3 of another species such as mouse, Rhesus macaque or cynomolgus monkey.

In some embodiments, an antibody or an antigen-binding fragment thereof is said to specifically bind an antigen when it preferentially recognizes its target antigen in a complex mixture of proteins and/or macromolecules. An antibody is said to specifically bind an antigen when the equilibrium dissociation constant is ≤10⁻⁷ or ≤10⁻⁸ M.

An antibody molecule or an antigen-binding fragment thereof is said to exhibit “specific binding” or “preferential binding” if it reacts or associates more frequently, more rapidly, with greater duration and/or with greater affinity with a particular antigen than it does with alternative antigens. An antibody molecule or an antigen-binding fragment thereof specifically binds or preferentially binds to a target if it binds with greater affinity, avidity, more readily, and/or with greater duration than it binds to other substances. For example, an antibody that specifically or preferentially binds to a DLL3 is an antibody that binds a DLL3 proetien with greater affinity, avidity, more readily, and/or with greater duration than it binds to other DLL3 from another species or non-DLL3 antigens. It is also understood that specific binding or preferential binding does not necessarily require (although it can include) exclusive binding. Methods to determine such specific or preferential binding are also well known, e.g., an immunoassay.

In some embodiments, the antibodies, such as the anti-DLL3 antibodies, e.g., the human antibodies, specifically bind to a particular epitope or region of DLL3, such as generally an extracellular epitope or region.

DLL3 is a type I transmembrane Delta-like protein typically expressed exclusively on intracellular membranes, such as the Golgi apparatus. Human DLL3 has an extracellular region that has various extracellular domains, including an N-terminus of Notch ligand domain, a Delta, Serrate, Lag2 (DSL) domain, and six epidermal growth factor (EGF)-like repeats (EGF repeats; EGF1-6 in human DLL3); a transmembrane domain and a cytoplasmic region. With reference to human DLL3 isoform 1 precursor sequence set forth in SEQ ID NO:120 (e.g., UNIPROT Q9NYJ7-1; Genbank NP_058637.1; mature polypeptide without signal sequence set forth in SEQ ID NO:122), amino acid residues 1-26 correspond to the signal peptide, amino acid residues 27-84 correspond to the N-terminus of Notch ligand domain, amino acid residues 176-215 correspond to the DSL domain, amino acid residues 216-249 correspond to the EGF-like 1 domain, amino acid residues 274-310 correspond to the EGF-like 2 domain, amino acid residues 312-351 correspond to the EGF-like 3 domain, amino acid residues 353-389 correspond to the EGF-like 4 domain, amino acid residues 391-427 correspond to the EGF-like 5 domain, amino acid residues 429-465 correspond to the EGF-like 6 domain, amino acid residues 493-513 correspond to the transmembrane domain, and amino acid residues 514-618 correspond to the cytoplasmic domain. Human DLL3 isoform 2 differs from isoform 1 in that amino acid residues 587-618, with reference to the human DLL3 isoform 1 precursor sequence set forth in SEQ ID NO:120, is replaced with an alanine residue (human DLL3 isoform 2 precursor sequence set forth in SEQ ID NO:121; e.g., UNIPROT Q9NYJ7-2; Genbank NP_982353.1; mature polypeptide without signal sequence set forth in SEQ ID NO:123). DLL3 has low sequence homology to other cell surface Notch ligands in the family: 35% homology to DLL1 and 38% homology to DLL4.

In some embodiments, the DLL3 binding molecules, such as antibodies or antigen-binding fragments thereof, bind, such as specifically bind, to human DLL3, such as to one or more epitopes or region of human DLL3, such as the mature human DLL3 sequence set forth in SEQ ID NO:122 or 123, or an allelic variant or splice variant thereof. In some embodiments, the antibodies or antigen-binding fragment thereof specifically binds to one or more epitopes within a human DLL3. In some embodiments, the human DLL3 comprises the sequence of amino acids forth in SEQ ID NO:120. In some embodiments, the human DLL3 comprises the sequence of amino acids forth in SEQ ID NO:121. In some embodiments, the human DLL3 isoform (mature, isoform 1) comprises the sequence of amino acids forth in SEQ ID NO:122. In some embodiments, the human DLL3 isoform (mature, isoform 2) comprises the sequence of amino acids forth in SEQ ID NO:123. In some embodiments, the antibodies or antigen-binding fragment thereof bind to the extracellular region DLL3, such as to one or more extracellular epitopes present within the extracellular region of mature human DLL3, e.g., corresponding to residues 27-492 of the human DLL3 precursor sequence set forth in SEQ ID NO:120.

In some embodiments, the antibodies or antigen-binding fragment thereof bind one or more epitope of DLL3, such as a human DLL3. In some embodiments, the antibodies or antigen-binding fragment thereof bind a linear epitope of DLL3, such as a human DLL3. In some embodiments, the one or more epitopes comprises a conformational epitope. In some embodiments, the antibodies or antigen-binding fragment thereof bind one or more conformational epitopes of DLL3, such as a human DLL3. In some embodiments, the antibodies or antigen-binding fragment thereof bind one or more conformational epitopes of DLL3, such as a human DLL3.

In some embodiments, the DLL3 binding molecules, such as antibodies or antigen-binding fragments thereof, bind to an epitope comprising amino acid residues within the N-terminus of Notch ligand domain, DSL domain, EGF-like 1 domain, EGF-like 2 domain, EGF-like 3 domain, EGF-like 4 domain, EGF-like 5 domain, or EGF-like 6 domain.

In some embodiments, the antibodies or antigen-binding fragment thereof bind one or more epitopes of human DLL3, such as one or more epitopes comprising 2, 3, 4, 5, 6, 7, 8, 9, 10, 11, 12, 13, 14, 15, or more contiguous amino acid residues within human DLL3 (for example, human DLL3 precursor sequence set forth in SEQ ID NO:120), or two or more epitopes, each comprising 1, 2, 3, 4, 5, 6, 7, 8, 9, 10, 11, 12, 13, 14, 15, or more contiguous amino acid residues within human DLL3 (for example, human DLL3 precursor sequence set forth in SEQ ID NO:120). In some aspects, the one or more epitopes include 2, 3, 4, 5, 6, 7, 8, 9, 10, 11, 12, 13, 14, 15, or more contiguous amino acid residues within amino acid residues 27-84 correspond to the N-terminus of Notch ligand domain, amino acid residues 176-215 correspond to the DSL domain, amino acid residues 216-249 correspond to the EGF-like 1 domain, amino acid residues 274-310 correspond to the EGF-like 2 domain, amino acid residues 312-351 correspond to the EGF-like 3 domain, amino acid residues 353-389 correspond to the EGF-like 4 domain, amino acid residues 391-427 correspond to the EGF-like 5 domain, or amino acid residues 429-465 correspond to the EGF-like 6 domain, with reference to the human DLL3 precursor sequence set forth in SEQ ID NO:120.

In some embodiments, the antibody or antigen-binding fragment binds to an epitope within amino acid residues 27-84 with reference to SEQ ID NO:120, corresponding to the N-terminus of Notch ligand domain. In some embodiments, the antibody or antigen-binding fragment binds to an epitope within amino acid residues 176-215 with reference to SEQ ID NO:120, corresponding to the DSL domain. In some embodiments, the antibody or antigen-binding fragment binds to an epitope within amino acid residues 216-249 with reference to SEQ ID NO:120, corresponding to the EGF-like 1 domain. In some embodiments, the antibody or antigen-binding fragment binds to an epitope within amino acid residues 274-310 with reference to SEQ ID NO:120, corresponding to the EGF-like 2 domain. In some embodiments, the antibody or antigen-binding fragment binds to an epitope within amino acid residues 312-351 with reference to SEQ ID NO:120, corresponding to the EGF-like 3 domain. In some embodiments, the antibody or antigen-binding fragment binds to an epitope within amino acid residues 353-389 with reference to SEQ ID NO:120, corresponding to the EGF-like 4 domain.

In some embodiments, the antibody or antigen-binding fragment binds to an epitope within amino acid residues 391-427 with reference to SEQ ID NO:120, corresponding to the EGF-like 5 domain. In some embodiments, the antibody or antigen-binding fragment binds to an epitope within amino acid residues 429-465 with reference to SEQ ID NO:120, corresponding to the EGF-like 6 domain. In some embodiments, the antibody or antigen-binding fragment binds to an epitope within amino acid residues 493-513 with reference to SEQ ID NO: 120, corresponding to the transmembrane domain.

In some embodiments, the provided antibodies are capable of binding DLL3, such as human DLL3, with at least a certain affinity, as measured by any of a number of known methods. In some embodiments, the affinity is represented by an equilibrium dissociation constant (K_{D}). In some embodiments, the affinity is represented by EC₅₀.

A variety of assays are known for assessing binding affinity, equilibrium dissociation constant (K_{D}), equilibrium association constant (K_{A}), EC₅₀, on-rate (association rate constant; kₒₙ or kₐ; units of 1/Ms or M⁻¹s⁻¹) and the off-rate (dissociation rate constant; k_{off} or k_{d}; units of 1/s or s⁻¹) and/or determining whether a binding molecule (e.g., an antibody or fragment thereof) specifically binds to a particular ligand (e.g., an antigen, such as a DLL3 protein). One can determine the binding affinity of a binding molecule, e.g., an antibody or an antigen-binding fragment thereof, for an antigen, e.g., DLL3, such as human DLL3 or cynomolgus DLL3 or mouse DLL3, such as by using any of a number of binding assays that are well known. For example, in some embodiments, a BIAcore® instrument can be used to determine the binding kinetics and constants of a complex between two proteins (e.g., an antibody or fragment thereof, and an antigen, such as a DLL3 protein), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation rate constant (k_{off} or k_{d}), the association rate constant (kₒₙ or kₐ) and/or equilibrium dissociation constant (K_{D}) and/or equilibrium association constant (K_{A}) for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing, genetic reporter assays, flow cytometry, and other methods for detection of expressed nucleic acids or binding of proteins.

In some embodiments, the binding molecule, e.g., antibody or fragment thereof, binds, such as specifically binds, to an antigen, e.g., a DLL3 protein or an epitope therein, with an affinity or K_{A} (i.e., an equilibrium association constant of a particular binding interaction with units of 1/M or M⁻¹; equal to the ratio of the on-rate [kₒₙ or kₐ] to the off-rate [k_{off} or k_{d}] for this association reaction, assuming bimolecular interaction) equal to or greater than 10⁵ M⁻¹. In some embodiments, the peptide binding molecule binds, such as specifically binds, to an epitope of an antigen, e.g., human DLL3, with an affinity or K_{A} (i.e., an equilibrium association constant of a particular binding interaction with units of 1/M or M⁻¹) equal to or greater than 10⁵ M⁻¹ (which equals the ratio of the on-rate [kₒₙ] to the off-rate [k_{off}] for this association reaction). In some embodiments, the binding molecule, e.g., antibody or antigen-binding fragment thereof, exhibits a binding affinity for a T cell epitope of the target polypeptide with an affinity or K_{A} ranging from at or about 10⁶ M⁻¹ to at or about 10⁷ M⁻¹. In some embodiments, binding affinity may be classified as high affinity or as low affinity. For example, in some cases, a binding molecule, e.g., antibody or antigen-binding fragment thereof, that exhibits high affinity binding to a particular epitope interacts with such epitope with a K_{A} of at or about 10⁷ M⁻¹. In some cases, a binding molecule, e.g., antibody or antigen-binding fragment thereof, that exhibits low affinity binding exhibits a K_{A} of up to 10⁷ M⁻¹. In certain embodiments, any of the foregoing K_{A} values or ranges are determined by SPR. In certain embodiments, any of the foregoing K_{A} values or ranges are determined by SPR as described in Example 3.

Alternatively, affinity can be defined as an equilibrium dissociation constant (K_{D}) of a particular binding interaction with units of M (e.g., 10⁻⁵ M to 10⁻¹³ M). In some embodiments, the antibody or fragment thereof exhibits a binding affinity for the epitope with a K_{D} (i.e., an equilibrium dissociation constant of a particular binding interaction with units of M; equal to the ratio of the off-rate [k_{off} or k_{d}] to the on-rate [kₒₙ or kₐ] for this association reaction, assuming bimolecular interaction) of equal to or less than 10⁻⁵ M. For example, the equilibrium dissociation constant K_{D} can range from 10⁻⁵ M to 10⁻⁸ M. In certain embodiments, any of the foregoing K_{D} values or ranges are determined by SPR. In certain embodiments, any of the foregoing K_{D} values or ranges are determined by SPR as described in Example 3.

The on-rate (association rate constant; kₒₙ or k_{d}; units of 1/Ms or M⁻¹s⁻¹) and the off-rate (dissociation rate constant; k_{off} or k_{d}; units of 1/s or s⁻¹) can be determined using any of the known assay methods, for example, surface plasmon resonance (SPR), or other methods described herein for measuring the binding of one protein to another.

In some embodiments, the equilibrium dissociation constant (K_{D}) of the antibody to DLL3, such as human DLL3, is from at or about 50 nM to at or about 500 nM, from at or about 50 nM to at or about 100 nM or from at or about 100 nM to at or about 500 nM. In certain embodiments, the equilibrium dissociation constant (K_{D}) of the antibody to DLL3, such as human DLL3, is at or about 100 nM, 90 nM, 80 nM, 70 nM, 60 nM, or 50 nM, or a range defined by any of the foregoing. In some embodiments, the equilibrium dissociation constant, K_{D}, of the binding molecule, e.g., anti-DLL3 antibody or fragment thereof, to a DLL3 protein, such as a human DLL3, is from at or about 50 nM to 500 nM, 50 nM to 100 nM or 100 nM to 500 nM. In certain embodiments, the equilibrium dissociation constant, K_{D}, of the binding molecule, e.g., anti-DLL3 antibody or fragment thereof, to a DLL3 protein, such as a human DLL3, is at or about 1 M, 500 nM, 100 nM, 90 nM, 80 nM, 70 nM, 60 nM, or 50 nM, or a range defined by any of the foregoing. In certain embodiments, any of the foregoing K_{D} values or ranges are determined by SPR. In certain embodiments, any of the foregoing K_{D} values or ranges are determined by SPR as described in Example 3.

In some embodiments, the K_{D} of the binding molecule, e.g., anti-DLL3 antibody or fragment thereof, to a DLL3 protein, is between at or about 50 nM and at or about 60 nM. In certain embodiments, the K_{D} of the binding molecule, e.g., anti-DLL3 antibody or fragment thereof, to a DLL3 protein, such as a human DLL3, is at or about 50 nM, 60 nM, 70 nM, 80 nM, 90 nM or 100 nM, or a range defined by any of the foregoing. In certain embodiments, the K_{D} of the binding molecule, e.g., anti-DLL3 antibody or fragment thereof, to a DLL3 protein, such as a human DLL3, is at or about 50 nM, 55 nM, 60 nM, or 65 nM, or a range defined by any of the foregoing. In certain embodiments, any of the foregoing K_{D} values or ranges are determined by SPR. In certain embodiments, any of the foregoing K_{D} values or ranges are determined by SPR as described in Example 3.

In some embodiments, the binding affinity (EC₅₀) of the antibody to DLL3, such as human DLL3, is from at or about 10 nM to 15 nM, 10 nM to 20 nM, 10 nM ot 30 nM, 50 nM to at or about 500 nM, from at or about 50 nM to at or about 100 nM or from at or about 100 nM to at or about 500 nM. In certain embodiments, the binding affinity (EC₅₀) of the antibody to DLL3, such as human DLL3, is at or about 100 nM, 90 nM, 80 nM, 70 nM, 60 nM, 50 nM, 40 nM, 30 nM, 20 nM, or 10 nM, or a range defined by any of the foregoing. In some embodiments, binding affinity (EC₅₀), of the binding molecule, e.g., anti-DLL3 antibody or fragment thereof, to a DLL3 protein, such as a human DLL3, is from at or about 50 nM to 500 nM, 50 nM to 100 nM or 100 nM to 500 nM. In certain embodiments, the binding affinity (EC₅₀) of the binding molecule, e.g., anti-DLL3 antibody or fragment thereof, to a DLL3 protein, such as a human DLL3, is at or about 1 M, 500 nM, 100 nM, 90 nM, 80 nM, 70 nM, 60 nM, 50 nM, 40 nM, 30 nM, 20 nM, or 10 nM, or a range defined by any of the foregoing. In certain embodiments, the EC₅₀ values or ranges are determined by the experimental conditions described in Example 4.

In some embodiments, the binding affinity (EC₅₀) of the binding molecule, e.g., anti-DLL3 antibody or fragment thereof, to a DLL3 protein, is between at or about 10 nM and at or about 20 nM. In certain embodiments, the EC₅₀ values or ranges are determined by the experimental conditions described in Example 4.

In some of any of the provided embodiments, the antibody or antigen-binding fragment thereof does not bind to, is not cross-reactive to, or binds at a lower extent, level or degree or affinity to a related Notch ligand protein, such as DLL1 or DLL4, e.g., a human DLL1 or a human DLL4.

In some embodiments, the extent of binding of an anti-DLL3 antibody to a different non-DLL3 Notch ligand protein, such as DLL1 or DLL4 protein, e.g., a human DLL1 or a human DLL4, or other non-DLL3 protein, is less than at or about 50%, 40%, 30%, 20% or 10% of the binding of the antibody to human DLL3 as measured. In some embodiments, the antibodies or antigen-binding fragments thereof do not bind to DLL1 or a DLL4. In some embodiments, the antibodies or antigen-binding fragments do not bind to, is not cross-reactive to, or binds at a lower level or degree or affinity to a DLL1 or a DLL4. In some embodiments, the extent, level or degree or affinity of binding of the provided anti-DLL3 antibody or antigen-binding fragment thereof to a DLL1 or a DLL4 is at least at or about 75%, 80%, 90%, 95% or 99% less than the extent, level or degree or affinity of binding to a human DLL3. In some embodiments, the antibody or antigen-bining fragment thereof does not bind to a human DLL1. In some embodiments, the antibody or antigen-bining fragment thereof is not cross-reactive to a human DLL1. In some embodiments, the antibody or antigen-bining fragment thereof binds at a lower level or degree or affinity to a human DLL1. In some embodiments, the extent, level or degree or affinity of binding of the anti-DLL3 antibody or antigen-binding fragment thereof to a human DLL1 is at least at or about 75%, 80%, 90%, 95% or 99% less than the extent, level or degree or affinity of binding to a human DLL3. In some embodiments, the antibody or antigen-bining fragment thereof does not bind to a human DLL4. In some embodiments, the antibody or antigen-bining fragment thereof is not cross-reactive to a human DLL4. In some embodiments, the antibody or antigen-bining fragment thereof binds at a lower level or degree or affinity to a human DLL4. In some embodiments, the extent, level or degree or affinity of binding of the anti-DLL3 antibody or antigen-binding fragment thereof to a human DLL4 is at least at or about 75%, 80%, 90%, 95% or 99% less than the extent, level or degree or affinity of binding to a human DLL3.

In some aspects, the observation that an antibody or other binding molecule binds to DLL3 or specifically binds to DLL3 does not necessarily mean that it binds to DLL3 from every species. For example, in some embodiments, features of binding to DLL3, such as the ability to specifically bind thereto and/or to compete for binding thereto with a reference antibody, and/or to bind with a particular affinity or compete to a particular degree, in some embodiments, refers to the ability with respect to a human DLL3 and the antibody may not have this feature with respect to a DLL3 of another species such as mouse, Rhesus macaque or cynomolgus monkey. In some embodiments, the antibody binds to human DLL3 and binds to DLL3 of another species, such as Rhesus macaque or cynomolgus monkey. In some embodiments, the antibody or an antigen-binding fragment thereof binds to human DLL3 and does not bind to DLL3 of another species, such as mouse. In some embodiments, the antibody binds to human DLL3 and binds to DLL3 of another species, such as mouse.

In some embodiments, the antibody binds to non-human DLL3, such as monkey, rabbit, rat, mouse, or other species of DLL3. In some embodiments, the antibody or antigen-binding fragment thereof binds to non-human DLL3, such as cynomolgus monkey (Macaca fascicularis) DLL3, such as to an epitope or region of cynomolgus monkey DLL3, such as the cynomolgus monkey DLL3 set forth in SEQ ID NO:125 (NCBI Reference Sequence: XM_005589196.2). In some embodiments, the antibody or antigen-binding fragment thereof binds to mouse (Mus musculus) DLL3, such as to an epitope or region of mouse DLL3, such as the mouse DLL3 set forth in SEQ ID NO:124 (GenBank No. NP_031892.2).

In some embodiments, the antibody or antigen-binding fragment thereof binds to human DLL3 and binds to cynomolgus monkey DLL3, such as the cynomolgus monkey DLL3 set forth in SEQ ID NO:125. In some embodiments, the extent of binding of some of the provided anti-DLL3 antibodies or fragments thereof to a non-human DLL3, such as cynomolgus monkey DLL3, is at least at or about 75%, 80%, 90%, 95%, 100%, 110%, 120%, 130%, 140%, 150% or more of the binding of the antibody or antigen-binding fragment thereof to human DLL3.

In some of any of the provided embodiments, the antibody or antigen-binding fragment thereof does not bind to, is not cross-reactive to, or binds at a lower extent, level or degree or affinity to a non-human DLL3, such as a mouse DLL3, such as the mouse DLL3 set forth in SEQ ID NO:124. In some embodiments, the extent of binding of an anti-DLL3 antibody to an unrelated, non-DLL3 protein or to a non-human DLL3, such as a mouse DLL3 protein, or other non-DLL3 protein, is less than at or about 50%, 40%, 30%, 20% or 10% of the binding of the antibody to human DLL3 as measured. In some embodiments, the antibodies or antigen-binding fragments thereof do not bind to mouse DLL3, such as the mouse DLL3 set forth in SEQ ID NO:124. In some embodiments, the antibodies or antigen-binding fragments do not bind to, is not cross-reactive to, or binds at a lower level or degree or affinity to a mouse DLL3. In some embodiments, the extent, level or degree or affinity of binding of the provided anti-DLL3 antibody or antigen-binding fragment thereof to a mouse DLL3 is at least at or about 75%, 80%, 90%, 95% or 99% less than the extent, level or degree or affinity of binding to a human DLL3.

In some embodiments, the binding affinity of a binding molecule, such as an anti-DLL3 antibody, for different antigens, e.g., DLL3 proteins from different species can be compared to determine the species cross-reactivity. For example, species cross-reactivity can be classified as high cross reactivity or low cross reactivity. In some embodiments, the equilibrium dissociation constant, K_{D}, for different antigens, e.g., DLL3 proteins from different species such as human, cynomolgus monkey or mouse, can be compared to determine species cross-reactivity. In some embodiments, the species cross-reactivity of an anti-DLL3 antibody can be high, e.g., the anti-DLL3 antibody binds to human DLL3 and a species variant DLL3 to a similar degree, e.g., the ratio of K_{D} for human DLL3 and K_{D} for the species variant DLL3 is or is about 1. In some embodiments, the species cross-reactivity of an anti-DLL3 antibody can be low, e.g., the anti-DLL3 antibody has a high affinity for human DLL3 but a low affinity for a species variant DLL3, or vice versa. For example, the ratio of K_{D} for the species variant DLL3 and K_{D} for the human DLL3 is more than 10, 15, 20, 25, 30, 40, 50, 60, 70, 80, 90, 100, 200, 500, 1000, 2000 or more, and the anti-DLL3 antibody has low species cross-reactivity. The degree of species cross-reactivity can be compared with the species cross-reactivity of a known antibody, such as a reference antibody.

In some embodiments, the provided antibodies or antigen-binding fragments thereof bind to a similar degree to a human DLL3 and a non-human DLL3. For example, in some embodiments, the provided antibodies or antigen-binding fragments thereof bind to a human DLL3, or an allelic variant or splice variant thereof, with a specific an equilibrium dissociation constant (K_{D}), and to a non-human DLL3, such as a cynomolgus monkey DLL3, with a K_{D} that is similar, or about the same, or less than 2-fold different, or less than 5-fold different.

In some embodiments, the total binding capacity (Rₘₐₓ), as measured using particular surface plasmon resonance (SPR) conditions, is used to determine the ability or capacity of binding of the provided antibody or antigen-binding fragment thereof, to the antigen, e.g., a DLL3 protein, such as a human DLL3. For SPR analysis, the “ligand” is the immobilized target molecule on the surface of the sensor, for example, a DLL3 protein, and the “analyte” is the tested molecule, e.g., antibody, for binding to the “ligand”. For example, the “analyte” can be any of the provided antibodies or antigen-binding fragments thereof, that binds to a DLL3 protein. For a particular ligand and analyte pair in SPR, the Rₘₐₓ can be determined assuming a 1:1 binding stoichiometry model, for a particular condition. In some embodiments, binding capacity (Rₘₐₓ) can be determined using the following formula: Rₘₐₓ (RU)=(analyte molecular weight)/(ligand molecular weight)×immobilized ligand level (RU). In particular aspects of SPR conditions, the Rₘₐₓ of binding between any of the provided antibody or antigen-binding fragment thereof and a DLL3 protein, such as a human DLL3 or a cynomolgus DLL3, is at least or at least about 50 resonance units (RU), such as about 25 RU, 20 RU, 15 RU, 10 RU, 5 RU or 1 RU.

### B. Immunoconjugates

In some embodiments, the DLL3-binding molecules, e.g., antibodies or antigen-binding fragment thereof, is or is part of an immunoconjugate, in which the antibody is conjugated to one or more heterologous molecule(s), such as, but not limited to, a cytotoxic or an imaging agent. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; antibiotics; toxins such as small molecule toxins or enzymatically active toxins. In some embodiments, the antibody is conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes. Among the immunoconjugates are antibody-drug conjugates (ADCs), in which an antibody is conjugated to one or more drugs. In certain embodiments, an antibody or or antigen-binding fragment thereof described herein can be used in an ADC. In certain embodiments, an ADC comprises an antibody or or antigen-binding fragment thereof described herein conjugated to a small molecule chemical compound. In certain embodiments, an ADC comprises an antibody or or antigen-binding fragment thereof described herein conjugated to a chemotherapeutic agent. Also among the immunoconjugates are those in which the antibody is conjugated to a toxin, such as an enzymatically active toxin or fragment thereof. In certain embodiments, an immunoconjugate comprises an antibody or or antigen-binding fragment thereof described herein and a toxin such as an enzymatically active toxin or fragment thereof. Immunoconjugates can also comprise an antibody or antigen-binding fragment thereof and a peptide or protein. In certain embodiments, an immunoconjugate comprises an antibody or or antigen-binding fragment thereof described herein and a peptide or protein.

Also among the immunoconjugates are those in which the antibody is conjugated to a radioactive atom to form a radioconjugate. Exemplary radioactive isotopes include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu.

Conjugates of an antibody and an agent, such as a drug, small molecule chemical compound, toxin, protein, or peptide, may be made using any of a number of known protein coupling agents, e.g., linkers, (see Vitetta et al., Science 238:1098 (1987)), WO94/11026. The linker may be a “cleavable linker” facilitating release of a cytotoxic drug in the cell, such as acid-labile linkers, peptidase-sensitive linkers, photolabile linkers, dimethyl linkers, and disulfide-containing linkers (Chari et al., Cancer Res. 52:127-131 (1992); U.S. Pat. No. 5,208,020).

### C. T Cell Engagers

In some embodiments, the provided DLL3-binding molecule is a molecule for T cell-engaging therapy. In some embodiments, the provided antibody or antigen-binding fragment thereof is comprised in a molecule for T cell-enggaging therapy. In some embodiments, the molecule for T cell-engaging therapy is or comprises a binding molecule capable of binding to a surface molecule expressed on a T cell. In some embodiments, the surface molecule is an activating component of a T cell, such as a component of the T cell receptor complex. In some embodiments, the surface molecule is CD3 or is CD2. In some embodiments, the molecule for T cell-engaging therapy is or comprises an antibody or antigen-binding fragment, such as an antibody or antigen-binding fragment that binds to CD3 or CD2, in addition to any of the antibody or antigen-binding fragment thereof that binds to DLL3 as described herein.

In some embodiments, the molecule for T cell-engaging therapy is a bispecific antibody containing at least one antigen-binding domain binding to an activating component of the T cell (e.g. a T cell surface molecule, e.g. CD3 or CD2) and at least one antigen-binding domain binding to a surface antigen, such as DLL3, on a cancer cell. In some embodiments, the molecule for T-cell engaging therapy is a bi-specific T cell engagers (BiTE). In certain embodiments, the antigen-binding domain binding to a surface antigen on a cancer cell is or comprises any of the DLL3-binding molecules described herein. In certain embodiments, the antigen-binding domain binding to a surface antigen on a cancer cell is an anti-DLL3 scFv comprising (i) a VH domain comprising the sequence set forth in SEQ ID NO: 90 and (ii) a VL domain comprising the sequence set forth in SEQ ID NO: 91. In certain embodiments, the antigen-binding domain binding to a surface antigen on a cancer cell is an anti-DLL3 scFv comprising the sequence set forth in SEQ ID NO:89. In some embodiments, the simultaneous or near simultaneous binding of such an antibody to both of its targets can result in a temporary interaction between the target cancer cell and T cell, thereby resulting in activation, e.g. cytotoxic activity, of the T cell and subsequent lysis of the target cancer cell.

In some embodiments, bi-specific T cell engagers (BiTE) are used in connection with the provided methods, uses, articles of manufacture. In some embodiments, bi-specific T cell engagers have specificity toward two particular antigens (or markers or ligands). In some embodiments, the antigens are expressed on the surface of a particular type of cell. In particular embodiments, the first antigen is associated with an immune cell or an engineered immune cell, and the second antigen is DLL3 expressed on the surface of a cancer cell.

Numerous methods of producing bi-specific T cell engagers are known, including fusion of two different hybridomas (Milstein and Cuello, Nature 1983; 305:537-540), and chemical tethering though heterobifunctional cross linkers (Staerz et al. Nature 1985; 314:628-631). Among such exemplary bi-specific antibody T cell-engaging molecules such as bispecific T cell engager (BiTE) molecules, are those which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010).

In certain embodiments, the bi-specific T cell engager is or comprises a polypeptide construct or a fusion protein. In certain embodiments, the polypeptide construct contains a first component comprising an antigen-binding domain binding to an activating component of an immune cell or engineered immune cell, and a second component comprising an antigen-binding domain binding to a surface antigen associated with certain cancers, such as DLL3. In some embodiments, the first and second components are coupled by a linker.

In some embodiments, the antigen-binding domain of the first component of the bi-specific T cell engager engages a receptor on an endogenous immune cell in the periphery of the tumor. In some embodiments, the endogenous immune cell is a T cell. In some aspects, the engagement of the endogenous T cell receptor redirects the endogenous T cells to the tumor. In some aspects, the engagement of the endogenous T cell receptor recruits tumor infiltrating lymphocytes (TILs) to the tumor. In some aspects, the engagement of the endogenous T cell receptor activates the endogenous immune repertoire.

In some embodiments, the simultaneous or near simultaneous binding of the bi-specific T cell engager to both of its targets (e.g. the immune cell and the cancer cell expressing DLL3) can result in a temporary interaction between the DLL3-expressing cancer cell and T cell, thereby resulting in activation (e.g. cytotoxic activity, cytokine release), of the T cell and subsequent lysis of the cancer cell.

In some embodiments, the first component of the bi-specific T cell engager is or comprises an antigen-binding domain that binds to an activating component of a T cell. In some embodiments, the activating component of the T cell is a surface molecule. In some embodiments, the surface molecule is or comprises a T-cell antigen. Exemplary T-cell antigens include but are not limited to CD2, CD3, CD4, CD5, CD6, CD8, CD25, CD28, CD30, CD40, CD44, CD45, CD69 and CD90. In some aspects, the binding of the bispecific T cell engaging molecule with the T cell antigen stimulates and/or activates the T cell. In some aspects, an activating component of the T cell is a T cell surface molecule, such as CD3 or CD2.

In some embodiments, the anti-T cell binding domain includes an antibody or an antigen-binding fragment thereof selected from the group consisting of a Fab fragment, a F(ab′)2 fragment, an Fv fragment, an scFv, a scAb, a dAb, a single domain heavy chain antibody, and a single domain light chain antibody.

In some embodiments, the T cell binding domain on the BiTE is an anti-CD3. In some aspects, the anti-CD3 domain is an scFv. In some embodiments, the anti-CD3 domain of the BiTE binds to a subunit of the CD3 complex on a receptor on a T cell. In some aspects, the receptor is on an endogenous T cell. In some embodiments, the receptor is on an engineered immune cell further expressing a recombinant receptor. The effects of CD3 engagement of T cells is well known in the art, and include but are not limited to T cell activation and other downstream cell signaling. Any of such BiTEs can be used in the provided disclosure herein.

In some embodiments, the second component of the BiTE comprises an antigen-binding domain that binds to DLL3 expressed on the surface of a cancer cell. In some embodiments, the surface antigen of the target cell is DLL3. In some embodiments, the second component of the BiTE comprises any of the antibodies or antigen-binding domains that binds to DLL3 described herein. In certain embodiments, the antigen-binding domain that binds to DLL3 is an anti-DLL3 scFv comprising (i) a VH domain comprising the sequence set forth in SEQ ID NO: 90 and (ii) a VL domain comprising the sequence set forth in SEQ ID NO: 91. In certain embodiments, the antigen-binding domain that binds to DLL3 is an anti-DLL3 scFv comprising the sequence set forth in SEQ ID NO:89.

In some embodiments, both antigen-binding domains, including the first antigen-binding domain and the second antigen-binding domain, comprise an antibody or an antigen-binding fragment.

In some embodiments, the polypeptide is a construct containing from N-terminus to C-terminus: a first component comprising an antigen-binding domain binding to an activating component of the T cell, a peptide linker, and a second component comprising an antigen-binding domain binding to a surface antigen associated with certain cancers, such as DLL3. In certain embodiments, the first antigen-binding domain of the BiTE is an anti-CD3 scFv. In certain embodiments, the second antigen-binding domain of the BiTE is an anti-DLL3 scFv. In certain embodiments, the anti-DLL3 scFv comprises the sequence set forth in SEQ ID NO:89.

In some aspects, the BiTE polypeptide constructs contain a linker that joins the first component comprising the antigen-binding domain that binds to an activating portion of the T cell, to the second component comprising an antigen-binding domain binding to DLL3 on the cancer cell. In some aspects, the linker is a short, medium or long linker. In some aspects, the linker can be any peptide linkers described herein. In some embodiments, the peptide linker is or comprises a cleavable peptide linker.

### D. Multispecific Antibodies

In certain embodiments, the DLL3-binding molecules, e.g., antibodies or antigen-binding fragment thereof, or fusion proteins, such as recombinant receptors containing the same, are multispecific. Among the multispecific binding molecules are multispecific antibodies, including, e.g. bispecific. Multispecific binding partners, e.g., antibodies, have binding specificities for at least two different sites, which may be in the same or different antigens. In certain embodiments, one of the binding specificities is for DLL3 and the other is for another antigen. In certain embodiments, bispecific antibodies may bind to two different epitopes of DLL3. Bispecific antibodies may also be used to localize cytotoxic agents to cells which express DLL3. Bispecific antibodies can be prepared as full length antibodies or antibody fragments. Among the multispecific antibodies are multispecific single-chain antibodies, e.g., diabodies, triabodies, and tetrabodies, tandem di-scFvs, and tandem tri-scFvs. Also provided are multispecific chimeric receptors, such as multispecific CARs, containing the antibodies. Also provided are multispecific cells containing the antibodies or polypeptides including the same, such as cells containing a cell surface protein including the anti-DLL3 antibody and an additional cell surface protein, such as an additional chimeric receptor, which binds to a different antigen or a different epitope on DLL3.

### E. Recombinant Receptors

Among the provided binding molecules, e.g., DLL3 binding molecules, are cell surface proteins, such as recombinant receptors, such as those that include one of the provided antibodies or antigen-binding fragments. Also provided are polynucleotides that encode all or a portion of such cell surface proteins, e.g., receptors. The receptors include antigen receptors and other chimeric receptors that specifically bind to DLL3, such as receptors containing the provided anti-DLL3 antibodies, e.g., antigen-binding fragments. Among the antigen receptors are functional non-TCR antigen receptors, such as chimeric antigen receptors (CARs). The DLL3-binding receptors generally contain antibodies (e.g., antigen-binding fragments), and/or other binding polypeptides that specifically bind to DLL3, such as to DLL3 proteins, such as a human DLL3. Also provided are cells expressing the recombinant receptors, compositions containing such cells and uses thereof in adoptive cell therapy, such as treatment of diseases and disorders associated with DLL3 expression, compositions and articles of manufacture and uses of the same.

Provided are chimeric antigen receptors (CARs) specific for DLL3, and polynucleotides containing nucleic acid sequences encoding all or a portion, fragment, domain or chain of any of the CARs described herein. In some embodiments, the CARs contain one of the DLL3-binding antibody fragments, such as those described in Section I.A and/or Table 2. In some embodiments, the CARs are among those described in each row of Tables E1-E4 and E8 and/or Table 3.

Among the provided polynucleotides are those that encode recombinant receptors, such as antigen receptors, that specifically bind DLL3. In some aspects, the encoded receptors, such as those containing DLL3-binding polypeptides, and compositions and articles of manufacture and uses of the same, also are provided. The provided polynucleotides can be incorporated into constructs, such as deoxyribonucleic acid (DNA) or RNA constructs, such as those that can be introduced into cells for expression of the encoded recombinant DLL3-binding receptors. Also provided herein are polynucleotides encoding the CARs. In some embodiments, the CAR can be encoded by more than one different polynucleotides, such as two or more polynucleotides. In some of any such embodiments, two or more polynucleotides can each contain nucleic acids encoding a portion, fragment, domain or chain of the CAR.

### 1. Chimeric Antigen Receptors (CARs)

In some aspects, provided are DLL3-binding cell surface proteins, such as receptors. In some aspects, the provided DLL3-binding cell surface proteins, such as receptors, generally contain an extracellular antigen-binding domain and an intracellular signaling region. Among the provided receptors, e.g., recombinant receptors, such as chimeric antigen receptors (CARs) containing the provided antibodies or antigen-binding fragment thereof, such as one or more of the provided anti-DLL3 antibody or fragment thereof. In some embodiments, the provided cell surface proteins specifically bind to DLL3, such as a human DLL3.

Among the antigen receptors are chimeric and/or functional non-TCR antigen receptors, such as chimeric antigen receptors (CARs). The chimeric receptors, such as CARs, generally include an extracellular antigen-binding domain that includes, is, or is comprises an anti-DLL3 antibody, such as an anti-DLL3 antibody or fragment thereof described herein. In some embodiments, the chimeric receptors, e.g., CARs, include an intracellular signaling domain. In some embodiments, the chimeric receptors also include a spacer and/or a transmembrane domain. In some embodiments, the spacer is located between the extracellular antigen-binding domain and the transmembrane domain. In some embodiments, the CAR comprises an extracellular antigen-binding domain, a spacer, a transmembrane domain and an intracellular signaling region. Exemplary CARs provided herein include those containing an antigen-binding domain comprising an antibody or antigen-binding fragment thereof described herein, e.g., in Section I.A and/or in Table 2, or those described in Section I.E herein, in Table 3 and/or in Tables E1-E4 and E8. Also provided are CARs encoded by the polynucleotides described in Section I.F herein, in Table 3 and/or in Tables E1-E4 and E8.

In some cases, CARs are referred to as first, second, and/or third generation CARs. In some aspects, a first generation CAR is one that solely provides a CD3-chain induced signal upon antigen-binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the CAR comprises an extracellular antigen-binding domain, in some cases comprising an antibody, e.g., an antibody fragment that binds DLL3, a transmembrane domain that is or comprises a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR comprises an extracellular antigen-binding domain, in some cases comprising an antibody, e.g., antibody fragment that binds DLL3, a transmembrane domain that is or comprises a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g., an IgG4 hinge, such as a hinge-only spacer.

In some embodiments, the chimeric antigen receptor includes an extracellular portion containing the antibody or fragment thereof described herein. In some aspects, the chimeric antigen receptor includes an extracellular portion containing the antibody or fragment thereof described herein and an intracellular signaling domain. In some embodiments, the antibody or fragment thereof includes an scFv and the intracellular signaling region comprises an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain.

Other exemplary antigen receptors, including CARs, and methods for engineering and introducing such receptors into cells, include those described, for example, in WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No. 7,446,190, and those described in WO/2014055668 A1. Exemplary of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, e.g., and in which the antigen-binding portion, e.g., scFv, is replaced by an antibody, e.g., as provided herein.

Other DLL3-targeting CARs are described, for example, by Hudecek et al., Clin Cancer Res, 19(12), 3153-3164 (2013) and Baskar et al. MAbs. 4(3): 349-361 (2012). See also WO2014031687; US2012/20058051.

a. Extracellular Antigen-Binding Domain

Among the chimeric receptors are chimeric antigen receptors (CARs). The chimeric receptors, such as CARs, generally include an extracellular antigen-binding domain that includes, is, or is comprised within, one or more of the provided anti-DLL3 antibodies or antigen-binding fragments. Thus, the chimeric receptors, e.g., CARs, typically include in their extracellular portions one or more DLL3-binding molecules, such as one or more antigen-binding fragment, domain, or portion, or one or more antibody variable domains and/or antibody molecules, such as those described herein. In some embodiments, the CAR includes a DLL3-binding portion or portions of the antibody molecule, such as a variable heavy (V_{H}) chain region and/or variable light (V_{L}) chain region of the antibody, e.g., an scFv. In some embodiments, the CAR includes a DLL3-binding portion or portions of the antibody molecule, such as a variable heavy (V_{H}) chain region and a variable light (V_{L}) chain region of the antibody, e.g., an scFv. In some aspects, the CAR includes one or more of any of the DLL3-binding antibodies or antigen-binding fragments thereof described herein, e.g., in Section I.A.

In some embodiments, Table 2 provides the SEQ ID NOS: of exemplary antigen-binding domains, such as antibodies or antigen-binding fragments, that can be comprised in the provided DLL3-binding receptors, such as anti-DLL3 chimeric antigen receptors (CARs). In some aspects, the CAR comprises an scFv described in Table 2, for example, as a part of the extracellular antigen-binding domain. In some aspects, the CAR comprises a V_{H} region described in Table 2, for example, as a part of the extracellular antigen-binding domain. In some aspects, the CAR comprises a V_{L} region described in Table 2, for example, as a part of the extracellular antigen-binding domain. In some embodiments, the DLL3-binding receptor comprises a DLL3-binding antibody or fragment thereof, comprising a V_{H} region that comprises a CDR-H1, a CDR-H2 and a CDR-H3 sequence and a V_{L} region that comprises a CDR-L1, a CDR-L2 and a CDR-L3 sequence set forth in the SEQ ID NOS: listed in each row of Table 2. In some embodiments, the DLL3-binding receptor comprises a DLL3-binding antibody or fragment thereof, comprising a V_{H} region sequence and a V_{L} region sequence set forth in the SEQ ID NOS: listed in each row of Table 2, or an antibody comprising a V_{H} and V_{L} region amino acid sequence that has at least 95%, 96%, 97%, 98%, or 99% sequence identity to the V_{H} region sequence and the V_{L} region sequence set forth in the SEQ ID NOS: listed in each row of Table 2. In some embodiments, the DLL3-binding receptor comprises a DLL3-binding antibody or fragment thereof, comprising a V_{H} region sequence and a V_{L} region sequence set forth in the SEQ ID NOS: listed in each row of Table 2. In some embodiments, the DLL3-binding receptor comprises a DLL3-binding antibody or fragment thereof, comprising an scFv sequence set forth in the SEQ ID NOS: listed in each row of Table 2, or an antibody comprising an scFv amino acid sequence that has at least 95%, 96%, 97%, 98%, or 99% sequence identity to the scFv sequence set forth in the SEQ ID NOS: listed in each row of Table 2. In some embodiments, the DLL3-binding receptor comprises a DLL3-binding antibody or fragment thereof, comprising an scFv sequence set forth in the SEQ ID NOS: listed in each row of Table 2.

In some embodiments, the provided CARs can include an extracellular antigen-binding domain that comprises all or a portion of an antibody, including polyclonal and monoclonal antibodies, including intact antibodies and functional (antigen-binding) antibody fragments, including fragment antigen-binding (Fab) fragments, F(ab′)2 fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody) fragments. In some aspects, the antibody or fragment thereof contained in the CARs include genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv.

b. Spacer

In some embodiments, the recombinant receptor such as the CAR, such as the antibody portion thereof, further includes a spacer (in some cases also called a spacer region), which may be or include at least a portion of an immunoglobulin constant region or variant or modified version thereof, such as a hinge region, e.g., an IgG4 hinge region, and/or a C_{H}1/C_{L}, C_{H}2 and/or C_{H}3 and/or Fc region. In some embodiments, the constant region or portion is of a human IgG, such as IgG4, IgG2 or IgG1. In some aspects, the portion of the constant region serves as a spacer between the antigen-recognition component, e.g., scFv, and transmembrane domain.

In some embodiments, the length of the spacer is adjusted to optimize the biophysical synapse distance between the CAR-expressing cell, such as a CAR-expressing cell, and the target of the CAR, such as a DLL3-expressing tumor cell. In some embodiments, the CAR is expressed by a T cell, and the length of the spacer is adjusted to a length that is compatible for T cell activation or to optimize CAR T-cell performance.

In some embodiments, the spacer can be of a length that provides for increased responsiveness of the cell following antigen-binding, as compared to in the absence of the spacer or as compared to an alternative spacer of a different length (e.g. longer in length). In some examples, the spacer is at or about 12 amino acids in length or is no more than at or about 12 amino acids in length. In some examples, the spacer is at or about 15 amino acids in length or is no more than at or about 15 amino acids in length.

Exemplary spacers include those having at least at or about 10 to at or about 300 amino acids, at or about 10 to at or about 229 amino acids, at or about 10 to at or about 200 amino acids, at or about 10 to at or about 175 amino acids, at or about 10 to at or about 150 amino acids, at or about 10 to at or about 125 amino acids, at or about 10 to at or about 100 amino acids, at or about 10 to at or about 75 amino acids, at or about 10 to at or about 50 amino acids, at or about 10 to at or about 40 amino acids, at or about 10 to at or about 30 amino acids, at or about 10 to at or about 20 amino acids, or at or about 10 to at or about 15 amino acids in length, and including any integer between the endpoints of any of the listed ranges. Exemplary spacers include those having at least at or about at or about 50 to at or about 175 amino acids, at or about 50 to at or about 150 amino acids, at or about 10 to at or about 125 amino acids, at or about 50 to at or about 100 amino acids, at or about 100 to at or about 300 amino acids, at or about 100 to at or about 250 amino acids, at or about 125 to at or about 250 amino acids, or at or about 200 to at or about 250 amino acids, and including any integer between the endpoints of any of the listed ranges. In some embodiments, a spacer is at least at or about 12 amino acids, at least at or about 119 amino acids, at least at or about 125 amino acids, at least at or about 200 amino acids, or at least at or about 220 amino acids, or at least at or about 225 amino acids in length. In some embodiments, a spacer is at least at or about 13 amino acids, at least at or about 120 amino acids, at least at or about 125 amino acids, at least at or about 200 amino acids, or at least at or about 220 amino acids, or at least at or about 229 amino acids in length. In some embodiments, a spacer is at or about 11, 12, 13, 14, 15, 16, 17, 18, 19, 20, 21, 22, 23, 24, 25 amino acids or less in length. In some embodiments, the spacer is at least at or about 100 amino acids in length, such as at least at or about 110, 111, 112, 113, 114, 115, 116, 117, 118, 119, 120, 121, 122, 123, 124, 125, 130, 135, 140, 145, 150, 160, 170, 180, 190, 200, 210, 220, 230, 240, or 250 amino acids in length.

In some embodiments, the spacer is at least at or about 125 to at or about 300 amino acids, at or about 125 to at or about 250 amino acids, at or about 125 to at or about 230 amino acids, at or about 125 to at or about 200 amino acids, at or about 125 to at or about 180 amino acids, at or about 125 to at or about 150 amino acids, at or about 150 to at or about 300 amino acids, at or about 150 to at or about 250 amino acids, at or about 150 to at or about 230 amino acids, at or about 150 to at or about 200 amino acids, at or about 150 to at or about 180 amino acids, at or about 180 to at or about 300 amino acids, at or about 180 to at or about 250 amino acids, at or about 180 to at or about 230 amino acids, at or about 180 to at or about 200 amino acids, at or about 200 to at or about 300 amino acids, at or about 200 to at or about 250 amino acids, at or about 200 to at or about 230 amino acids, at or about 230 to at or about 300 amino acids, at or about 230 to at or about 250 amino acids in length or 250 to at or about 300 amino acids in length. In some embodiments, the spacer is at least at or about 129, 130, 140, 150, 160, 170, 180, 190, 200, 210, 220, 221, 222, 223, 224, 225, 226, 227, 228, 229 or 230 amino acids in length, or a length between any of the foregoing.

Exemplary spacers include an IgG hinge alone, an IgG hinge linked to one or more of a C_{H}2 and C_{H}3 domain, or IgG hinge linked to the C_{H}3 domain. In some embodiments, the spacer includes an IgG hinge alone. In some embodiments, the IgG hinge, C_{H}2 and/or C_{H}3 can be derived all or in part from IgG4 or IgG2, such as all or in part from human IgG4 or human IgG2. In some embodiments, the spacer can be a chimeric polypeptide containing one or more of a hinge, C_{H}2 and/or C_{H}3 sequence(s) derived from IgG4, IgG2, and/or IgG2 and IgG4. In some embodiments, the hinge region comprises all or a portion of an IgG4 hinge region. In some embodiments, the hinge region comprises all or a portion of an IgG4 hinge region and/or of an IgG2 hinge region, wherein the IgG4 hinge region is optionally a human IgG4 hinge region and the IgG2 hinge region is optionally a human IgG2 hinge region; the C_{H}2 region comprises all or a portion of an IgG4 C_{H}2 region and/or of an IgG2 C_{H}2 region, wherein the IgG4 C_{H}2 region is optionally a human IgG4 C_{H}2 region and the IgG2 C_{H}2 region is optionally a human IgG2 C_{H}2 region; and/or the C_{H}3 region comprises all or a portion of an IgG4 C_{H}3 region and/or of an IgG2 C_{H}3 region, wherein the IgG4 C_{H}3 region is optionally a human IgG4 C_{H}3 region and the IgG2 C_{H}3 region is optionally a human IgG2 C_{H}3 region. In some embodiments, the hinge, C_{H}2 and C_{H}3 comprises all or a portion of each of a hinge region, C_{H}2 and C_{H}3 from IgG4. In some embodiments, the hinge region is chimeric and comprises a hinge region from human IgG4 and human IgG2; the C_{H}2 region is chimeric and comprises a C_{H}2 region from human IgG4 and human IgG2; and/or the C_{H}3 region is chimeric and comprises a C_{H}3 region from human IgG4 and human IgG2. In some embodiments, the spacer comprises an IgG4/2 chimeric hinge or a modified IgG4 hinge comprising at least one amino acid replacement compared to human IgG4 hinge region; an human IgG2/4 chimeric C_{H}2 region; and a human IgG4 C_{H}3 region.

In some embodiments, the spacer comprises or consists of all or a portion of an immunoglobulin hinge or a modified version thereof. In some embodiments, the spacer is at or about 15 amino acids or less in length. In some embodiments, the spacer comprises or consists of all or a portion of an immunoglobulin hinge, optionally an IgG4 hinge, or a modified version thereof and/or comprises about 15 amino acids or less. In some embodiments, the spacer is at or about 13 amino acids in length and/or comprises or consists of all or a portion of an immunoglobulin hinge, optionally an IgG4, or a modified version thereof. In some embodiments, the spacer is at or about 12 amino acids in length and/or comprises or consists of all or a portion of an immunoglobulin hinge, optionally an IgG4, or a modified version thereof. In some embodiments, the spacer comprises the formula X₁PPX₂P (SEQ ID NO:32), where X₁ is glycine, cysteine or arginine and X₂ is cysteine or threonine. In some embodiments, the spacer does not comprise a CD28 extracellular region or a CD8 extracellular region. In certain cases, the spacer has a methionine residue at the C-terminus. In some embodiments, the spacer comprises or consists of the sequence of SEQ ID NO:33-39, or a variant thereof having at least 85%, 86%, 87%, 88%, 89%, 90%, 91%, 92%, 93%, 94%, 95%, 96%, 97%, 98%, 99% or more sequence identity thereto. In some embodiments, the spacer comprises an amino acid sequence set forth in any of SEQ ID NOs:33-39.

In some embodiments, the spacer comprises or consists of the sequence of SEQ ID NO:1 or 3, or a variant thereof having at least 85%, 86%, 87%, 88%, 89%, 90%, 91%, 92%, 93%, 94%, 95%, 96%, 97%, 98%, 99% or more sequence identity thereto.

In some embodiments, the spacer comprises or consists of SEQ ID NO:3, or a variant thereof having at least 85%, 86%, 87%, 88%, 89%, 90%, 91%, 92%, 93%, 94%, 95%, 96%, 97%, 98%, 99% or more sequence identity to SEQ ID NO:3. In some embodiments, the spacer comprises or consists of the sequence set forth in SEQ ID NO:3. In some embodiments, the spacer comprises the sequence set forth in SEQ ID NO:3. In some embodiments, the spacer consists of the sequence set forth in SEQ ID NO:3. In some embodiments, the spacer comprises or consists of the amino acid sequence encoded by SEQ ID NO:4 or a nucleic acid sequence having at least at or about 90%, 91%, 92%, 93%, 94%, 95%, 96%, 97%, 98% or 99% sequence identity to SEQ ID NO:4. In some embodiments, the spacer comprises the amino acid sequence encoded by SEQ ID NO:4. In some embodiments, the spacer consists of the amino acid sequence encoded by SEQ ID NO:4.

In some embodiments, the spacer comprises or consists of SEQ ID NO:1, or a variant thereof having at least 85%, 86%, 87%, 88%, 89%, 90%, 91%, 92%, 93%, 94%, 95%, 96%, 97%, 98%, 99% or more sequence identity to SEQ ID NO:1. In some embodiments, the spacer comprises or consists of the sequence set forth in SEQ ID NO:1. In some embodiments, the spacer comprises the sequence set forth in SEQ ID NO:1. In some embodiments, the spacer consists of the sequence set forth in SEQ ID NO: 1. In some embodiments, the spacer comprises or consists of the amino acid sequence encoded by SEQ ID NO:2 or a nucleic acid sequence having at least at or about 90%, 91%, 92%, 93%, 94%, 95%, 96%, 97%, 98% or 99% sequence identity to SEQ ID NO:2. In some embodiments, the spacer comprises the amino acid sequence encoded by SEQ ID NO:2. In some embodiments, the spacer consists of the amino acid sequence encoded by SEQ ID NO:2.

In some embodiments, the spacer comprises or consists of the amino acid sequence encoded by SEQ ID NO:80 or a nucleic acid sequence having at least at or about 90%, 91%, 92%, 93%, 94%, 95%, 96%, 97%, 98% or 99% sequence identity to SEQ ID NO:80. In some embodiments, the spacer comprises the amino acid sequence encoded by SEQ ID NO:80. In some embodiments, the spacer consists of the amino acid sequence encoded by SEQ ID NO:80.

In some embodiments, the spacer is or comprises IgG hinge linked to the C_{H}3 domain, e.g., of a human immunoglobulin, such as IgG4 and/or IgG2. In some aspects, the spacer is at or about 111, 112, 113, 114, 115, 116, 117, 118, 119, 120, 121, 122, 123, 124 or 125 amino acids in length, or has a length between any of the foregoing. In some aspects, the spacer is at or about 119 or 120 amino acids in length.

In some embodiments, the spacer comprises or consists of the sequence of SEQ ID NO:5 or 58, or a variant thereof having at least 85%, 86%, 87%, 88%, 89%, 90%, 91%, 92%, 93%, 94%, 95%, 96%, 97%, 98%, 99% or more sequence identity thereto.

In some embodiments, the spacer comprises or consists of SEQ ID NO:5, or a variant thereof having at least 85%, 86%, 87%, 88%, 89%, 90%, 91%, 92%, 93%, 94%, 95%, 96%, 97%, 98%, 99% or more sequence identity to SEQ ID NO:5. In some embodiments, the spacer comprises or consists of the sequence set forth in SEQ ID NO:5. In some embodiments, the spacer comprises the sequence set forth in SEQ ID NO:5. In some embodiments, the spacer consists of the sequence set forth in SEQ ID NO:5. In some embodiments, the spacer comprises or consists of the amino acid sequence encoded by SEQ ID NO:57 or 81 or a nucleic acid sequence having at least at or about 90%, 91%, 92%, 93%, 94%, 95%, 96%, 97%, 98% or 99% sequence identity to SEQ ID NO:57 or 81. In some embodiments, the spacer comprises the amino acid sequence encoded by SEQ ID NO:57 or 81. In some embodiments, the spacer consists of the amino acid sequence encoded by SEQ ID NO:57 or 81.

In some embodiments, the spacer comprises or consists of SEQ ID NO:58, or a variant thereof having at least 85%, 86%, 87%, 88%, 89%, 90%, 91%, 92%, 93%, 94%, 95%, 96%, 97%, 98%, 99% or more sequence identity to SEQ ID NO:58. In some embodiments, the spacer comprises or consists of the sequence set forth in SEQ ID NO:58. In some embodiments, the spacer comprises the sequence set forth in SEQ ID NO:58. In some embodiments, the spacer consists of the sequence set forth in SEQ ID NO:58. In some embodiments, the spacer comprises or consists of the amino acid sequence encoded by SEQ ID NO:59 or 63 or a nucleic acid sequence having at least at or about 90%, 91%, 92%, 93%, 94%, 95%, 96%, 97%, 98% or 99% sequence identity to SEQ ID NO:59 or 63. In some embodiments, the spacer comprises the amino acid sequence encoded by SEQ ID NO:59 or 63. In some embodiments, the spacer consists of the amino acid sequence encoded by SEQ ID NO:59 or 63.

In some embodiments, the spacer is or comprises IgG hinge linked to a C_{H}2 domain and a C_{H}3 domain, e.g., of a human immunoglobulin, such as IgG4 and/or IgG2. In some aspects, the spacer is at or about 221, 222, 223, 224, 225, 226, 227, 228, 229, 230, 231, 232, 233, 234 or 235 amino acids in length, or has a length between any of the foregoing. In some aspects, the spacer is at or about 228 or 229 amino acids in length.

In some embodiments, the spacer can be from all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fc sequence set forth in SEQ ID NO:56 or an N176Q at position 176, in the C_{H}2 region, of the full-length IgG2 Fc sequence set forth in SEQ ID NO:55.

In some embodiments, the spacer is or comprises an IgG4/2 chimeric hinge or a modified IgG4 hinge; an IgG2/4 chimeric C_{H}2 region; and an IgG4 C_{H}3 region. In some embodiments, the spacer is about 228 or 229 amino acids in length, such as a spacer set forth in SEQ ID NO:42 or 82.

In some embodiments, the spacer comprises or consists of the sequence of SEQ ID NO:42 or 82, or a variant thereof having at least 85%, 86%, 87%, 88%, 89%, 90%, 91%, 92%, 93%, 94%, 95%, 96%, 97%, 98%, 99% or more sequence identity thereto.

In some embodiments, the spacer is or comprises an Ig hinge or a hinge; a chimeric C_{H}2 region; and a C_{H}3 region, such as set forth in SEQ ID NO:41 or is encoded in a sequence such as set forth in SEQ ID NO:40.

In some embodiments, the spacer comprises or consists of the sequence of SEQ ID NO:41, or a variant thereof having at least 85%, 86%, 87%, 88%, 89%, 90%, 91%, 92%, 93%, 94%, 95%, 96%, 97%, 98%, 99% or more sequence identity thereto. In some embodiments, the spacer incoded by a sequence which comprises or consists of the sequence of SEQ ID NO:40, or a variant thereof having at least 85%, 86%, 87%, 88%, 89%, 90%, 91%, 92%, 93%, 94%, 95%, 96%, 97%, 98%, 99% or more sequence identity thereto

In some embodiments, the spacer comprises or consists of SEQ ID NO:42, or a variant thereof having at least 85%, 86%, 87%, 88%, 89%, 90%, 91%, 92%, 93%, 94%, 95%, 96%, 97%, 98%, 99% or more sequence identity to SEQ ID NO:42. In some embodiments, the spacer comprises or consists of the sequence set forth in SEQ ID NO:42. In some embodiments, the spacer comprises the sequence set forth in SEQ ID NO:42. In some embodiments, the spacer consists of the sequence set forth in SEQ ID NO:42. In some embodiments, the spacer comprises or consists of the amino acid sequence encoded by SEQ ID NO:43 or 60 or a nucleic acid sequence having at least at or about 90%, 91%, 92%, 93%, 94%, 95%, 96%, 97%, 98% or 99% sequence identity to SEQ ID NO:43 or 60. In some embodiments, the spacer comprises the amino acid sequence encoded by SEQ ID NO:43 or 60. In some embodiments, the spacer consists of the amino acid sequence encoded by SEQ ID NO:43 or 60.

In some embodiments, the spacer comprises or consists of a IgD hinge. embodiments, the spacer comprises or consists of SEQ ID NO:7, or a variant thereof having at least 85%, 86%, 87%, 88%, 89%, 90%, 91%, 92%, 93%, 94%, 95%, 96%, 97%, 98%, 99% or more sequence identity to SEQ ID NO:7. In some embodiments, the spacer comprises or consists of the sequence set forth in SEQ ID NO:7. In some embodiments, the spacer comprises the sequence set forth in SEQ ID NO:7. In some embodiments, the spacer consists of the sequence set forth in SEQ ID NO:7.

In some embodiments, the spacer comprises or consists of SEQ ID NO:82, or a variant thereof having at least 85%, 86%, 87%, 88%, 89%, 90%, 91%, 92%, 93%, 94%, 95%, 96%, 97%, 98%, 99% or more sequence identity to SEQ ID NO:82. In some embodiments, the spacer comprises or consists of the sequence set forth in SEQ ID NO:82. In some embodiments, the spacer comprises the sequence set forth in SEQ ID NO:82. In some embodiments, the spacer consists of the sequence set forth in SEQ ID NO:82. In some embodiments, the spacer comprises or consists of the amino acid sequence encoded by SEQ ID NO:83 or 84 or a nucleic acid sequence having at least at or about 90%, 91%, 92%, 93%, 94%, 95%, 96%, 97%, 98% or 99% sequence identity to SEQ ID NO:83 or 84. In some embodiments, the spacer comprises the amino acid sequence encoded by SEQ ID NO:83 or 84. In some embodiments, the spacer consists of the amino acid sequence encoded by SEQ ID NO:83 or 84.

Additional exemplary spacers include, but are not limited to, those described in Hudecek et al. (2013) Clin. Cancer Res., 19:3153, Hudecek et al. (2015) Cancer Immunol. Res., 3(2):125-50, or WO2014031687. In some embodiments, the nucleotide sequence of the spacer is optimized to reduce RNA heterogeneity upon expression. In some embodiments, the nucleotide sequence of the spacer is optimized to reduce cryptic splice sites or reduce the likelihood of a splice event at a splice site.

In some embodiments, the spacer is encoded by a polynucleotide that has been optimized for codon usage and/or to reduce RNA heterogeneity, e.g., by removing cryptic splice sites. In some embodiments, the spacer is encoded by a polynucleotide that has been optimized for codon expression and/or to eliminate splice sites such as cryptic splice sites. In some embodiments, the coding sequence for the spacer comprises the nucleic acid sequence set forth in SEQ ID NO:2. In some embodiments, the coding sequence for the spacer comprises the nucleic acid sequence set forth in SEQ ID NO:4. In some embodiments, the coding sequence for the spacer comprises the nucleic acid sequence set forth in SEQ ID NO:57. In some embodiments, the coding sequence for the spacer comprises the nucleic acid sequence set forth in SEQ ID NO:59. In some embodiments, the coding sequence for the spacer comprises the nucleic acid sequence set forth in SEQ ID NO:60. In some embodiments, the coding sequence for the spacer comprises the nucleic acid sequence set forth in SEQ ID NO:84.

In some embodiments, the spacer has the amino acid sequence set forth in SEQ ID NO:1, and is encoded by the polynucleotide sequence set forth in SEQ ID NO:2. In some embodiments, the spacer has the amino acid sequence set forth in SEQ ID NO:3, and is encoded by the polynucleotide sequence set forth in SEQ ID NO:4. In some embodiments, the spacer has the amino acid sequence set forth in SEQ ID NO:5, and is encoded by the polynucleotide sequence set forth in SEQ ID NO:6. In some embodiments, the spacer has the amino acid sequence set forth in SEQ ID NO:58, and is encoded by the polynucleotide sequence set forth in SEQ ID NO:59. In some embodiments, the spacer has the amino acid sequence set forth in SEQ ID NO:42, and is encoded by the polynucleotide sequence set forth in SEQ ID NO:43. In some embodiments, the spacer has the amino acid sequence set forth in SEQ ID NO:82, and is encoded by the polynucleotide sequence set forth in SEQ ID NO:84.

In some embodiments, the spacer has an amino acid sequence that exhibits at least at or about 85%, 86%, 87%, 88%, 89%, 90%, 91%, 92%, 93%, 94%, 95%, 96%, 97%, 98% or 99% sequence identity to SEQ ID NO:1 or 3 and is encoded by a polynucleotide that has been optionally optimized for codon usage and/or to reduce RNA heterogeneity. In some embodiments, the spacer has an amino acid sequence that exhibits at least at or about 85%, 86%, 87%, 88%, 89%, 90%, 91%, 92%, 93%, 94%, 95%, 96%, 97%, 98% or 99% sequence identity to SEQ ID NO:5 or 58, and is encoded by a polynucleotide that has been optionally optimized for codon usage and/or to reduce RNA heterogeneity. In some embodiments, the spacer has an amino acid sequence that exhibits at least at or about 85%, 86%, 87%, 88%, 89%, 90%, 91%, 92%, 93%, 94%, 95%, 96%, 97%, 98% or 99% sequence identity to SEQ ID NO:42 or 82, and is encoded by a polynucleotide that has been optionally optimized for codon usage and/or to reduce RNA heterogeneity.

c. Transmembrane Domain

The antigen-recognition component generally is linked to one or more intracellular signaling components, such as signaling components that mimic activation through an antigen receptor complex, such as a TCR complex, in the case of a CAR, and/or signal via another cell surface receptor. Thus, in some embodiments, a DLL3-binding molecule (e.g., antibody or antigen-binding fragment thereof) is linked to one or more transmembrane domains such as those described herein and intracellular signaling domains comprising one or more intracellular components such as those described herein. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane domains include those derived from (i.e. comprise at least the transmembrane domain(s) of) the alpha, beta or zeta chain of the T-cell receptor, CD3 epsilon, CD4, CD5, CD8, CD9, CD16, CD22, CD28, CD33, CD37, CD45, CD64, CD80, CD86, CD134, CD137, and/or CD154. In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or a 28-amino acid sequence, or is a transmembrane domain that comprises the sequence of amino acids set forth in SEQ ID NO:10 or 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:10 or 12.

In some embodiments, the transmembrane domain comprises or consists of the sequence of SEQ ID NO:10 or 12, or a variant thereof having at least 85%, 86%, 87%, 88%, 89%, 90%, 91%, 92%, 93%, 94%, 95%, 96%, 97%, 98%, 99% or more sequence identity thereto.

In some embodiments, the transmembrane domain comprises or consists of SEQ ID NO:10, or a variant thereof having at least 85%, 86%, 87%, 88%, 89%, 90%, 91%, 92%, 93%, 94%, 95%, 96%, 97%, 98%, 99% or more sequence identity to SEQ ID NO:10. In some embodiments, the transmembrane domain comprises or consists of the sequence set forth in SEQ ID NO:10. In some embodiments, the transmembrane domain comprises the sequence set forth in SEQ ID NO:10. In some embodiments, the transmembrane domain consists of the sequence set forth in SEQ ID NO:10. In some embodiments, the transmembrane domain comprises or consists of the amino acid sequence encoded by SEQ ID NO:11 or a nucleic acid sequence having at least at or about 90%, 91%, 92%, 93%, 94%, 95%, 96%, 97%, 98% or 99% sequence identity to SEQ ID NO:11. In some embodiments, the transmembrane domain comprises the amino acid sequence encoded by SEQ ID NO:11. In some embodiments, the transmembrane domain consists of the amino acid sequence encoded by SEQ ID NO:11.

In some embodiments, the transmembrane domain comprises or consists of SEQ ID NO:12, or a variant thereof having at least 85%, 86%, 87%, 88%, 89%, 90%, 91%, 92%, 93%, 94%, 95%, 96%, 97%, 98%, 99% or more sequence identity to SEQ ID NO:12. In some embodiments, the transmembrane domain comprises or consists of the sequence set forth in SEQ ID NO:12. In some embodiments, the transmembrane domain comprises the sequence set forth in SEQ ID NO:12. In some embodiments, the transmembrane domain consists of the sequence set forth in SEQ ID NO:12. In some embodiments, the transmembrane domain comprises or consists of the amino acid sequence encoded by SEQ ID NO:13 or a nucleic acid sequence having at least at or about 90%, 91%, 92%, 93%, 94%, 95%, 96%, 97%, 98% or 99% sequence identity to SEQ ID NO:13. In some embodiments, the transmembrane domain comprises the amino acid sequence encoded by SEQ ID NO:13. In some embodiments, the transmembrane domain consists of the amino acid sequence encoded by SEQ ID NO:13. In some embodiments, the transmembrane domain comprises or consists of the amino acid sequence encoded by SEQ ID NO:14 or a nucleic acid sequence having at least at or about 90%, 91%, 92%, 93%, 94%, 95%, 96%, 97%, 98% or 99% sequence identity to SEQ ID NO:14. In some embodiments, the transmembrane domain comprises the amino acid sequence encoded by SEQ ID NO:14. In some embodiments, the transmembrane domain consists of the amino acid sequence encoded by SEQ ID NO:14.

In some embodiments, the transmembrane domain comprises or consists of an amino acid sequence encoded by a nucleic acid sequence having at least at or about 90%, 91%, 92%, 93%, 94%, 95%, 96%, 97%, 98% or 99% sequence identity to SEQ ID NOs: 62, 77, or 85. In some embodiments, the transmembrane domain comprises an amino acid sequence encoded by a nucleic acid sequence encoded by SEQ ID NOs: 62, 77, or 85. In some embodiments, the transmembrane domain consists of an amino acid sequence encoded by a nucleic acid sequence encoded by SEQ ID NO: 62, 77, or 85.

In some embodiments, the transmembrane domain is encoded by a polynucleotide that has been optionally optimized for codon usage and/or to reduce RNA heterogeneity, e.g., by removing cryptic splice sites. In some embodiments, the transmembrane domain has the amino acid sequence set forth in SEQ ID NO:10 or 12, and is encoded by the polynucleotide sequence set forth in SEQ ID NO:11 or 13, or a polynucleotide sequence having at least 90% sequence identity thereto. In certain cases, the transmembrane domain has a methionine residue at the N-terminus. In some embodiments, the transmembrane domain can be a CD28 transmembrane domain that comprises the sequence of amino acids set forth in SEQ ID NO:10, encoded by the nucleic acid sequence set forth in SEQ ID NO:11. In some embodiments, the transmembrane domain can be a CD28 transmembrane domain that comprises the sequence of amino acids set forth in SEQ ID NO:12, encoded by the nucleic acid sequence set forth in SEQ ID NO:13.

In some aspects, a particular combination of spacer and transmembrane domain sequences can be used.

In some embodiments, the spacer of the recombinant receptor, e.g., CAR, comprises the sequence set forth in SEQ ID NO:1, and the transmembrane domain of the recombinant receptor, e.g., CAR, comprises the sequence set forth in SEQ ID NO:10. In some embodiments, the spacer of the recombinant receptor, e.g., CAR, consists of the sequence set forth in SEQ ID NO:1, and the transmembrane domain of the recombinant receptor, e.g., CAR, consists of the sequence set forth in SEQ ID NO:10.

In some embodiments, the spacer of the recombinant receptor, e.g., CAR, comprises the sequence set forth in SEQ ID NO:3, and the transmembrane domain of the recombinant receptor, e.g., CAR, comprises the sequence set forth in SEQ ID NO:12. In some embodiments, the spacer of the recombinant receptor, e.g., CAR, consists of the sequence set forth in SEQ ID NO:3, and the transmembrane domain of the recombinant receptor, e.g., CAR, consists of the sequence set forth in SEQ ID NO:12.

In some embodiments, the spacer of the recombinant receptor, e.g., CAR, comprises the sequence set forth in SEQ ID NO:58, and the transmembrane domain of the recombinant receptor, e.g., CAR, comprises the sequence set forth in SEQ ID NO:10. In some embodiments, the spacer of the recombinant receptor, e.g., CAR, consists of the sequence set forth in SEQ ID NO:58, and the transmembrane domain of the recombinant receptor, e.g., CAR, consists of the sequence set forth in SEQ ID NO:10.

In some embodiments, the spacer of the recombinant receptor, e.g., CAR, comprises the sequence set forth in SEQ ID NO:5, and the transmembrane domain of the recombinant receptor, e.g., CAR, comprises the sequence set forth in SEQ ID NO:12. In some embodiments, the spacer of the recombinant receptor, e.g., CAR, consists of the sequence set forth in SEQ ID NO:5, and the transmembrane domain of the recombinant receptor, e.g., CAR, consists of the sequence set forth in SEQ ID NO:12.

In some embodiments, the spacer of the recombinant receptor, e.g., CAR, comprises the sequence set forth in SEQ ID NO:82, and the transmembrane domain of the recombinant receptor, e.g., CAR, comprises the sequence set forth in SEQ ID NO:10. In some embodiments, the spacer of the recombinant receptor, e.g., CAR, consists of the sequence set forth in SEQ ID NO:82, and the transmembrane domain of the recombinant receptor, e.g., CAR, consists of the sequence set forth in SEQ ID NO:10.

In some embodiments, the spacer of the recombinant receptor, e.g., CAR, comprises the sequence set forth in SEQ ID NO:42, and the transmembrane domain of the recombinant receptor, e.g., CAR, comprises the sequence set forth in SEQ ID NO:12. In some embodiments, the spacer of the recombinant receptor, e.g., CAR, consists of the sequence set forth in SEQ ID NO:42, and the transmembrane domain of the recombinant receptor, e.g., CAR, consists of the sequence set forth in SEQ ID NO:12.

In some aspects, alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

d. Intracellular Signaling Components

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the intracellular signaling domain of the CAR.

The receptor, e.g., the CAR, generally includes an intracellular signaling region comprising at least one intracellular signaling component or components. In some embodiments, the receptor includes an intracellular component or signaling domain of a TCR complex, such as a TCR CD3 chain that mediates T-cell activation and cytotoxicity, e.g., CD3 zeta (CD3-ζ) chain. Thus, in some aspects, the DLL3-binding antibody is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR includes a chimeric molecule between CD3-zeta (CD3-ζ) or Fc receptor γ and CD8, CD4, CD25 or CD16.

In some embodiments, upon ligation of the CAR, the cytoplasmic domain or intracellular signaling region of the CAR stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such classes of cytoplasmic signaling sequences.

In some aspects, the CAR includes a primary cytoplasmic signaling sequence that regulates primary stimulation and/or activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, CD3 gamma, CD3 delta and CD3 epsilon. In some embodiments, the intracellular signaling region in the CAR contains a cytoplasmic signaling domain, portion thereof, or sequence derived from CD3 zeta.

In some embodiments, the intracellular signaling domain comprises a human CD3 zeta stimulatory signaling domain or functional variant thereof, such as an 112 AA cytoplasmic domain of isoform 3 of human CD3ζ (Accession No.: P20963.2) or a CD3 zeta signaling domain as described in U.S. Pat. No. 7,446,190 or U.S. Pat. No. 8,911,993.

In some embodiments, the intracellular signaling domain comprises a human CD3 zeta signaling domain that comprises or consists of the sequence of SEQ ID NO:19, 21, or 22, or a variant thereof having at least 85%, 86%, 87%, 88%, 89%, 90%, 91%, 92%, 93%, 94%, 95%, 96%, 97%, 98%, 99% or more sequence identity to SEQ ID NO:19, 21, or 22.

In some embodiments, the intracellular signaling domain comprises or consists of a human CD3z signaling domain set forth in SEQ ID NO:19, or a variant thereof having at least 85%, 86%, 87%, 88%, 89%, 90%, 91%, 92%, 93%, 94%, 95%, 96%, 97%, 98%, 99% or more sequence identity to a human CD3z signaling domain set forth in SEQ ID NO:19. In some embodiments, the intracellular signaling domain comprises or consists of a human CD3z signaling domain set forth in SEQ ID NO:19. In some embodiments, the intracellular signaling domain comprises a human CD3z signaling domain set forth in SEQ ID NO:19. In some embodiments, the intracellular signaling domain consists of a human CD3z signaling domain set forth in SEQ ID NO:19. In some embodiments, the intracellular signaling domain comprises or consists of the amino acid sequence encoded by SEQ ID NO:20 or a nucleic acid sequence having at least at or about 90%, 91%, 92%, 93%, 94%, 95%, 96%, 97%, 98% or 99% sequence identity to SEQ ID NO:20. In some embodiments, the intracellular signaling domain comprises the amino acid sequence encoded by SEQ ID NO:20. In some embodiments, the intracellular signaling domain consists of the amino acid sequence encoded by SEQ ID NO:20.

In some embodiments, the intracellular signaling domain comprises or consists of SEQ ID NO:19, or a variant thereof having at least 85%, 86%, 87%, 88%, 89%, 90%, 91%, 92%, 93%, 94%, 95%, 96%, 97%, 98%, 99% or more sequence identity to SEQ ID NO:19. In some embodiments, the intracellular signaling domain comprises or consists of the sequence set forth in SEQ ID NO:19. In some embodiments, the intracellular signaling domain comprises the sequence set forth in SEQ ID NO:19. In some embodiments, the intracellular signaling domain consists of the sequence set forth in SEQ ID NO: 19. In some embodiments, the intracellular signaling domain comprises or consists of the amino acid sequence encoded by SEQ ID NO:20 or a nucleic acid sequence having at least at or about 90%, 91%, 92%, 93%, 94%, 95%, 96%, 97%, 98% or 99% sequence identity to SEQ ID NO:20. In some embodiments, the intracellular signaling domain comprises the amino acid sequence encoded by SEQ ID NO:20. In some embodiments, the intracellular signaling domain consists of the amino acid sequence encoded by SEQ ID NO:20.

In some embodiments, the intracellular signaling domain comprises or consists of the amino acid sequence encoded by SEQ ID NO:76 or a nucleic acid sequence having at least at or about 90%, 91%, 92%, 93%, 94%, 95%, 96%, 97%, 98% or 99% sequence identity to SEQ ID NO:76. In some embodiments, the intracellular signaling domain comprises the amino acid sequence encoded by SEQ ID NO:76. In some embodiments, the intracellular signaling domain consists of the amino acid sequence encoded by SEQ ID NO:76

In some embodiments, the intracellular signaling domain comprises or consists of SEQ ID NO:21, or a variant thereof having at least 85%, 86%, 87%, 88%, 89%, 90%, 91%, 92%, 93%, 94%, 95%, 96%, 97%, 98%, 99% or more sequence identity to SEQ ID NO:21. In some embodiments, the intracellular signaling domain comprises or consists of the sequence set forth in SEQ ID NO:21. In some embodiments, the intracellular signaling domain comprises the sequence set forth in SEQ ID NO:21. In some embodiments, the intracellular signaling domain consists of the sequence set forth in SEQ ID NO:21.

In some embodiments, the intracellular signaling domain comprises or consists of SEQ ID NO:22, or a variant thereof having at least 85%, 86%, 87%, 88%, 89%, 90%, 91%, 92%, 93%, 94%, 95%, 96%, 97%, 98%, 99% or more sequence identity to SEQ ID NO:22. In some embodiments, the intracellular signaling domain comprises or consists of the sequence set forth in SEQ ID NO:22. In some embodiments, the intracellular signaling domain comprises the sequence set forth in SEQ ID NO:22. In some embodiments, the intracellular signaling domain consists of the sequence set forth in SEQ ID NO:22.

In some embodiments the CD3 zeta comprises the sequence of amino acids set forth in SEQ ID NO:19, encoded by the nucleic acid sequence set forth in SEQ ID NO:20. In some embodiments, the CD3 zeta is encoded by a polynucleotide that has been optionally optimized for codon usage and/or to reduce RNA heterogeneity, e.g., by removing cryptic splice sites. In some embodiments, the CD3 zeta has the amino acid sequence set forth in SEQ ID NO:19, and is encoded by the polynucleotide sequence set forth in SEQ ID NO:20.

In some embodiments, the CAR includes a signaling domain (e.g., an intracellular or cytoplasmic signaling domain) and/or transmembrane portion of a costimulatory molecule, such as a T cell costimulatory molecule. Exemplary costimulatory molecules include CD28, 4-1BB, OX40, DAP10, and ICOS.

In some embodiments, the intracellular domain comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO:17 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:17.

In some embodiments, the intracellular signaling domain comprises or consists of a human 4-1BB costimulatory signaling domain set forth in SEQ ID NO:17, or a variant thereof having at least 85%, 86%, 87%, 88%, 89%, 90%, 91%, 92%, 93%, 94%, 95%, 96%, 97%, 98%, 99% or more sequence identity to a human 4-1BB costimulatory signaling domain set forth in SEQ ID NO:17. In some embodiments, the intracellular signaling domain comprises or consists of a human 4-1BB costimulatory signaling domain set forth in SEQ ID NO:17. In some embodiments, the intracellular signaling domain comprises a human 4-1BB costimulatory signaling domain set forth in SEQ ID NO:17. In some embodiments, the intracellular signaling domain consists of a human 4-1BB costimulatory signaling domain set forth in SEQ ID NO:17. In some embodiments, the intracellular signaling domain comprises or consists of the amino acid sequence encoded by SEQ ID NO:18 or a nucleic acid sequence having at least at or about 90%, 91%, 92%, 93%, 94%, 95%, 96%, 97%, 98% or 99% sequence identity to SEQ ID NO:18.

In some embodiments, the intracellular signaling domain comprises the amino acid sequence encoded by SEQ ID NO: 18. In some embodiments, the intracellular signaling domain consists of the amino acid sequence encoded by SEQ ID NO:18.

In some embodiments, the intracellular signaling domain comprises or consists of SEQ ID NO:17, or a variant thereof having at least 85%, 86%, 87%, 88%, 89%, 90%, 91%, 92%, 93%, 94%, 95%, 96%, 97%, 98%, 99% or more sequence identity to SEQ ID NO:17. In some embodiments, the intracellular signaling domain comprises or consists of the sequence set forth in SEQ ID NO:17. In some embodiments, the intracellular signaling domain comprises the sequence set forth in SEQ ID NO:17. In some embodiments, the intracellular signaling domain consists of the sequence set forth in SEQ ID NO: 17. In some embodiments, the intracellular signaling domain comprises or consists of the nucleic acid sequence encoded by SEQ ID NO:18 or a nucleic acid sequence having at least at or about 90%, 91%, 92%, 93%, 94%, 95%, 96%, 97%, 98% or 99% sequence identity to SEQ ID NO:18. In some embodiments, the intracellular signaling domain comprises the amino acid sequence encoded by SEQ ID NO:18. In some embodiments, the intracellular signaling domain consists of the amino acid sequence encoded by SEQ ID NO:18.

In some embodiments, the intracellular signaling domain comprises or consists of the nucleic acid sequence encoded by SEQ ID NO:67 or a nucleic acid sequence having at least at or about 90%, 91%, 92%, 93%, 94%, 95%, 96%, 97%, 98% or 99% sequence identity to SEQ ID NO:67. In some embodiments, the intracellular signaling domain comprises the amino acid sequence encoded by SEQ ID NO:67. In some embodiments, the intracellular signaling domain consists of the amino acid sequence encoded by SEQ ID NO:67.

In some embodiments, the costimulatory molecule from 4-1BB is encoded by a polynucleotide that has been optionally optimized for codon usage and/or to reduce RNA heterogeneity, e.g., by removing cryptic splice sites. In some embodiments, the costimulatory domain from 4-1BB has the amino acid sequence set forth in SEQ ID NO:17, and is encoded by the polynucleotide sequence set forth in SEQ ID NO:18.

In some embodiments, a costimulatory domain can be derived from CD28. In some aspects, a costimulatory domain can comprise the amino acid sequence set forth in SEQ ID NO:15. In some embodiments, the intracellular signaling domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO:15 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:15 or 16.

In some aspects, the same CAR includes both the stimulatory or activating components (e.g., cytoplasmic signaling sequence) and costimulatory components.

In some embodiments, the CAR encompasses one or more, e.g., two or more, costimulatory domains and a stimulatory or an activation domain, e.g., primary activation domain, in the cytoplasmic portion. Exemplary CARs include intracellular components of CD3-zeta, CD28, and 4-1BB.

In some aspects, the transmembrane domain comprises a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor comprises an intracellular domain of a T cell costimulatory molecule, such as between the transmembrane domain and intracellular signaling domain. In some aspects, the T cell costimulatory molecule is CD28 or 4-1BB.

In some embodiments, the stimulatory or activating components are included within one CAR, whereas the costimulatory component is provided by another CAR recognizing another antigen. In some embodiments, the CARs include activating or stimulatory CARs, and costimulatory CARs, both expressed on the same cell (see WO 2014/055668). In some aspects, the DLL3-targeting CAR is the stimulatory or activating CAR; in other aspects, it is the costimulatory CAR. In some embodiments, the cells further include inhibitory CARs (iCARs, see Fedorov et al., Sci. Transl. Medicine, 5(215) (December, 2013), such as a CAR recognizing an antigen other than DLL3, whereby a stimulatory or an activating signal delivered through the DLL3-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM⁻³, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In certain embodiments, the intracellular signaling region comprises a CD28 transmembrane and signaling domain linked to a CD3 (e.g., CD3-zeta) intracellular domain. In some embodiments, the intracellular signaling domain comprises a chimeric CD28 and 4-1BB (CD137; TNFRSF9) co-stimulatory domains, linked to a CD3 zeta intracellular domain.

e. Exemplary CARs

In some embodiments, provided anti-DLL3 CAR comprises an extracellular antigen-binding domain containing any of the anti-DLL3 antibody or antigen-binding fragments described herein, such as in Section I.A.1 and/or Table 2.

In some embodiments, provided embodiments of anti-DLL3 CAR comprises an extracellular antigen-binding domain containing any of the anti-DLL3 antibody or antigen-binding fragments described herein, such as in Section I.A.1 and/or Table 2; a spacer set forth in SEQ ID NO:1; a transmembrane domain, such as a transmembrane domain from a human CD28 set forth in SEQ ID NO:10; and an intracellular signaling region comprising a cytoplasmic signaling domain of a CD3-zeta (CD3ζ) chain and a costimulatory signaling domain, such as a signaling domain of a costimulatory molecule. In some embodiments, provided embodiments of anti-DLL3 CAR comprises an extracellular antigen-binding domain containing any of the anti-DLL3 antibody or antigen-binding fragments described herein, such as in Section I.A.1 and/or Table 2; a spacer set forth in SEQ ID NO:3; a transmembrane domain, such as a transmembrane domain from a human CD28 set forth in SEQ ID NO:12; and an intracellular signaling region comprising a cytoplasmic signaling domain of a CD3-zeta (CD3ζ) chain and a costimulatory signaling domain.

In some embodiments, provided anti-DLL3 CAR comprises an extracellular antigen-binding domain containing any of the anti-DLL3 antibody or antigen-binding fragments described herein, such as in Section I.A.1 and/or Table 2; a spacer comprising an IgG4/2 chimeric hinge or a modified IgG4 hinge, such as one that is about 12 amino acids in length, or a spacer set forth in SEQ ID NO:3, such as encoded by the nucleotide sequence set forth in SEQ ID NO:4; a transmembrane domain, such as a transmembrane domain from a human CD28, or a transmembrane domain set forth in SEQ ID NO:12, such as encoded by the nucleotide sequence set forth in SEQ ID NO:13; and an intracellular signaling region comprising a cytoplasmic signaling domain of a CD3-zeta (CD3ζ) chain and a costimulatory signaling domain, such as a costimulatory signaling domain from a human 4-1BB, for example, as set forth in SEQ ID NO:17, such as encoded by the nucleotide sequence set forth in SEQ ID NO:18.

In some embodiments, provided embodiments of anti-DLL3 CAR comprises an extracellular antigen-binding domain containing any of the anti-DLL3 antibody or antigen-binding fragments described herein, such as in Section I.A.1 and/or Table 2; a spacer comprising an IgG4/2 chimeric hinge or a modified IgG4 hinge, such as one that is about 13 amino acids in length, or a spacer set forth in SEQ ID NO:1, such as encoded by the nucleotide sequence set forth in SEQ ID NO:2, or a transmembrane domain set forth in SEQ ID NO:10, such as encoded by the nucleotide sequence set forth in SEQ ID NO:11; a transmembrane domain, such as a transmembrane domain from a human CD28; and an intracellular signaling region comprising a cytoplasmic signaling domain of a CD3-zeta (CD3ζ) chain and a costimulatory signaling domain, such as a costimulatory signaling domain from a human 4-1BB, for example, as set forth in SEQ ID NO:17, such as encoded by the nucleotide sequence set forth in SEQ ID NO:18.

In some embodiments, provided embodiments of anti-DLL3 CAR comprises an extracellular antigen-binding domain containing any of the anti-DLL3 antibody or antigen-binding fragments described herein, such as in Section I.A.1 and/or Table 2; a spacer comprising a modified IgG4 hinge-C_{H}3, such as one that is about 119 amino acids in length, or a spacer set forth in SEQ ID NO:5, such as encoded by the nucleotide sequence set forth in SEQ ID NO:6; a transmembrane domain, such as a transmembrane domain from a human CD28, or a transmembrane domain set forth in SEQ ID NO:12, such as encoded by the nucleotide sequence set forth in SEQ ID NO:13; and an intracellular signaling region comprising a cytoplasmic signaling domain of a CD3-zeta (CD3ζ) chain and a costimulatory signaling domain, such as a costimulatory signaling domain from a human 4-1BB, for example, as set forth in SEQ ID NO:17, such as encoded by the nucleotide sequence set forth in SEQ ID NO:18.

In some embodiments, provided embodiments of anti-DLL3 CAR comprises an extracellular antigen-binding domain containing any of the anti-DLL3 antibody or antigen-binding fragments described herein, such as in Section I.A.1 and/or Table 2; a spacer comprising a modified IgG4 hinge-C_{H}3, such as one that is about 120 amino acids in length, or a spacer set forth in SEQ ID NO:58, such as encoded by the nucleotide sequence set forth in SEQ ID NO:59; a transmembrane domain, such as a transmembrane domain from a human CD28, or a transmembrane domain set forth in SEQ ID NO:10, such as encoded by the nucleotide sequence set forth in SEQ ID NO:11; and an intracellular signaling region comprising a cytoplasmic signaling domain of a CD3-zeta (CD3ζ) chain and a costimulatory signaling domain, such as a costimulatory signaling domain from a human 4-1BB, for example, as set forth in SEQ ID NO:17, such as encoded by the nucleotide sequence set forth in SEQ ID NO:18.

In some embodiments, provided embodiments of anti-DLL3 CAR comprises an extracellular antigen-binding domain containing any of the anti-DLL3 antibody or antigen-binding fragments described herein, such as in Section I.A.1 and/or Table 2; a spacer comprising an IgG4/2 chimeric hinge or a modified IgG4 hinge; an IgG2/4 chimeric C_{H}2 region; and an IgG4 C_{H}3 region, such as one that is about 228 amino acids in length, or a spacer set forth in SEQ ID NO:42, such as encoded by the nucleotide sequence set forth in SEQ ID NO:43; a transmembrane domain, such as a transmembrane domain from a human CD28, or a transmembrane domain set forth in SEQ ID NO:12, such as encoded by the nucleotide sequence set forth in SEQ ID NO:13; and an intracellular signaling region comprising a cytoplasmic signaling domain of a CD3-zeta (CD3ζ) chain and a costimulatory signaling domain, such as a costimulatory signaling domain from a human 4-1BB, for example, as set forth in SEQ ID NO:17, such as encoded by the nucleotide sequence set forth in SEQ ID NO:18.

In some embodiments, provided embodiments of anti-DLL3 CAR comprises an extracellular antigen-binding domain containing any of the anti-DLL3 antibody or antigen-binding fragments described herein, such as in Section I.A.1 and/or Table 2; a spacer comprising an IgG4/2 chimeric hinge or a modified IgG4 hinge; an IgG2/4 chimeric C_{H}2 region; and an IgG4 C_{H}3 region, such as one that is about 229 amino acids in length, or a spacer set forth in SEQ ID NO:82, such as encoded by the nucleotide sequence set forth in SEQ ID NO:83; a transmembrane domain, such as a transmembrane domain from a human CD28, or a transmembrane domain set forth in SEQ ID NO:10, such as encoded by the nucleotide sequence set forth in SEQ ID NO:11; and an intracellular signaling region comprising a cytoplasmic signaling domain of a CD3-zeta (CD3ζ) chain and a costimulatory signaling domain, such as a costimulatory signaling domain from a human 4-1BB, for example, as set forth in SEQ ID NO:17, such as encoded by the nucleotide sequence set forth in SEQ ID NO:18.

In some of any such embodiments, the transmembrane domain is or comprises the sequence set forth in SEQ ID NO:10. In some of any such embodiments, the transmembrane domain is or comprises the sequence set forth in SEQ ID NO:12. In some of any such embodiments, the costimulatory signaling domain is an intracellular signaling domain of human CD28, human 4-1BB or human ICOS or a signaling portion thereof. In particular embodiments, the intracellular signaling domain is an intracellular signaling domain of human 4-1BB. In some of any such embodiments, the intracellular signaling domain is or comprises the sequence set forth in SEQ ID NO:17. In some of any such embodiments, the cytoplasmic signaling domain is a human CD3-zeta cytoplasmic signaling domain, such as set forth in SEQ ID NO:19. In some of any such embodiments, the intracellular signaling region comprises the sequences set forth in SEQ ID NO:17 and SEQ ID NO:19. In some of any such embodiments, those described in Section I.E herein, in Table 3 and/or in Tables E1-E4 and E8. Also provided are CARs encoded by the polynucleotides described in Section I.F herein, in Table 3 and/or in Tables E1-E4 and E8. Also provided are polynucleotides that contain any of the nucleotide sequences described herein, e.g., encoding all of a portion of the provided binding molecules. In certain cases, the transmembrane domain has a methionine residue at the N-terminus. In certain cases, the spacer has a methionine residue at the C-terminus.

In some of any of the provided embodiments, the anti-DLL3 chimeric antigen receptor is or comprises the sequence set forth in SEQ ID NO:117 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO:117. In some of any of the provided embodiments, the anti-DLL3 chimeric antigen receptor is or comprises the sequence set forth in SEQ ID NO:117 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO:117.

In some embodiments, the anti-DLL3 chimeric antigen receptor comprises or consists of SEQ ID NO:117, or a variant thereof having at least 85%, 86%, 87%, 88%, 89%, 90%, 91%, 92%, 93%, 94%, 95%, 96%, 97%, 98%, 99% or more sequence identity to SEQ ID NO:117. In some embodiments, the anti-DLL3 chimeric antigen receptor comprises or consists of the sequence set forth in SEQ ID NO:117. In some embodiments, the anti-DLL3 chimeric antigen receptor comprises the sequence set forth in SEQ ID NO:117. In some embodiments, the anti-DLL3 chimeric antigen receptor consists of the sequence set forth in SEQ ID NO:117. In some embodiments, the anti-DLL3 chimeric antigen receptor comprises or consists of the amino acid sequence encoded by SEQ ID NO:116 or a nucleic acid sequence having at least at or about 90%, 91%, 92%, 93%, 94%, 95%, 96%, 97%, 98% or 99% sequence identity to SEQ ID NO:116. In some embodiments, the anti-DLL3 chimeric antigen receptor comprises the amino acid sequence encoded by SEQ ID NO:116. In some embodiments, the anti-DLL3 chimeric antigen receptor consists of the amino acid sequence encoded by SEQ ID NO:116.

### 2. Exemplary Features

In some of any of the provided embodiments, the recombinant receptors, e.g., CARs, containing an DLL3-binding antibody or an antigen-binding fragment thereof, binds, such as specifically binds to DLL3, such as DLL3 on the surface of a cancer cell or a tumor cell. In any of the embodiments, an antibody or antigen-binding fragment, in the provided CARs, specifically binds DLL3, such as a human DLL3. Exemplary DLL3-binding recombinant receptors, e.g., CARs, can exhibit any binding affinity, binding specificity and/or other features of antigen recognition, such as species cross-reactivity, as described herein, such as in Section I.A.2. In some embodiments, a CAR containing an extracellular antigen-binding domain comprising the antibodies or fragments thereof exhibit similar or substantially the same binding properties and features as the antibody or fragment thereof. In some aspects, exemplary features of the antigen-binding portion of the CAR, e.g., comprising any of the antibody or antigen-binding fragment thereof, are similar to the affinities as described regarding the antibody or antigen-binding fragment thereof, such as in Section I.A.3. In some cases, the CAR containing an extracellular antigen-binding domain comprising the antibodies or fragment thereof exhibit different binding properties and features as the antibody or fragment thereof.

In some embodiments, the provided CARs specifically bind to a Delta-like ligand 3 (DLL3) protein. In some of any of the embodiments herein, DLL3 refers to human DLL3. The observation that an antibody or other binding molecule, e.g., CAR, binds to DLL3 or specifically binds to DLL3 does not necessarily mean that it binds to DLL3 from every species. For example, in some embodiments, features of binding to DLL3, such as the ability to specifically bind thereto and/or to compete for binding thereto with a reference receptor, e.g., reference CAR, containing a reference antibody, and/or to bind with a particular affinity or compete to a particular degree, in some embodiments, refers to the ability with respect to a human DLL3 and the antibody may not have this feature with respect to a DLL3 of another species such as mouse. In some embodiments, the CAR binds to human DLL3 and binds to DLL3 of another species, such as Rhesus macaque or macaque. In some embodiments, the CAR or an antigen-binding fragment thereof binds to human DLL3 and does not bind to DLL3 of another species, such as mouse. In some embodiments, the CAR binds to human DLL3 and binds to DLL3 of another species, such as mouse.

In some embodiments, the CARs bind, such as specifically bind, to human DLL3, such as to one or more epitopes or region of human DLL3, such as the mature human DLL3 sequence set forth in SEQ ID NO:122 or 123, or an allelic variant or splice variant thereof. In some embodiments, the antibodies or antigen-binding fragment thereof specifically binds to one or more epitopes within a human DLL3. In some embodiments, the human DLL3 comprises the sequence of amino acids forth in SEQ ID NO:120. In some embodiments, the human DLL3 comprises the sequence of amino acids forth in SEQ ID NO:121. In some embodiments, the human DLL3 isoform (mature, isoform 1) comprises the sequence of amino acids forth in SEQ ID NO:122. In some embodiments, the human DLL3 isoform (mature, isoform 2) comprises the sequence of amino acids forth in SEQ ID NO:123. In some embodiments, the antibodies or antigen-binding fragment thereof bind to the extracellular region DLL3, such as to one or more extracellular epitopes present within the extracellular region of mature human DLL3, e.g., corresponding to residues 27-492 of the human DLL3 precursor sequence set forth in SEQ ID NO:120.

In some embodiments, the CARs bind one or more epitope of DLL3, such as a linear epitope of DLL3, such as a human DLL3 or one or more conformational epitopes of DLL3, such as a human DLL3.

In some embodiments, the CARs bind to an epitope comprising amino acid residues within the N-terminus of Notch ligand domain, DSL domain, EGF-like 1 domain, EGF-like 2 domain, EGF-like 3 domain, EGF-like 4 domain, EGF-like 5 domain, or EGF-like 6 domain.

In some embodiments, the CARs bind one or more epitopes of human DLL3, such as one or more epitopes comprising 2, 3, 4, 5, 6, 7, 8, 9, 10, 11, 12, 13, 14, 15, or more contiguous amino acid residues within human DLL3 (for example, human DLL3 precursor sequence set forth in SEQ ID NO:120), or two or more epitopes, each comprising 1, 2, 3, 4, 5, 6, 7, 8, 9, 10, 11, 12, 13, 14, 15, or more contiguous amino acid residues within human DLL3 (for example, human DLL3 precursor sequence set forth in SEQ ID NO:120). In some aspects, the one or more epitopes include 2, 3, 4, 5, 6, 7, 8, 9, 10, 11, 12, 13, 14, 15, or more contiguous amino acid residues within amino acid residues 27-84 correspond to the N-terminus of Notch ligand domain, amino acid residues 176-215 correspond to the DSL domain, amino acid residues 216-249 correspond to the EGF-like 1 domain, amino acid residues 274-310 correspond to the EGF-like 2 domain, amino acid residues 312-351 correspond to the EGF-like 3 domain, amino acid residues 353-389 correspond to the EGF-like 4 domain, amino acid residues 391-427 correspond to the EGF-like 5 domain, or amino acid residues 429-465 correspond to the EGF-like 6 domain, with reference to the human DLL3 precursor sequence set forth in SEQ ID NO:120.

In some embodiments, the CAR binds to an epitope within amino acid residues 27-84 with reference to SEQ ID NO:120, corresponding to the N-terminus of Notch ligand domain. In some embodiments, the CAR binds to an epitope within amino acid residues 176-215 with reference to SEQ ID NO:120, corresponding to the DSL domain. In some embodiments, the CAR binds to an epitope within amino acid residues 216-249 with reference to SEQ ID NO:120, corresponding to the EGF-like 1 domain. In some embodiments, the CAR binds to an epitope within amino acid residues 274-310 with reference to SEQ ID NO:120, corresponding to the EGF-like 2 domain. In some embodiments, the CAR binds to an epitope within amino acid residues 312-351 with reference to SEQ ID NO:120, corresponding to the EGF-like 3 domain. In some embodiments, the CAR binds to an epitope within amino acid residues 353-389 with reference to SEQ ID NO:120, corresponding to the EGF-like 4 domain. In some embodiments, the CAR binds to an epitope within amino acid residues 391-427 with reference to SEQ ID NO:120, corresponding to the EGF-like 5 domain. In some embodiments, the CAR binds to an epitope within amino acid residues 429-465 with reference to SEQ ID NO: 120, corresponding to the EGF-like 6 domain. In some embodiments, the CAR binds to an epitope within amino acid residues 493-513 with reference to SEQ ID NO:120, corresponding to the transmembrane domain.

In some embodiments, the CAR binds to non-human DLL3, such as monkey, rabbit, rat, mouse, or other species of DLL3. In some embodiments, the CAR binds to non-human DLL3, such as cynomolgus monkey (Macaca fascicularis) DLL3, such as to an epitope or region of cynomolgus monkey DLL3, such as the cynomolgus monkey DLL3 set forth in SEQ ID NO:125 (NCBI Reference Sequence: XM_005589196.2). In some embodiments, the CAR binds to mouse (Mus musculus) DLL3, such as to an epitope or region of mouse DLL3, such as the mouse DLL3 set forth in SEQ ID NO:124 (GenBank No. NP_031892.2).

In some embodiments, the provided CARs are capable of binding DLL3, such as human DLL3, with at least a certain affinity, as measured by any of a number of known methods. In some embodiments, the affinity is represented by an equilibrium dissociation constant (K_{D}). In some embodiments, the affinity is represented by EC₅₀. In some aspects, the provided CAR exhibits a K_{D} or an EC₅₀ as described herein, for example, in Section I.A.3.

In some embodiments, the binding affinity of a binding molecule, such as an anti-DLL3 CAR, for different antigens, e.g., DLL3 proteins from different species can be compared to determine the species cross-reactivity. For example, species cross-reactivity can be classified as high cross reactivity or low cross reactivity. In some embodiments, the equilibrium dissociation constant, K_{D}, for different antigens, e.g., DLL3 proteins from different species such as human, cynomolgus monkey or mouse, can be compared to determine species cross-reactivity. In some embodiments, the species cross-reactivity of an anti-DLL3 CAR can be high, e.g., the anti-DLL3 CAR binds to human DLL3 and a species variant DLL3 to a similar degree, e.g., the ratio of K_{D} for human DLL3 and K_{D} for the species variant DLL3 is or is about 1. In some embodiments, the species cross-reactivity of an anti-DLL3 CAR can be low, e.g., the anti-DLL3 CAR has a high affinity for human DLL3 but a low affinity for a species variant DLL3, or vice versa. For example, the ratio of K_{D} for the species variant DLL3 and K_{D} for the human DLL3 is more than 10, 15, 20, 25, 30, 40, 50, 60, 70, 80, 90, 100, 200, 500, 1000, 2000 or more, and the anti-DLL3 CAR has low species cross-reactivity. The degree of species cross-reactivity can be compared with the species cross-reactivity of a known CAR, such as a reference CAR.

Among the provided CARs are CARs that exhibit antigen-dependent activity or signaling, i.e. signaling activity that is measurably absent or at background levels in the absence of antigen, e.g. DLL3, and/or in the presence of non-specific antigen. Thus, in some aspects, provided CARs do not exhibit, or exhibit no more than background or a tolerable or low level of, tonic signaling or antigen-independent activity or signaling in the absence of antigen, e.g. DLL3, being present. In some embodiments, the provided anti-DLL3 CAR-expressing cells exhibit biological activity or function, including cytotoxic activity, cytokine production, and ability to proliferate. In some embodiments, the provided CARs receptor exhibits the same, substantially the same or higher antigen-specific signaling and/or antigen dependent activity or signaling compared to a reference DLL3-specific chimeric antigen receptor, optionally under the same or substantially the same conditions or assay.

In some embodiments, the provided CARs exhibit absent, reduced or lower levels of activity or signaling in the absence of antigen, e.g., DLL3. In some embodiments, the provided CARs exhibit reduced, lower or is nearly absent of or completely absent of tonic signaling or antigen-independent signaling or activity, e.g., signaling or activity in the absence of antigen, such that the signaling or activity is less than or at or about 30%, 20% or 10%, such as less than at or about 10%, of the signaling or activity of the CAR in the presence of human DLL3. In some embodiments, the provided CARs exhibit the same, substantially the same or lower tonic signaling and/or antigen independent activity or signaling compared to a reference DLL3-specific chimeric antigen receptor (CAR), optionally under the same or substantially the same conditions or assay. In some aspects, among a plurality or population of cells that are engineered to express the CARs, less than at or about 10%, at or about 9%, at or about 8%, at or about 7%, at or about 5%, at or about 4%, at or about 3%, at or about 2% or at or about 1% of the cells in the plurality comprise a chimeric antigen receptor that exhibits tonic signaling and/or antigen independent activity or signaling.

In some embodiments, engineered cells expressing the provided anti-DLL3 CARs exhibit improved biological activity or functional activity, such as anti-tumor activity, tumor growth inhibition, tumor volume reduction, persistence, expansion, or prolonged survival of the subject, when administered to a subject for adoptive cell therapy. In some embodiments, engineered cells expressing the provided CARs exhibit improved biological activity or functional activity, such as anti-tumor activity, tumor growth inhibition, tumor volume reduction, persistence, expansion, or prolonged survival of the subject, compared to engineered cells expressing a reference DLL3-specific chimeric antigen receptor, optionally under the same or substantially the same conditions or assay. In some embodiments, biological activity or functional activity of a chimeric receptor, such as cytotoxic activity, can be measured using any of a number of known methods. The activity can be assessed or determined either in vitro or in vivo. In some embodiments, activity can be assessed once the cells are administered to the subject (e.g., human). Parameters to assess include specific binding of an engineered or natural T cell or other immune cell to antigen, e.g., in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as interlekukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or TGF-beta (TGFO). Assays to measure cytokines are well known, and include but are not limited to, ELISA, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample. In some aspects the biological activity can be measured using an animal model of the disease or condition, such as a tumor xenograft model, and assessing the reduction in tumor burden or load and/or survival. In some aspects the biological activity is measured by assessing clinical outcome, such as reduction in tumor burden or load.

In some embodiments, administration of engineered cells expressing the provided anti-DLL3 CARs exhibit substantially improved tumor growth inhibition, in vivo persistence and/or prolonged survival of the subject with a tumor, compared to administration of engineered cells expressing reference CAR. In some aspects, such improvement is observed in subjects having various types of DLL3-expressing cancers, including, but not limited to, small cell lung cancer (SCLC), large cell neuroendocrine carcinoma (LCNEC), melanomas, gliomas, and glioblastomas.

In some embodiments, engineered cells expressing the provided anti-DLL3 CARs exhibit improved persistence and expansion when administered to a subject for adoptive cell therapy. In some embodiments, engineered cells expressing the provided CARs exhibit the same, substantially the same or higher persistence and expansion compared to engineered cells expressing a reference DLL3-specific chimeric antigen receptor, optionally under the same or substantially the same conditions or assay. In some aspects, the proliferation, expansion and/or persistence of an engineered cell expressing any of the provided receptors, e.g., CARs, can be assessed by determining the exposure, number, concentration, persistence and proliferation of the engineered cells, e.g., cells administered for adoptive cell therapy. In some embodiments, the exposure, number or level of engineered T cells, e.g., T cells administered for the T cell based therapy, or subset thereof, such as CD3⁺ cells, CD4⁺ cells, CD8⁺ cells, CD3⁺ CAR⁺ cells, CD4⁺ CAR⁺ cells or CD8⁺ CAR⁺ cells can be assessed, e.g., from a subject, such as a human subject or an animal subject, that had been administered engineered cells. In some aspects, the exposure, number, concentration, persistence and proliferation relate to pharmacokinetic parameters. In some cases, pharmacokinetics can be assessed by measuring such parameters as the maximum (peak) plasma concentration (Cₘₐₓ), the peak time (i.e. when maximum plasma concentration (Cₘₐₓ) occurs; Tₘₐₓ), the minimum plasma concentration (i.e. the minimum plasma concentration between doses of a therapeutic agent, e.g., CAR+ T cells; Cₘᵢₙ), the elimination half-life (T_{1/2}) and area under the curve (i.e. the area under the curve generated by plotting time versus plasma concentration of the therapeutic agent CAR⁺ T cells; AUC), following administration. The concentration of a particular therapeutic agent, e.g., CAR⁺ T cells, in the plasma following administration can be measured using any known methods suitable for assessing concentrations of therapeutic agents, e.g., CAR⁺ T cells, in samples of blood, or any methods described herein. For example, nucleic acid-based methods, such as quantitative PCR (qPCR) or flow cytometry-based methods, or other assays, such as an immunoassay, ELISA, or chromatography/mass spectrometry-based assays can be used.

In some cases, to assess the specificity, cross-reactivity and/or antigen dependency of a particular activity, signaling or function of the receptor, e.g., CAR, any of the described assays for assessing activity, signaling or biological function of the receptors can be assessed in the presence and absence of the specific target antigen, e.g., human DLL3, or in the presence of the specific target antigen, e.g., human DLL3, and in the presence of a different, non-specific antigen or a non-human DLL3, such as a mouse DLL3.

In some of any embodiments, the provided anti-DLL3 CAR exhibits the same, substantially the same or lower tonic signaling and/or antigen independent activity or signaling compared to a reference DLL3-specific CAR, e.g., under the same or substantially the same conditions or assay. For example, in some embodiments, the provided anti-DLL3 CAR exhibits a level or degree of tonic signaling and/or antigen independent activity or signaling that is at least at or about 75%, 80%, 90%, 95% or 99% less than the level or degree of tonic signaling and/or antigen independent activity of a reference DLL3-specific CAR, e.g., under the same or substantially the same conditions or assay.

In some embodiments, the provided anti-DLL3 CARs exhibit improved expression on the surface of cells, such as compared to an alternative CAR that has an identical amino acid sequence but that is encoded by non-splice site eliminated and/or a codon-optimized nucleotide sequence. In some embodiments, the expression of the recombinant receptor on the surface of the cell can be assessed. Approaches for determining expression of the recombinant receptor on the surface of the cell may include use of chimeric antigen receptor (CAR)-specific antibodies (e.g., Brentjens et al., Sci. Transl. Med. 2013 March; 5(177): 177ra38), Protein L (Zheng et al., J. Transl. Med. 2012 February; 10:29), epitope tags, and monoclonal antibodies that specifically bind to a CAR polypeptide (see WO2014190273). In some embodiments, the expression of the recombinant receptor on the surface of the cell, e.g., primary T cell, can be assessed, for example, by flow cytometry, using binding molecules that can bind to the recombinant receptor or a portion thereof that can be detected. In some embodiments, the binding molecules used for detecting expression of the recombinant receptor an anti-idiotypic antibody, e.g., an anti-idiotypic agonist antibody specific for a binding domain, e.g., scFv, or a portion thereof. In some embodiments, the binding molecule is or comprises an isolated or purified antigen, e.g., recombinantly expressed antigen.

### F. Polynucleotides Encoding Binding Molecules

Also provided are polynucleotides encoding the binding molecules, such as anti-DLL3 antibodies, antigen-binding fragments thereof, recombinant receptors (e.g., chimeric antigen receptors) and/or portions, e.g., chains or fragments, thereof. Among the provided polynucleotides are those encoding the anti-DLL3 antibodies (e.g., antigen-binding fragment) or chimeric antigen receptors described herein. The polynucleotides may include those encompassing natural and/or non-naturally occurring nucleotides and bases, e.g., including those with backbone modifications. The terms “nucleic acid molecule”, “nucleic acid”, “sequence of nucleotides”, and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

Provided are polynucleotides that contain a nucleic acid encoding any of the anti-DLL3 antibody or antigen-binding domain thereof described herein, or any portion, fragment, chain or domain thereof. In some embodiments, the antibody or antigen-binding fragment thereof contain multiple domains or chains (e.g., heavy chain and a light chain), and all of the antibody or antigen-binding fragment thereof is encoded in one polynucleotide. Also provided are polynucleotides that contain a nucleic acid encoding any of the single chain cell surface proteins described herein. Also provided are polynucleotides that contain a nucleic acid encoding any of the conjugate described herein. Also provided are polynucleotides that contain a nucleic acid encoding any of the anti-DLL3 chimeric antigen receptors described herein. In some embodiments, the binding molecule, such as the antibody or antigen-binding fragment thereof or recombinant receptors, contain multiple domains or chains (e.g., a heavy chain and a light chain), and all of the binding molecule is encoded in more than one polynucleotide, such as two or more polynucleotides. In some embodiments, the polynucleotides are comprised in a vector.

In some aspects, provided are polynucleotides that contain nucleic acid sequences encoding any of the binding molecules provided herein, for example, in Section I.A and I.E. In some embodiments, provided are polynucleotides that contain nucleic acid sequences encoding a portion, fragment, chain or domain of any of the binding molecules provided herein, for example, in Section I.A and I.E.

In some cases, the polynucleotide encoding the DLL3-binding molecules, such as an antibody or antigen-binding fragment thereof or a recombinant receptor (e.g., CAR) comprises a signal sequence that encodes a signal peptide, in some cases encoded upstream of the nucleic acid sequences encoding the DLL3-binding molecules, such as an antibody or antigen-binding fragment thereof or a recombinant receptor (e.g., CAR), or joined at the 5′ terminus of the nucleic acid sequences encoding the antigen-binding domain. In some cases, the polynucleotide containing nucleic acid sequences encoding the DLL3-binding molecules, such as an antibody or antigen-binding fragment thereof or a recombinant receptor (e.g., CAR), comprises a signal sequence that encodes a signal peptide. In some aspects, the signal sequence may encode a signal peptide derived from a native polypeptide. In other aspects, the signal sequence may encode a heterologous or non-native signal peptide. In some aspects, non-limiting exemplary signal peptide include a signal peptide of the IgG kappa chain set forth in SEQ ID NO:50 or encoded by the nucleotide sequence set forth in SEQ ID NO:51. In some aspects, a non-limiting exemplary signal peptide includes a signal peptide of a GMCSFR alpha chain set forth in SEQ ID NO:52 and encoded by the nucleotide sequence set forth in SEQ ID NO:53. In some aspects, a non-limiting exemplary signal peptide includes a signal peptide of a CD8 alpha signal peptide set forth in SEQ ID NO:54. In some aspects, a non-limiting exemplary signal peptide includes a signal peptide of a CD33 signal peptide set forth in SEQ ID NO:48 and encoded by the nucleotide sequence set forth in SEQ ID NO:49. In some aspects, a nucleotide sequence encoding a CD33 signal peptide is set forth in SEQ ID NO:78 or 79. In some cases, the polynucleotide encoding the DLL3-binding molecules, such as an antibody or antigen-binding fragment thereof or a recombinant receptor (e.g., CAR) can contain nucleic acid sequence encoding additional molecules, such as a surrogate marker or other markers, or can contain additional components, such as promoters, regulatory elements and/or multicistronic elements. In some embodiments, the nucleic acid sequence encoding the DLL3-binding molecules, such as an antibody or antigen-binding fragment thereof or a recombinant receptor (e.g., CAR) can be operably linked to any of the additional components.

In some embodiments, provided are polynucleotides contain nucleic acid sequences encoding a variable heavy chain domain (V_{H}) of an antibody or an antigen-binding fragment thereof, or a recombinant receptor containing an antibody or an antigen-binding fragment thereof. In some embodiments, provided are polynucleotides contain nucleic acid sequences encoding a variable light chain domain (V_{L}) of an antibody or an antigen-binding fragment thereof, or a recombinant receptor containing an antibody or an antigen-binding fragment thereof. In some embodiments, provided are polynucleotides that contain nucleic acid sequences encoding a variable heavy chain domain (V_{H}) and a variable light chain domain (V_{L}) of an antibody or an antigen-binding fragment thereof, or a recombinant receptor containing an antibody or an antigen-binding fragment thereof.

In some of any embodiments, the provided polynucleotide comprises a nucleic acid encoding the V_{H} comprising the sequence set forth in SEQ ID NO:87, or a nucleic acid sequence having at least at or about 90%, 91%, 92%, 93%, 94%, 95%, 96%, 97%, 98% or 99% sequence identity to SEQ ID NO:87. In some of any embodiments, the provided polynucleotide comprises a nucleic acid encoding the V_{H} comprising the sequence set forth in SEQ ID NO:87.

In some of any embodiments, the provided polynucleotide comprises a nucleic acid encoding the V_{L} comprising the sequence set forth in SEQ ID NO:88, or a nucleic acid sequence having at least at or about 90%, 91%, 92%, 93%, 94%, 95%, 96%, 97%, 98% or 99% sequence identity to SEQ ID NO:88. n some of any embodiments, the provided polynucleotide comprises a nucleic acid encoding the V_{L} comprising the sequence set forth in SEQ ID NO:88.

In some of any embodiments, the provided polynucleotide comprises a nucleic acid encoding the V_{H} comprising the sequence set forth in SEQ ID NO:87 or a nucleic acid sequence having at least at or about 90%, 91%, 92%, 93%, 94%, 95%, 96%, 97%, 98% or 99% sequence identity to SEQ ID NO:87, and a nucleic acid encoding the V_{L} comprising the sequence set forth in SEQ ID NO:88, or a nucleic acid sequence having at least at or about 90%, 91%, 92%, 93%, 94%, 95%, 96%, 97%, 98% or 99% sequence identity to SEQ ID NO:88. In some of any embodiments, the provided polynucleotide comprises a nucleic acid encoding the V_{H} comprising the sequence set forth in SEQ ID NO:87, and a nucleic acid encoding the V_{L} Comprising the sequence set forth in SEQ ID NO:88.

In some embodiments, the polynucleotide comprises sequences encoding the antibody or antigen-binding fragment that is an scFv comprising a heavy chain variable (V_{H}) region and a light chain variable (V_{L}) region. In some embodiments, polynucleotide comprises sequences encoding one or more linkers joining two antibody domains or regions, such as a V_{H} region and a V_{L} region. Exemplary encoded linkers include peptide linkers that are rich in glycine and serine and/or in some cases threonine.

In some embodiments, the nucleic acid encoding the V_{H} region may be 5′ to the nucleic acid encoding the V_{L} region. In some embodiments, the nucleic acid encoding the V_{H} region may be 3′ to the nucleic acid encoding the V_{L} region. In particular embodiments, the nucleic acid encoding the scFv, may include nucleic acid encoding the V_{H} region or portion thereof, followed by nucleic acid encoding a linker, followed by nucleic acid encoding the V_{L} region or portion thereof. In particular embodiments, the nucleic acid encoding the scFv, may include nucleic acid encoding the V_{L} region or portion thereof, followed by nucleic acid encoding a linker, followed by nucleic acid encoding the V_{H} region or portion thereof.

In some of any embodiments, the polynucleotide comprises a nucleic acid encoding the scFv comprising the sequence set forth in SEQ ID NO:86 or a nucleic acid sequence having at least at or about 90%, 91%, 92%, 93%, 94%, 95%, 96%, 97%, 98% or 99% sequence identity to SEQ ID NO:86. In some of any embodiments, the polynucleotide comprises a nucleic acid encoding the scFv comprising the sequence set forth in SEQ ID NO:86.

In some aspects, the polynucleotides comprise nucleic acids encoding one or more components of the recombinant receptor, e.g., CAR.

In some embodiments, the polynucleotide comprises a nucleic acid encoding the spacer comprising the sequence set forth in SEQ ID NO:2 or a nucleic acid sequence having at least at or about 90%, 91%, 92%, 93%, 94%, 95%, 96%, 97%, 98% or 99% sequence identity to SEQ ID NO:2. In some embodiments, the polynucleotide comprises the sequence set forth in SEQ ID NO:2.

In some embodiments, the polynucleotide comprises a nucleic acid encoding the spacer comprising the sequence set forth in SEQ ID NO:4 or a nucleic acid sequence having at least at or about 90%, 91%, 92%, 93%, 94%, 95%, 96%, 97%, 98% or 99% sequence identity to SEQ ID NO:4. In some embodiments, the polynucleotide comprises the sequence set forth in SEQ ID NO:4.

In some embodiments, the polynucleotide comprises a nucleic acid encoding the spacer comprising the sequence set forth in SEQ ID NO:6 or a nucleic acid sequence having at least at or about 90%, 91%, 92%, 93%, 94%, 95%, 96%, 97%, 98% or 99% sequence identity to SEQ ID NO:6. In some embodiments, the polynucleotide comprises the sequence set forth in SEQ ID NO:6.

In some embodiments, the polynucleotide comprises a nucleic acid encoding the spacer comprising the sequence set forth in SEQ ID NO:59 or a nucleic acid sequence having at least at or about 90%, 91%, 92%, 93%, 94%, 95%, 96%, 97%, 98% or 99% sequence identity to SEQ ID NO:59. In some embodiments, the polynucleotide comprises the sequence set forth in SEQ ID NO:59.

In some embodiments, the polynucleotide comprises a nucleic acid encoding the spacer comprising the sequence set forth in SEQ ID NO:60 or a nucleic acid sequence having at least at or about 90%, 91%, 92%, 93%, 94%, 95%, 96%, 97%, 98% or 99% sequence identity to SEQ ID NO:60. In some embodiments, the polynucleotide comprises the sequence set forth in SEQ ID NO:60.

In some embodiments, the polynucleotide comprises a nucleic acid encoding the spacer comprising the sequence set forth in SEQ ID NO:84 or a nucleic acid sequence having at least at or about 90%, 91%, 92%, 93%, 94%, 95%, 96%, 97%, 98% or 99% sequence identity to SEQ ID NO:84. In some embodiments, the polynucleotide comprises the sequence set forth in SEQ ID NO:84.

Also provided are polynucleotides that have been optimized for codon usage and/or to eliminate splice sites, such as cryptic splice sites. In some embodiments, the polynucleotides are modified to optimize codon usage. In some embodiments, the polynucleotides are codon optimized for expression in a human cell such as a human T cell such as a primary human T cell. In some embodiments, the polynucleotides, such as those encoding any of the antibodies, receptors (such as antigen receptors such as chimeric antigen receptors) and/or DLL3-specific binding proteins provided herein, are or have been modified to reduce heterogeneity or contain one or more nucleic acid sequences observed herein (such as by the optimization methods) to result in improved features of the polypeptides, such as the CARs, as compared to those containing distinct, reference, sequences or that have not been optimized. In some embodiments, the polynucleotide is optimized by splice site elimination. Among such features include improvements in RNA heterogeneity, such as that resulting from the presence of one or more splice sites, such as one or more cryptic splice sites, and/or improved expression and/or surface expression of the encoded protein, such as increased levels, uniformity, or consistency of expression among cells or different therapeutic cell compositions engineered to express the polypeptides. In some embodiments, the polynucleotides can be codon optimized for expression in human cells.

Transcribed nucleic acid sequences generally, in nature, in a mammalian cell, undergo processing co-transcriptionally or immediately following transcription, wherein a nascent precursor messenger ribonucleic acid (pre-mRNA), transcribed from a genomic deoxyribonucleic acid (DNA) sequence, is in some cases edited by way of splicing, to remove introns, followed by ligation of the exons in eukaryotic cells. Consensus sequences for splice sites are known, but in some aspects, specific nucleotide information defining a splice site may be complex and may not be readily apparent based on available methods. Cryptic splice sites are splice sites that are not predicted based on the standard consensus sequences and are variably activated. Hence, variable splicing of pre-mRNA at cryptic splice sites leads to heterogeneity in the transcribed mRNA products upon expression in eukaryotic cells.

Polynucleotides generated for the expression of transgenes are typically constructed from nucleic acid sequences, such as complementary DNA (cDNA), or portions thereof, that do not contain introns. Thus, splicing of such sequences is not expected to occur. However, the presence of cryptic splice sites within the cDNA sequence can lead to unintended or undesired splicing reactions and heterogeneity in the transcribed mRNA. Such heterogeneity results in translation of unintended protein products, such as truncated protein products with variable amino acid sequences that exhibit modified expression and/or activity.

In some embodiments, eliminating splice sites, such as cryptic splice sites, can improve or optimize expression of a transgene product, such as a polypeptide translated from the transgene, such as an anti-DLL3 CAR polypeptide. Splicing at cryptic splice sites of an encoded transgene, such as an encoded DLL3 CAR molecule, can lead to reduced protein expression, e.g., expression on cell surfaces, and/or reduced function, e.g., reduced intracellular signaling. Provided herein are polynucleotides, encoding anti-DLL3 CAR proteins that have been optimized to reduce or eliminate cryptic splice sites. Also provided herein are polynucleotides encoding anti-DLL3 CAR proteins that have been optimized for codon expression and/or in which one or more sequence, such as one identified by the methods or observations herein regarding splice sites, is present, and/or in which an identified splice site, such as any of the identified splice sites herein, is not present. Among the provided polynucleotides are those exhibiting below a certain degree of RNA heterogeneity or splice forms when expressed under certain conditions and/or introduced into a specified cell type, such as a human T cell, such as a primary human T cell, and cells and compositions and articles of manufacture containing such polypeptides and/or exhibiting such properties. In some embodiments, the RNA heterogeneity of transcribed RNA is reduced by greater than or greater than about 10%, 15%, 20%, 25%, 30%, 40%, 50% or more compared to a polynucleotide that has not been modified to remove cryptic splice sites and/or by codon optimization. In some embodiments, the provided polynucleotides encoding an anti-DLL3 CAR exhibit RNA homogeneity of transcribed RNA that is at least 70%, 75%, 80%, 85%, 90%, or 95% or greater.

RNA heterogeneity can be determined by any of a number of methods provided herein or described or known. In some embodiments, RNA heterogeneity of a transcribed nucleic acid is determined by amplifying the transcribed nucleic acid, such as by reverse transcriptase polymerase chain reaction (RT-PCR) followed by detecting one or more differences, such as differences in size, in the one or more amplified products. In some embodiments, the RNA heterogeneity is determined based on the number of differently sized amplified products, or the proportion of various differently sized amplified products. In some embodiments, RNA, such as total RNA or cytoplasmic polyadenylated RNA, is harvested from cells, expressing the transgene to be optimized, and amplified by reverse transcriptase polymerase chain reaction (RT-PCR) using a primer specific to the 5′ untranslated region (5′ UTR), in some cases corresponding to a portion of the promoter sequence in the expression vector, located upstream of the transgene in the transcribed RNA, and a primer specific to the 3′ untranslated region (3′ UTR), located downstream of the expressed transgene in the transcribed RNA sequence or a primer specific to a sequence within the transgene. In particular embodiments, at least one primer complementary to a sequence in the 5′ untranslated region (UTR) and at least one primer complementary to a sequence in the 3′ untranslated region (UTR) are employed to amplify the transgene. One can resolve RNA, such as messenger RNA, and analyze the heterogeneity thereof by several methods. Non-limiting, exemplary methods include agarose gel electrophoresis, chip-based capillary electrophoresis, analytical centrifugation, field flow fractionation, and chromatography, such as size exclusion chromatography or liquid chromatography.

In some embodiments, a provided polynucleotide encoding an anti-DLL3 CAR provided herein, or a construct provided herein, includes modifications to remove one or more splice donor and/or acceptor site that may contribute to splice events and/or reduced expression and/or increased RNA heterogeneity. In some embodiments, provided polynucleotides are modified in one or more polynucleotides in the spacer region to eliminate or reduce splice events.

In some embodiments, the polynucleotides are modified polynucleotides, including polynucleotides that were modified for codon optimization (O) and/or splice site elimination (SSE). Examples of the SEQ ID NOS: for such polynucleotides are set forth in Table 3, wherein nucleotide (nt) sequences for the components of the CAR constructs prior to splice site elimination and codon optimization (N/O), nucleic acid (nt) sequences for the components of the CAR constructs following splice site elimination and optimization (O/SSE), and the corresponding amino acid (aa) sequences encoded by the nucleic acid sequences are provided. The components include the anti-DLL3 scFv, spacer, transmembrane (tm) domain, 4-1BB costimulatory signaling domain sequence (costim), CD3ζ signaling domain (CD3ζ). The full sequences of the CARs following splice site elimination and optimization (CAR) are also provided. In some cases, the polynucleotide also includes the CD33 signal sequence (ss), a T2A ribosomal skip element (T2A) and truncated EGF receptor (EGFRt) sequence.

In some of any embodiments, the polynucleotide comprises a nucleic acid encoding the V_{H} comprising the sequence set forth in SEQ ID NO:87, and a nucleic acid encoding the V_{L} comprising the sequence set forth in SEQ ID NO:88.

In some of any embodiments, the polynucleotide comprises a nucleic acid encoding the scFv comprising the sequence set forth in SEQ ID NO:86.

In some of any embodiments, the polynucleotide comprises a nucleic acid encoding the spacer comprising the sequence set forth in SEQ ID NO:2. In some of any embodiments, the polynucleotide comprises a nucleic acid encoding the spacer comprising the sequence set forth in SEQ ID NO:4. In some of any embodiments, the polynucleotide comprises a nucleic acid encoding the spacer comprising the sequence set forth in SEQ ID NO:59. In some of any embodiments, the polynucleotide comprises a nucleic acid encoding the spacer comprising the sequence set forth in SEQ ID NO:84.

In some embodiments, polynucleotide sequences of an anti-DLL3 CAR are set forth in SEQ ID NO:116, encoding the amino acid sequences set forth in SEQ ID NO:117.

In some of any embodiments, the polynucleotide comprises the sequence set forth in SEQ ID NO:116 or a sequence that exhibits at least 90%, 91%, 92%, 93%, 94%, 95%, 96%, 97%, 98%, or 99% sequence identity to the sequence set forth in SEQ ID NO:116. In some of any embodiments, the polynucleotide comprises the sequence set forth in SEQ ID NO:116.

In some of any embodiments, the polynucleotide comprises a nucleic acid encoding the sequence set forth in SEQ ID NO:117 or a sequence that encodes a polypeptide sequence that exhibits at least 90%, 91%, 92%, 93%, 94%, 95%, 96%, 97%, 98%, or 99% sequence identity to the sequence set forth in SEQ ID NO:117. In some of any embodiments, the polynucleotide comprises a nucleic acid encoding the sequence set forth in SEQ ID NO:117.

In some of any embodiments, the polynucleotide comprises the sequence set forth in SEQ ID NO:118 or a sequence that exhibits at least 90%, 91%, 92%, 93%, 94%, 95%, 96%, 97%, 98%, or 99% sequence identity to the sequence set forth in SEQ ID NO:118. In some of any embodiments, the polynucleotide comprises the sequence set forth in SEQ ID NO:118.

In some of any embodiments, the polynucleotide comprises a nucleic acid encoding the sequence set forth in SEQ ID NO:119 or a sequence that encodes a polypeptide sequence that exhibits at least 90%, 91%, 92%, 93%, 94%, 95%, 96%, 97%, 98%, or 99% sequence identity to the sequence set forth in SEQ ID NO: 119. In some of any embodiments, the polynucleotide comprises a nucleic acid encoding the sequence set forth in SEQ ID NO: 119.

TABLE 3
Exemplary DLL3-binding CAR components (SEQ ID NOS:)

| | | scFv (excluding | | | | | CAR (excluding |
|---|---|---|---|---|---|---|---|
| CAR# | scFv# | signal sequence) | spacer | TM | 4-1BB | CD3z | signal sequence) |
| | | Nucleotides (nt) | | | | | |
| CAR-A | from | 86 | 2 | 11 | 18 | 20 | 116 |
| | DLL3- | | | | | | |
| | mAb-1 | | | | | | |
| CAR-A | from | 86 | 4 | 13 | 18 | 20 | 116 |
| | DLL3- | | | | | | |
| | mAb-1 | | | | | | |
| | | Amino acids (aa) | | | | | |
| CAR-A | from | 89 | 1 | 10 | 17 | 19 | 117 |
| | DLL3- | | | | | | |
| | mAb-1 | | | | | | |
| CAR-A | from | 89 | 3 | 12 | 17 | 19 | 117 |
| | DLL3- | | | | | | |
| | mAb-1 | | | | | | |

### II. ENGINEERED CELLS

Also provided are cells such as engineered cells that comprise or express any of the provided DLL3-binding molecules, such as an antibody or an antigen-binding fragment thereof or a recombinant receptor (e.g., a chimeric antigen receptor) such as one that comprises an extracellular domain including an anti-DLL3 antibody or fragment as described herein. Also provided are cells such as engineered cells that comprise any of the provided polynucleotides encoding any of the provided DLL3-binding molecules, such as a DLL3-binding antibodies or antigen-binding fragments thereof or a recombinant receptor (e.g., CAR).

Also provided are populations of such cells, compositions containing such cells and/or enriched for such cells, such as in which cells expressing the DLL3-binding molecule make up at least 50%, 60%, 70%, 80%, 90%, 91%, 92%, 93%, 94%, 95%, 96%, 97%, 98%, 9%, or more of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Thus also provided are genetically engineered cells expressing the recombinant receptors containing the antibodies, e.g., cells containing the CARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more polynucleotides introduced via genetic engineering, and thereby express recombinant or genetically engineered products of such polynucleotides. In some embodiments, the polynucleotides are heterologous, i.e., normally not present in a cell or sample obtained from the cell, such as one obtained from another organism or cell, which for example, is not ordinarily found in the cell being engineered and/or an organism from which such cell is derived. In some embodiments, the polynucleotides are not naturally occurring, such as a polynucleotide not found in nature, including one comprising chimeric combinations of polynucleotides encoding various domains from multiple different cell types. In some embodiments, the cells (e.g., engineered cells) comprise a vector (e.g., a viral vector, expression vector, etc.) as described herein such as a vector comprising a nucleic acid encoding a recombinant receptor described herein.

### A. Polynucleotides, Vectors and Methods for Genetic Engineering

Also provided are polynucleotides encoding the antibodies, antigen-binding fragment thereof and/or receptors, such as CARs, comprising the antibodies or antigen-binding fragment thereof. Among the provided polynucleotides are those encoding the anti-DLL3 antibodies (e.g., antigen-binding fragment) described herein. Also provided are polynucleotides encoding one or more antibodies and/or portions thereof, e.g., those encoding one or more of the anti-DLL3 antibodies (e.g., antigen-binding fragment) described herein and/or other antibodies and/or portions thereof, e.g., antibodies and/or portions thereof that binds other target antigens. Also provided are methods, polynucleotides, compositions, and kits, for expressing the binding molecules (e.g., DLL3 binding molecules), including recombinant receptors (e.g., CARs) comprising the binding molecules, and for producing the genetically engineered cells expressing such binding molecules. In some embodiments, one or more binding molecules, including recombinant receptors (e.g., CARs) can be genetically engineered into cells or plurality of cells. The genetic engineering generally involves introduction of a nucleic acid encoding the recombinant or engineered component into the cell, such as by retroviral transduction, transfection, or transformation.

The nucleic acids may include those encompassing natural and/or non-naturally occurring nucleotides and bases, e.g., including those with backbone modifications. The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide. In some aspects, the nucleic acid sequence encoding at least a portion of the antibody or antigen-binding fragment thereof, such as an scFv, conjugates, receptors (e.g., CARs) provided herein can be optimized, for example, codon-optimized for expression in a human cell and/or optimized to reduce or eliminate cryptic splice sites.

In some embodiments, the polynucleotides also include one or more additional sequences, such as those encoding one or more additional molecules, such as a marker, or promoters, regulatory elements and/or multicistronic elements. In some embodiments, the provided polynucleotides include any of the polynucleotides described herein, e.g., in Section I.F.

Also provided are vectors containing the polynucleotides and host cells containing the vectors, e.g., for producing the antibodies or antigen-binding fragments thereof. Also provided are methods for producing the antibodies or antigen-binding fragments thereof. The polynucleotide may encode an amino acid sequence comprising the V_{L} region and/or an amino acid sequence comprising the V_{H} region of the antibody (e.g., the light and/or heavy chains of the antibody). The polynucleotide may encode one or more amino acid sequence comprising the V_{L} region and/or an amino acid sequence comprising the V_{H} region of the antibody (e.g., the light and/or heavy chains of the antibody). In some embodiments, the polynucleotide, e.g., polynucleotide encodes one or more V_{H} region and/or one or more V_{L} region of the antibody, in any order or orientation. In some embodiments, the polynucleotide, e.g., polynucleotide encodes a V_{H} region and a V_{L} region, and the coding sequence for the V_{H} region is upstream of the coding sequence for the V_{L} region. In some embodiments, the polynucleotide, e.g., polynucleotide encodes a V_{H} region and a V_{L} region, and the coding sequence for the V_{L} region is upstream of the coding sequence for the V_{H} region.

Also provided are vectors containing the polynucleotides and engineered cells containing the vectors, e.g., engineered immune cells expressing the binding molecules such as recombinant receptors. Also provided are methods for engineering cells, such as immune cells, to express the anti-DLL3 binding molecule, such as a recombinant receptor, e.g., a chimeric antigen receptor (CAR). The polynucleotide may encode an amino acid sequence comprising the V_{L} region and/or an amino acid sequence comprising the V_{H} region of the antibody (e.g., the light and/or heavy chains of the antibody) as the extracellular antigen-binding domain; a transmembrane domain and intracellular domains, such as a CD3 zeta and a costimulatory signaling domain.

In a further embodiment, one or more vectors (e.g., expression vectors) comprising such polynucleotides are provided. In a further embodiment, a host cell comprising such polynucleotides is provided. In one such embodiment, a host cell comprises (e.g., has been transformed with) a vector comprising a polynucleotide that encodes an amino acid sequence comprising the V_{H} region of the antibody. In another such embodiment, a host cell comprises (e.g., has been transformed with) (1) a vector comprising a polynucleotide that encodes an amino acid sequence comprising the V_{L} region of the antibody and an amino acid sequence comprising the V_{H} region of the antibody, or (2) a first vector comprising a polynucleotide that encodes an amino acid sequence comprising the V_{L} region of the antibody and a second vector comprising a polynucleotide that encodes an amino acid sequence comprising the V_{H} region of the antibody. In some embodiments, a host cell comprises (e.g., has been transformed with) one or more vectors comprising one or more polynucleotide that encodes one or more an amino acid sequence comprising one or more antibodies and/or portions thereof, e.g., antigen-binding fragments thereof. In some embodiments, one or more such host cells are provided. In some embodiments, a composition containing one or more such host cells are provided. In some embodiments, the one or more host cells can express different antibodies, or the same antibody. In some embodiments, each of the host cells can express more than one antibody.

Also provided are methods of making the anti-DLL3 chimeric antigen receptors. For recombinant production of the chimeric receptors, a polynucleotide sequence encoding a chimeric receptor antibody, e.g., as described herein, may be isolated and inserted into one or more vectors for further cloning and/or expression in a host cell. Such polynucleotide sequences may be readily isolated and sequenced using conventional procedures (e.g., by using oligonucleotide probes that are capable of binding specifically to genes encoding the heavy and light chains of the antibody). In some embodiments, a method of making the anti-DLL3 chimeric antigen receptor is provided, wherein the method comprises culturing a host cell comprising a polynucleotide sequence encoding the antibody, as provided above, under conditions suitable for expression of the receptor.

Also provided are methods of making the anti-DLL3 antibodies (including antigen-binding fragments). For recombinant production of the anti-DLL3 antibody, a polynucleotide sequence or a polynucleotide encoding an antibody, e.g., as described above, may be isolated and inserted into one or more vectors for further cloning and/or expression in a host cell. Such polynucleotide sequences may be readily isolated and sequenced using conventional procedures (e.g., by using oligonucleotide probes that are capable of binding specifically to genes encoding the heavy and light chains of the antibody). In some embodiments, a method of making the anti-DLL3 antibody is provided, wherein the method comprises culturing a host cell comprising a polynucleotide sequence encoding the antibody, as provided above, under conditions suitable for expression of the antibody, and optionally recovering the antibody from the host cell (or host cell culture medium).

In addition to prokaryotes, eukaryotic microbes such as filamentous fungi or yeast are suitable cloning or expression hosts for antibody-encoding vectors, including fungi and yeast strains whose glycosylation pathways have been modified to mimic or approximate those in human cells, resulting in the production of an antibody with a partially or fully human glycosylation pattern. See Gerngross, Nat. Biotech. 22:1409-1414 (2004), and Li et al., Nat. Biotech. 24:210-215 (2006).

Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, the antibody heavy chains and/or light chains (e.g., V_{H} region and/or V_{L} region) may be expressed in yeast. See, e.g., U.S. Publication No. US 2006/0270045 A1. In some embodiments, a particular eukaryotic host cell is selected based on its ability to make desired post-translational modifications to the heavy chains and/or light chains (e.g., V_{H} region and/or V_{L} region). For example, in some embodiments, CHO cells produce polypeptides that have a higher level of sialylation than the same polypeptide produced in 293 cells.

In some embodiments, the antibody or antigen-binding fragment provided herein is produced in a cell-free system. Exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

The provided embodiments further include vectors and host cells and other expression systems for expressing and producing the antibodies and other antigen-binding proteins, including eukaryotic and prokaryotic host cells, including bacteria, filamentous fungi, and yeast, as well as mammalian cells such as human cells, as well as cell-free expression systems.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example, in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphoribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells further are engineered to promote expression of cytokines or other factors. Various methods for the introduction of genetically engineered components, e.g., antigen receptors, e.g., CARs, are well known and may be used with the provided methods and compositions. Exemplary methods include those for transfer of polynucleotides encoding the receptors, including via viral, e.g., retroviral or lentiviral, transduction, transposons, and electroporation.

In some embodiments, recombinant polynucleotides are transferred into cells using recombinant infectious virus particles, such as, e.g., vectors derived from simian virus 40 (SV40), adenoviruses, adeno-associated virus (AAV). In some embodiments, recombinant polynucleotides are transferred into T cells using recombinant lentiviral vectors, such as HIV-1 lentivirus-based vectors (lentivectors; see, e.g., Amado et al., Science. 1999 Jul. 30; 285(5428):674-676), or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557).

In some embodiments, the retroviral vector or lentiviral vector has a long terminal repeat sequence (LTR). In some embodiments the vector is derived from the Moloney murine leukemia virus (MoMLV), myeloproliferative sarcoma virus (MPSV), murine embryonic stem cell virus (MESV), murine stem cell virus (MSCV), spleen focus forming virus (SFFV), human immunodeficiency virus type 1 (HIV-1) or human immunodeficiency virus type 2 (HIV-2/SIV). In some embodiments, the vectors are self-inactivating (SIN). In some embodiments, the vectors are conditionally replicating (mobilizable) vectors. Most lentiviral vectors are derived from human, feline or simian lentiviruses. Most retroviral vectors are derived from murine retroviruses. In some embodiments, the lentiviruses or retroviruses include those derived from any avian or mammalian cell source. The lentiviruses or retroviruses typically are amphotropic, meaning that they are capable of infecting host cells of several species, including humans. In one embodiment, the gene to be expressed replaces the retroviral gag, pol and/or env sequences. Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. A number of illustrative retroviral systems have also been described (e.g., Amado et al., (1999) Science 285(5428):674-676, U.S. Pat. Nos. 5,219,740; 6,207,453; 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

In some embodiments, recombinant polynucleotides are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant polynucleotides are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston (1990) Nature 346: 776-777); and strontium phosphate DNA co-precipitation (Brash et al., (1987) Mol. Cell Biol.7: 2031-2034). Other approaches and vectors for transfer of the polynucleotides encoding the recombinant products are those described, e.g., in WO2014055668, and U.S. Pat. No. 7,446,190.

Among additional polynucleotides, e.g., genes for introduction are those to improve the outcome of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

In some embodiments the vector or construct can contain a promoter and/or enhancer or regulatory elements to regulate expression of the encoded recombinant receptor. In some examples the promoter and/or enhancer or regulatory elements can be condition-dependent promoters, enhancers, and/or regulatory elements. In some examples these elements drive expression of the transgene. In some examples, the CAR transgene can be operatively linked to a promoter, such as an EFlalpha promoter with an HTLV1 enhancer (SEQ ID NO:70). In some examples, the CAR transgene is operatively linked to a Woodchuck Hepatitis Virus (WHP) Posttranscriptional Regulatory Element (WPRE; SEQ ID NO:71), located downstream of the transgene.

In some embodiments, the vector or construct can contain a single promoter that drives the expression of one or more nucleic acid molecules. In some embodiments, such nucleic acid molecules, e.g., transcripts, can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g., encoding a first and second chimeric receptor) by a message from a single promoter. For example, in some embodiments, the vector or construct can contain a nucleic acid encoding an anti-DLL3 receptor (e.g., an anti-DLL3 CAR) provided herein and a nucleic acid encoding a different molecule, separated by an IRES, under the regulation of a single promoter.

Alternatively, in some cases, a single promoter may direct expression of an RNA that comprises, in a single open reading frame (ORF), two or three genes (e.g. encoding a first and second binding molecules, e.g., antibody recombinant receptor) separated from one another by sequences encoding a self-cleavage peptide (e.g., 2A cleavage sequences) or a protease recognition site (e.g., furin). The ORF thus encodes a single polypeptide, which, either during (in the case of T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and polynucleotides disclosed herein, without limitation, 2A sequences from the foot-and-mouth disease virus (F2A, e.g., SEQ ID NO:28 or 74), equine rhinitis A virus (E2A, e.g., SEQ ID NO:27 or 73), Thosea asigna virus (T2A, e.g., SEQ ID NO:8, 24 or 75, or encoded in SEQ ID NO:61), and porcine teschovirus-1 (P2A, e.g., SEQ ID NO:25 or 26) as described in U.S. Patent Publication No. 20070116690. In some embodiments, the one or more different or separate promoters drive the expression of one or more nucleic acid molecules encoding the one or more binding molecules, e.g., recombinant receptors.

Any of the recombinant receptors provided herein, e.g., anti-DLL3 recombinant receptors and/or the additional recombinant receptors, can be encoded by polynucleotides containing one or more nucleic acid molecules encoding the receptors, in any combinations or arrangements. For example, one, two, three or more polynucleotides can encode one, two, three or more different receptors or domains. In some embodiments, one vector or construct comprises nucleic acid molecules encoding one or more recombinant receptor(s), and a separate vector or construct comprises nucleic acid molecules encoding an additional binding molecule, e.g., antibody and/or recombinant receptor, such as an anti-DLL3 receptor (e.g., anti-DLL3 CAR).

In some embodiments, the nucleic acid molecules can also encode one or more surrogate marker(s), such as fluorescent protein (e.g., green fluorescent protein (GFP)) or a cell surface marker (e.g., a truncated surface marker such as truncated EGFR (tEGFR), which may be used to confirm transduction or engineering of the cell to express the receptor. For example, in some aspects, extrinsic marker genes are utilized in connection with engineered cell therapies to permit detection or selection of cells and, in some cases, also to promote cell suicide by ADCC. Exemplary marker genes include truncated epidermal growth factor receptor (EGFRt), which can be co-expressed with a transgene of interest (e.g., a CAR or TCR) in transduced cells (see, e.g., U.S. Pat. No. 8,802,374). EGFRt comprises an epitope recognized by the antibody cetuximab (Erbitux®). For this reason, Erbitux® can be used to identify or select cells that have been engineered with the EGFRt construct, including in cells also co-engineered with another recombinant receptor, such as a chimeric antigen receptor (CAR).

In some embodiments, the nucleic acid encoding the binding molecules further contain comprises a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:9, 23 or 66) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR).

In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), 0-galactosidase, chloramphenicol acetyltransferase (CAT), 0-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO:9, 23, or 66 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:9, 23, or 66. An exemplary polypeptide for a truncated EGFR (e.g. tEGFR) is encoded by the sequence of nucleic acids set forth in SEQ ID NO:64 or 65 or a sequence of nucleic acids that exhibits at least 85%, 86%, 87%, 88%, 89%, 90%, 91%, 92%, 93%, 94%, 95%, 96%, 97%, 98%, 99% or more sequence identity to SEQ ID NO:64 or 65.

In some embodiments, T2A comprises the sequence of amino acids set forth in SEQ ID NO: 8, 24, 72, 75 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8, 24, 72, 75. In some embodiments, T2A is encoded by the sequence of nucleic acids set forth in SEQ ID NO: 61 or a sequence of nucleic acids that exhibits at least 85%, 86%, 87%, 88%, 89%, 90%, 91%, 92%, 93%, 94%, 95%, 96%, 97%, 98%, 99% or more sequence identity to SEQ ID NO: 61.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

Also provided are compositions containing one or more of the nucleic acid molecules, vectors or constructs, such as any described above. In some embodiments, the nucleic acid molecules, vectors, constructs or compositions can be used to engineer cells, such as T cells, to express any of the binding molecules, e.g., antibody or recombinant receptor, and/or the additional binding molecules.

### B. Preparation of Cells for Engineering

In some embodiments, preparation of the engineered cells includes one or more culture and/or preparation steps. The cells for introduction of the recombinant receptor (e.g., CAR) may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using anti-CD3/anti-CD28 conjugated magnetic beads (e.g., DYNABEADS® M⁻⁴⁵⁰ CD3/CD28 T Cell Expander, MACSiBeads™, etc.).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase certain features, such as to improve long-term survival, expansion, and/or engraftment following administration, which in some aspects is particularly robust in such sub-populations (see Terakura et al. (2012) Blood.1:72-82; Wang et al. (2012) J Immunother. 35(9):689-701). In some embodiments, combining TCM-enriched CD8+ T cells and CD4+ T cells further enhances response.

In embodiments, memory T cells are present in both CD62L+ and CD62L− subsets of CD8+ peripheral blood lymphocytes. PBMC can be enriched for or depleted of CD62L−CD8+ and/or CD62L+CD8+ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA, and positive selection based on a marker characteristic of central memory T cells, such as CD62L or CCR7, where the positive and negative selections are carried out in either order.

CD4+ T helper cells are sorted into naïve, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO−.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher © Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynabeads® or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084, are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS®) (Miltenyi Biotec, Auburn, CA). Magnetic Activated Cell Sorting (MACS®) systems are capable of high-purity selection of cells having magnetized particles attached thereto. In certain embodiments, MACS® operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in WO2009/072003 or US 20110003380.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS® system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS® system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy® system (Miltenyi Biotec). The CliniMACS Prodigy@system in some aspects is equipped with a cell processing unity that permits automated washing and fractionation of cells by centrifugation. The CliniMACS Prodigy® system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy® system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope (see, e.g., Klebanoff et al. (2012) J Immunother. 35(9): 651-660, Terakura et al. (2012) Blood.1:72-82, and Wang et al. (2012) J Immunother. 35(9):689-701).

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° C. per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/mL). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood.1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC) (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen.

For example, antigen-specific T cell lines or clones can be generated to cytomegalovirus antigens by isolating T cells from infected subjects and stimulating the cells in vitro with the same antigen.

### C. Engineered Cells, Vectors and Compositions for Multi-Targeting

Also provided are cells such as engineered cells that can bind to and/or target multiple antigens. In some embodiments, improved selectivity and specificity is achieved through strategies targeting multiple antigens. Such strategies generally involve multiple antigen-binding domains, which typically are present on distinct genetically engineered antigen receptors and specifically bind to distinct antigens. In some embodiments, the cells are engineered with the ability to bind more than one antigen. For example, in some embodiments, the cells are engineered to express multispecific binding molecules. In some embodiments, the cells express multiple binding molecules, e.g., recombinant receptors, each of which can target one antigen or multiple antigens, e.g., one receptor that targets DLL3, such as any described herein, and another receptor that targets another antigen, e.g., tumor antigen. In some aspects, a plurality of genetically engineered antigen receptors are introduced into the cell, which specifically bind to different antigens, each expressed in or on the disease or condition to be targeted with the cells or tissues or cells thereof. Such features can in some aspects address or reduce the likelihood of off-target effects or increase efficacy. For example, where a single antigen expressed in a disease or condition is also expressed on or in non-diseased or normal cells, such multi-targeting approaches can provide selectivity for desired cell types by requiring binding via multiple antigen receptors in order to activate the cell or induce a particular effector function. In some embodiments, a plurality of cells can be engineered to express one or more different binding molecules, e.g., recombinant receptors, each of which can target one antigen or multiple antigens.

Also provided are multispecific cells containing any of the binding molecules described herein, such as cells containing a cell surface protein including the anti-DLL3 antibody or an antigen-binding fragment thereof and an additional cell surface protein, such as an additional chimeric receptor, which binds to a different antigen or a different epitope on DLL3. In some embodiments, provided are compositions of cells that express recombinant receptors, wherein one or more of the binding molecules, multispecific binding molecules and/or recombinant receptors bind and/or target DLL3. Also provided are compositions of cells containing a plurality of cells that express one or more different binding molecules, e.g., recombinant receptors that can target one or multiple antigens. In some embodiments, the multispecific binding molecules and/or recombinant receptors target one or more different epitopes on DLL3.

In some embodiments, provided are composition of cells, wherein each type of cell expresses one or more binding molecules, e.g., recombinant receptors. In some embodiments, the cell comprises (e.g., has been transformed with) one or more vectors comprising one or more nucleic acid that encodes one or more an amino acid sequence comprising one or more antibodies and/or portions thereof, e.g., antigen-binding fragments thereof. In some embodiments, one or more such cells are provided. In some embodiments, a composition containing one or more such cells is provided. In some embodiments, the one or more cells can express different antibodies, or the same antibody. In some embodiments, each of the cells expresses one or more antibodies, such as more than one antibody. In some embodiments, each of the cells expresses a multispecific binding molecule, e.g., a multispecific receptor, e.g., CAR.

In some embodiments, the cells include multi-targeting strategies that target DLL3 and a second or additional antigen associated with a particular disease or condition. In some embodiments, the second or additional antigen is targeted by a multispecific binding molecule and/or multiple binding molecules and/or a plurality of cells, e.g., one or more cells, each engineered to express one or more recombinant receptors. In some embodiments, a recombinant receptor targeting a second or additional antigen is expressed on the same cell as a DLL3 binding molecule, or on a different cell.

In some embodiments, among the second or additional antigens for multi-targeting strategies includes those in which at least one of the antigens is a universal tumor antigen, or a family member thereof. In some embodiments, the second or additional antigen is an antigen expressed on a tumor. In some embodiments, the DLL3-binding molecules provided herein target an antigen on the same tumor type as the second or additional antigen. In some embodiments, the second or additional antigen may also be a universal tumor antigen or may be a tumor antigen specific to a tumor type. In some embodiments, the cell further comprises an additional genetically engineered antigen receptor that recognizes a second or additional antigen expressed on a disease or condition to be treated and induces a stimulatory or activating signal.

In some embodiments, the plurality of antigens, e.g., the first antigen, e.g., DLL3, and the second or additional antigens, are expressed on the cell, tissue, or disease or condition being targeted, such as on the cancer cell. In some aspects, the cell, tissue, disease or condition is or is associated with a cancer or a tumor. One or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is/are achieved.

In some embodiments, the cells and methods include multi-targeting strategies, such as expression of two or more genetically engineered receptors on the cell, each recognizing a different antigen and typically each including a different intracellular signaling component. Such multi-targeting strategies are described, for example, in WO 2014055668 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

In some embodiments, a plurality of cells, each engineered to express one or more recombinant receptors, are provided. For example, in some embodiments, one cell is engineered to express a binding molecule that binds and/or targets DLL3, and another cell is engineered to express a binding molecule that binds and/or targets an additional or second antigen. In some embodiments, the cells can each express a multispecific binding molecule, e.g., a multispecific recombinant receptor, where one or more of the target antigen is DLL3. In some of such embodiments, the plurality of cells can be administered together or separately. In some embodiments, some of the plurality of cells are administered simultaneously or concurrently with other cells, e.g., administered on the same day, and/or sequentially with or intermittently with, in any order, another engineered cell in the plurality. For example, in some embodiments, an engineered cell expressing a DLL3-binding molecule, e.g., CAR, is administered simultaneously with or sequentially with, in any order, another engineered cell expressing a binding molecule that binds a different target antigen or a different epitope on DLL3. In some embodiments, the plurality of cells can be in the same composition or in different compositions. Exemplary compositions of the cells include compositions described in Section III below.

### III. PHARMACEUTICAL COMPOSITIONS

Also provided are compositions including the DLL3-binding molecules, immunoconjugates, recombinant receptors, and engineered cells, including pharmaceutical compositions and formulations. Also provided are compositions comprising engineered cells that express the DLL3-binding molecules provided herein, such as recombinant receptors (e.g., CARs), including pharmaceutical compositions and formulations.

Provided are pharmaceutical formulations comprising a DLL3-binding molecule (e.g., antibody), an immunoconjugate, a recombinant receptor (e.g., chimeric antigen receptor), engineered cells expressing the molecules (e.g., antibody or recombinant receptor), a plurality of engineered cells expressing the molecules (e.g., recombinant receptor) and/or additional agents for combination treatment or therapy. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell, binding molecule, and/or antibody, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

Formulations of the antibodies described herein can include lyophilized formulations and aqueous solutions.

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the cells or antibodies are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the binding molecules and/or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

There may be administered using standard administration techniques, formulations, and/or devices. Provided are formulations and devices, such as syringes and vials, for storage and administration of the compositions. Administration of the cells can be autologous or heterologous. For example, immune cells, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immune cell such as a T cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, intracranial, intrathoracic, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the binding molecule in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

Also provided are pharmaceutical compositions for combination therapy. Any of the additional agents for combination therapy described herein can be prepared and administered as one or more pharmaceutical compositions, with the DLL3-binding molecule (e.g., antibody), immunoconjugate, recombinant receptor (e.g., chimeric antigen receptor) and/or engineered cells expressing the molecules (e.g., recombinant receptor) described herein. The combination therapy can be administered in one or more pharmaceutical compositions, e.g., where the binding molecules, recombinant receptors and/or cells are in the same pharmaceutical composition as the additional agent, or in separate pharmaceutical compositions. For example, in some embodiments, the additional agent is an additional engineered cell, e.g., cell engineered to express a different recombinant receptor that targets a different antigen or a different epitope on DLL3, and is administered in the same composition or in a separate composition. In some embodiments, each of the pharmaceutical composition is formulated in a suitable formulation according to the particular binding molecule, recombinant receptor, cell, e.g., engineered cell, and/or additional agent, and the particular dosage regimen and/or method of delivery.

### IV. METHODS AND USES

Also provided methods, such as methods of treatment, of using and uses of the DLL3-binding molecules, immunoconjugates, recombinant receptors, engineered cells, and pharmaceutical compositions and formulations thereof, such as in the treatment of diseases, conditions, and disorders in which DLL3 is expressed, and/or detection, diagnostic, and prognostic methods. Also provided are methods of combination therapy and/or treatment.

### A. Therapeutic and Prophylactic Methods and Uses

Also provided are methods of administering and uses, such as therapeutic and prophylactic uses, of the DLL3-binding molecules, including the anti-DLL3 antibodies, e.g., antibody fragments and proteins containing the same such as the recombinant receptors (e.g., CARs), engineered cells expressing the recombinant receptors (e.g., CARs), plurality of engineered cells expressing the receptors, and/or compositions comprising the same. Such methods and uses include therapeutic methods and uses, for example, involving administration of the molecules (e.g., DLL3-binding molecules, recombinant receptors), cells (e.g., engineered cells), or compositions containing the same, to a subject having a disease, condition, or disorder associated with DLL3 such as a disease, condition, or disorder associated with DLL3 expression, and/or in which cells or tissues express, e.g., specifically express DLL3. In some embodiments, the molecule, cell, and/or composition is administered in an effective amount to effect treatment of the disease or disorder. Provided herein are uses of the binding molecules (e.g., anti-DLL3 antibodies or antigen-binding fragments thereof), recombinant receptors (e.g., CARs), and cells (e.g., engineered cells) in such methods and treatments, and in the preparation of a medicament in order to carry out such therapeutic methods. Uses include uses of the binding molecules, CARs, antibodies, and cells in such methods and treatments, and in the preparation of a medicament in order to carry out such therapeutic methods. In some embodiments, the methods are carried out by administering the binding molecules or cells, or compositions comprising the same, to the subject having, having had, or suspected of having the disease or condition. In some embodiments, the methods thereby treat the disease or condition or disorder in the subject. Also provided herein are of use of any of the compositions, such as pharmaceutical compositions provided herein, for the treatment of a disease or disorder associated with DLL3, for example, a DLL3-expressing cancer.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided molecules and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, an antibody or composition or cell which suppresses tumor growth reduces the rate of growth of the tumor compared to the rate of growth of the tumor in the absence of the antibody or composition or cell.

An “effective amount” of an agent, e.g., a pharmaceutical formulation, binding molecule, antibody, or cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, antibody, or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the molecules, cells, and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

As used herein, a “subject” is a mammal, such as a human or other animal, and typically is human.

Among the diseases to be treated are any DLL3-associated disease or condition or disease or condition in which DLL3 is specifically expressed. In certain diseases and conditions, DLL3 is expressed on malignant cells and cancers. In some embodiments, the disease or condition is a DLL3-expressing cancer. In some aspects, in certain diseases and conditions, DLL3 is expressed on malignant cells and cancers, including cancers associated with a solid tumor. In some embodiments among the disease or disorder to be treated is a cancer, such as a DLL3-expressing cancer. In some embodiments, the cancer is associated with a solid tumor.

In some embodiments, the cancer is associated with a DLL3-expressing solid tumor. Among the DLL3-associated diseases or conditions that can be treated include, but are not limited to cancers or tumors of neuroendocrine origin, small cell lung cancer (SCLC), large cell neuroendocrine carcinoma (LCNEC), melanoma, medullary carcinoma, lung cancer, extrapulmonary small cell carcinomas, goblet cell carcinoma, breast cancer, glioma, glioblastoma, glioblastoma multiforme, bladder cancer, small cell bladder cancer, metastatic castration-resistant prostate cancer, prostate cancer, castration-resistant neuroendocrine prostate cancer, stomach cancer, thyroid cancer, carcinoids, pancreatic cancer, urinary tract carcinoid tumor, ovarian cancer, testicular cancer, neuroendocrine carcinoma, Merkel cell carcinoma, and neuroendocrine lung tumors. In some aspects, the disease or condition to be treated according to the provided embodiments is selected from SCLC and LCNEC. In some aspects, the disease or condition to be treated according to the provided embodiments is SCLC. In some embodiments, the disease or condition to be treated is an ovarian cancer. In some embodiments, the disease or condition to be treated is a lung cancer. In some embodiments, the disease or condition to be treated is a pancreatic cancer. In some embodiments, the disease or condition to be treated is a lung cancer, and the lung cancer is a small cell lung cancer (SCLC). In some embodiments, the disease or condition to be treated is a large cell neuroendocrine carcinoma (LCNEC). In some embodiments, the disease or condition to be treated is a melanoma. In some embodiments, the disease or condition to be treated is a glioma. In some embodiments, the disease or condition to be treated is a glioblastoma.

In some embodiments, the methods include administering engineered cells (e.g. T cells) expressing a provided binding molecule to a subject having a DLL3-associated disease or condition, such as a DLL3-expressing cancer. In particular embodiments, the engineered cells (e.g. T cells) express a recombinant receptor, such as a CAR, containing as an antigen-binding domain a provided antibody or antigen-binding fragment thereof (e.g. scFv). In some embodiments, the engineered cells are autologous to the subject being treated. In some embodiments, the engineered cells are allogeneic to the subject being treated, in which case the cells are obtained from the diseased subject and engineered with the recombinant receptor (e.g. CAR) prior to administration of the engineered cells.

In some embodiments, the methods may identify a subject who has, is suspected to have, or is at risk for developing a DLL3-associated disease or disorder, such as a small cell lung cancer (SCLC) or a large cell neuroendocrine carcinoma (LCNEC). Hence, provided are methods for identifying subjects with diseases or disorders associated with elevated DLL3 expression and selecting them for treatment with a provided DLL3 binding molecule, including any of the anti-DLL3 antibodies, e.g., antibody fragments and proteins containing the same such as the chimeric receptors, and/or engineered cells expressing the recombinant receptors.

For example, a subject may be screened for the presence of a disease or disorder associated with elevated DLL3 expression, such as a DLL3-expressing cancer, such as a small cell lung cancer (SCLC) or a large cell neuroendocrine carcinoma (LCNEC). In some embodiments, the methods include screening for or detecting the presence of a DLL3-associated disease, e.g. a tumor. Thus, in some aspects, a sample may be obtained from a patient suspected of having a disease or disorder associated with elevated DLL3 expression and assayed for the expression level of DLL3. In some aspects, a subject who tests positive for a DLL3-associated disease or disorder may be selected for treatment by the present methods, and may be administered a therapeutically effective amount of an anti-DLL3 antibody, a DLL3-targeting CAR, cells containing a CAR or a pharmaceutical composition thereof as described herein. In some embodiments, the methods can be used to monitor the size or density of a DLL3-expressing tissue, e.g. tumor, over time, e.g., before, during, or after treatment by the methods.

In some embodiments, the subject has persistent or relapsed disease, e.g., following treatment with another DLL3-specific antibody and/or cells expressing a DLL3-targeting chimeric receptor and/or other therapy, including chemotherapy, radiation, and/or hematopoietic stem cell transplantation (HSCT), e.g., allogenic HSCT. In some embodiments, the administration effectively treats the subject despite the subject having become resistant to another DLL3-targetetd therapy. In some embodiments, the subject has not relapsed but is determined to be at risk for relapse, such as at a high risk of relapse, and thus the compound or composition is administered prophylactically, e.g., to reduce the likelihood of or prevent relapse.

In some embodiments, the treatment does not induce an immune response by the subject to the therapy, and/or does not induce such a response to a degree that prevents effective treatment of the disease or condition. In some aspects, the degree of immunogenicity and/or graft versus host response is less than that observed with a different but comparable treatment. For example, in the case of adoptive cell therapy using cells expressing CARs including the provided anti-DLL3 antibodies, the degree of immunogenicity in some embodiments is reduced compared to CARs including a different antibody that binds to a similar, e.g., overlapping epitope and/or that competes for binding to DLL3 with the provided antibody, such as a mouse or rabbit or humanized antibody.

In some embodiments, the methods include adoptive cell therapy, whereby genetically engineered cells expressing the provided DLL3-targeting receptors (e.g., containing DLL3-targeting antibody or fragment thereof) are administered to subjects. Such administration can promote activation of the cells (e.g., T cell activation) in a DLL3-targeting manner, such that the cells of the disease or disorder are targeted for destruction.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition. In some aspects, the methods thereby treat, e.g., ameliorate one or more symptom of the disease or condition, such as by lessening tumor burden in a DLL3-expressing cancer.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS).

### V. ARTICLES OF MANUFACTURE OR KITS

Also provided are articles of manufacture or kit containing the provided binding molecules (e.g., antibodies), recombinant receptors (e.g., CARs), genetically engineered cells, and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., DLL3-expressing cancer). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating the DLL3-expressing or DLL3-associated disease, disorder or condition in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a DLL3-expressing or DLL3-associated disease, disorder or condition in an individual. In some aspects, the label or package insert can include instructions for use, for example instructions for administering the antibody or antigen-binding fragment thereof, the single chain cell surface protein, the conjugate, the chimeric antigen receptor, the cell or the composition, in some aspects in accord with any of the methods or uses described herein.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the antibody (e.g., anti-DLL3 antibody) or antigen-binding fragment thereof or recombinant receptor (e.g., CAR); and (b) a second container with a composition contained therein. In some aspects, the article or kit further comprises instructions on the label or package insert for treating the subject in an effective amount.

### VI. DEFINITIONS

As used herein, reference to a “corresponding form” of an antibody means that when comparing a property or activity of two antibodies, the property is compared using the same form of the antibody. For example, if it is stated that an antibody has greater activity compared to the activity of the corresponding form of a first antibody, that means that a particular form, such as an scFv of that antibody, has greater activity compared to the scFv form of the first antibody.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The terms “full length antibody,” “intact antibody,” and “whole antibody” are used herein interchangeably to refer to an antibody having a structure substantially similar to a native antibody structure or having heavy chains that contain an Fc region as defined herein.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-DLL3 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Appropriate parameters for aligning sequences can be determined, including using any algorithms needed to achieve maximal alignment over the full length of the sequences being compared.

In some aspects, corresponding positions of the one or more modifications, such as one or more substitutions, can be determined in reference to positions of a reference amino acid sequence or a reference nucleotide sequence. As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm or other available algorithms. By aligning the sequences, corresponding residues can be identified, for example, using conserved and identical amino acid residues as guides. In general, to identify corresponding positions, the sequences of amino acids are aligned so that the highest order match is obtained (see, e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073). Alignment for determining corresponding positions can be obtained in various ways, for instance, using publicly available computer software such as BLAST, BLAST-2, ALIGN or Megalign (DNASTAR) software. Appropriate parameters for aligning sequences can be determined, including any algorithms needed to achieve maximal alignment over the full length of the sequences being compared. For example, corresponding residues can be determined by alignment of a reference sequence that is a wild-type Cas protein by available alignment methods. By aligning the sequences, one skilled in the art can identify corresponding residues, for example, using conserved and/or identical amino acid residues as guides.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen-binding, decreased immunogenicity, or improved ADCC or CDC.

Amino acids generally can be grouped according to the following common side-chain properties:
(1) hydrophobic: Norleucine, Met, Ala, Val, Len, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. In some embodiments, “about” may refer to ±1% to ±5%.

As used herein, a “composition” refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting the antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting the antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

### VII. EXEMPLARY EMBODIMENTS

Among the provided embodiments are:

1. An anti-Delta-like ligand 3 (DLL3) antibody or antigen-binding fragment thereof comprising a heavy chain variable (V_{H}) region and a light chain variable (V_{L}) region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:90, and
the V_{L} region comprises a light chain complementarity determining region 1 (CDR-L1), a light chain complementarity determining region 2 (CDR-L2) and a light chain complementarity determining region 3 (CDR-L3) contained within SEQ ID NO:91.

2. An anti-DLL3 antibody or antigen-binding fragment thereof, comprising a heavy chain variable (V_{H}) region, and a light chain variable (V_{L}) region, wherein:
the V_{H} region comprises a heavy chain complementarity determining region 1 (CDR-H1) comprising the sequence set forth in SEQ ID NO:92, a heavy chain complementarity determining region 2 (CDR-H2) comprising the sequence set forth in SEQ ID NO:93, and a heavy chain complementarity determining region 3 (CDR-H3) comprising the sequence set forth in SEQ ID NO:94, and
the V_{L} region comprises a light chain complementarity determining region 1 (CDR-L1) comprising the sequence set forth in SEQ ID NO:95, a light chain complementarity determining region 2 (CDR-L2) comprising the sequence set forth in SEQ ID NO:96, and a light chain complementarity determining region 3 (CDR-L3) comprising the sequence set forth in SEQ ID NO:97.

3. The anti-DLL3 antibody or antigen-binding fragment thereof of embodiment 1 or 2, wherein:
the V_{H} region comprises an amino acid sequence having at least at or about 95%, 96%, 97%, 98% or 99% sequence identity to SEQ ID NO:90, and
the V_{L} region comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:91.

4. An anti-DLL3 antibody or antigen-binding fragment thereof comprising a heavy chain variable (V_{H}) region and a light chain variable (V_{L}) region, wherein:
the V_{H} region comprises an amino acid sequence having at least at or about 95%, 96%, 97%, 98% or 99% sequence identity to SEQ ID NO:90, and
the V_{L} region comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:91.

5. The anti-DLL3 antibody or antigen-binding fragment thereof of embodiment 4, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:90, and
the V_{L} region comprises a light chain complementarity determining region 1 (CDR-L1), a light chain complementarity determining region 2 (CDR-L2) and a light chain complementarity determining region 3 (CDR-L3) contained within SEQ ID NO:91.

6. The anti-DLL3 antibody or antigen-binding fragment thereof of embodiment 4 or 5, wherein:
the V_{H} region comprises a heavy chain complementarity determining region 1 (CDR-H1) comprising the sequence set forth in SEQ ID NO:92, a heavy chain complementarity determining region 2 (CDR-H2) comprising the sequence set forth in SEQ ID NO:93, and a heavy chain complementarity determining region 3 (CDR-H3) comprising the sequence set forth in SEQ ID NO:94, and
the V_{L} region comprises a light chain complementarity determining region 1 (CDR-L1) comprising the sequence set forth in SEQ ID NO:95, a light chain complementarity determining region 2 (CDR-L2) comprising the sequence set forth in SEQ ID NO:96, and a light chain complementarity determining region 3 (CDR-L3) comprising the sequence set forth in SEQ ID NO:97.

7. The anti-DLL3 antibody or antigen-binding fragment thereof of any of embodiments 1-6, wherein:
the V_{H} region comprises the sequence set forth in SEQ ID NO:90, and
the V_{L} region comprises the sequence set forth in SEQ ID NO:91.

8. An anti-DLL3 antibody or antigen-binding fragment thereof, comprising a heavy chain variable (V_{H}) region and a light chain variable (V_{L}) region, wherein:
the V_{H} region comprises the sequence set forth in SEQ ID NO:90, and
the V_{L} region comprises the sequence set forth in SEQ ID NO:91.

9. The anti-DLL3 antibody or antigen-binding fragment thereof of any of embodiments 1-8, wherein the anti-DLL3 antibody or antigen-binding fragment thereof is recombinant.

10. The anti-DLL3 antibody or antigen-binding fragment thereof of any of embodiments 1-9, wherein the V_{H} region and the V_{L} region is human or is from a human protein.

11. The anti-DLL3 antibody or antigen-binding fragment thereof of any of embodiments 1-10, wherein the antibody further comprises a heavy chain constant region (C_{H}) and/or a light chain constant region (C_{L}).

12. The anti-DLL3 antibody or antigen-binding fragment thereof of embodiment 11, wherein:
the C_{H} comprises the sequence set forth in SEQ ID NO:126, or an amino acid sequence having at least at or about 95%, 96%, 97%, 98% or 99% sequence identity to SEQ ID NO:126; and/or
the C_{L} comprises the sequence set forth in SEQ ID NO:127, or an amino acid sequence having at least at or about 95%, 96%, 97%, 98% or 99% sequence identity to SEQ ID NO:127.

13. The anti-DLL3 antibody or antigen-binding fragment thereof of embodiment 11 or 12, wherein the antibody or antigen-binding fragment thereof comprises:
a heavy chain comprising the sequence set forth in SEQ ID NO:128; and/or
a light chain comprising the sequence set forth in SEQ ID NO:129.

14. The anti-DLL3 antibody or antigen-binding fragment thereof of any of embodiments 1-13, wherein the antibody or antigen-binding fragment thereof comprises:
a heavy chain comprising the sequence set forth in SEQ ID NO:128; and
a light chain comprising the sequence set forth in SEQ ID NO:129.

15. The anti-DLL3 antibody or antigen-binding fragment thereof of any of embodiments 1-14, wherein the antibody is a full length antibody.

16. The anti-DLL3 antibody or antigen-binding fragment thereof of any of embodiments 1-14, wherein the antibody is an antigen-binding fragment.

17. The anti-DLL3 antibody or antigen-binding fragment thereof of any of embodiments 1-10 and 16, wherein the antigen-binding fragment thereof comprises a single chain Fv (scFv).

18. The anti-DLL3 antibody or antigen-binding fragment thereof of any of embodiments 1-10, 16, and 17, wherein the V_{H} region is amino-terminal to the V_{L} region.

19. The anti-DLL3 antibody or antigen-binding fragment thereof of any of embodiments 1-10, 16, and 17, wherein the V_{H} region is carboxy-terminal to the V_{L} region.

20. The anti-DLL3 antibody or antigen-binding fragment thereof of any of embodiments 1-10 and 17-19, wherein the V_{H} region and the V_{L} region are joined by a flexible linker.

21. The anti-DLL3 antibody or antigen-binding fragment thereof of embodiment 20, wherein the flexible linker comprises the sequence set forth in SEQ ID NO:46.

22. The anti-DLL3 antibody or antigen-binding fragment thereof of any of embodiments 17-21, wherein the scFv comprises the sequence set forth in SEQ ID NO:89.

23. The anti-DLL3 antibody or antigen-binding fragment thereof of any of embodiments 1-22, wherein the anti-DLL3 antibody or antigen-binding fragment thereof specifically binds to a human DLL3.

24. The anti-DLL3 antibody or antigen-binding fragment thereof of embodiment 23, wherein the human DLL3 comprises an amino acid sequence set forth in SEQ ID NO:120 or 121.

25. The anti-DLL3 antibody or antigen-binding fragment thereof of any of embodiments 1-24, wherein the anti-DLL3 antibody or antigen-binding fragment thereof does not bind to, is not cross-reactive to, or binds at a lower level or degree or affinity to a DLL1 or a DLL4 protein, optionally a human DLL1 or a human DLL4.

26. The anti-DLL3 antibody or antigen-binding fragment thereof of any of embodiments 1-25, wherein the extent, level or degree or affinity of binding of the anti-DLL3 antibody or antigen-binding fragment thereof to a human DLL1 or a human DLL4 is at least at or about 75%, 80%, 90%, 95% or 99% less than the extent, level or degree or affinity of binding to a human DLL3.

27. The anti-DLL3 antibody or antigen-binding fragment thereof of any of embodiments 1-26, wherein the antibody or antigen-binding fragment thereof binds to human DLL3 with an equilibrium dissociation constant (K_{D}) of from about 1×10⁻¹¹ M to about 1×10⁻⁷ M.

28. A single chain cell surface protein, comprising the anti-DLL3 antibody or antigen-binding fragment thereof of any of embodiments 1-27.

29. A conjugate, comprising the anti-DLL3 antibody or antigen-binding fragment thereof of any of embodiments 1-27 and a heterologous molecule or moiety.

30. The conjugate of embodiment 29, wherein the heterologous molecule or moiety is a therapeutic moiety.

31. The conjugate of embodiment 29 or 30, wherein the heterologous molecule or moiety is a small molecule chemical compound, protein, peptide, or toxin.

32. An anti-DLL3 chimeric antigen receptor (CAR) comprising an extracellular antigen-binding domain comprising the anti-DLL3 antibody or antigen-binding fragment thereof of any of embodiments 1-27, a transmembrane domain and an intracellular signaling region.

33. The anti-DLL3 CAR of embodiment 32, further comprising a spacer between the extracellular antigen-binding domain and the transmembrane domain.

34. The anti-DLL3 CAR of embodiment 33, wherein the spacer comprises at least a portion of an immunoglobulin or a variant thereof.

35. The anti-DLL3 CAR of embodiment 33 or 34, wherein the spacer comprises at least a portion of a hinge region of an immunoglobulin or a variant thereof.

36. The anti-DLL3 CAR of embodiment 35, wherein the at least a portion of a hinge region comprises all or a portion of an IgG4 hinge region, optionally a human IgG4 hinge region, or a variant thereof, optionally wherein the variant IgG4 hinge region comprises a substitution of amino acids CPSC to CPPC compared to the wild-type IgG4 hinge region.

37. The anti-DLL3 CAR of any of embodiments 33-36, wherein the spacer is less than at or about 15 amino acids in length.

38. The anti-DLL3 CAR of any of embodiments 33-37, wherein the spacer is between 12 and 15 amino acids in length.

39. The anti-DLL3 CAR of any of embodiments 33-38, wherein the spacer comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:1.

40. The anti-DLL3 CAR of any of embodiments 33-39, wherein the spacer comprises the sequence set forth in SEQ ID NO:1.

41. The anti-DLL3 CAR of any of embodiments 33-38, wherein the spacer comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:3.

42. The anti-DLL3 CAR of any of embodiments 33-38 and 41, wherein the spacer comprises the sequence set forth in SEQ ID NO:3.

43. The anti-DLL3 CAR of any of embodiments 33-36, wherein the spacer comprises at least a portion of a hinge region and at least a portion of a CH3 region of an immunoglobulin or a variant thereof.

44. The anti-DLL3 CAR of any of embodiments 33-36 and 43, wherein the spacer is at or about 111, 112, 113, 114, 115, 116, 117, 118, 119, 120, 121, 122, 123, 124 or 125 amino acids in length, or has a length between any of the foregoing, optionally wherein the spacer comprises the sequence set forth in SEQ ID NO:5 or 58 or an amino acid sequence having at least at or about 85%, 86%, 87%, 88%, 89%, 90%, 91%, 92%, 93%, 94%, 95%, 96%, 97%, 98% or 99% sequence identity to SEQ ID NO:5 or 58.

45. The anti-DLL3 CAR of any of embodiments 33-36, wherein the spacer comprises at least a portion of a hinge region, at least a portion of a CH2 and at least a portion of a CH3 region of an immunoglobulin or a variant thereof.

46. The anti-DLL3 CAR of any of embodiments 33-36 and 45, wherein the spacer is at or about 130, 140, 150, 160, 170, 180, 190, 200, 210, 220, 221, 222, 223, 224, 225, 226, 227, 228, 229 or 230 amino acids in length, or has a length between any of the foregoing, optionally wherein the spacer comprises the sequence set forth in SEQ ID NO:42 or 82 or an amino acid sequence having at least at or about 85%, 86%, 87%, 88%, 89%, 90%, 91%, 92%, 93%, 94%, 95%, 96%, 97%, 98% or 99% sequence identity to SEQ ID NO:42 or 82.

47. The anti-DLL3 CAR of any of embodiments 32-46, wherein the transmembrane domain comprises a transmembrane domain from CD4, CD28, or CD8.

48. The anti-DLL3 CAR of any of embodiments 32-47, wherein the transmembrane domain comprises a transmembrane domain from CD28, optionally a human CD28.

49. The anti-DLL3 CAR of any of embodiments 32-48, wherein the transmembrane domain comprises SEQ ID NO:10 or 12 or an amino acid sequence having at least at or about 85%, 86%, 87%, 88%, 89%, 90%, 91%, 92%, 93%, 94%, 95%, 96%, 97%, 98% or 99% sequence identity to SEQ ID NO:10 or 12.

50. The anti-DLL3 CAR of any of embodiments 33-49, wherein the transmembrane domain comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:10.

51. The anti-DLL3 CAR of any of embodiments 33-50, wherein the transmembrane domain comprises the sequence set forth in SEQ ID NO:10.

52. The anti-DLL3 CAR of any of embodiments 33-49, wherein the transmembrane domain comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:12.

53. The anti-DLL3 CAR of any of embodiments 33-49 and 52, wherein the transmembrane domain comprises the sequence set forth in SEQ ID NO:12.

54. The anti-DLL3 CAR of any of embodiments 32-53, wherein intracellular signaling region comprises an intracellular signaling domain capable of inducing a primary activation signal in a T cell, is a T cell receptor (TCR) component and/or comprises an immunoreceptor tyrosine-based activation motif (ITAM).

55. The anti-DLL3 CAR of any of embodiments 32-54, wherein the intracellular signaling domain comprises a cytoplasmic signaling domain of a CD3-zeta (CD3ζ) chain, optionally a human CD3ζ chain.

56. The anti-DLL3 CAR of any of embodiments 32-55, wherein the intracellular signaling domain comprises the sequence set forth in SEQ ID NO:19, or an amino acid sequence having at least at or about 90%, 91%, 92%, 93%, 94%, 95%, 96%, 97%, 98% or 99% sequence identity to SEQ ID NO:19.

57. The anti-DLL3 CAR of any of embodiments 32-56, wherein the intracellular signaling domain comprises the sequence set forth in SEQ ID NO:19.

58. The anti-DLL3 CAR of any of embodiments 32-57, wherein the intracellular signaling region further comprises a costimulatory domain.

59. The anti-DLL3 CAR of embodiment 58, wherein the costimulatory domain is between the transmembrane domain and the intracellular signaling domain.

60. The anti-DLL3 CAR of embodiment 58 or 59, wherein the costimulatory domain comprises an intracellular signaling domain of a T cell costimulatory molecule or a signaling portion thereof.

61. The anti-DLL3 CAR of any of embodiments 58-60, wherein the costimulatory domain comprises an intracellular signaling domain of CD28, 4-1BB, or ICOS.

62. The anti-DLL3 CAR of any of embodiments 58-61, wherein the costimulatory domain comprises an intracellular signaling domain of 4-1BB, optionally a human 4-1BB.

63. The anti-DLL3 CAR of any of embodiments 58-63, wherein the costimulatory domain comprises the sequence set forth in SEQ ID NO:17 or an amino acid sequence having at least at or about 90%, 91%, 92%, 93%, 94%, 95%, 96%, 97%, 98% or 99% sequence identity to SEQ ID NO:17.

64. The anti-DLL3 CAR of any of embodiments 58-61, wherein the costimulatory domain comprises an intracellular signaling domain of CD28, optionally a human CD28.

65. The anti-DLL3 CAR of any of embodiments 58-61 and 64, wherein the costimulatory domain comprises the sequence set forth in SEQ ID NO:15 or 16 or an amino acid sequence having at least at or about 90%, 91%, 92%, 93%, 94%, 95%, 96%, 97%, 98% or 99% sequence identity to SEQ ID NO:15 or 16.

66. The anti-DLL3 CAR of any of embodiments 32-65, wherein the CAR comprises from its N to C terminus in order: the extracellular antigen-binding domain, the spacer, the transmembrane domain and the intracellular signaling region.

67. The anti-DLL3 CAR of any of embodiments 33-38, 54-63, and 66, wherein the CAR comprises, from its N to C terminus in order:
an extracellular antigen-binding domain comprising the scFv;
a spacer comprising a modified IgG4 hinge, optionally comprising the sequence set forth in SEQ ID NO:1;
a transmembrane domain, optionally a transmembrane domain from a human CD28, optionally comprising the sequence set forth in SEQ ID NO:10; and
an intracellular signaling region comprising a cytoplasmic signaling domain of a CD3-zeta (CD3ζ) chain and an intracellular signaling domain of a costimulatory molecule, optionally comprising an intracellular signaling domain of 4-1BB.

68. The anti-DLL3 CAR of any of embodiments 33-38, 54-63, and 66, wherein the CAR comprises, from its N to C terminus in order:
an extracellular antigen-binding domain comprising the scFv;
a spacer comprising a modified IgG4 hinge, optionally comprising the sequence set forth in SEQ ID NO:3;
a transmembrane domain, optionally a transmembrane domain from a human CD28, optionally comprising the sequence set forth in SEQ ID NO:12; and
an intracellular signaling region comprising a cytoplasmic signaling domain of a CD3-zeta (CD3ζ) chain and an intracellular signaling domain of a costimulatory molecule, optionally comprising an intracellular signaling domain of 4-1BB.

69. An anti-DLL3 chimeric antigen receptor (CAR) comprising:
an extracellular antigen-binding domain comprising an scFv comprising
a heavy chain variable (V_{H}) region and a light chain variable (V_{L}) region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:90, and
the V_{L} region comprises a light chain complementarity determining region 1 (CDR-L1), a light chain complementarity determining region 2 (CDR-L2) and a light chain complementarity determining region 3 (CDR-L3) contained within SEQ ID NO:91,
a spacer comprising the sequence set forth in SEQ ID NO:1;
a transmembrane domain comprising the sequence set forth in SEQ ID NO:10; and
an intracellular signaling region comprising a cytoplasmic signaling domain of a CD3-zeta (CD3ζ) chain comprising the sequence set forth in SEQ ID NO:19, and an intracellular signaling domain of a costimulatory molecule comprising the sequence set forth in SEQ ID NO:17.

70. An anti-DLL3 chimeric antigen receptor (CAR) comprising:
an extracellular antigen-binding domain comprising an scFv comprising
a heavy chain variable (V_{H}) region and a light chain variable (V_{L}) region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:90, and
the V_{L} region comprises a light chain complementarity determining region 1 (CDR-L1, a light chain complementarity determining region 2 (CDR-L2) and a light chain complementarity determining region 3 (CDR-L3) contained within SEQ ID NO:91,
a spacer comprising the sequence set forth in SEQ ID NO:3;
a transmembrane domain comprising the sequence set forth in SEQ ID NO:12; and
an intracellular signaling region comprising a cytoplasmic signaling domain of a CD3-zeta (CD3ζ) chain comprising the sequence set forth in SEQ ID NO:19, and an intracellular signaling domain of a costimulatory molecule comprising the sequence set forth in SEQ ID NO:17.

71. The anti-DLL3 CAR of any of embodiments 33-38, 54-63, 66, 67, and 69, wherein the CAR comprises, from its N to C terminus in order:
an extracellular antigen-binding domain comprising the scFv comprising the sequence set forth in SEQ ID NO:89;
a spacer comprising the sequence set forth in SEQ ID NO:1;
a transmembrane domain comprising the sequence set forth in SEQ ID NO:10; and
an intracellular signaling region comprising a cytoplasmic signaling domain of a CD3-zeta (CD3ζ) chain comprising the sequence set forth in SEQ ID NO:19, and an intracellular signaling domain of a costimulatory molecule comprising the sequence set forth in SEQ ID NO:17.

72. The anti-DLL3 CAR of any of embodiments 33-38, 54-63, 66, 68 and 70, wherein the CAR comprises, from its N to C terminus in order:
an extracellular antigen-binding domain comprising the scFv comprising the sequence set forth in SEQ ID NO:89;
a spacer comprising the sequence set forth in SEQ ID NO:3;
a transmembrane domain comprising the sequence set forth in SEQ ID NO:12; and
an intracellular signaling region comprising a cytoplasmic signaling domain of a CD3-zeta (CD3ζ) chain comprising the sequence set forth in SEQ ID NO:19, and an intracellular signaling domain of a costimulatory molecule comprising the sequence set forth in SEQ ID NO:17.

73. The anti-DLL3 CAR of any of embodiments 52-72, wherein the anti-DLL3 CAR comprises the sequence set forth in SEQ ID NO:117 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO:117.

74. The anti-DLL3 CAR of any of embodiments 52-73, wherein the anti-DLL3 CAR comprises the sequence set forth in SEQ ID NO:117.

75. A polynucleotide comprising a nucleic acid encoding the anti-DLL3 antibody or antigen-binding domain thereof of any of embodiments 1-27.

76. A polynucleotide comprising a nucleic acid encoding the single chain cell surface protein of embodiment 28.

77. A polynucleotide comprising a nucleic acid encoding the conjugate of any of embodiments 29-31.

78. A polynucleotide comprising a nucleic acid encoding the anti-DLL3 chimeric antigen receptor of any of embodiments 32-74.

79. The polynucleotide of any of embodiments 75-78, wherein the polynucleotide comprises:
a nucleic acid encoding the V_{H}, comprising the sequence set forth in SEQ ID NO:87, or a nucleic acid sequence having at least at or about 90%, 91%, 92%, 93%, 94%, 95%, 96%, 97%, 98% or 99% sequence identity to SEQ ID NO:87, and
a nucleic acid encoding the V_{L}, comprising the sequence set forth in SEQ ID NO:88, or a nucleic acid sequence having at least at or about 90%, 91%, 92%, 93%, 94%, 95%, 96%, 97%, 98% or 99% sequence identity to SEQ ID NO:88.

80. The polynucleotide of any of embodiments 75-79, wherein the polynucleotide comprises:
a nucleic acid encoding the V_{H}, comprising the sequence set forth in SEQ ID NO:87, and
a nucleic acid encoding the V_{L}, comprising the sequence set forth in SEQ ID NO:88.

81. The polynucleotide of any of embodiments 75-80, wherein the antigen-binding fragment is a single chain Fv (scFv), and the polynucleotide comprises a nucleic acid encoding the scFv, comprising the sequence set forth in SEQ ID NO:86 or a nucleic acid sequence having at least at or about 90%, 91%, 92%, 93%, 94%, 95%, 96%, 97%, 98% or 99% sequence identity to SEQ ID NO:86.

82. The polynucleotide of any of embodiments 75-81, wherein the antigen-binding fragment is a single chain Fv (scFv), and the polynucleotide comprises a nucleic acid encoding the scFv, comprising the sequence set forth in SEQ ID NO:86.

83. The polynucleotide of any of embodiments 78-82, wherein the polynucleotide comprises a nucleic acid encoding the spacer comprising the sequence set forth in SEQ ID NO:2 or a nucleic acid sequence having at least at or about 90%, 91%, 92%, 93%, 94%, 95%, 96%, 97%, 98% or 99% sequence identity to SEQ ID NO:2.

84. The polynucleotide of any of embodiments 78-83, wherein the polynucleotide comprises a nucleic acid encoding the spacer comprising the sequence set forth in SEQ ID NO:2.

85. The polynucleotide of any of embodiments 78-82, wherein the polynucleotide comprises a nucleic acid encoding the spacer comprising the sequence set forth in SEQ ID NO:4 or a nucleic acid sequence having at least at or about 90%, 91%, 92%, 93%, 94%, 95%, 96%, 97%, 98% or 99% sequence identity to SEQ ID NO:4.

86. The polynucleotide of any of embodiments 78-82 and 85, wherein the polynucleotide comprises a nucleic acid encoding the spacer comprising the sequence set forth in SEQ ID NO:4.

87. The polynucleotide of any of embodiments 78-84, wherein the polynucleotide comprises a nucleic acid encoding the transmembrane domain comprising the sequence set forth in SEQ ID NO:11 or a nucleic acid sequence having at least at or about 90%, 91%, 92%, 93%, 94%, 95%, 96%, 97%, 98% or 99% sequence identity to SEQ ID NO:11.

88. The polynucleotide of any of embodiments 78-84 and 87, wherein the polynucleotide comprises a nucleic acid encoding the transmembrane domain, comprising the sequence set forth in SEQ ID NO:11.

89. The polynucleotide of any of embodiments 78-82, 85 and 86, wherein the polynucleotide comprises a nucleic acid encoding the transmembrane domain comprising the sequence set forth in SEQ ID NO:13 or a nucleic acid sequence having at least at or about 90%, 91%, 92%, 93%, 94%, 95%, 96%, 97%, 98% or 99% sequence identity to SEQ ID NO:13.

90. The polynucleotide of any of embodiments 78-82, 85, 86, and 89, wherein the polynucleotide comprises a nucleic acid encoding the transmembrane domain comprising the sequence set forth in SEQ ID NO:13.

91. The polynucleotide of any of embodiments 78-90, wherein the nucleic acid encoding the anti-DLL3 chimeric antigen receptor comprises the sequence set forth in SEQ ID NO:116 or a sequence that exhibits at least 90%, 91%, 92%, 93%, 94%, 95%, 96%, 97%, 98%, or 99% sequence identity to the sequence set forth in SEQ ID NO:116.

92. The polynucleotide of any of embodiments 78-91, wherein the a nucleic acid encoding the anti-DLL3 chimeric antigen receptor comprises the sequence set forth in SEQ ID NO:116.

93. The polynucleotide of any of embodiments 75-92, wherein the polynucleotide is optimized by splice site elimination.

94. The polynucleotide of any of embodiments 75-93, wherein the polynucleotide is codon-optimized for expression in a human cell.

95. A vector, comprising the polynucleotide of any of embodiments 75-94.

96. The vector of embodiment 95, wherein the vector is a viral vector.

97. The vector of embodiment 96, wherein the viral vector is a retroviral vector or a lentiviral vector.

98. The vector of embodiment 96, wherein the viral vector is an adeno-associated virus (AAV) vector.

99. A cell comprising the anti-DLL3 antibody or antigen-binding fragment thereof of any of embodiments 1-27, the single chain cell surface protein of embodiment 28 or the conjugate of any of embodiments 29-31.

100. A cell comprising the anti-DLL3 chimeric antigen receptor of any of embodiments 32-74.

101. A cell comprising the polynucleotide of any of embodiments 75-94, or the vector of any of embodiments 95-98.

102. The cell of any of embodiments 99-101, wherein the cell is a lymphocyte.

103. The cell of any of embodiments 99-102, wherein the cell is an NK cell or a T cell.

104. The cell of any of embodiments 99-103, wherein the cell is a T cell and the T cell is a CD4+ T cell or a CD8+ T cell.

105. The cell of any of embodiments 99-104, wherein the cell is a primary cell obtained from a subject.

106. The cell of any of embodiments 100-105, wherein, among a plurality of the cells, less than at or about 10%, at or about 9%, at or about 8%, at or about 7%, at or about 5%, at or about 4%, at or about 3%, at or about 2% or at or about 1% of the cells in the plurality comprise an anti-DLL3 chimeric antigen receptor that exhibits tonic signaling and/or antigen independent activity or signaling.

107. A composition comprising the cell of any of embodiments 99-106.

108. A composition comprising the anti-DLL3 antibody or antigen-binding fragment thereof of any of embodiments 1-27, the single chain cell surface protein of embodiment 28, the conjugate of any of embodiments 29-31 or the anti-DLL3 chimeric antigen receptor of any of embodiments 32-74.

109. The composition of embodiment 107 or 108, further comprising a pharmaceutically acceptable excipient.

110. The composition of any of embodiments 107-109, wherein the composition comprises CD4+ and CD8+ T cells and the ratio of CD4+ to CD8+ T cells is from at or about 1:3 to 3:1, optionally at or about 1:2 to 2:1, optionally at or about 1:1.

111. The composition of any of embodiments 107-110, wherein, among a plurality of the cells in the composition, less than at or about 10%, at or about 9%, at or about 8%, at or about 7%, at or about 5%, at or about 4%, at or about 3%, at or about 2% or at or about 1% of the cells in the plurality comprise an anti-DLL3 chimeric antigen receptor that exhibits tonic signaling and/or antigen independent activity or signaling.

112. A method of treatment, comprising administering the anti-DLL3 antibody or antigen-binding fragment thereof of any of embodiments 1-27, the single chain cell surface protein of embodiment 28, the conjugate of any of embodiments 29-31, the anti-DLL3 chimeric antigen receptor of any of embodiments 32-74, the polynucleotide of any of embodiments 75-94, the vector of any of embodiments 95-98, the cell of any of embodiments 99-106, or the composition of any of embodiments 107-111 to a subject having a disease or disorder associated with DLL3.

113. The method of embodiment 112, wherein the disease or disorder associated with DLL3 is a cancer.

114. The method of embodiment 113, wherein the cancer is a DLL3-expressing cancer.

115. The method of any of embodiments 112-114, wherein the disease or disorder associated with DLL3 is selected from among a neuroendocrine tumor, a small cell lung cancer (SCLC), a large cell neuroendocrine carcinoma (LCNEC), a melanoma, a glioma, or a glioblastoma.

116. The method of any of embodiments 112-115, wherein the disease or disorder associated with DLL3 is a neuroendocrine tumor.

117. The method of any of embodiments 112-116, wherein the disease or disorder associated with DLL3 is a small cell lung cancer (SCLC).

118. The method of any of embodiments 112-117, wherein the disease or disorder associated with DLL3 is a large cell neuroendocrine carcinoma (LCNEC).

119. The anti-DLL3 antibody or antigen-binding fragment thereof of any of embodiments 1-27, the single chain cell surface protein of embodiment 28, the conjugate of any of embodiments 29-31, the anti-DLL3 chimeric antigen receptor of any of embodiments 32-74, the polynucleotide of any of embodiments 75-94, the vector of any of embodiments 95-98, the cell of any of embodiments 99-106, or the composition of any of embodiments 107-111 for use in treating a disease or disorder associated with DLL3.

120. Use of the anti-DLL3 antibody or antigen-binding fragment thereof of any of embodiments 1-27, the single chain cell surface protein of embodiment 28, the conjugate of any of embodiments 29-31, the anti-DLL3 chimeric antigen receptor of any of embodiments 32-74, the polynucleotide of any of embodiments 75-94, the vector of any of embodiments 95-98, the cell of any of embodiments 99-106, or the composition of any of embodiments 107-111 for the manufacture of a medicament for treating a disease or disorder associated with DLL3.

121. Use of the anti-DLL3 antibody or antigen-binding fragment thereof of any of embodiments 1-27, the single chain cell surface protein of embodiment 28, the conjugate of any of embodiments 29-31, the anti-DLL3 chimeric antigen receptor of any of embodiments 32-74, the polynucleotide of any of embodiments 75-94, the vector of any of embodiments 95-98, the cell of any of embodiments 99-106, or the composition of any of embodiments 107-111 for the treatment of a disease or disorder associated with DLL3.

122. The antibody or antigen-binding fragment thereof, single chain cell surface protein, conjugate, chimeric antigen receptor, polynucleotide, vector, cell or composition for use or the use of any of embodiments 119-121 wherein the disease or disorder associated with DLL3 is a cancer.

123. The antibody or antigen-binding fragment thereof, single chain cell surface protein, conjugate, chimeric antigen receptor, polynucleotide, vector, cell or composition for use or the use of embodiment 122, wherein the cancer is a DLL3-expressing cancer.

124. The antibody or antigen-binding fragment thereof, single chain cell surface protein, conjugate, chimeric antigen receptor, polynucleotide, vector, cell or composition for use or the use of any of embodiments 119-123, wherein the disease or disorder associated with DLL3 is a neuroendocrine tumor, a small cell lung cancer (SCLC), a large cell neuroendocrine carcinoma (LCNEC), a melanoma, a glioma, or a glioblastoma.

125. The cell, composition, antibody or antigen-binding fragment thereof, single chain cell surface protein, conjugate, chimeric antigen receptor, polynucleotide or vector for use or the use of any of embodiments 119-124, wherein the disease or disorder associated with DLL3 is a neuroendocrine tumor.

126. The cell, composition, antibody or antigen-binding fragment thereof, single chain cell surface protein, conjugate, chimeric antigen receptor, polynucleotide or vector for use or the use of any of embodiments 119-124, wherein the disease or disorder associated with DLL3 is a small cell lung cancer (SCLC).

127. The cell, composition, antibody or antigen-binding fragment thereof, single chain cell surface protein, conjugate, chimeric antigen receptor, polynucleotide or vector for use or the use of any of embodiments 119-124, wherein the disease or disorder associated with DLL3 is a large cell neuroendocrine carcinoma (LCNEC).

128. A kit comprising the anti-DLL3 antibody or antigen-binding fragment thereof of any of embodiments 1-27, the single chain cell surface protein of embodiment 28, the conjugate of any of embodiments 29-31, the anti-DLL3 chimeric antigen receptor of any of embodiments 32-74, the polynucleotide of any of embodiments 75-94, the vector of any of embodiments 95-98, the cell of any of embodiments 99-106 or the composition of any of embodiments 107-111, and instructions for use.

129. The kit of embodiment 128, wherein the instructions are for administering the anti-DLL3 antibody or antigen-binding fragment thereof, the single chain cell surface protein, the conjugate, the anti-DLL3 chimeric antigen receptor, the cell or the composition to a subject having a disease or disorder associated with DLL3.

130. An article of manufacture comprising the anti-DLL3 antibody or antigen-binding fragment thereof of any of embodiments 1-27, the single chain cell surface protein of embodiment 28, the conjugate of any of embodiments 29-31, the anti-DLL3 chimeric antigen receptor of any of embodiments 32-74, the polynucleotide of any of embodiments 75-94, the vector of any of embodiments 95-98, the cell of any of embodiments 99-106, the composition of any of embodiments 107-111, or the kit of embodiment 128 or 129.

### VIII. EXAMPLES

The following examples are included for illustrative purposes only and are not intended to limit the scope of the invention.

### Example 1: Generation of Anti-DLL3 Antibodies

Fully human antibodies against human DLL3 were generated by genetically immunized OmniFlic (Ligand Pharmaceuticals) transgenic rats having a common light chain. 6 OmniFlic rats were immunized with cells engineered to express the extracellular domain (ECD) of human DLL3. Lymph nodes from immunoreactive animals were harvested and the cells underwent next-generation sequencing to identify the sequences of the heavy chain and light chain pairs. 329 heavy chain sequences paired with a fixed human kappa light chain were selected for high-throughput gene assembly and expression for further analysis.

### Example 2: High-Throughput Transient Expression of Anti-DLL3 Antibodies

The ExpiCHO™ expression system (ThermoFisher Scientific, Waltham, MA) was used for the high-throughput expression of anti-DLL3 monoclonal antibodies (mAbs) identified as described in Example 1. All heavy chains and the common light chain were cloned separately into the pTT5 expression vector (NRCC, Montreal, Canada; by TeneoBio, Newark, CA). Expression was conducted in ExpiCHO-S cells in 24-well pyramidal bottom deep-well plates (DWP) at a scale of 4 mL per well. The seed-train was maintained as described in the ExpiCHO™ expression system user guide. One day prior to transfection (day −1), the cells were passaged at a viable cell density of approximately 3-4×10⁶ cells/mL at an appropriate scale. One g of each heavy chain and the common light chain DNA (2 g total/transfection) were then diluted in into a 24-DWP with 150 μL of Optipro SFM transfection media using an automated liquid handler, sealed with aluminum plate covers, and stored at 4° C. overnight.

The cells were transfected on day 0 according to the user guide. Briefly, the cells were diluted to a viable cell density of 6×10⁶ cells/mL. The expifectamine reagent (12.8 uL/well) was diluted in Optipro SFM (150 uL/well) at an appropriate volume and 160 uL was immediately dispensed into the DNA plates. Within 5 minutes of reagent addition, the diluted cells were aseptically transferred to the plates (3.6 mL/well) using a multichannel pipette. The plates were sealed with sterile breathable film and incubated under the following conditions: 37° C., 5% CO2 and an agitation speed of 500 RPM with a 3 mm orbital diameter. On day 2, 24 μL of enhancer and 960 μL of feed were added into each well of the transfection plates. Supernatants were harvested on day 7 by centrifuging at 3720 RCF, 4° C., for 30 minutes. Expression titers were quantified using biolayer interferometry with the Octet® RED384 System and protein A biosensors using an IgG of known concentration as a reference standard. The supernatants were normalized to 10 μg/mL and stored at 4° C. for further characterization.

### Example 3: Surface Plasmon Resonance Analysis of AnTi-DLL3 Antibody

Kinetics of the binding of the anti-DLL3 antibody DLL3-mAb-1, identified as described in Examples 1 and 2, was assessed by surface plasmon resonance. DLL3-mAb-1 has the amino acid sequences described in Tables E1-E4 below.

TABLE E1
Amino Acid Sequence Identification Numbers (SEQ ID NO:)

| | | CDR-H1 | CDR-H2 | CDR-H3 | | CDR-L1 | CDR-L2 | CDR-L3 |
|---|---|---|---|---|---|---|---|---|
| Antibody | V_{H} | (Kabat) | (Kabat) | (Kabat) | V_{L} | (Kabat) | (Kabat) | (Kabat) |
| DLL3- | 90 | 92 | 93 | 94 | 91 | 95 | 96 | 97 |
| mAb-1 | | | | | | | | |

TABLE E2
Amino Acid Sequence Identification Numbers (SEQ ID NO:)

| | | CDR-H1 | CDR-H2 | CDR-H3 | | CDR-L1 | CDR-L2 | CDR-L3 |
|---|---|---|---|---|---|---|---|---|
| Antibody | VH | (Chothia) | (Chothia) | (Chothia) | VL | (Chothia) | (Chothia) | (Chothia) |
| DLL3- | 90 | 98 | 99 | 100 | 91 | 101 | 102 | 103 |
| mAb-1 | | | | | | | | |

TABLE E3
Amino Acid Sequence Identification Numbers (SEQ ID NO:)

| | | CDR-H1 | CDR-H2 | CDR-H3 | | CDR-L1 | CDR-L2 | CDR-L3 |
|---|---|---|---|---|---|---|---|---|
| Antibody | VH | (AbM) | (AbM) | (AbM) | VL | (AbM) | (AbM) | (AbM) |
| DLL3- | 90 | 110 | 111 | 112 | 91 | 113 | 114 | 115 |
| mAb-1 | | | | | | | | |

TABLE E4
Amino Acid Sequence Identification Numbers (SEQ ID NO:)

| | | CDR-H1 | CDR-H2 | CDR-H3 | | CDR-L1 | CDR-L2 | CDR-L3 |
|---|---|---|---|---|---|---|---|---|
| Antibody | VH | (IMGT) | (IMGT) | (IMGT) | VL | (IMGT) | (IMGT) | (IMGT) |
| DLL3- | 90 | 104 | 105 | 106 | 91 | 107 | 108 | 109 |
| mAb-1 | | | | | | | | |

The Biacore SPR 8K machine was used for kinetics binding analysis. The sensor surface of a protein G chip (series s Cat #29179315) was used to capture the ligands. 2 μg/ml of purified anti-DLL3 antibody was diluted in running buffer (0.01M HEPES, 0.15 NaCl, 3 mM EDTA, 0.005% Tween-20). The diluted antibody was then injected at a flow rate of 10 μL/min for 60 seconds. Human DLL3 (hDLL3) antigen was diluted in the same running buffer to 6 concentrations (30, 10, 3.33, 1.11, 0.37 and 0 nM). hDLL3 was then injected on all channels (with each channel having its own reference point) at a flow rate of 30 uL/min for an association phase of 300 seconds, followed by 600 seconds of dissociation. 6 cycles of running analyte were repeated according to analyte concentrations in ascending order. After each cycle of interaction analysis, the sensor chip surface was regenerated completely with Glycine-HCL pH 1.5 at a flow rate of 30 L/min for 60 s. The binding of the anti-DLL3 antibody and hDLL3 protein was analyzed by multi-cycle method. The binding data for DLL3-mAb-1 is shown in Table E5 below.

TABLE E5

| Capture | Analyte | ka (1/Ms) | kd (1/s) | K_{D} (M) | Rₘₐₓ (RU) | chi2 % |
|---|---|---|---|---|---|---|
| DLL3- | hDLL3 | 2.33E+05 | 1.37E−02 | 5.88E−08 | 640.4 | 93.59305 |
| mAb-1 | | | | | | |

### Example 4: Dose Response of Anti-DLL3 Antibody for Target Cell Surface Binding

Human DLL3 or cynomolgus macaque (cyno) DLL3 expressing cell lines were established by transfecting 293H cells with human DLL3 ORF-GFP or cyno DLL3 ORF-GFP plasmids and selecting stable single clone. A dose response curve for the anti-DLL3 mAb-1 binding to 293H cells expressing either human DLL3 or cyno DLL3 was generated by flow cytometry. Cells were incubated with increasing concentrations of anti-DLL3 mAb-1 in the range of 0.6 to 200 nM. Cells were washed then incubated with a secondary antibody (Alexa 647 conjugated anti-human IgG1) to measure the amount of antibody bound to the 293H cells. A dose response curve was fitted from the data using the non-linear regression function in GraphPad Prism version 8. Half-maximal concentration (EC₅₀) value was calculated from the dose response curve. The EC₅₀ value for DLL3-mAb-1 is shown in Table E6 below.

TABLE E6

| | Human DLL3 | Cyno DLL3 cell |
|---|---|---|
| Antibody | cell binding (nM) | binding (nM) |
| DLL3- | 15.59 | 10.44 |
| mAb-1 | | |

### Example 5: Anti-DLL3 Chimeric Antigen Receptor (CAR) Generation

CARs were constructed having an scFv comprising the VH and VL domains of an anti-DLL3 antibody generated from the immunized rats as described above in Example 1, and that were each found to bind DLL3 using methods as described in Examples 2-4. Each CAR was designed with the scFv in a V_{H} and V_{L} chain orientation (V_{H}-V_{L}) or a V_{L}-V_{H} orientation, the V_{H} and V_{L} chains separated by a linker (e.g., SEQ ID NO:46). For each CAR, synthesized scFv-encoding sequences were cloned into a polynucleotide construct to generate a polynucleotide encoding the CAR containing the scFv as the antigen-binding domain, in which the polynucleotide encoding the CAR contained in order, 5′ to 3′: a signal peptide; anti-DLL3 scFv; a spacer; a human CD28 transmembrane domain (e.g. SEQ ID NO:10); a human 4-1BB intracellular signaling region (e.g. SEQ ID NO:17); and a human CD3-zeta intracellular signaling region (e.g. SEQ ID NO:19). The spacer had one of the following sequences set forth in Table E7.

TABLE E7
Amino Acid Sequence Identification Numbers (SEQ ID NO:)

| SEQ ID | | |
|---|---|---|
| NO: | Amino Acid Sequence of Spacers | Feature |
| 1 | ESKYGPPCPPCPM | IgG4 hinge |
| | | spacer (aa) |
| 58 | ESKYGPPCPPCPGQPREPQVYTLPPSQEEMTKNQVSLTCLVK | IgG4 hinge- |
| | GFYPSDIAVEWESNGQPENNYKTTPPVLDSDGSFFLYSRLTV | CH3 spacer |
| | DKSRWQEGNVFSCSVMHEALHNHYTQKSLSLSLGKM | (aa) |
| 82 | ESKYGPPCPPCPAPPVAGPSVFLFPPKPKDTLMISRTPEVTCVV | IgG4/IgG2 |
| | VDVSQEDPEVQFNWYVDGVEVHNAKTKPREEQFQSTYRVV | hinge- |
| | SVLTVLHQDWLNGKEYKCKVSNKGLPSSIEKTISKAKGQPRE | IgG2/IgG4 |
| | PQVYTLPPSQEEMTKNQVSLTCLVKGFYPSDIAVEWESNGQP | CH2-IgG4 |
| | ENNYKTTPPVLDSDGSFFLYSRLTVDKSRWQEGNVFSCSVM | CH3 spacer |
| | HEALHNHYTQKSLSLSLGKM | (aa) |

### Example 6: Anti-DLL3 Chimeric Antigen Receptor (CAR) Screening in Cell Lines

The generated anti-DLL3 CAR-encoding constructs described in Example 5 above, were individually introduced by viral transduction into a Jurkat T cell line containing a Nur77 knock-in reporter (see e.g. WO 2019/089982). The Nur77 knock-in cell line contained nucleic acid sequences encoding a reporter molecule (tdTomato) knocked-in at the endogenous Nur77 locus, which is an immediate-early response gene induced by stimulation of signal from the T cell receptor and/or via molecules containing immunoreceptor tyrosine-based activation motif (ITAM). The Jurkat reporter cells were assessed for cell surface expression of the CAR (e.g. by GFP expression), antigen-dependent and antigen-independent signaling, binding of soluble target antigen DLL3, and cross-reactivity to DLL3 from different species (mouse, cynomolgus monkey) and related but distinct antigens DLL1 and DLL4.

To assess antigen-dependent signaling, transduced reporter cells were evaluated for the expression of red fluorescent protein after co-culture with target cells, K562 cells transduced to express a human DLL3 (hDLL3), K562 cells transduced to express a cynomolgus DLL3 (cDLL3), and DLL3-expressing human small cell lung cancer cell line NCI-H69 (H69; ATCC® HTB-119™). To assess antigen-independent activity, transduced reporter cells were cultured with untransduced (parental) K562 human myelogenous leukemia cells (DLL3 negative) or were cultured alone in the absence of antigen-expressing target cells (media only). To assess potential non-specific binding, transduced reporter cells were cultured with K562 cells transduced to express the alternative canonical Notch ligands, human DLL1 or human DLL4.

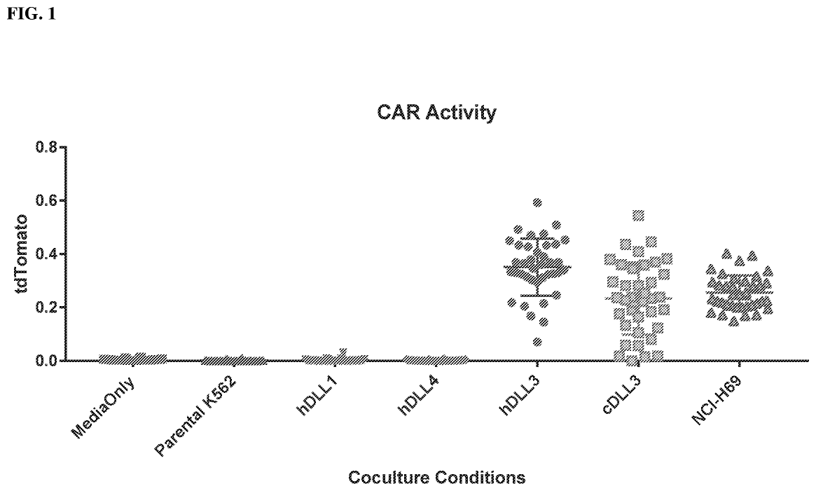

Exemplary results for antigen-dependent and antigen-independent signaling from the Jurkat reporter cell screening is depicted in FIG. 1. Jurkat reporter cells transduced with the generated anti-DLL3 CARs exhibited varying levels of antigen-dependent activity, as indicated by reporter expression after co-culture with human and cynomolgus DLL3-expressing target cells, as well as by co-culture with H69 cells known to express DLL3. In contrast, a lack of antigen-independent (tonic) signaling was observed by many of the generated anti-DLL3 CARs, as indicated by the lack of reporter expression after co-culture with parental K562 cells not expressing DLL3 or after culture without target cells. Likewise, the generated anti-DLL3 CARs generally did not exhibit non-specific activity, as indicated by the lack of reporter expression after co-culture with K652 cells expressing the non-specific antigen DLL1 or DLL4.

Some of the tested CARs, including a CAR having an scFv from the DLL3-mAb-1 antibody were observed to exhibit high DLL3 antigen-dependent activity, low tonic signaling, high DLL3 binding and low DLL1 and DLL4 cross-reactivity. CARs with these activities were selected for assessment in primary human T cells.

### Example 7: Assessment of Anti-DLL3 Chimeric Antigen Receptors (CARs) in Primary T Cells

Anti-DLL3 CARs selected based on the Jurkat cell line screening as described in Example 6 above, were assessed for expression and activity in primary human T cells.

To assess activity of the anti-DLL3 CARs, primary T cells were separately transduced with a construct encoding a CAR and monitored for antigen-dependent cytokine production and cytolytic anti-tumor activity. CD4+ and CD8+ T cells were isolated by immunoaffinity-based enrichment from leukapheresis samples of two human donors. Isolated CD4+ and CD8+ T cells were mixed at approximately 1:1 ratio, stimulated with anti-CD3/anti-CD28 coated beads, and transduced with lentiviral preparations encoding one of the anti-DLL3 CARs and cultivated under conditions for expansion for approximately 1 week. Primary T cells not expressing a CAR (mock) were used as controls.

For assessing antigen-dependent cytokine production, the CAR-expressing primary T cells were stimulated by co-culture with DLL3-expressing human small cell lung cancer NCI-H82 (H82; ATCC® HTB-175™; expressing medium levels of DLL3) cells, at an effector:target (E:T) ratio of 1:4 and were assessed for production of the cytokine interleukin-2 (IL-2) in the supernatant at 24 hours of co-culture.

Anti-tumor activity of engineered T cells expressing the anti-DLL3 CAR was assessed in a 3D tumor model using a spheroid killing assay. The engineered T cells were incubated with H82 tumor cell spheroids engineered to express a red fluorescent protein to permit monitoring of tumor spheroid lysis using the live cell microscopy, at an E:T ratio of 1:2 and 10,000 target cells.

Among the assessed anti-DLL3 CARs, eighteen (18) were selected based on the ability to produce the greatest amount of IL-2 and/or the highest degree of tumor killing in the spheroid killing assay, following the antigen-dependent stimulation with tumor-expressing cells.

The constructs encoding the 18 selected candidate CARs (designated CAR-A to CAR-R) were subject to codon-optimization (CO) and assessed for potential splice sites and modified in a conservative manner, such as by not altering the encoded amino acid sequence, including elimination of potential predicted splice sites (SSE) in the polynucleotide construct encoding the CAR.

Among the eighteen (18) candidates, a CAR having the scFv from the DLL3-mAb-1 antibody and a spacer containing a modified IgG4 hinge region (SEQ ID NO:1), designated CAR-A, exhibited high IL-2 production and spheroid killing. Table E8 lists sequence identifiers (SEQ ID NO:) corresponding to sequences of CAR-A.

TABLE E8
Sequence identifier (SEQ ID NO:) for CAR and Components

| CAR# | scFv # | Order | V_{H} | linker | V_{L} | scFv | spacer | TM | costim | CD3z | CAR |
|---|---|---|---|---|---|---|---|---|---|---|---|
| | | | Nucleotides (nt) | | | | | | | | |
| CAR-A | from | V_{H}- | 87 | 47 | 88 | 86 | 2 | 11 | 18 | 20 | 116 |
| | DLL3- | V_{L} | | | | | | | | | |
| | mAb-1 | | | | | | | | | | |
| | | | Amino acids (aa) | | | | | | | | |
| CAR-A | from | V_{H}- | 90 | 46 | 91 | 89 | 1 | 10 | 17 | 19 | 117 |
| | DLL3- | V_{L} | | | | | | | | | |
| | mAb-1 | | | | | | | | | | |

### Example 8: Further Assessment of Cytokine Production of Anti-DLL3 Chimeric Antigen Receptors (CARs)

The activity of selected anti-DLL3 CARs as described in Example 7 above, including CAR-A, were further assessed in primary human T cells.

CD4+ and CD8+ T cells isolated by immunoaffinity-based enrichment from leukapheresis samples from three (3) human donor subjects were stimulated and transduced with lentiviral preparations encoding one of the selected CARs. Primary T cells not expressing a CAR (mock) were assessed as controls.

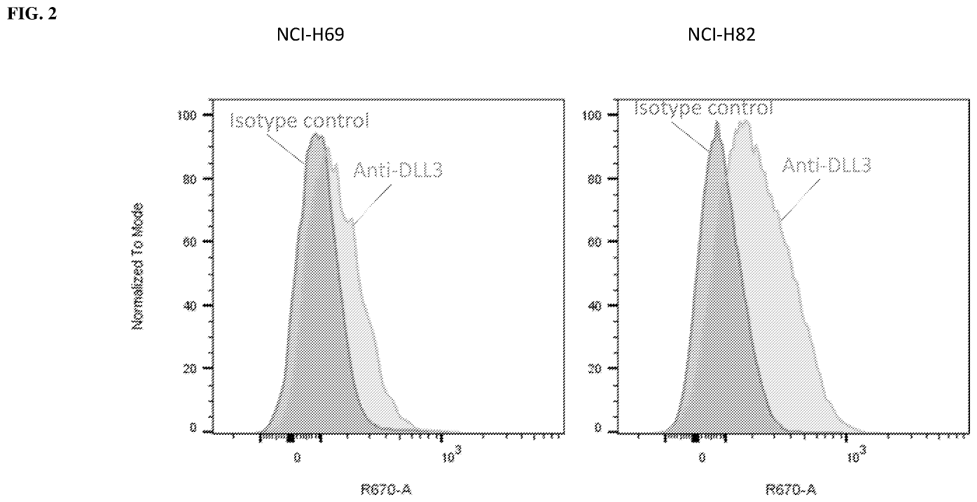

The 18 selected CAR-expressing primary T cells from 3 different donors were stimulated by co-culture with DLL3-expressing H69 (low DLL3) tumor cell spheroids or H82 (medium DLL3) tumor cell spheroids at an effector:target (E:T) ratio of 1:4, and were assessed for production of the cytokine interleukin-2 (IL-2) in the supernatant (pg/ml) at 24 hours of co-culture. FIG. 2 shows the DLL3 expression levels in H69 and H82 cells.

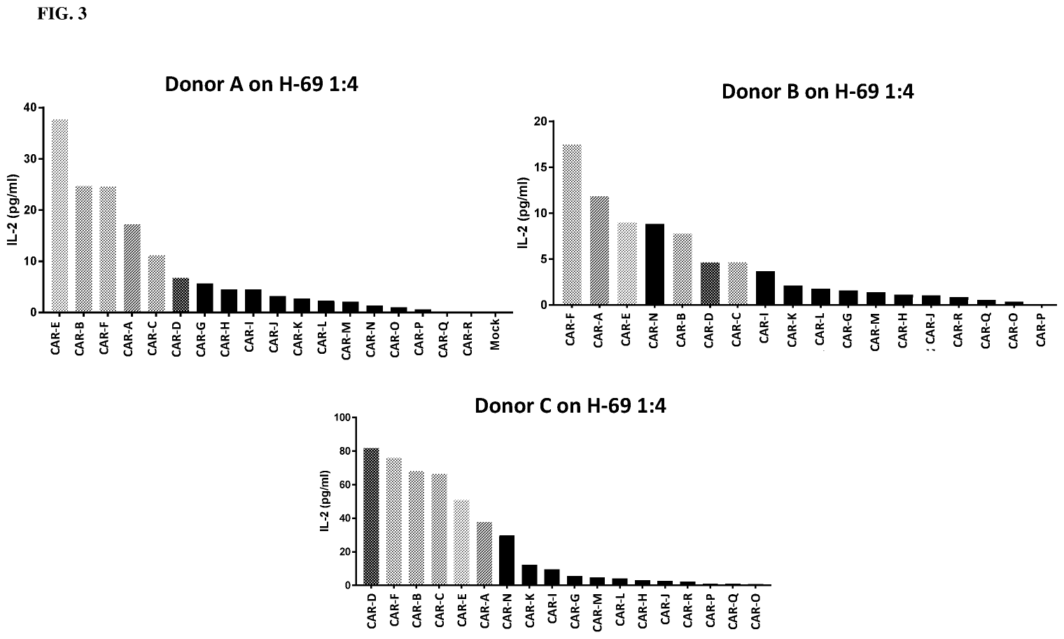

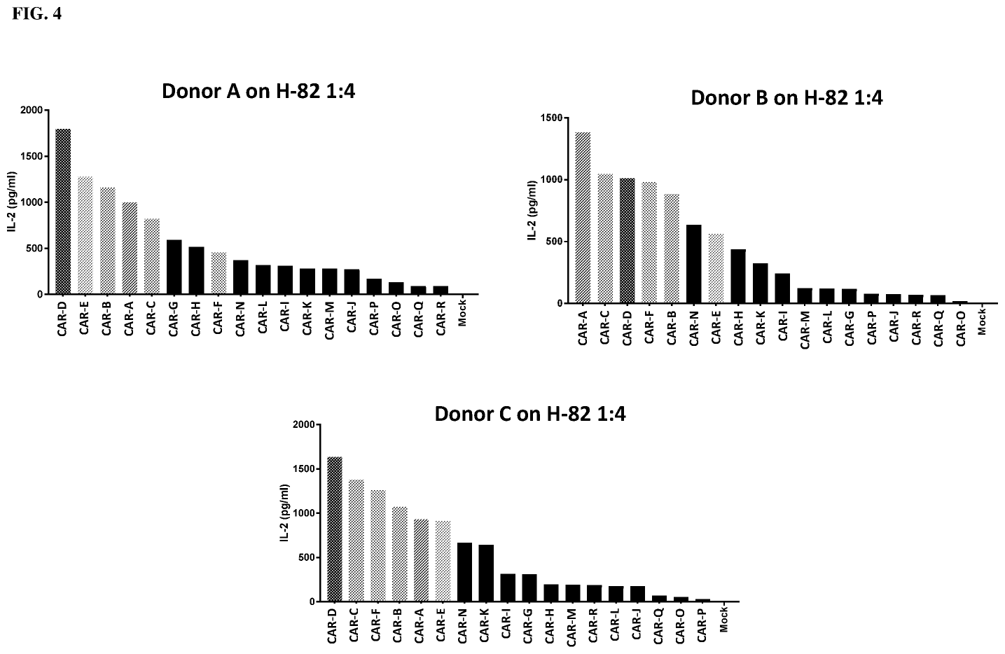

T cells expressing the selected anti-DLL3 CARs tested showed varying levels of IL-2 production when co-cultured with tumor cell spheroids expressing different levels of DLL3, with T cells expressing anti-DLL3 CAR-A producing among the highest levels of IL-2 using primary cells from the three (3) donors, in some cases the highest, as shown in FIG. 3 (H69, low DLL3) and FIG. 4 (H-82, medium DLL3). In general, higher IL-2 production was observed when co-cultured with H82 tumor cells, consistent with their higher expression of DLL3 compared to H69 tumor cells.

### Example 9: Further Assessment of Cytokine Production and Anti-Tumor Activity of Anti-DLL3 Chimeric Antigen Receptors (CARs)

Five (5) CARs from those tested in Examples 7 and 8 were selected for further assessment.

### A. Cytokine Production

Primary T cells from two (2) donors expressing one of the five (5) selected CARs (CAR-A described above and CAR-B, CAR-C, CAR-D, and CAR-E) were stimulated by co-culture with DLL3-expressing H69 (low DLL3) target cells or H82 (medium DLL3) target cells at an effector:target (E:T) ratio of 1:4, and were assessed for production of the cytokine interleukin-2 (IL-2) in the supernatant (pg/ml).

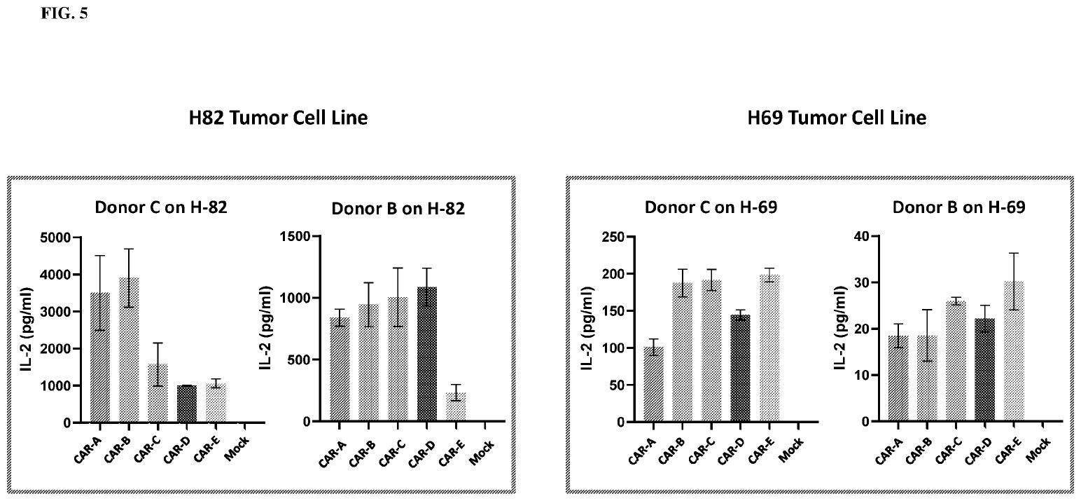

Results are shown in FIG. 5. Similar to results described in Example 8, T cells from both donors expressing CAR-A produced high IL-2 when co-cultured with H69 or H82 cells, as shown in FIG. 5.

### B. Spheroid Killing Assay

The anti-tumor activity of the five (5) selected CAR-expressing primary T cells was assessed in 3D tumor model using a spheroid killing assay. The engineered T cells were incubated with H69 (low DLL3) or H82 (medium DLL3) tumor cell spheroids, at an E:T ratio of 1:2. The DLL3-expressing tumor target cells were engineered with a red fluorescent protein to permit monitoring of tumor cell lysis. About 10,000 H69 cells or H82 cells were plated 48 hours prior to addition of 5,000 anti-DLL3 CAR T cells (1:2 E:T ratio).

Tumor spheroid size was measured over approximately 350 hours by measuring the red image mean (μm²/image) using live cell microscopy.

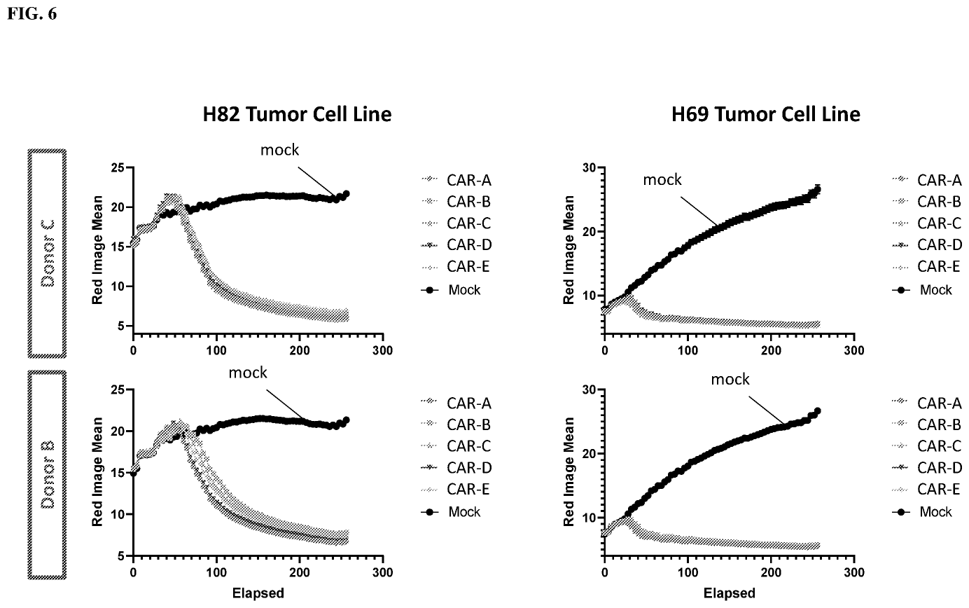

As shown in FIG. 6, the tested anti-DLL3 CARs showed spheroid killing activity, as indicated by the reduction of red image mean over time and a lower AUC compared to mock control. T cells expressing anti-DLL3 CAR A exhibited among the highest spheroid killing activity as indicated by lower red image mean and lower AUC, particularly when co-cultured with H82 tumor cells.

### Example 10: Multiple Dose Level Anti-Tumor Effect and Expansion of Anti-DLL3 CAR-Expressing T Cells after Adoptive Transfer in Vito in an Animal Model

The in vivo anti-tumor effects of exemplary engineered anti-DLL3 CAR-expressing primary human T cells were assessed by monitoring tumors following adoptive transfer of cells in tumor-bearing mouse xenograft models of small cell lung cancer. The studies were carried out with human small cell lung cancer H82 cells. Sixty (60) NSG mice were each injected subcutaneously with approximately 5×10⁶ human small cell lung cancer H82 (expressing medium levels of DLL3) cells. Fourteen (14) days after tumor implant, when the average tumor volume at staging was approximately 150-200 mm³, five (5) mice in each group received a single intravenous (i.v.) injection of engineered primary human T cells expressing one of anti-DLL3 CAR-A, CAR-B, CAR-C, CAR-D, or CAR-E as described in Examples 8-9 above, at two different dose levels: 1×10⁶ cells (low dose) or 4×10⁶ cells (high dose). As a control, mice were administered 4×10⁶ cells not expressing a CAR (mock) or were untreated (tumor only). Tumor volume was assessed twice per week out to approximately 70 days and the number of circulating CAR engineered T cells in the blood were assessed on day 7 and 21 after administration.

### A. Tumor Volume

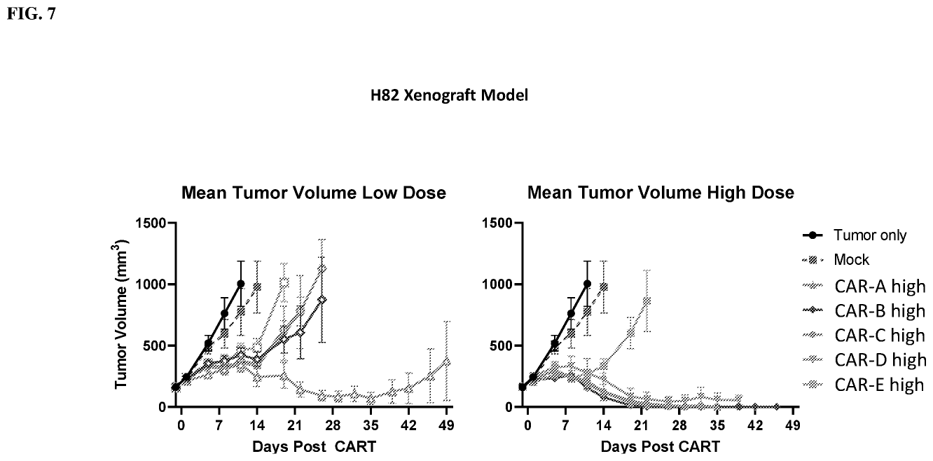

FIG. 7 depicts the mean tumor volume assessed over time in the treated mice; in this depiction, tumor curves were terminated after the first mouse of a group succumbed to disease. As shown in FIG. 7, primary human T cells expressing an anti-DLL3 CAR showed anti-tumor activity at both the low dose and high dose, as shown by reduction in tumor volume. Anti-DLL3 CAR-A showed superior anti-tumor efficacy at the low dose compared to CAR-B, CAR-C, CAR-D, and CAR-E and resulted in nearly complete elimination of mean tumor volume at around day 35.

### B. In Vivo Persistence

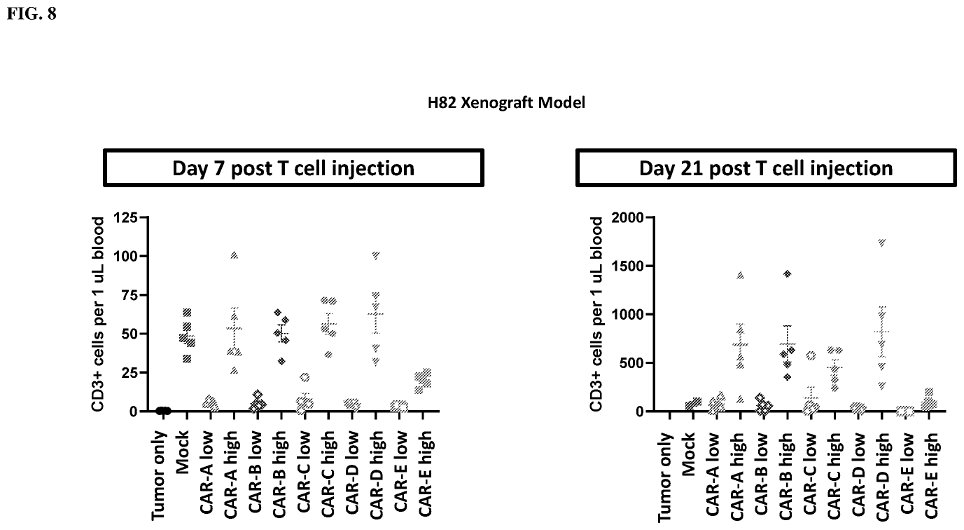

The average number of CAR-expressing T cells in the blood of each mouse was determined at day 7 and day 21 after administration, to assess the in vivo expansion and persistence of the administered CAR-expressing T cells. Cell number was quantified as total number of CD3+ cells per 1 μL blood. As shown in FIG. 8, T cells expressing anti-DLL3 CAR-A, CAR-B, CAR-C, CAR-D, or CAR-E were detectable at day 21 in the high dose groups.

### Example 11: Anti-Tumor Effect and Expansion of Anti-DLL3 CAR-Expressing T Cells After Adoptive Transfer In Vivo in an Animal Model

CAR-A and CAR-B, which showed the most robust anti-tumor efficacy and CAR T cell expansion in the H82 xenograft tumor model, were tested in the H69 xenograft tumor model, which expresses lower levels of DLL3. Sixty-four (64) NSG mice were each injected subcutaneously with approximately 5×10⁶ human small cell lung cancer H69 (expressing low levels of DLL3) cells. Twenty-one (21) days after tumor implant, when the average tumor volume at staging was approximately 150-200 mm³, eight (8) mice in each group received a single intravenous (i.v.) injection of engineered primary human T cells expressing one of anti-DLL3 CAR-A or CAR-B at three different dose levels: 1×10⁶ cells (low), 2.5×10⁶ cells (mid), or 5×10⁶ cells (high). As a control, mice were administered 5×10⁶ cells not expressing a CAR (mock) or were untreated (tumor only). Survival was assessed over 56 days. Tumor volume was assessed twice per week over 56 days, and the number of circulating CAR engineered T cells in the blood were assessed on days 7, 14, 21, and 28 after CAR-expressing T cell administration.

### A. Tumor Volume

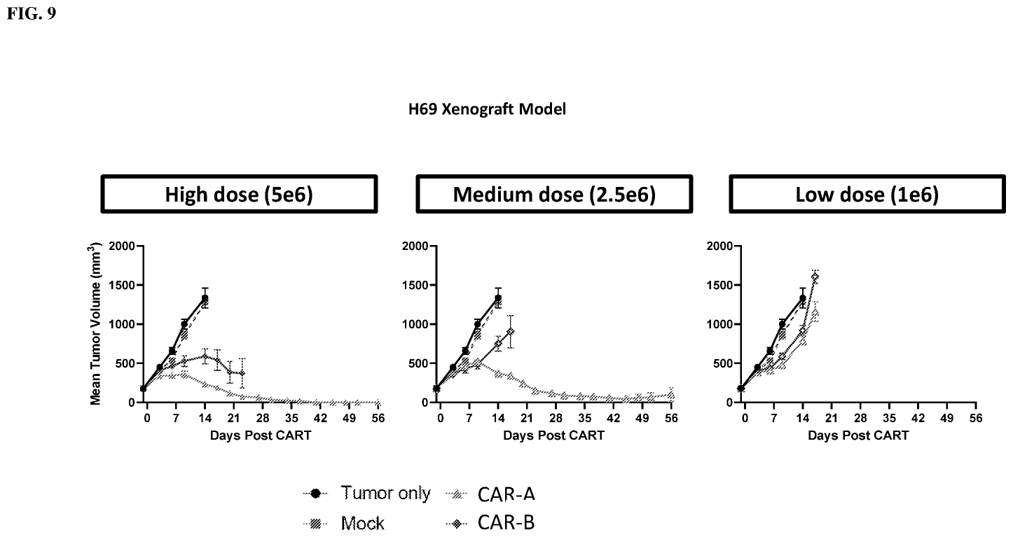

FIG. 9 depicts the mean tumor volume assessed over time in the treated mice. As shown in FIG. 9, primary human T cells expressing the anti-DLL3 CAR-A or CAR-B showed anti-tumor activity at all dose levels, as shown by reduction in tumor volume. Anti-DLL3 CAR-A showed superior anti-tumor efficacy at all dose levels compared to CAR-B and resulted in nearly complete elimination of mean tumor volume at the mid and high doses.

### B. Survival

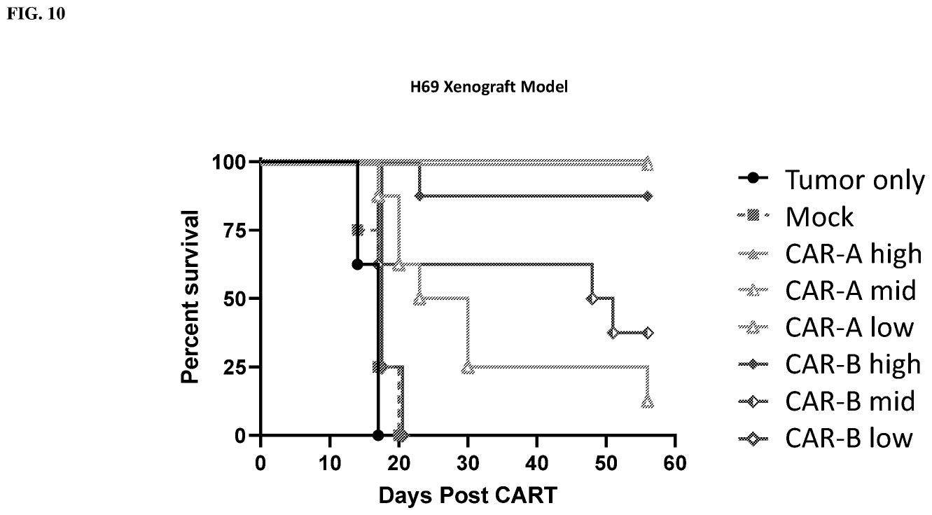

Kaplan-Meier survival curves of each of the groups are shown in FIG. 10. As shown in FIG. 10, administration of primary human T cells expressing anti-DLL3 CAR-A provided a survival benefit in all three dose levels compared to administration of cells not expressing a CAR (mock) and untreated mice (tumor only). Administration of primary human T cells expressing anti-DLL3 CAR-B provided a survival benefit in the mid and high dose levels compared to administration of cells not expressing a CAR (mock) and untreated mice (tumor only).

### C. In Vivo Persistence

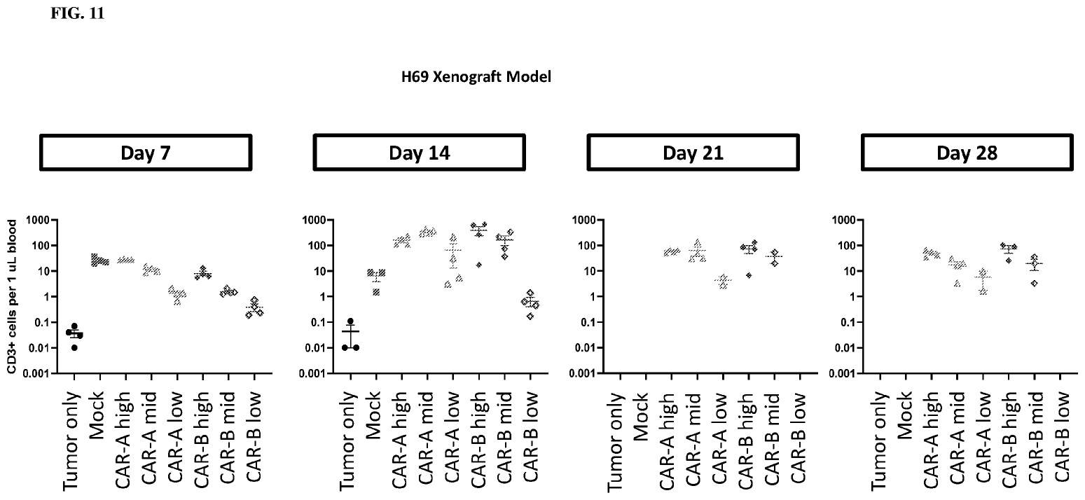

The average number of CAR-expressing T cells in the blood of each mouse was determined at days 7, 14, 21, and 28 after administration, to assess the in vivo expansion of the administered CAR-expressing T cells. Cell expansion was quantified as total number of CD3+ cells per 1 uL blood. As shown in FIG. 11, T cells expressing CAR-A or CAR-B was detected in the blood even up to day 28 after administration.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | Sequence | Description |
|---|---|---|
| 1 | ESKYGPPCPPCPM | Spacer (IgG4 hinge) |
| | | (aa) |
| 2 | GAGTCTAAATACGGACCGCCTTGTCCTCCTTGTCCCATG | Spacer (IgG4 |
| | | hinge)(nt) |
| 3 | ESKYGPPCPPCP | Spacer (IgG4 hinge) |
| | | (aa) |
| 4 | GAGTCTAAATACGGACCGCCTTGTCCTCCTTGTCCC | Spacer (IgG4 |
| | | hinge)(nt) |
| 5 | ESKYGPPCPPCPGQPREPQVYTLPPSQEEMTKNQVSLTCLVKGFYPSDIAVEWESNGQ | Hinge-CH3 spacer |
| | PENNYKTTPPVLDSDGSFFLYSRLTVDKSRWQEGNVFSCSVMHEALHNHYTQKSLSLS | (aa) |
| | LGK | |
| 6 | GAATCTAAGTACGGACCGCCTTGTCCTCCATGTCCTGGCCAGCCAAGAGAACCCCAGG | Hinge-CH3 spacer |
| | TGTACACACTGCCTCCAAGCCAAGAGGAAATGACCAAGAACCAGGTGTCCCTGACCTG | (nt) |
| | CCTGGTCAAGGGCTTCTACCCTTCCGATATCGCCGTGGAATGGGAGAGCAATGGCCAG | |
| | CCTGAGAACAACTACAAGACCACACCTCCTGTGCTGGACAGCGACGGCTCATTCTTCC | |
| | TGTACAGCCGGCTGACCGTGGACAAGAGCAGATGGCAAGAGGGCAACGTGTTCAGCTG | |
| | CAGCGTGATGCACGAGGCCCTGCACAACCACTACACCCAGAAGTCTCTGAGCCTGAGC | |
| | CTGGGCAAG | |
| 7 | RWPESPKAQASSVPTAQPQAEGSLAKATTAPATTRNTGRGGEEKKKEKEKEEQEERET | IgD-hinge-Fc |
| | KTPECPSHTQPLGVYLLTPAVQDLWLRDKATFTCFVVGSDLKDAHLTWEVAGKVPTGG | |
| | VEEGLLERHSNGSQSQHSRLTLPRSLWNAGTSVTCTLNHPSLPPQRLMALREPAAQAP | |
| | VKLSLNLLASSDPPEAASWLLCEVSGFSPPNILLMWLEDQREVNTSGFAPARPPPQPG | |
| | STTFWAWSVLRVPAPPSPQPATYTCVVSHEDSRTLLNASRSLEVSYVTDH | |
| 8 | LEGGGEGRGSLLTCGDVEENPGPR | T2A |
| 9 | MLLLVTSLLLCELPHPAFLLIPRKVCNGIGIGEFKDSLSINATNIKHFKNCTSISGDL | tEGFR |
| | HILPVAFRGDSFTHTPPLDPQELDILKTVKEITGELLIQAWPENRTDLHAFENLEIIR | |
| | GRTKQHGQFSLAVVSLNITSLGLRSLKEISDGDVIISGNKNLCYANTINWKKLFGTSG | |
| | QKTKIISNRGENSCKATGQVCHALCSPEGCWGPEPRDCVSCRNVSRGRECVDKCNLLE | |
| | GEPREFVENSECIQCHPECLPQAMNITCTGRGPDNCIQCAHYIDGPHCVKTCPAGVMG | |
| | ENNTLVWKYADAGHVCHLCHPNCTYGCTGPGLEGCPTNGPKIPSIATGMVGALLLLLV | |
| | VALGIGLEM | |
| 10 | FWVLVVVGGVLACYSLLVTVAFIIFWV | Human CD28 |
| | | transmembrane (aa) |
| 11 | TTCTGGGTGCTCGTGGTCGTTGGCGGAGTGCTGGCCTGTTACAGCCTGCTGGTTACCG | Human CD28 |
| | TGGCCTTCATCATCTTTTGGGTC | transmembrane (nt) |
| 12 | MFWVLVVVGGVLACYSLLVTVAFIIFWV | Human CD28 |
| | | transmembrane (aa) |
| 13 | ATGTTCTGGGTGCTCGTGGTCGTTGGCGGAGTGCTGGCCTGTTACAGCCTGCTGGTTA | Human CD28 |
| | CCGTGGCCTTCATCATCTTTTGGGTC | transmembrane (nt) |
| 14 | IEVMYPPPYLDNEKSNGTIIHVKGKHLCPSPLFPGPSKPFWVLVVVGGVLACYSLLVT | CD28 (amino acids |
| | VAFIIFWV | 114-179 of |
| | | Accession No. |
| | | P10747) |
| 15 | RSKRSRLLHSDYMNMTPRRPGPTRKHYQPYAPPRDFAAYRS | CD28 (amino acids |
| | | 180-220 of P10747) |
| 16 | RSKRSRGGHSDYMNMTPRRPGPTRKHYQPYAPPRDFAAYRS | CD28 (LL to GG) |
| 17 | KRGRKKLLYIFKQPFMRPVQTTQEEDGCSCRFPEEEEGGCEL | Human 4-1BB endo |
| | | domain (aa) |
| 18 | AAGCGGGGCAGAAAGAAGCTGCTCTACATCTTCAAGCAGCCCTTCATGCGGCCCGTGC | Human 4-1BB endo |
| | AGACCACACAAGAGGAAGATGGCTGCTCCTGCAGATTCCCCGAGGAAGAAGAAGGCGG | domain (nt) |
| | CTGCGAGCTG | |
| 19 | RVKFSRSADAPAYQQGQNQLYNELNLGRREEYDVLDKRRGRDPEMGGKPRRKNPQEGL | Human CD3-zeta |
| | YNELQKDKMAEAYSEIGMKGERRRGKGHDGLYQGLSTATKDTYDALHMQALPPR | endodomain (aa) |
| 20 | AGAGTGAAGTTCAGCAGATCCGCCGACGCTCCAGCCTATCAGCAGGGCCAAAACCAGC | Human CD3-zeta |
| | TGTACAACGAGCTGAACCTGGGGAGAAGAGAAGAGTACGACGTGCTGGATAAGCGGAG | endodomain (nt) |
| | AGGCAGAGATCCTGAAATGGGCGGCAAGCCCAGACGGAAGAATCCTCAAGAGGGCCTG | |
| | TATAATGAGCTGCAGAAAGACAAGATGGCCGAGGCCTACAGCGAGATCGGAATGAAGG | |
| | GCGAGCGCAGAAGAGGCAAGGGACACGATGGACTGTACCAGGGCCTGAGCACCGCCAC | |
| | CAAGGATACCTATGACGCACTGCACATGCAGGCCCTGCCACCTAGA | |
| 21 | RVKFSRSAEPPAYQQGQNQLYNELNLGRREEYDVLDKRRGRDPEMGGKPRRKNPQEGL | CD3 zeta |
| | YNELQKDKMAEAYSEIGMKGERRRGKGHDGLYQGLSTATKDTYDALHMQALPPR | |
| 22 | RVKFSRSADAPAYKQGQNQLYNELNLGRREEYDVLDKRRGRDPEMGGKPRRKNPQEGL | CD3 zeta |
| | YNELQKDKMAEAYSEIGMKGERRRGKGHDGLYQGLSTATKDTYDALHMQALPPR | |
| 23 | RKVCNGIGIGEFKDSLSINATNIKHFKNCTSISGDLHILPVAFRGDSFTHTPPLDPQE | tEGFR |
| | LDILKTVKEITGFLLIQAWPENRTDLHAFENLEIIRGRTKQHGQFSLAVVSLNITSLG | |
| | LRSLKEISDGDVIISGNKNLCYANTINWKKLFGTSGQKTKIISNRGENSCKATGQVCH | |
| | ALCSPEGCWGPEPRDCVSCRNVSRGRECVDKCNLLEGEPREFVENSECIQCHPECLPQ | |
| | AMNITCTGRGPDNCIQCAHYIDGPHCVKTCPAGVMGENNTLVWKYADAGHVCHLCHPN | |
| | CTYGCTGPGLEGCPTNGPKIPSIATGMVGALLLLLVVALGIGLEM | |
| 24 | EGRGSLLTCGDVEENPGP | T2A |
| 25 | GSGATNFSLLKQAGDVEENPGP | P2A |
| 26 | ATNFSLLKQAGDVEENPGP | P2A |
| 27 | QCTNYALLKLAGDVESNPGP | E2A |
| 28 | VKQTLNFDLLKLAGDVESNPGP | F2A |
| 29 | PGGG-(SGGGG)5-P- wherein P is proline, G is glycine and S | linker |
| | is serine | |
| 30 | GSADDAKKDAAKKDGKS | Linker |
| 31 | GSTSGSGKPGSGEGSTKG | Linker |
| 32 | X₁PPX₂P X₁ is glycine, cysteine or arginine X₂ is cysteine | Hinge |
| | or threonine | |
| 33 | EPKSCDKTHTCPPCP | Hinge |
| 34 | ERKCCVECPPCP | Hinge |
| 35 | ELKTPLGDTHTCPRCPEPKSCDTPPPCPRCPEPKSCDTPPPCPRCPEPKSCDTPPPCP | Hinge |
| | RCP | |
| 36 | ESKYGPPCPSCP | Hinge |
| 37 | YGPPCPPCP | Hinge |
| 38 | KYGPPCPPCP | Hinge |
| 39 | EVVVKYGPPCPPCP | Hinge |
| 40 | GAATCTAAGTACGGACCGCCTTGTCCTCCATGTCCTGCTCCTCCAGTTGCCGGACCTT | Hinge-CH2-CH3 |
| | CCGTGTTCCTGTTTCCTCCAAAGCCTAAGGACACCCTGATGATCAGCAGAACCCCTGA | spacer (nt) |
| | AGTGACCTGCGTGGTGGTGGACGTGTCCCAAGAGGATCCTGAGGTGCAGTTCAACTGG | |
| | TATGTGGACGGCGTGGAAGTGCACAACGCCAAGACCAAGCCTAGAGAGGAACAGTTCC | |
| | AGAGCACCIACAGAGTGGTGTCCGTGCTGACAGTGCTGCACCAGGATTGGCTGAACGG | |
| | CAAAGAGTACAAGTGCAAGGTGTCCAACAAGGGCCTGCCTAGCAGCATCGAGAAAACC | |
| | ATCAGCAAGGCCAAGGGCCAGCCAAGAGAACCCCAGGTGTACACACTGCCTCCAAGCC | |
| | AAGAGGAAATGACCAAGAACCAGGTGTCCCTGACCTGCCTGGTCAAGGGCTTCTACCC | |
| | TTCCGATATCGCCGTGGAATGGGAGAGCAATGGCCAGCCTGAGAACAACTACAAGACC | |
| | ACACCTCCTGTGCTGGACAGCGACGGCTCATTCTTCCTGTACAGCCGGCTGACCGTGG | |
| | ACAAGAGCAGATGGCAAGAGGGCAACGTGTTCAGCTGCAGCGTGATGCACGAGGCCCT | |
| | GCACAACCACTACACCCAGAAGTCTCTGAGCCTGAGCCTGGGCAAG | |
| 41 | ESKYGPPCPPCPAPEFLGGPSVFLFPPKPKDTLMISRTPEVTCVVVDVSQEDPEVQFN | Hinge-CH2-CH3 |
| | WYVDGVEVHNAKTKPREEQFNSTYRVVSVLTVLHQDWLNGKEYKCKVSNKGLPSSIEK | spacer (aa) |
| | TISKAKGQPREPQVYTLPPSQEEMTKNQVSLTCLVKGFYPSDIAVEWESNGQPENNYK | |
| | TTPPVLDSDGSFFLYSRLTVDKSRWQEGNVFSCSVMHEALHNHYTQKSLSLSLGK | |
| 42 | ESKYGPPCPPCPAPPVAGPSVFLFPPKPKDTLMISRTPEVTCVVVDVSQEDPEVQENW | IgG4/IgG2 hinge- |
| | YVDGVEVHNAKTKPREEQFQSTYRVVSVLTVLHQDWLNGKEYKCKVSNKGLPSSIEKT | IgG2/IgG4 CH2- |
| | ISKAKGQPREPQVYTLPPSQEEMTKNQVSLTCLVKGFYPSDIAVEWESNGQPENNYKT | IgG4 CH3 spacer |
| | TPPVLDSDGSFFLYSRLTVDKSRWQEGNVFSCSVMHEALHNHYTQKSLSLSLGK | (aa) |
| 43 | gaatctaagtacggaccgccctgccctccctgccctgctcctcctgtggctggaccaa | IgG4/IgG2 hinge- |
| | gcgtgttcctgtttccacctaagcctaaagataccctgatgatttcccgcacacctga | IgG2/IgG4 CH2- |
| | agtgacttgcgtggtcgtggacgtgagccaggaggatccagaagtgcagttcaactgg | IgG4 CH3 spacer |
| | tacgtggacggcgtggaagtccacaatgctaagactaaaccccgagaggaacagtttc | (nt) |
| | agtcaacttaccgggtcgtgagcgtgctgaccgtcctgcatcaggattggctgaacgg | |
| | gaaggagtataagtgcaaagtgtctaataagggactgcctagctccatcgagaaaaca | |
| | attagtaaggcaaaagggcagcctcgagaaccacaggtgtataccctgccccctagcc | |
| | aggaggaaatgaccaagaaccaggtgtccctgacatgtctggtcaaaggcttctatcc | |
| | aagtgacatcgccgtggagtgggaatcaaatgggcagcccgagaacaattacaagacc | |
| | acaccacccgtgctggactctgatggaagtttctttctgtattccaggctgaccgtgg | |
| | ataaatctcgctggcaggagggcaacgtgttctcttgcagtgtcatgcacgaagccct | |
| | gcacaatcattatacacagaagtcactgagcctgtccctgggcaaa | |
| 44 | GGGGS | 4GS linker (aa) |
| 45 | GGGS | 3GS linker (aa) |
| 46 | GGGGSGGGGSGGGGS | (4GS)3 linker (aa) |
| 47 | GGAGGCGGAGGATCTGGTGGCGGAGGAAGTGGCGGAGGCGGTTCT | (4GS)3 linker (nt) |
| 48 | MPLLLLLPLLWAGALA | Human CD33 Signal |
| | | Peptide (aa) |
| 49 | ATGCCGCTGCTGCTACTGCTGCCCCTGCTGTGGGCAGGGGCTCTAGCT | Human CD33 Signal |
| | | Peptide (nt) |
| 50 | MVLQTQVFISLLLWISGAYG | human IgG-kappa |
| | | signal peptide (aa) |
| 51 | atggtgctgcagacccaggtgttcatcagcctgctgctgtggatctccggagcatacg | human IgG-kappa |
| | ga | signal sequence (nt) |
| 52 | MLLLVTSLLLCELPHPAFLLIP | GMCSFR alpha |
| | | chain signal peptide |
| | | (aa) |
| 53 | atgcttctcctggtgacaagccttctgctctgtgagttaccacacccagcattcctcc | GMCSFR alpha |
| | tgatccca | chain signal |
| | | sequence (nt) |
| 54 | MALPVTALLLPLALLLHA | CD8 alpha signal |
| | | peptide |
| 55 | ASTKGPSVFPLAPCSRSTSESTAALGCLVKDYFPEPVTVSWNSGALTSGVHTFPAVLQ | Human IgG2 Fc |
| | SSGLYSLSSVVTVPSSNFGTQTYTCNVDHKPSNTKVDKTVERKCCVECPPCPAPPVAG | (Uniprot P01859) |
| | PSVFLFPPKPKDTLMISRTPEVTCVVVDVSHEDPEVQFNWYVDGVEVHNAKTKPREEQ | |
| | FNSTFRVVSVLTVVHQDWLNGKEYKCKVSNKGLPAPIEKTISKTKGQPREPQVYTLPP | |
| | SREEMTKNQVSLTCLVKGFYPSDISVEWESNGQPENNYKTTPPMLDSDGSFFLYSKLT | |
| | VDKSRWQQGNVFSCSVMHEALHNHYTQKSLSLSPGK | |
| 56 | ASTKGPSVFPLAPCSRSTSESTAALGCLVKDYFPEPVTVSWNSGALTSGVHTFPAVLQ | Human IgG4 Fc |
| | SSGLYSLSSVVTVPSSSLGTKTYTCNVDHKPSNTKVDKRVESKYGPPCPSCPAPEFLG | (Uniprot P01861) |
| | GPSVFLFPPKPKDTLMISRTPEVTCVVVDVSQEDPEVQFNWYVDGVEVHNAKTKPREE | |
| | QFNSTYRVVSVLTVLHQDWLNGKEYKCKVSNKGLPSSIEKTISKAKGQPREPQVYTLP | |
| | PSQEEMTKNQVSLTCLVKGFYPSDIAVEWESNGQPENNYKTTPPVLDSDGSFFLYSRL | |
| | TVDKSRWQEGNVFSCSVMHEALHNHYTQKSLSLSLGK | |
| 57 | gagtctaaatacggaccgccttgtcctccttgtcccggccagccaagagagccccagg | Hinge-CH3 spacer |
| | tttacacactgcctccaagccaagaggaaatgaccaagaatcaggtgtccctgacatg | (nt) |
| | cctggtcaagggcttctacccctccgatatcgccgtggaatgggagagcaatggccag | |
| | cctgagaacaactacaagaccacacctcctgtgctggacagcgacggcagtttcttcc | |
| | tgtatagtagactcaccgtggataaatcaagatggcaagagggcaacgtgttcagctg | |
| | cagcgtgatgcacgaggccctgcacaaccactacacccagaaaagcctgagcctgtct | |
| | ctgggcaag | |
| 58 | ESKYGPPCPPCPGQPREPQVYTLPPSQEEMTKNQVSLTCLVKGFYPSDIAVEWESNGQ | Hinge-CH3 spacer |
| | PENNYKTTPPVLDSDGSFFLYSRLTVDKSRWQEGNVFSCSVMHEALHNHYTQKSLSLS | (aa) |
| | LGKM | |
| 59 | gagtctaaatacggaccgccttgtcctccttgtcccggccagccaagagagccccagg | Hinge-CH3 spacer |
| | tttacacactgcctccaagccaagaggaaatgaccaagaatcaggtgtccctgacatg | (nt) |
| | cctggtcaagggcttctacccctccgatatcgccgtggaatgggagagcaatggccag | |
| | cctgagaacaactacaagaccacacctcctgtgctggacagcgacggcagtttcttcc | |
| | tgtatagtagactcaccgtggataaatcaagatggcaagagggcaacgtgttcagctg | |
| | cagcgtgatgcacgaggccctgcacaaccactacacccagaaaagcctgagcctgtct | |
| | ctgggcaagatg | |
| 60 | gagtctaaatacggaccgccttgtcctccttgtcccgctcctcctgttgccggacctt | IgG4/IgG2 hinge- |
| | ccgtgttcctgtttcctccaaagcctaaggacaccctgatgatcagcaggacccctga | IgG2/IgG4 CH2- |
| | agtgacctgcgtggtggtggatgtgtcccaagaggatcccgaggtgcagttcaactgg | IgG4 CH3 spacer |
| | tatgtggacggcgtggaagtgcacaacgccaagaccaagcctagagaggaacagttcc | (nt) |
| | agagcacctacagagtggtgtccgtgctgacagtgctgcaccaggattggctgaacgg | |
| | caaagagtacaagtgcaaggtgtccaacaagggcctgcctagcagcatcgagaaaacc | |
| | atctccaaggccaagggccagccaagagagccccaggtttacacactgcctccaagcc | |
| | aagaggaaatgaccaagaatcaggtgtccctgacatgcctggtcaagggcttctaccc | |
| | ctccgatatcgccgtggaatgggagagcaatggccagcctgagaacaactacaagacc | |
| | acacctcctgtgctggacagcgacggcagtttcttcctgtatagtagactcaccgtgg | |
| | ataaatcaagatggcaagagggcaacgtgttcagctgcagcgtgatgcacgaggccct | |
| | gcacaaccactacacccagaaaagcctgagcctgtctctgggcaag | |
| 61 | cttgaaggtggtggcgaaggcagaggcagcctgcttacatgcggagatgtggaagaga | T2A (nt) |
| | accccggacctaga | |
| 62 | atgttttgggtgctggtcgtggtcggaggggtgctggcctgttacagcctgctggtga | CD28 |
| | cagtcgctttcatcatcttctgggtg | transmembrane |
| | | domain (nt) |
| 63 | GAATCTAAGTACGGACCGCCTTGTCCTCCATGTCCTGGCCAGCCAAGAGAACCCCAGG | Hinge-CH3 spacer |
| | TGTACACACTGCCTCCAAGCCAAGAGGAAATGACCAAGAACCAGGTGTCCCTGACCTG | (nt) |
| | CCTGGTCAAGGGCTTCTACCCTTCCGATATCGCCGTGGAATGGGAGAGCAATGGCCAG | |
| | CCTGAGAACAACTACAAGACCACACCTCCTGTGCTGGACAGCGACGGCTCATTCTTCC | |
| | TGTACAGCCGGCTGACCGTGGACAAGAGCAGATGGCAAGAGGGCAACGTGTTCAGCTG | |
| | CAGCGTGATGCACGAGGCCCTGCACAACCACTACACCCAGAAGTCTCTGAGCCTGAGC | |
| | CTGGGCAAGATG | |
| 64 | atgcttctcctggtgacaagccttctgctctgtgagttaccacacccagcattcctcc | truncated EGFR |
| | tgatcccacgcaaagtgtgtaacggaataggtattggtgaatttaaagactcactctc | (tEGFR) sequence |
| | cataaatgctacgaatattaaacacttcaaaaactgcacctccatcagtggcgatctc | (nt) |
| | cacatcctgccggtggcatttaggggtgactccttcacacatactcctcctctggatc | |
| | cacaggaactggatattctgaaaaccgtaaaggaaatcacagggtttttgctgattca | |
| | ggcttggcctgaaaacaggacggacctccatgcctttgagaacctagaaatcatacgc | |
| | ggcaggaccaagcaacatggtcagttttctcttgcagtcgtcagcctgaacataacat | |
| | ccttgggattacgctccctcaaggagataagtgatggagatgtgataatttcaggaaa | |
| | caaaaatttgtgctatgcaaatacaataaactggaaaaaactgtttgggacctccggt | |
| | cagaaaaccaaaattataagcaacagaggtgaaaacagctgcaaggccacaggccagg | |
| | tctgccatgccttgtgctcccccgagggctgctggggcccggagcccagggactgcgt | |
| | ctcttgccggaatgtcagccgaggcagggaatgcgtggacaagtgcaaccttctggag | |
| | ggtgagccaagggagtttgtggagaactctgagtgcatacagtgccacccagagtgcc | |
| | tgcctcaggccatgaacatcacctgcacaggacggggaccagacaactgtatccagtg | |
| | tgcccactacattgacggcccccactgcgtcaagacctgcccggcaggagtcatggga | |
| | gaaaacaacaccctggtctggaagtacgcagacgccggccatgtgtgccacctgtgcc | |
| | atccaaactgcacctacggatgcactgggccaggtcttgaaggctgtccaacgaatgg | |
| | gcctaagatcccgtccatcgccactgggatggtgggggccctcctcttgctgctggtg | |
| | gtggccctggggatcggcctcttcatgtga | |
| 65 | atgctgctcctcgtgacaagcctgctcctgtgtgaactccctcatccagcttttctgc | truncated EGFR |
| | tcattcctcggaaagtgtgcaacggcatcggcatcggagagttcaaggacagcctgag | (tEGFR) sequence |
| | catcaatgccaccaacatcaagcacttcaagaattgcaccagcatcagcggcgacctg | (nt) |
| | cacattctgcctgtggcctttagaggcgacagcttcacccacacacctccactggatc | |
| | cccaagagctggatatcctgaaaaccgtgaaagagattaccggattcctcctgatcca | |
| | agcctggccagagaacagaaccgatctgcacgccttcgagaacctcgagatcatcaga | |
| | ggccggaccaaacagcacggccagtttagcctggctgtggtgtctctgaacatcacca | |
| | gtctgggcctgagaagcctgaaagaaatctccgacggcgacgtgatcatctccggaaa | |
| | caagaacctgtgctacgccaacaccatcaactggaagaagctgttcggcacctccggc | |
| | cagaaaaaaagatcatctctaaccggggcgagaacagctgcaaggccaccggacaag | |
| | tttgtcacgccctgtgtagccctgaaggctgttggggacccgaacctagagactgtgt | |
| | gtcctgccggaatgtgtcccggggcagagaatgtgtggataagtgcaacctgctggaa | |
| | ggcgagccccgcgagtttgtggaaaacagcgagtgcatccagtgtcaccccgagtgtc | |
| | tgccccaggccatgaacattacatgcaccggcagaggccccgacaactgtattcagtg | |
| | cgcccactacatcgacggccctcactgcgtgaaaacatgtccagctggcgtgatggga | |
| | gagaacaacaccctcgtgtggaagtatgccgacgccggacatgtgtgccacctgtgtc | |
| | accctaattgcacctacggctgtaccggacctggcctggaaggatgccctacaaacgg | |
| | ccctaagatccccagcattgccaccggaatggttggagccctgctgcttctgttggtg | |
| | gtggccctcggaatcggcctgttcatgtga | |
| 66 | MLLLVTSLLLCELPHPAFLLIPRKVCNGIGIGEFKDSLSINATNIKHFKNCTSISGDL | truncated EGFR |
| | HILPVAFRGDSFTHTPPLDPQELDILKTVKEITGELLIQAWPENRTDLHAFENLEIIR | (tEGFR) sequence |
| | GRIKQHGQFSLAVVSLNITSLGLRSLKEISDGDVIISGNKNLCYANTINWKKLFGTSG | (aa) |
| | QKTKIISNRGENSCKATGQVCHALCSPEGCWGPEPRDCVSCRNVSRGRECVDKCNLLE | |
| | GEPREFVENSECIQCHPECLPQAMNITCTGRGPDNCIQCAHYIDGPHCVKTCPAGVMG | |
| | ENNTLVWKYADAGHVCHLCHPNCTYGCTGPGLEGCPTNGPKIPSIATGMVGALLLLLV | |
| | VALGIGLFM | |
| 67 | aagcgggggagaaagaaactgctgtatattttcaaacagccctttatgagacctgtgc | 4-1BB intracellular |
| | agactacccaggaggaagacggatgcagctgtaggtttcccgaggaagaggaaggagg | co-signaling |
| | ctgtgagctg | sequence (nt) |
| 68 | SRGGGGSGGGGSGGGGSLEMA | linker |
| 69 | GSRGGGGSGGGGSGGGGSLEMA | linker |
| 70 | ggatctgcgatcgctccggtgcccgtcagtgggcagagcgcacatcgcccacagtccc | EF1 alpha promoter |
| | cgagaagttggggggaggggtcggcaattgaaccggtgcctagagaaggtggcgcggg | with HTLV1 |
| | gtaaactgggaaagtgatgtcgtgtactggctccgcctttttcccgagggtgggggag | ehancer |
| | aaccgtatataagtgcagtagtcgccgtgaacgttctttttcgcaacgggtttgccgc | |
| | cagaacacagctgaagcttcgaggggctcgcatctctccttcacgcgcccgccgccct | |
| | acctgaggccgccatccacgccggttgagtcgcgttctgccgcctcccgcctgtggtg | |
| | cctcctgaactgcgtccgccgtctaggtaagtttaaagctcaggtcgagaccgggcct | |
| | ttgtccggcgctcccttggagcctacctagactcagccggctctccacgctttgcctg | |
| | accctgcttgctcaactctacgtctttgtttcgttttctgttctgcgccgttacagat | |
| | ccaagctgtgaccggcgcctac | |
| 71 | aatcaacctctggattacaaaatttgtgaaagattgactggtattcttaactatgttg | Woodchuck |
| | ctccttttacgctatgtggatacgctgctttaatgcctttgtatcatgctattgcttc | Hepatitis Virus |
| | ccgtatggctttcattttctcctccttgtataaatcctggttgctgtctctttatgag | (WHP) |
| | gagttgtggcccgttgtcaggcaacgtggcgtggtgtgcactgtgtttgctgacgcaa | Posttranscriptional |
| | cccccactggttggggcattgccaccacctgtcagctcctttccgggactttcgcttt | Regulatory Element |
| | ccccctccctattgccacggcggaactcatcgccgcctgccttgcccgctgctggaca | (WPRE) |
| | ggggctcggctgttgggcactgacaattccgtggtgttgtcggggaaatcatcgtcct | |
| | ttccttggctgctcgcctgtgttgccacctggattctgcgcgggacgtccttctgcta | |
| | cgtcccttcggccctcaatccagcggaccttccttcccgcggcctgctgccggctctg | |
| | cggcctcttccgcgtcttcgccttcgccctcagacgagtcggatctccctttgggccg | |
| | cctccccgc | |
| 72 | GSGEGRGSLLTCGDVEENPGP | T2A peptide (aa) |
| 73 | GSGQCTNYALLKLAGDVESNPGP | E2A peptide (aa) |
| 74 | GSGVKQTLNFDLLKLAGDVESNPGP | F2A peptide (aa) |
| 75 | ctcgagggcggcggagagggcagaggaagtcttctaacatgcggtgacgtggaggaga | T2A peptide (nt) |
| | atcccggccctagg | |
| 76 | agagtcaagttttccaggtccgccgacgctccagcctaccagcaggggcagaaccagc | CD3-zeta derived |
| | tgtacaacgagctgaacctgggcagaagggaagagtacgacgtcctggataagcggag | intracellular |
| | aggccgggaccctgagatgggcggcaagcctcggcggaagaacccccaggaaggcctg | signaling domain |
| | tataacgaactgcagaaagacaagatggccgaggcctacagcgagatcggcatgaagg | (nt) |
| | gcgagcggaggcggggcaagggccacgacggcctgtatcagggcctgtccaccgccac | |
| | caaggatacctacgacgccctgcacatgcaggccctgcccccaagg | |
| 77 | aggagtaagaggagcaggctcctgcacagtgactacatgaacatgactccccgccgcc | CD28 endodomain |
| | ccgggcccacccgcaagcattaccagccctatgccccaccacgcgacttcgcagccta | (nt) |
| | tcgctcc | |
| 78 | atgcctctgctgctgcttctgcctcttctttgggctggtgctctggct | CD33 signal |
| | | sequence (nt) |
| 79 | atgccgctgctgctactgctgcccctgctgtgggcaggggccctggct | CD33 signal |
| | | sequence (nt) |
| | | GenBank: |
| | | M23197.1 |
| 80 | gaatctaagtacggaccgccctgccccccttgccctatg | Spacer (IgG4 |
| | | hinge)(nt) |
| 81 | GAATCTAAGTACGGACCGCCTTGTCCTCCATGTCCTGGCCAGCCAAGAGAACCCCAGG | Hinge-CH3 spacer |
| | TGTACACACTGCCTCCAAGCCAAGAGGAAATGACCAAGAACCAGGTGTCCCTGACCTG | (nt) |
| | CCTGGTCAAGGGCTTCTACCCTTCCGATATCGCCGTGGAATGGGAGAGCAATGGCCAG | |
| | CCTGAGAACAACTACAAGACCACACCTCCTGTGCTGGACAGCGACGGCTCATTCTTCC | |
| | TGTACAGCCGGCTGACCGTGGACAAGAGCAGATGGCAAGAGGGCAACGTGTTCAGCTG | |
| | CAGCGTGATGCACGAGGCCCTGCACAACCACTACACCCAGAAGTCTCTGAGCCTGAGC | |
| | CTGGGCAAG | |
| 82 | ESKYGPPCPPCPAPPVAGPSVFLFPPKPKDTLMISRTPEVTCVVVDVSQEDPEVQFNW | IgG4/IgG2 hinge- |
| | YVDGVEVHNAKTKPREEQFQSTYRVVSVLTVLHQDWLNGKEYKCKVSNKGLPSSIEKT | IgG2/IgG4 CH2- |
| | ISKAKGQPREPQVYTLPPSQEEMTKNQVSLTCLVKGFYPSDIAVEWESNGQPENNYKT | IgG4 CH3 spacer |
| | TPPVLDSDGSFFLYSRLTVDKSRWQEGNVFSCSVMHEALHNHYTQKSLSLSLGKM | (aa) |
| 83 | gaatctaagtacggaccgccctgccctccctgccctgctcctcctgtggctggaccaa | IgG4/IgG2 hinge- |
| | gcgtgttcctgtttccacctaagcctaaagataccctgatgatttcccgcacacctga | IgG2/IgG4 CH2- |
| | agtgacttgcgtggtcgtggacgtgagccaggaggatccagaagtgcagttcaactgg | IgG4 CH3 spacer |
| | tacgtggacggcgtggaagtccacaatgctaagactaaaccccgagaggaacagtttc | (nt) |
| | agtcaacttaccgggtcgtgagcgtgctgaccgtcctgcatcaggattggctgaacgg | |
| | gaaggagtataagtgcaaagtgtctaataagggactgcctagctccatcgagaaaaca | |
| | attagtaaggcaaaagggcagcctcgagaaccacaggtgtataccctgccccctagcc | |
| | aggaggaaatgaccaagaaccaggtgtccctgacatgtctggtcaaaggcttctatcc | |
| | aagtgacatcgccgtggagtgggaatcaaatgggcagcccgagaacaattacaagacc | |
| | acaccacccgtgctggactctgatggaagtttctttctgtattccaggctgaccgtgg | |
| | ataaatctcgctggcaggagggcaacgtgttctcttgcagtgtcatgcacgaagccct | |
| | gcacaatcattatacacagaagtcactgagcctgtccctgggcaaaatg | |
| 84 | gagtctaaatacggaccgccttgtcctccttgtcccgctcctcctgttgccggacctt | IgG4/IgG2 hinge- |
| | ccgtgttcctgtttcctccaaagcctaaggacaccctgatgatcagcaggacccctga | IgG2/IgG4 CH2- |
| | agtgacctgcgtggtggtggatgtgtcccaagaggatcccgaggtgcagttcaactgg | IgG4 CH3 spacer |
| | tatgtggacggcgtggaagtgcacaacgccaagaccaagcctagagaggaacagttcc | (nt) |
| | agagcacctacagagtggtgtccgtgctgacagtgctgcaccaggattggctgaacgg | |
| | caaagagtacaagtgcaaggtgtccaacaagggcctgcctagcagcatcgagaaaacc | |
| | atctccaaggccaagggccagccaagagagccccaggtttacacactgcctccaagcc | |
| | aagaggaaatgaccaagaatcaggtgtccctgacatgcctggtcaagggcttctaccc | |
| | ctccgatatcgccgtggaatgggagagcaatggccagcctgagaacaactacaagacc | |
| | acacctcctgtgctggacagcgacggcagtttcttcctgtatagtagactcaccgtgg | |
| | ataaatcaagatggcaagagggcaacgtgttcagctgcagcgtgatgcacgaggccct | |
| | gcacaaccactacacccagaaaagcctgagcctgtctctgggcaagatg | |
| 85 | ttttgggtgctggtcgtggtcggaggggtgctggcctgttacagcctgctggtgacag | CD28 |
| | tcgctttcatcatcttctgggtg | transmembrane |
| | | domain (nt) |
| 86 | CAGGTTCAGCTGCAAGAGTCTGGCCCTGGCCTGGTCAAGCCTAGCCAAACACTGAGCC | DLL3-mAb-1 scFv |
| | TGACCTGTACCGTGTCCGGCGATAGCATCAGCAGCGACGGCTACTACTGGTCCTGGAT | (VH-VL) (nt) |
| | CAGACAGCACCCTGGCAAAGGCCTGGAATGGATCGGCTACATCTACTACAGCGGCAAC | |
| | ACCTACTACAACCCCAGCCTGAAGTCCAGAGTGACCATCAGCGTGGACACCAGCAAGA | |
| | ACCAGTTCTCCCTGAAGCTGACCAGCGTGACAGCCGCCGATACCGCCATCTACTATTG | |
| | CGCCATCGATACCGCTAGCGCCCTGGCTTATTGGGGCCAGGGAACACTGGTCACAGTG | |
| | TCTAGCGGAGGCGGAGGATCTGGTGGCGGAGGAAGTGGCGGAGGCGGTTCTGAGATTG | |
| | TGATGACACAGAGCCCCGCCACACTGTCCGTTTCTCCCGGCGAAAGAGCCACACTGAG | |
| | CTGCAGAGCCAGCCAGAGCGTGTCCTCTAACCTGGCTTGGTATCAGCAGAAGCCCGGA | |
| | CAGGCTCCCAGACTGCTGATCTATGGCGCCTCTACAAGAGCCACCGGCATTCCCGCCC | |
| | GGTTTTCTGGCTCTGGAAGCGGCACCGAGTTCACCCTGACAATCAGCAGCCTGCAGAG | |
| | CGAGGACTTCGCCGTGTACTACTGCCAGCAGTACAACAACTGGCCTTGGACCTTTGGC | |
| | CAGGGCACCAAAGTGGAAATCAAGCGG | |
| 87 | CAGGTTCAGCTGCAAGAGTCTGGCCCTGGCCTGGTCAAGCCTAGCCAAACACTGAGCC | DLL3-mAb-1 VH |
| | TGACCTGTACCGTGTCCGGCGATAGCATCAGCAGCGACGGCTACTACTGGTCCTGGAT | (nt) |
| | CAGACAGCACCCTGGCAAAGGCCTGGAATGGATCGGCTACATCTACTACAGCGGCAAC | |
| | ACCTACTACAACCCCAGCCTGAAGTCCAGAGTGACCATCAGCGTGGACACCAGCAAGA | |
| | ACCAGTTCTCCCTGAAGCTGACCAGCGTGACAGCCGCCGATACCGCCATCTACTATTG | |
| | CGCCATCGATACCGCTAGCGCCCTGGCTTATTGGGGCCAGGGAACACTGGTCACAGTG | |
| | TCTAGC | |
| 88 | GAGATTGTGATGACACAGAGCCCCGCCACACTGTCCGTTTCTCCCGGCGAAAGAGCCA | DLL3-mAb-1 VL |
| | CACTGAGCTGCAGAGCCAGCCAGAGCGTGTCCTCTAACCTGGCTTGGTATCAGCAGAA | (nt) |
| | GCCCGGACAGGCTCCCAGACTGCTGATCTATGGCGCCTCTACAAGAGCCACCGGCATT | |
| | CCCGCCCGGTTTTCTGGCTCTGGAAGCGGCACCGAGTTCACCCTGACAATCAGCAGCC | |
| | TGCAGAGCGAGGACTTCGCCGTGTACTACTGCCAGCAGTACAACAACTGGCCTTGGAC | |
| | CTTTGGCCAGGGCACCAAAGTGGAAATCAAGCGG | |
| 89 | QVQLQESGPGLVKPSQTLSLTCTVSGDSISSDGYYWSWIRQHPGKGLEWIGYIYYSGN | DLL3-mAb-1 scFv |
| | TYYNPSLKSRVTISVDTSKNQFSLKLTSVTAADTAIYYCAIDTASALAYWGQGTLVTV | (VH-VL) (aa) |
| | SSGGGGGGGGSGGGGSEIVMTQSPATLSVSPGERATLSCRASQSVSSNLAWYQQKPG | |
| | QAPRLLIYGASTRATGIPARFSGSGSGTEFTLTISSLQSEDFAVYYCQQYNNWPWTFG | |
| | QGTKVEIKR | |
| 90 | QVQLQESGPGLVKPSQTLSLTCTVSGDSISSDGYYWSWIRQHPGKGLEWIGYIYYSGN | DLL3-mAb-1 VH |
| | TYYNPSLKSRVTISVDTSKNQFSLKLTSVTAADTAIYYCAIDTASALAYWGQGTLVTV | (aa) |
| | SS | |
| 91 | EIVMTQSPATLSVSPGERATLSCRASQSVSSNLAWYQQKPGQAPRLLIYGASTRATGI | DLL3-mAb-1 VL |
| | PARFSGSGSGTEFTLTISSLQSEDFAVYYCQQYNNWPWTFGQGTKVEIKR | (aa) |
| 92 | SDGYYWS | DLL3-mAb-1 CDR- |
| | | H1 (aa) (Kabat) |
| 93 | YIYYSGNTYYNPSLKS | DLL3-mAb-1 CDR- |
| | | H2 (aa) (Kabat) |
| 94 | DTASALAY | DLL3-mAb-1 CDR- |
| | | H3 (aa) (Kabat) |
| 95 | RASQSVSSNLA | DLL3-mAb-1 CDR- |
| | | L1 (aa) (Kabat) |
| 96 | GASTRAT | DLL3-mAb-1 CDR- |
| | | L2 (aa) (Kabat) |
| 97 | QQYNNWPWT | DLL3-mAb-1 CDR- |
| | | L3 (aa) (Kabat) |
| 98 | GDSISSDGY | DLL3-mAb-1 CDR- |
| | | H1 (aa) (Chothia) |
| 99 | YYSGN | DLL3-mAb-1 CDR- |
| | | H2 (aa) (Chothia) |
| 100 | DTASALAY | DLL3-mAb-1 CDR- |
| | | H3 (aa) (Chothia) |
| 101 | RASQSVSSNLA | DLL3-mAb-1 CDR- |
| | | L1 (aa) (Chothia) |
| 102 | GASTRAT | DLL3-mAb-1 CDR- |
| | | L2 (aa) (Chothia) |
| 103 | QQYNNWPWT | DLL3-mAb-1 CDR- |
| | | L3 (aa) (Chothia) |
| 104 | GDSISSDGY | DLL3-mAb-1 CDR- |
| | | H1 (aa) (IMGT) |
| 105 | IYYSGNT | DLL3-mAb-1 CDR- |
| | | H2 (aa) (IMGT) |
| 106 | AIDTASALAY | DLL3-mAb-1 CDR- |
| | | H3 (aa) (IMGT) |
| 107 | QSVSSN | DLL3-mAb-1 CDR- |
| | | L1 (aa) (IMGT) |
| 108 | GA | DLL3-mAb-1 CDR- |
| | | L2 (aa) (IMGT) |
| 109 | QQYNNWPWT | DLL3-mAb-1 CDR- |
| | | L3 (aa) (IMGT) |
| 110 | GDSISSDGYYWS | DLL3-mAb-1 CDR- |
| | | H1 (aa) (AbM) |
| 111 | YIYYSGNTY | DLL3-mAb-1 CDR- |
| | | H2 (aa) (AbM) |
| 112 | DTASALAY | DLL3-mAb-1 CDR- |
| | | H3 (aa) (AbM) |
| 113 | RASQSVSSNLA | DLL3-mAb-1 CDR- |
| | | L1 (aa) (AbM) |
| 114 | GASTRAT | DLL3-mAb-1 CDR- |
| | | L2 (aa) (AbM) |
| 115 | QQYNNWPWT | DLL3-mAb-1 CDR- |
| | | L3 (aa) (AbM) |
| 116 | CAGGTTCAGCTGCAAGAGTCTGGCCCTGGCCTGGTCAAGCCTAGCCAAACACTGAGCC | CAR A (nt) |
| | TGACCTGTACCGTGTCCGGCGATAGCATCAGCAGCGACGGCTACTACTGGTCCTGGAT | |
| | CAGACAGCACTGGCAAAGGCCTGGAATGGATCGGCTACATCTACTACAGCGGCAACAC | |
| | CTACTACAACCCCAGCCTGAAGTCCAGAGTGACCATCAGCGTGGACACCAGCAAGAAC | |
| | CAGTTCTCCCTGAAGCTGACCGCGTGACAGCCGCCGATACCGCCATCTACTATTGCGC | |
| | CATCGATACCGCTAGCGCCCTGGCTTATTGGGGCCAGGGAACACTGGTCACAGTGTCT | |
| | AGCGGAGGCGGAGGATCTGGTGGCGGAGGAAGTGGCGGAGGCGGTTCTGAGATTGTGA | |
| | TGACACAGAGCCCCGCCACACTGTCCGTTTCTCCCGGCGAAAGAGCCACACTGAGCTG | |
| | CAGAGCCAGCCAGAGCGTGTCCTCTAACCTGGCTTGGTATCAGCAGAAGCCCGGACAG | |
| | GCTCCCAGACTGCTGATCTATGGCGCCTCTACAAGAGCCACCGGCATTCCCGCCCGGT | |
| | TTTCTGGCTCTGGAAGCGGCACCGAGTTCACCCTGACAATCAGCAGCCTGCAGAGCGA | |
| | GGACTTCGCCGTGTACTACTGCCAGCAGTACAACAACTGGCCTTGGACCTTTGGCCAG | |
| | GGCACCAAaGTGGAAATCAAGCGGgagtctaaatacggaccgccttgtcctccttgtc | |
| | ccatgttctgggtgctcgtggtcgttggcggagtgctggcctgttacagcctgctggt | |
| | taccgtggccttcatcatcttttgggtcaagcggggcagaaagaagctgctctacatc | |
| | ttcaagcagcccttcatgcggcccgtgcagaccacacaagaggaagatggctgctcct | |
| | gcagattccccgaggaagaagaagcggctgcgagctgagagtgaagttcagcagatcc | |
| | gccgacgctccagcctatcagcagggccaaaaccagctgtacaacgagctgaacctgg | |
| | ggagaagagaagagtacgacgtgctggataagcggagaggcagagatcctgaaatggg | |
| | cggcaagcccagacggaagatcctcaagagggcctgtataatgagctgcagaaagaca | |
| | agatggccgaggcctacagcgagatcggaatgaagggcgagcgcagaagaggcaaggg | |
| | acacgatggactgtaccagggcctgagcaccgccaccaaggatacctatgacgcactg | |
| | cacatgcaggccctgccacctaga | |
| 117 | QVQLQESGPGLVKPSQTLSLTCTVSGDSISSDGYYWSWIRQHPGKGLEWIGYIYYSGN | CAR A (aa) |
| | TYYNPSLKSRVTISVDTSKNQFSLKLTSVTAADTAIYYCAIDTASALAYWGQGTLVTV | |
| | SSGGGGSGGGGSGGGGSEIVMTQSPATLSVSPGERATLSCRASQSVSSNLAWYQQKPG | |
| | QAPRLLIYGASTRATGIPARFSGSGSGTEFTLTISSLQSEDFAVYYCQQYNNWPWTFG | |
| | QGTKVEIKRESKYGPPCPPCPMFWVLVVVGGVLACYSLLVTVAFIIFWVKRGRKKLLY | |
| | IFKQPFMRPVQTTQEEDGCSCRFPEEEEGGCELRVKFSRSADAPAYQQGQNQLYNELN | |
| | LGRREEYDVLDKRRGRDPEMGGKPRRKNPQEGLYNELQKDKMAEAYSEIGMKGERRRG | |
| | KGHDGLYQGLSTATKDTYDALHMQALPPR | |
| 118 | ATGCCGCTGCTGCTACTGCTGCCCCTGCTGTGGGCAGGGGCTCTAGCTCAGGTTCAGC | CAR A including |
| | TGCAAGAGTCTGGCCCTGGCCTGGTCAAGCCTAGCCAAACACTGAGCCTGACCTGTAC | signal sequence (nt) |
| | CGTGTCCGGCGATAGCATCAGCAGCGACGGCTACTACTGGTCCTGGATCAGACAGCAC | |
| | CCTGGCAAAGGCCTGGAATGGATCGGCTACATCTACTACAGCGGCAACACCTACTACA | |
| | ACCCCAGCCTGAAGTCCAGAGTGACCATCAGCGTGGACACCAGCAAGAACCAGTTCTC | |
| | CCTGAAGCTGACCAGCGTGACAGCCGCCGATACCGCCATCTACTATTGCGCCATCGAT | |
| | ACCGCTAGCGCCCTGGCTTATTGGGGCCAGGGAACACTGGTCACAGTGTCTAGCGGAG | |
| | GCGGAGGATCTGGTGGCGGAGGAAGTGGCGGAGGCGGTTCTGAGATTGTGATGACACA | |
| | GAGCCCCGCCACACTGTCCGTTTCTCCCGGCGAAAGAGCCACACTGAGCTGCAGAGCC | |
| | AGCCAGAGCGTGTCCTCTAACCTGGCTTGGTATCAGCAGAAGCCCGGACAGGCTCCCA | |
| | GACTGCTGATCTATGGCGCCTCTACAAGAGCCACCGGCATTCCCGCCCGGTTTTCTGG | |
| | CTCTGGAAGCGGCACCGAGTTCACCCTGACAATCAGCAGCCTGCAGAGCGAGGACTTC | |
| | GCCGTGTACTACTGCCAGCAGTACAACAACTGGCCTTGGACCTTTGGCCAGGGCACCA | |
| | AAGTGGAAATCAAGCGGGAGTCTAAATACGGACCGCCTTGTCCTCCTTGTCCCATGTT | |
| | CTGGGTGCTCGTGGTCGTTGGCGGAGTGCTGGCCTGTTACAGCCTGCTGGTTACCGTG | |
| | GCCTTCATCATCTTTTGGGTCAAGCGGGGCAGAAAGAAGCTGCTCTACATCTTCAAGC | |
| | AGCCCTTCATGCGGCCCGTGCAGACCACACAAGAGGAAGATGGCTGCTCCTGCAGATT | |
| | CCCCGAGGAAGAAGAAGGCGGCTGCGAGCTGAGAGTGAAGTTCAGCAGATCCGCCGAC | |
| | GCTCCAGCCTATCAGCAGGGCCAAAACCAGCTGTACAACGAGCTGAACCTGGGGAGAA | |
| | GAGAAGAGTACGACGTGCTGGATAAGCGGAGAGGCAGAGATCCTGAAATGGGCGGCAA | |
| | GCCCAGACGGAAGAATCCTCAAGAGGGCCTGTATAATGAGCTGCAGAAAGACAAGATG | |
| | GCCGAGGCCTACAGCGAGATCGGAATGAAGGGCGAGCGCAGAAGAGGCAAGGGACACG | |
| | ATGGACTGTACCAGGGCCTGAGCACCGCCACCAAGGATACCTATGACGCACTGCACAT | |
| | GCAGGCCCTGCCACCTAGA | |
| 119 | MPLLLLLPLLWAGALAQVQLQESGPGLVKPSQTLSLTCTVSGDSISSDGYYWSWIRQH | CAR A including |
| | PGKGLEWIGYIYYSGNTYYNPSLKSRVTISVDTSKNQFSLKLTSVTAADTAIYYCAID | signal sequence (aa) |
| | TASALAYWGQGTLVTVSSGGGGSGGGGSGGGGSEIVMTQSPATLSVSPGERATLSCRA | |
| | SQSVSSNLAWYQQKPGQAPRLLIYGASTRATGIPARFSGSGSGTEFTLTISSLQSEDE | |
| | AVYYCQQYNNWPWTFGQGTKVEIKRESKYGPPCPPCPMFWVLVVVGGVLACYSLLVTV | |
| | AFIIFWVKRGRKKLLYIFKQPFMRPVQTTQEEDGCSCRFPEEEEGGCELRVKFSRSAD | |
| | APAYQQGQNQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| 120 | MVSPRMSGLLSQTVILALIFLPQTRPAGVFELQIHSFGPGPGPGAPRSPCSARLPCRL | Human DLL3 |
| | FFRVCLKPGLSEEAAESPCALGAALSARGPVYTEQPGAPAPDLPLPDGLLQVPERDAW | isoform 1 precursor |
| | PGTFSFIIETWREELGDQIGGPAWSLLARVAGRRRLAAGGPWARDIQRAGAWELRFSY | (aa) |
| | RARCEPPAVGTACTRLCRPRSAPSRCGPGLRPCAPLEDECEAPLVCRAGCSPEHGFCE | |
| | QPGECRCLEGWTGPLCTVPVSTSSCLSPRGPSSATTGCLVPGPGPCDGNPCANGGSCS | |
| | ETPRSFECTCPRGFYGLRCEVSGVTCADGPCFNGGLCVGGADPDSAYICHCPPGFQGS | |
| | NCEKRVDRCSLQPCRNGGLCLDLGHALRCRCRAGFAGPRCEHDLDDCAGRACANGGTC | |
| | VEGGGAHRCSCALGFGGRDCRERADPCAARPCAHGGRCYAHFSGLVCACAPGYMGARC | |
| | EFPVHPDGASALPAAPPGLRPGDPQRYLLPPALGLLVAAGVAGAALLLVHVRRRGHSQ | |
| | DAGSRLLAGTPEPSVHALPDALNNLRTQEGSGDGPSSSVDWNRPEDVDPQGIYVISAP | |
| | SIYAREVATPLFPPLHTGRAGQRQHLLFPYPSSILSVK | |
| 121 | MVSPRMSGLLSQTVILALIFLPQTRPAGVFELQIHSFGPGPGPGAPRSPCSARLPCRL | Human DLL3 |
| | FFRVCLKPGLSEEAAESPCALGAALSARGPVYTEQPGAPAPDLPLPDGLLQVPERDAW | isoform 2 precursor |
| | PGTFSFIIETWREELGDQIGGPAWSLLARVAGRRRLAAGGPWARDIQRAGAWELRFSY | (aa) |
| | RARCEPPAVGTACTRLCRPRSAPSRCGPGLRPCAPLEDECEAPLVCRAGCSPEHGFCE | |
| | QPGECRCLEGWTGPLCTVPVSTSSCLSPRGPSSATTGCLVPGPGPCDGNPCANGGSCS | |
| | ETPRSFECTCPRGFYGLRCEVSGVTCADGPCFNGGLCVGGADPDSAYICHCPPGFQGS | |
| | NCEKRVDRCSLQPCRNGGLCLDLGHALRCRCRAGFAGPRCEHDLDDCAGRACANGGTC | |
| | VEGGGAHRCSCALGFGGRDCRERADPCAARPCAHGGRCYAHFSGLVCACAPGYMGARC | |
| | EFPVHPDGASALPAAPPGLRPGDPQRYLLPPALGLLVAAGVAGAALLLVHVRRRGHSQ | |
| | DAGSRLLAGTPEPSVHALPDALNNLRTQEGSGDGPSSSVDWNRPEDVDPQGIYVISAP | |
| | SIYAREA | |
| 122 | AGVFELQIHSFGPGPGPGAPRSPCSARLPCRLFFRVCLKPGLSEEAAESPCALGAALS | Human DLL3 |
| | ARGPVYTEQPGAPAPDLPLPDGLLQVPFRDAWPGTFSFIIETWREELGDQIGGPAWSL | isoform 1 mature |
| | LARVAGRRRLAAGGPWARDIQRAGAWELRFSYRARCEPPAVGTACTRLCRPRSAPSRC | (aa) |
| | GPGLRPCAPLEDECEAPLVCRAGCSPEHGFCEQPGECRCLEGWTGPLCTVPVSTSSCL | |
| | SPRGPSSATTGCLVPGPGPCDGNPCANGGSCSETPRSFECTCPRGFYGLRCEVSGVTC | |
| | ADGPCFNGGLCVGGADPDSAYICHCPPGFQGSNCEKRVDRCSLQPCRNGGLCLDLGHA | |
| | LRCRCRAGFAGPRCEHDLDDCAGRACANGGTCVEGGGAHRCSCALGFGGRDCRERADP | |
| | CAARPCAHGGRCYAHFSGLVCACAPGYMGARCEFPVHPDGASALPAAPPGLRPGDPQR | |
| | YLLPPALGLLVAAGVAGAALLLVHVRRRGHSQDAGSRLLAGTPEPSVHALPDALNNLR | |
| | TQEGSGDGPSSSVDWNRPEDVDPQGIYVISAPSIYAREVATPLFPPLHTGRAGQRQHL | |
| | LFPYPSSILSVK | |
| 123 | AGVFELQIHSFGPGPGPGAPRSPCSARLPCRLFFRVCLKPGLSEEAAESPCALGAALS | Human DLL3 |
| | ARGPVYTEQPGAPAPDLPLPDGLLQVPFRDAWPGTFSFIIETWREELGDQIGGPAWSL | isoform 2 mature |
| | LARVAGRRRLAAGGPWARDIQRAGAWELRFSYRARCEPPAVGTACTRLCRPRSAPSRC | (aa) |
| | GPGLRPCAPLEDECEAPLVCRAGCSPEHGFCEQPGECRCLEGWTGPLCTVPVSTSSCL | |
| | SPRGPSSATTGCLVPGPGPCDGNPCANGGSCSETPRSFECTCPRGFYGLRCEVSGVTC | |
| | ADGPCFNGGLCVGGADPDSAYICHCPPGFQGSNCEKRVDRCSLQPCRNGGLCLDLGHA | |
| | LRCRCRAGFAGPRCEHDLDDCAGRACANGGTCVEGGGAHRCSCALGFGGRDCRERADP | |
| | CAARPCAHGGRCYAHFSGLVCACAPGYMGARCEFPVHPDGASALPAAPPGLRPGDPQR | |
| | YLLPPALGLLVAAGVAGAALLLVHVRRRGHSQDAGSRLLAGTPEPSVHALPDALNNLR | |
| | TQEGSGDGPSSSVDWNRPEDVDPQGIYVISAPSIYAREA | |
| 124 | MVSLQVSPLSQTLILAFLLPQALPAGVFELQIHSFGPGPGLGTPRSPCNARGPCRLFF | Mus musculus |
| | RVCLKPGVSQEATESLCALGAALSTSVPVYTEHPGESAAALPLPDGLVRVPERDAWPG | DLL3 (aa) |
| | TFSLVIETWREQLGEHAGGPAWNLLARVVGRRRLAAGGPWARDVQRTGTWELHFSYRA | |
| | RCEPPAVGAACARLCRSRSAPSRCGPGLRPCTPFPDECEAPSVCRPGCSPEHGYCEEP | |
| | DECRCLEGWTGPLCTVPVSTSSCLNSRVPGPASTGCLLPGPGPCDGNPCANGGSCSET | |
| | SGSFECACPRGFYGLRCEVSGVTCADGPCFNGGLCVGGEDPDSAYVCHCPPGFQGSNC | |
| | EKRVDRCSLQPCQNGGLCLDLGHALRCRCRAGFAGPRCEHDLDDCAGRACANGGTCVE | |
| | GGGSRRCSCALGFGGRDCRERADPCASRPCAHGGRCYAHFSGLVCACAPGYMGVRCEF | |
| | AVRPDGADAVPAAPRGLRQADPQRFLLPPALGLLVAAGLAGAALLVIHVRRRGPGQDT | |
| | GTRLLSGTREPSVHTLPDALNNLRLQDGAGDGPSSSADWNHPEDGDSRSIYVIPAPSI | |
| | YAREA | |
| 125 | MVSPRMSRLLSQTVILALIFIPQARPAGVFELQIHSFGPGPGPGAPRSPCSARGPCRL | Macaca fascicularis |
| | FFRVCLKPGLSEEAAESPCALGAALSARGPVYTEQPEAPAPDLPLPNGLLQVPERDAW | DLL3 (aa) |
| | PGTFSLIIETWREELGDQIGGPAWSLLARVTRRRRLAAGGPWARDIQRAGAWELRFSY | |
| | RARCELPAVGTACTRLCRPRSAPSRCGPGLRPCAPLEDECEAPPVCRAGCSLEHGFCE | |
| | QPGECRCLEGWTGPLCMVPVSTSSCLGLRGPSSTTTGCLVPGPGPCDGNPCANGGSCS | |
| | ETPGSFECTCPRGFYGLRCEVSGVTCADGPCFNGGLCVGGADPDSAYICHCPPGFQGS | |
| | NCEKRVDRCSLQPCRNGGLCLDLGHALRCRCRAGFAGPRCEHDLDDCAGRACANGGTC | |
| | VEGGGAHRCSCALGFGGRNCRERADPCAARPCAHGGRCYAHFSGLVCACAPGYMGARC | |
| | EFPVHPDGVSALPAAPPGLRPGDPQRYLLPPALGLLVAAGVAGAALLLVHVRRRGHAQ | |
| | DAGSRLLAGTPEPSVHALPDALNNLRTQEGPGDVPSSSVDWNRPEDVDSRGIYVISAP | |
| | SIYAREVAMPLFPPLHTGRAGQRQNLLFPFPSSILSVK | |
| 126 | ASTKGPSVFPLAPSSKSTSGGTAALGCLVKDYFPEPVTVSWNSGALTSGVHTFPAVLQ | IgG1 heavy chain |
| | SSGLYSLSSVVTVPSSSLGTQTYICNVNHKPSNTKVDKKVEPKSCDKTHTCPPCPAPE | constant |
| | LLGGPSVFLFPPKPKDTLMISRTPEVTCVVVDVSHEDPEVKENWYVDGVEVHNAKTKP | |
| | REEQYNSTYRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLDSDGSFFLY | |
| | SKLTVDKSRWQQGNVFSCSVMHEALHNHYTQKSLSLSPGK | |
| 127 | RTVAAPSVFIFPPSDEQLKSGTASVVCLLNNFYPREAKVQWKVDNALQSGNSQESVTE | kappa light chain |
| | QDSKDSTYSLSSTLTLSKADYEKHKVYACEVTHQGLSSPVTKSENRGEC | constant |
| 128 | QVQLQESGPGLVKPSQTLSLTCTVSGDSISSDGYYWSWIRQHPGKGLEWIGYIYYSGN | DLL3-mAb-1 IgG1 |
| | TYYNPSLKSRVTISVDTSKNQFSLKLTSVTAADTAIYYCAIDTASALAYWGQGTLVTV | full length heavy |
| | SSASTKGPSVFPLAPSSKSTSGGTAALGCLVKDYFPEPVTVSWNSGALTSGVHTEPAV | chain |
| | LQSSGLYSLSSVVTVPSSSLGTQTYICNVNHKPSNTKVDKKVEPKSCDKTHTCPPCPA | |
| | PELLGGPSVFLFPPKPKDTLMISRTPEVTCVVVDVSHEDPEVKFNWYVDGVEVHNAKT | |
| | KPREEQYNSTYRVVSVLTVLHQDWLNGKEYKCKVSNKALPAPIEKTISKAKGQPREPQ | |
| | VYTLPPSREEMTKNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| 129 | EIVMTQSPATLSVSPGERATLSCRASQSVSSNLAWYQQKPGQAPRLLIYGASTRATGI | DLL3-mAb-1 IgG1 |
| | PARFSGSGSGTEFTLTISSLQSEDFAVYYCQQYNNWPWTFGQGTKVEIKRTVAAPSVE | full length light |
| | IFPPSDEQLKSGTASVVCLLNNFYPREAKVQWKVDNALQSGNSQESVTEQDSKDSTYS | chain |
| | LSSTLTLSKADYEKHKVYACEVTHQGLSSPVTKSENRGEC | |

1. An anti-Delta-like ligand 3 (DLL3) antibody or antigen-binding fragment thereof comprising a heavy chain variable (V_{H}) region and a light chain variable (V_{L}) region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:90, and
the V_{L} region comprises a light chain complementarity determining region 1 (CDR-L1), a light chain complementarity determining region 2 (CDR-L2) and a light chain complementarity determining region 3 (CDR-L3) contained within SEQ ID NO:91.

2. An anti-DLL3 antibody or antigen-binding fragment thereof, comprising a heavy chain variable (V_{H}) region, and a light chain variable (V_{L}) region, wherein:
the V_{H} region comprises a heavy chain complementarity determining region 1 (CDR-H1) comprising the sequence set forth in SEQ ID NO:92, a heavy chain complementarity determining region 2 (CDR-H2) comprising the sequence set forth in SEQ ID NO:93, and a heavy chain complementarity determining region 3 (CDR-H3) comprising the sequence set forth in SEQ ID NO:94, and
the V_{L} region comprises a light chain complementarity determining region 1 (CDR-L1) comprising the sequence set forth in SEQ ID NO:95, a light chain complementarity determining region 2 (CDR-L2) comprising the sequence set forth in SEQ ID NO:96, and a light chain complementarity determining region 3 (CDR-L3) comprising the sequence set forth in SEQ ID NO:97.

3. The anti-DLL3 antibody or antigen-binding fragment thereof of claim 1 or 2, wherein:
the V_{H} region comprises an amino acid sequence having at least at or about 95%, 96%, 97%, 98% or 99% sequence identity to SEQ ID NO:90, and
the V_{L} region comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:91.

4. An anti-DLL3 antibody or antigen-binding fragment thereof comprising a heavy chain variable (V_{H}) region and a light chain variable (V_{L}) region, wherein:
the V_{H} region comprises an amino acid sequence having at least at or about 95%, 96%, 97%, 98% or 99% sequence identity to SEQ ID NO:90, and
the V_{L} region comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:91.

5. The anti-DLL3 antibody or antigen-binding fragment thereof of claim 4, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:90, and
the V_{L} region comprises a light chain complementarity determining region 1 (CDR-L1), a light chain complementarity determining region 2 (CDR-L2) and a light chain complementarity determining region 3 (CDR-L3) contained within SEQ ID NO:91.

6. The anti-DLL3 antibody or antigen-binding fragment thereof of claim 4 or 5, wherein:
the V_{H} region comprises a heavy chain complementarity determining region 1 (CDR-H1) comprising the sequence set forth in SEQ ID NO:92, a heavy chain complementarity determining region 2 (CDR-H2) comprising the sequence set forth in SEQ ID NO:93, and a heavy chain complementarity determining region 3 (CDR-H3) comprising the sequence set forth in SEQ ID NO:94, and
the V_{L} region comprises a light chain complementarity determining region 1 (CDR-L1) comprising the sequence set forth in SEQ ID NO:95, a light chain complementarity determining region 2 (CDR-L2) comprising the sequence set forth in SEQ ID NO:96, and a light chain complementarity determining region 3 (CDR-L3) comprising the sequence set forth in SEQ ID NO:97.

7. The anti-DLL3 antibody or antigen-binding fragment thereof of any of claims 1-6, wherein:
the V_{H} region comprises the sequence set forth in SEQ ID NO:90, and
the V_{L} region comprises the sequence set forth in SEQ ID NO:91.

8. An anti-DLL3 antibody or antigen-binding fragment thereof, comprising a heavy chain variable (V_{H}) region and a light chain variable (V_{L}) region, wherein:
the V_{H} region comprises the sequence set forth in SEQ ID NO:90, and
the V_{L} region comprises the sequence set forth in SEQ ID NO:91.

9. The anti-DLL3 antibody or antigen-binding fragment thereof of any of claims 1-8, wherein the anti-DLL3 antibody or antigen-binding fragment thereof is recombinant.

10. The anti-DLL3 antibody or antigen-binding fragment thereof of any of claims 1-9, wherein the V_{H} region and the V_{L} region is human or is from a human protein.

11. The anti-DLL3 antibody or antigen-binding fragment thereof of any of claims 1-10, wherein the antibody further comprises a heavy chain constant region (C_{H}) and/or a light chain constant region (C_{L}).

12. The anti-DLL3 antibody or antigen-binding fragment thereof of claim 11, wherein:
the C_{H} comprises the sequence set forth in SEQ ID NO:126, or an amino acid sequence having at least at or about 95%, 96%, 97%, 98% or 99% sequence identity to SEQ ID NO:126; and/or
the C_{L} comprises the sequence set forth in SEQ ID NO:127, or an amino acid sequence having at least at or about 95%, 96%, 97%, 98% or 99% sequence identity to SEQ ID NO:127.

13. The anti-DLL3 antibody or antigen-binding fragment thereof of claim 11 or 12, wherein the antibody or antigen-binding fragment thereof comprises:
a heavy chain comprising the sequence set forth in SEQ ID NO:128; and/or
a light chain comprising the sequence set forth in SEQ ID NO:129.

14. The anti-DLL3 antibody or antigen-binding fragment thereof of any of claims 1-13, wherein the antibody or antigen-binding fragment thereof comprises:
a heavy chain comprising the sequence set forth in SEQ ID NO:128; and
a light chain comprising the sequence set forth in SEQ ID NO:129.

15. The anti-DLL3 antibody or antigen-binding fragment thereof of any of claims 1-14, wherein the antibody is a full length antibody.

16. The anti-DLL3 antibody or antigen-binding fragment thereof of any of claims 1-14, wherein the antibody is an antigen-binding fragment.

17. The anti-DLL3 antibody or antigen-binding fragment thereof of any of claims 1-10 and 16, wherein the antigen-binding fragment thereof comprises a single chain Fv (scFv).

18. The anti-DLL3 antibody or antigen-binding fragment thereof of any of claims 1-10, 16, and 17, wherein the V_{H} region is amino-terminal to the V_{L} region.

19. The anti-DLL3 antibody or antigen-binding fragment thereof of any of claims 1-10, 16, and 17, wherein the V_{H} region is carboxy-terminal to the V_{L} region.

20. The anti-DLL3 antibody or antigen-binding fragment thereof of any of claims 1-10 and 17-19, wherein the V_{H} region and the V_{L} region are joined by a flexible linker.

21. The anti-DLL3 antibody or antigen-binding fragment thereof of claim 20, wherein the flexible linker comprises the sequence set forth in SEQ ID NO:46.

22. The anti-DLL3 antibody or antigen-binding fragment thereof of any of claims 17-21, wherein the scFv comprises the sequence set forth in SEQ ID NO:89.

23. The anti-DLL3 antibody or antigen-binding fragment thereof of any of claims 1-22, wherein the anti-DLL3 antibody or antigen-binding fragment thereof specifically binds to a human DLL3.

24. The anti-DLL3 antibody or antigen-binding fragment thereof of claim 23, wherein the human DLL3 comprises an amino acid sequence set forth in SEQ ID NO:120 or 121.

25. The anti-DLL3 antibody or antigen-binding fragment thereof of any of claims 1-24, wherein the anti-DLL3 antibody or antigen-binding fragment thereof does not bind to, is not cross-reactive to, or binds at a lower level or degree or affinity to a DLL1 or a DLL4 protein, optionally a human DLL1 or a human DLL4.

26. The anti-DLL3 antibody or antigen-binding fragment thereof of any of claims 1-25, wherein the extent, level or degree or affinity of binding of the anti-DLL3 antibody or antigen-binding fragment thereof to a human DLL1 or a human DLL4 is at least at or about 75%, 80%, 90%, 95% or 99% less than the extent, level or degree or affinity of binding to a human DLL3.

27. The anti-DLL3 antibody or antigen-binding fragment thereof of any of claims 1-26, wherein the antibody or antigen-binding fragment thereof binds to human DLL3 with an equilibrium dissociation constant (K_{D}) of from about 1×10⁻¹¹ M to about 1×10⁻⁷ M.

28. A single chain cell surface protein, comprising the anti-DLL3 antibody or antigen-binding fragment thereof of any of claims 1-27.

29. A conjugate, comprising the anti-DLL3 antibody or antigen-binding fragment thereof of any of claims 1-27 and a heterologous molecule or moiety.

30. The conjugate of claim 29, wherein the heterologous molecule or moiety is a therapeutic moiety.

31. The conjugate of claim 29 or 30, wherein the heterologous molecule or moiety is a small molecule chemical compound, protein, peptide, or toxin.

32. An anti-DLL3 chimeric antigen receptor (CAR) comprising an extracellular antigen-binding domain comprising the anti-DLL3 antibody or antigen-binding fragment thereof of any of claims 1-27, a transmembrane domain and an intracellular signaling region.

33. The anti-DLL3 CAR of claim 32, further comprising a spacer between the extracellular antigen-binding domain and the transmembrane domain.

34. The anti-DLL3 CAR of claim 33, wherein the spacer comprises at least a portion of an immunoglobulin or a variant thereof.

35. The anti-DLL3 CAR of claim 33 or 34, wherein the spacer comprises at least a portion of a hinge region of an immunoglobulin or a variant thereof.

36. The anti-DLL3 CAR of claim 35, wherein the at least a portion of a hinge region comprises all or a portion of an IgG4 hinge region, optionally a human IgG4 hinge region, or a variant thereof, optionally wherein the variant IgG4 hinge region comprises a substitution of amino acids CPSC to CPPC compared to the wild-type IgG4 hinge region.

37. The anti-DLL3 CAR of any of claims 33-36, wherein the spacer is less than at or about 15 amino acids in length.

38. The anti-DLL3 CAR of any of claims 33-37, wherein the spacer is between 12 and 15 amino acids in length.

39. The anti-DLL3 CAR of any of claims 33-38, wherein the spacer comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:1.

40. The anti-DLL3 CAR of any of claims 33-39, wherein the spacer comprises the sequence set forth in SEQ ID NO:1.

41. The anti-DLL3 CAR of any of claims 33-38, wherein the spacer comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:3.

42. The anti-DLL3 CAR of any of claims 33-38 and 41, wherein the spacer comprises the sequence set forth in SEQ ID NO:3.

43. The anti-DLL3 CAR of any of claims 33-36, wherein the spacer comprises at least a portion of a hinge region and at least a portion of a CH3 region of an immunoglobulin or a variant thereof.

44. The anti-DLL3 CAR of any of claims 33-36 and 43, wherein the spacer is at or about 111, 112, 113, 114, 115, 116, 117, 118, 119, 120, 121, 122, 123, 124 or 125 amino acids in length, or has a length between any of the foregoing, optionally wherein the spacer comprises the sequence set forth in SEQ ID NO:5 or 58 or an amino acid sequence having at least at or about 85%, 86%, 87%, 88%, 89%, 90%, 91%, 92%, 93%, 94%, 95%, 96%, 97%, 98% or 99% sequence identity to SEQ ID NO:5 or 58.

45. The anti-DLL3 CAR of any of claims 33-36, wherein the spacer comprises at least a portion of a hinge region, at least a portion of a CH2 and at least a portion of a CH3 region of an immunoglobulin or a variant thereof.

46. The anti-DLL3 CAR of any of claims 33-36 and 45, wherein the spacer is at or about 130, 140, 150, 160, 170, 180, 190, 200, 210, 220, 221, 222, 223, 224, 225, 226, 227, 228, 229 or 230 amino acids in length, or has a length between any of the foregoing, optionally wherein the spacer comprises the sequence set forth in SEQ ID NO:42 or 82 or an amino acid sequence having at least at or about 85%, 86%, 87%, 88%, 89%, 90%, 91%, 92%, 93%, 94%, 95%, 96%, 97%, 98% or 99% sequence identity to SEQ ID NO:42 or 82.

47. The anti-DLL3 CAR of any of claims 32-46, wherein the transmembrane domain comprises a transmembrane domain from CD4, CD28, or CD8.

48. The anti-DLL3 CAR of any of claims 32-47, wherein the transmembrane domain comprises a transmembrane domain from CD28, optionally a human CD28.

49. The anti-DLL3 CAR of any of claims 32-48, wherein the transmembrane domain comprises SEQ ID NO:10 or 12 or an amino acid sequence having at least at or about 85%, 86%, 87%, 88%, 89%, 90%, 91%, 92%, 93%, 94%, 95%, 96%, 97%, 98% or 99% sequence identity to SEQ ID NO:10 or 12.

50. The anti-DLL3 CAR of any of claims 33-49, wherein the transmembrane domain comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:10.

51. The anti-DLL3 CAR of any of claims 33-50, wherein the transmembrane domain comprises the sequence set forth in SEQ ID NO:10.

52. The anti-DLL3 CAR of any of claims 33-49, wherein the transmembrane domain comprises an amino acid sequence having at least at or about 85%, 86%, 87%, 88%, 89%, 90%, 91%, 92%, 93%, 94%, 95%, 96%, 97%, 98% or 99% sequence identity to SEQ ID NO:12.

53. The anti-DLL3 CAR of any of claims 33-49 and 52, wherein the transmembrane domain comprises the sequence set forth in SEQ ID NO:12.

54. The anti-DLL3 CAR of any of claims 32-53, wherein intracellular signaling region comprises an intracellular signaling domain capable of inducing a primary activation signal in a T cell, is a T cell receptor (TCR) component and/or comprises an immunoreceptor tyrosine-based activation motif (ITAM).

55. The anti-DLL3 CAR of any of claims 32-54, wherein the intracellular signaling domain comprises a cytoplasmic signaling domain of a CD3-zeta (CD3ζ) chain, optionally a human CD3ζ chain.

56. The anti-DLL3 CAR of any of claims 32-55, wherein the intracellular signaling domain comprises the sequence set forth in SEQ ID NO:19, or an amino acid sequence having at least at or about 90%, 91%, 92%, 93%, 94%, 95%, 96%, 97%, 98% or 99% sequence identity to SEQ ID NO:19.

57. The anti-DLL3 CAR of any of claims 32-56, wherein the intracellular signaling domain comprises the sequence set forth in SEQ ID NO:19.

58. The anti-DLL3 CAR of any of claims 32-57, wherein the intracellular signaling region further comprises a costimulatory domain.

59. The anti-DLL3 CAR of claim 58, wherein the costimulatory domain is between the transmembrane domain and the intracellular signaling domain.

60. The anti-DLL3 CAR of claim 58 or 59, wherein the costimulatory domain comprises an intracellular signaling domain of a T cell costimulatory molecule or a signaling portion thereof.

61. The anti-DLL3 CAR of any of claims 58-60, wherein the costimulatory domain comprises an intracellular signaling domain of CD28, 4-1BB, or ICOS.

62. The anti-DLL3 CAR of any of claims 58-61, wherein the costimulatory domain comprises an intracellular signaling domain of 4-1BB, optionally a human 4-1BB.

63. The anti-DLL3 CAR of any of claims 58-62, wherein the costimulatory domain comprises the sequence set forth in SEQ ID NO:17 or an amino acid sequence having at least at or about 90%, 91%, 92%, 93%, 94%, 95%, 96%, 97%, 98% or 99% sequence identity to SEQ ID NO:17.

64. The anti-DLL3 CAR of any of claims 58-61, wherein the costimulatory domain comprises an intracellular signaling domain of CD28, optionally a human CD28.

65. The anti-DLL3 CAR of any of claims 58-61 and 64, wherein the costimulatory domain comprises the sequence set forth in SEQ ID NO:15 or 16 or an amino acid sequence having at least at or about 90%, 91%, 92%, 93%, 94%, 95%, 96%, 97%, 98% or 99% sequence identity to SEQ ID NO:15 or 16.

66. The anti-DLL3 CAR of any of claims 32-65, wherein the CAR comprises from its N to C terminus in order: the extracellular antigen-binding domain, the spacer, the transmembrane domain and the intracellular signaling region.

67. The anti-DLL3 CAR of any of claims 33-38, 54-63, and 66, wherein the CAR comprises, from its N to C terminus in order:
an extracellular antigen-binding domain comprising the scFv;
a spacer comprising a modified IgG4 hinge, optionally comprising the sequence set forth in SEQ ID NO:1;
a transmembrane domain, optionally a transmembrane domain from a human CD28, optionally comprising the sequence set forth in SEQ ID NO:10; and
an intracellular signaling region comprising a cytoplasmic signaling domain of a CD3-zeta (CD3ζ) chain and an intracellular signaling domain of a costimulatory molecule, optionally comprising an intracellular signaling domain of 4-1BB.

68. The anti-DLL3 CAR of any of claims 33-38, 54-63, and 66, wherein the CAR comprises, from its N to C terminus in order:
an extracellular antigen-binding domain comprising the scFv;
a spacer comprising a modified IgG4 hinge, optionally comprising the sequence set forth in SEQ ID NO:3;
a transmembrane domain, optionally a transmembrane domain from a human CD28, optionally comprising the sequence set forth in SEQ ID NO:12; and
an intracellular signaling region comprising a cytoplasmic signaling domain of a CD3-zeta (CD3ζ) chain and an intracellular signaling domain of a costimulatory molecule, optionally comprising an intracellular signaling domain of 4-1BB.

69. An anti-DLL3 chimeric antigen receptor (CAR) comprising:
an extracellular antigen-binding domain comprising an scFv comprising
a heavy chain variable (V_{H}) region and a light chain variable (V_{L}) region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:90, and
the V_{L} region comprises a light chain complementarity determining region 1 (CDR-L1), a light chain complementarity determining region 2 (CDR-L2) and a light chain complementarity determining region 3 (CDR-L3) contained within SEQ ID NO:91,
a spacer comprising the sequence set forth in SEQ ID NO:1;
a transmembrane domain comprising the sequence set forth in SEQ ID NO:10; and
an intracellular signaling region comprising a cytoplasmic signaling domain of a CD3-zeta (CD3ζ) chain comprising the sequence set forth in SEQ ID NO:19, and an intracellular signaling domain of a costimulatory molecule comprising the sequence set forth in SEQ ID NO:17.

70. An anti-DLL3 chimeric antigen receptor (CAR) comprising:
an extracellular antigen-binding domain comprising an scFv comprising
a heavy chain variable (V_{H}) region and a light chain variable (V_{L}) region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:90, and
the V_{L} region comprises a light chain complementarity determining region 1 (CDR-L1), a light chain complementarity determining region 2 (CDR-L2) and a light chain complementarity determining region 3 (CDR-L3) contained within SEQ ID NO:91,
a spacer comprising the sequence set forth in SEQ ID NO:3;
a transmembrane domain comprising the sequence set forth in SEQ ID NO:12; and
an intracellular signaling region comprising a cytoplasmic signaling domain of a CD3-zeta (CD3ζ) chain comprising the sequence set forth in SEQ ID NO:19, and an intracellular signaling domain of a costimulatory molecule comprising the sequence set forth in SEQ ID NO:17.

71. The anti-DLL3 CAR of any of claims 33-38, 54-63, 66, 67, and 69, wherein the CAR comprises, from its N to C terminus in order:
an extracellular antigen-binding domain comprising the scFv comprising the sequence set forth in SEQ ID NO:89;
a spacer comprising the sequence set forth in SEQ ID NO:1;
a transmembrane domain comprising the sequence set forth in SEQ ID NO:10; and
an intracellular signaling region comprising a cytoplasmic signaling domain of a CD3-zeta (CD3ζ) chain comprising the sequence set forth in SEQ ID NO:19, and an intracellular signaling domain of a costimulatory molecule comprising the sequence set forth in SEQ ID NO:17.

72. The anti-DLL3 CAR of any of claims 33-38, 54-63, 66, 68 and 70, wherein the CAR comprises, from its N to C terminus in order:
an extracellular antigen-binding domain comprising the scFv comprising the sequence set forth in SEQ ID NO:89;
a spacer comprising the sequence set forth in SEQ ID NO:3;
a transmembrane domain comprising the sequence set forth in SEQ ID NO:12; and
an intracellular signaling region comprising a cytoplasmic signaling domain of a CD3-zeta (CD3ζ) chain comprising the sequence set forth in SEQ ID NO:19, and an intracellular signaling domain of a costimulatory molecule comprising the sequence set forth in SEQ ID NO:17.

73. The anti-DLL3 CAR of any of claims 52-72, wherein the anti-DLL3 CAR comprises the sequence set forth in SEQ ID NO:117 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO:117.

74. The anti-DLL3 CAR of any of claims 52-73, wherein the anti-DLL3 CAR comprises the sequence set forth in SEQ ID NO:117.

75. A polynucleotide comprising a nucleic acid encoding the anti-DLL3 antibody or antigen-binding domain thereof of any of claims 1-27.

76. A polynucleotide comprising a nucleic acid encoding the single chain cell surface protein of claim 28.

77. A polynucleotide comprising a nucleic acid encoding the conjugate of any of claims 29-31.

78. A polynucleotide comprising a nucleic acid encoding the anti-DLL3 chimeric antigen receptor of any of claims 32-74.

79. The polynucleotide of any of claims 75-78, wherein the polynucleotide comprises:
a nucleic acid encoding the V_{H}, comprising the sequence set forth in SEQ ID NO:87, or a nucleic acid sequence having at least at or about 90%, 91%, 92%, 93%, 94%, 95%, 96%, 97%, 98% or 99% sequence identity to SEQ ID NO:87, and
a nucleic acid encoding the V_{L}, comprising the sequence set forth in SEQ ID NO:88, or a nucleic acid sequence having at least at or about 90%, 91%, 92%, 93%, 94%, 95%, 96%, 97%, 98% or 99% sequence identity to SEQ ID NO:88.

80. The polynucleotide of any of claims 75-79, wherein the polynucleotide comprises:
a nucleic acid encoding the V_{H}, comprising the sequence set forth in SEQ ID NO:87, and
a nucleic acid encoding the V_{L}, comprising the sequence set forth in SEQ ID NO:88.

81. The polynucleotide of any of claims 75-80, wherein the antigen-binding fragment is a single chain Fv (scFv), and the polynucleotide comprises a nucleic acid encoding the scFv, comprising the sequence set forth in SEQ ID NO:86 or a nucleic acid sequence having at least at or about 90%, 91%, 92%, 93%, 94%, 95%, 96%, 97%, 98% or 99% sequence identity to SEQ ID NO:86.

82. The polynucleotide of any of claims 75-81, wherein the antigen-binding fragment is a single chain Fv (scFv), and the polynucleotide comprises a nucleic acid encoding the scFv, comprising the sequence set forth in SEQ ID NO:86.

83. The polynucleotide of any of claims 78-82, wherein the polynucleotide comprises a nucleic acid encoding the spacer comprising the sequence set forth in SEQ ID NO:2 or a nucleic acid sequence having at least at or about 90%, 91%, 92%, 93%, 94%, 95%, 96%, 97%, 98% or 99% sequence identity to SEQ ID NO:2.

84. The polynucleotide of any of claims 78-83, wherein the polynucleotide comprises a nucleic acid encoding the spacer comprising the sequence set forth in SEQ ID NO:2.

85. The polynucleotide of any of claims 78-82, wherein the polynucleotide comprises a nucleic acid encoding the spacer comprising the sequence set forth in SEQ ID NO:4 or a nucleic acid sequence having at least at or about 90%, 91%, 92%, 93%, 94%, 95%, 96%, 97%, 98% or 99% sequence identity to SEQ ID NO:4.

86. The polynucleotide of any of claims 78-82 and 85, wherein the polynucleotide comprises a nucleic acid encoding the spacer comprising the sequence set forth in SEQ ID NO:4.

87. The polynucleotide of any of claims 78-84, wherein the polynucleotide comprises a nucleic acid encoding the transmembrane domain comprising the sequence set forth in SEQ ID NO:11 or a nucleic acid sequence having at least at or about 90%, 91%, 92%, 93%, 94%, 95%, 96%, 97%, 98% or 99% sequence identity to SEQ ID NO:11.

88. The polynucleotide of any of claims 78-84 and 87, wherein the polynucleotide comprises a nucleic acid encoding the transmembrane domain, comprising the sequence set forth in SEQ ID NO:11.

89. The polynucleotide of any of claims 78-82, 85 and 86, wherein the polynucleotide comprises a nucleic acid encoding the transmembrane domain comprising the sequence set forth in SEQ ID NO:13 or a nucleic acid sequence having at least at or about 90%, 91%, 92%, 93%, 94%, 95%, 96%, 97%, 98% or 99% sequence identity to SEQ ID NO:13.

90. The polynucleotide of any of claims 78-82, 85, 86, and 89, wherein the polynucleotide comprises a nucleic acid encoding the transmembrane domain comprising the sequence set forth in SEQ ID NO:13.

91. The polynucleotide of any of claims 78-90, wherein the nucleic acid encoding the anti-DLL3 chimeric antigen receptor comprises the sequence set forth in SEQ ID NO:116 or a sequence that exhibits at least 90%, 91%, 92%, 93%, 94%, 95%, 96%, 97%, 98%, or 99% sequence identity to the sequence set forth in SEQ ID NO:116.

92. The polynucleotide of any of claims 78-91, wherein the a nucleic acid encoding the anti-DLL3 chimeric antigen receptor comprises the sequence set forth in SEQ ID NO:116.

93. The polynucleotide of any of claims 75-92, wherein the polynucleotide is optimized by splice site elimination.

94. The polynucleotide of any of claims 75-93, wherein the polynucleotide is codon-optimized for expression in a human cell.

95. A vector, comprising the polynucleotide of any of claims 75-94.

96. The vector of claim 95, wherein the vector is a viral vector.

97. The vector of claim 96, wherein the viral vector is a retroviral vector or a lentiviral vector.

98. The vector of claim 96, wherein the viral vector is an adeno-associated virus (AAV) vector.

99. A cell comprising the anti-DLL3 antibody or antigen-binding fragment thereof of any of claims 1-27, the single chain cell surface protein of claim 28 or the conjugate of any of claims 29-31.

100. A cell comprising the anti-DLL3 chimeric antigen receptor of any of claims 32-74.

101. A cell comprising the polynucleotide of any of claims 75-94, or the vector of any of claims 95-98.

102. The cell of any of claims 99-101, wherein the cell is a lymphocyte.

103. The cell of any of claims 99-102, wherein the cell is an NK cell or a T cell.

104. The cell of any of claims 99-103, wherein the cell is a T cell and the T cell is a CD4+ T cell or a CD8+ T cell.

105. The cell of any of claims 99-104, wherein the cell is a primary cell obtained from a subject.

106. The cell of any of claims 100-105, wherein, among a plurality of the cells, less than at or about 10%, at or about 9%, at or about 8%, at or about 7%, at or about 5%, at or about 4%, at or about 3%, at or about 2% or at or about 1% of the cells in the plurality comprise an anti-DLL3 chimeric antigen receptor that exhibits tonic signaling and/or antigen independent activity or signaling.

107. A composition comprising the cell of any of claims 99-106.

108. A composition comprising the anti-DLL3 antibody or antigen-binding fragment thereof of any of claims 1-27, the single chain cell surface protein of claim 28, the conjugate of any of claims 29-31 or the anti-DLL3 chimeric antigen receptor of any of claims 32-74.

109. The composition of claim 107 or 108, further comprising a pharmaceutically acceptable excipient.

110. The composition of any of claims 107-109, wherein the composition comprises CD4+ and CD8+ T cells and the ratio of CD4+ to CD8+ T cells is from at or about 1:3 to 3:1, optionally at or about 1:2 to 2:1, optionally at or about 1:1.

111. The composition of any of claims 107-110, wherein, among a plurality of the cells in the composition, less than at or about 10%, at or about 9%, at or about 8%, at or about 7%, at or about 5%, at or about 4%, at or about 3%, at or about 2% or at or about 1% of the cells in the plurality comprise an anti-DLL3 chimeric antigen receptor that exhibits tonic signaling and/or antigen independent activity or signaling.

112. A method of treatment, comprising administering the anti-DLL3 antibody or antigen-binding fragment thereof of any of claims 1-27, the single chain cell surface protein of claim 28, the conjugate of any of claims 29-31, the anti-DLL3 chimeric antigen receptor of any of claims 32-74, the polynucleotide of any of claims 75-94, the vector of any of claims 95-98, the cell of any of claims 99-106, or the composition of any of claims 107-111 to a subject having a disease or disorder associated with DLL3.

113. The method of claim 112, wherein the disease or disorder associated with DLL3 is a cancer.

114. The method of claim 113, wherein the cancer is a DLL3-expressing cancer.

115. The method of any of claims 112-114, wherein the disease or disorder associated with DLL3 is selected from among a neuroendocrine tumor, a small cell lung cancer (SCLC), a large cell neuroendocrine carcinoma (LCNEC), a melanoma, a glioma, or a glioblastoma.

116. The method of any of claims 112-115, wherein the disease or disorder associated with DLL3 is a neuroendocrine tumor.

117. The method of any of claims 112-116, wherein the disease or disorder associated with DLL3 is a small cell lung cancer (SCLC).

118. The method of any of claims 112-117, wherein the disease or disorder associated with DLL3 is a large cell neuroendocrine carcinoma (LCNEC).

119. The anti-DLL3 antibody or antigen-binding fragment thereof of any of claims 1-27, the single chain cell surface protein of claim 28, the conjugate of any of claims 29-31, the anti-DLL3 chimeric antigen receptor of any of claims 32-74, the polynucleotide of any of claims 75-94, the vector of any of claims 95-98, the cell of any of claims 99-106, or the composition of any of claims 107-111 for use in treating a disease or disorder associated with DLL3.

120. Use of the anti-DLL3 antibody or antigen-binding fragment thereof of any of claims 1-27, the single chain cell surface protein of claim 28, the conjugate of any of claims 29-31, the anti-DLL3 chimeric antigen receptor of any of claims 32-74, the polynucleotide of any of claims 75-94, the vector of any of claims 95-98, the cell of any of claims 99-106, or the composition of any of claims 107-111 for the manufacture of a medicament for treating a disease or disorder associated with DLL3.

121. Use of the anti-DLL3 antibody or antigen-binding fragment thereof of any of claims 1-27, the single chain cell surface protein of claim 28, the conjugate of any of claims 29-31, the anti-DLL3 chimeric antigen receptor of any of claims 32-74, the polynucleotide of any of claims 75-94, the vector of any of claims 95-98, the cell of any of claims 99-106, or the composition of any of claims 107-111 for the treatment of a disease or disorder associated with DLL3.

122. The antibody or antigen-binding fragment thereof, single chain cell surface protein, conjugate, chimeric antigen receptor, polynucleotide, vector, cell or composition for use or the use of any of claims 119-121 wherein the disease or disorder associated with DLL3 is a cancer.

123. The antibody or antigen-binding fragment thereof, single chain cell surface protein, conjugate, chimeric antigen receptor, polynucleotide, vector, cell or composition for use or the use of claim 122, wherein the cancer is a DLL3-expressing cancer.

124. The antibody or antigen-binding fragment thereof, single chain cell surface protein, conjugate, chimeric antigen receptor, polynucleotide, vector, cell or composition for use or the use of any of claims 119-123, wherein the disease or disorder associated with DLL3 is a neuroendocrine tumor, a small cell lung cancer (SCLC), a large cell neuroendocrine carcinoma (LCNEC), a melanoma, a glioma, or a glioblastoma.

125. The cell, composition, antibody or antigen-binding fragment thereof, single chain cell surface protein, conjugate, chimeric antigen receptor, polynucleotide or vector for use or the use of any of claims 119-124, wherein the disease or disorder associated with DLL3 is a neuroendocrine tumor.

126. The cell, composition, antibody or antigen-binding fragment thereof, single chain cell surface protein, conjugate, chimeric antigen receptor, polynucleotide or vector for use or the use of any of claims 119-124, wherein the disease or disorder associated with DLL3 is a small cell lung cancer (SCLC).

127. The cell, composition, antibody or antigen-binding fragment thereof, single chain cell surface protein, conjugate, chimeric antigen receptor, polynucleotide or vector for use or the use of any of claims 119-124, wherein the disease or disorder associated with DLL3 is a large cell neuroendocrine carcinoma (LCNEC).

128. A kit comprising the anti-DLL3 antibody or antigen-binding fragment thereof of any of claims 1-27, the single chain cell surface protein of claim 28, the conjugate of any of claims 29-31, the anti-DLL3 chimeric antigen receptor of any of claims 32-74, the polynucleotide of any of claims 75-94, the vector of any of claims 95-98, the cell of any of claims 99-106 or the composition of any of claims 107-111, and instructions for use.

129. The kit of claim 128, wherein the instructions are for administering the anti-DLL3 antibody or antigen-binding fragment thereof, the single chain cell surface protein, the conjugate, the anti-DLL3 chimeric antigen receptor, the cell or the composition to a subject having a disease or disorder associated with DLL3.

130. An article of manufacture comprising the anti-DLL3 antibody or antigen-binding fragment thereof of any of claims 1-27, the single chain cell surface protein of claim 28, the conjugate of any of claims 29-31, the anti-DLL3 chimeric antigen receptor of any of claims 32-74, the polynucleotide of any of claims 75-94, the vector of any of claims 95-98, the cell of any of claims 99-106, the composition of any of claims 107-111, or the kit of claim 128 or 129.
